(19) United States
(12) Patent Application Publication
(10) Pub. No.: US 2018/0312534 A1
(43) Pub. Date: Nov. 1, 2018

(54) FLUORESCENT ANTICANCER PLATINUM DRUGS

(71) Applicant: INVICTUS ONCOLOGY PVT. LTD., Delhi (IN)

(72) Inventors: Arindam SARKAR, Delhi (IN); Swadhin Kumar MANDAL, Mohanpur, Kolkata (IN); Aniruddha SENGUPTA, Delhi (IN); Goutam BISWAS, Delhi (IN); Pradip DUTTA, Delhi (IN); Rupali SHARMA, Delhi (IN); Justin Paul RAJ, Delhi (IN); Hemant SURYAVANSHI, Delhi (IN); Smita KUMARI, Delhi (IN)

(73) Assignee: INVICTUS ONCOLOGY PVT. LTD., Delhi (IN)

(21) Appl. No.: 15/768,396

(22) PCT Filed: Oct. 14, 2016
(86) PCT No.: PCT/IB2016/056160

§ 371 Date: Apr. 13, 2018

(30) Foreign Application Priority Data
Oct. 16, 2015 (IN) 3343/DEL/2015
Jul. 5, 2016 (IN) 201611023131

(51) Int. Cl. C07F 15/00; A61K 49/00; A61P 35/00

(52) U.S. Cl. CPC C07F 15/0093; A61P 35/00; A61K 49/0052

(57) ABSTRACT

The present disclosure is in relation to the field of nanotechnology and cancer therapeutics. In particular, the present disclosure relates to fluorescent platinum based compounds. The disclosure further relates to synthesis of said fluorescent platinum based compounds, nanoparticles and compositions comprising said fluorescent platinum based compounds/nanoparticles. The disclosure also relates to methods of managing cancer by the fluorescence changes between aforesaid platinum based compounds and corresponding free ligands, nanoparticles and compositions.

### CROSS REFERENCE TO RELATED APPLICATIONS

This application claims benefit under one or more of 35 U.S.C. § 119(a)-119(d) of Indian Patent Application No. 3343/DEL/2015, filed Oct. 16, 2015 and Indian Patent Application No. 201611023131, filed Jul. 5, 2016, the content of both applications is incorporated herein by reference in its entirety.

### TECHNICAL FIELD

The present disclosure is in relation to the field of nanotechnology and cancer therapeutics. In particular, the present disclosure relates to fluorescent platinum based compounds. The disclosure further relates to synthesis of said fluorescent platinum based compounds, nanoparticles and compositions comprising said fluorescent platinum based compounds/nanoparticles. The disclosure also relates to methods of managing cancer by employing the fluorescence changes between aforesaid platinum based compounds and corresponding free ligands, nanoparticles and compositions.

### BACKGROUND

The area of anticancer drug development using fluorescent ligands is a relatively new area and only a few literature reports exist for fluorescent platinum drugs. To-date there has been many reports on phenalenyl based organic materials (A Sarkar et al, Chemistry of Materials, (2009), 21, 2226-2237; A. Sarkar et al Chemistry A European Journal, (2011) 17, 11576-11584; S. K. Mandal et al J. Am. Chem. Soc. 2005, 127, 8185-8196; K. D. Franz, J. Org. Chem., Vol. 44, No. 10, 1979; K. D. Franz et al Tetrahedron 1978, 34, 2147). On the other hand, fluorescent properties of a phenalenyl compound have been recently studied (Mitra et al., Sensors and Actuators B, Chemical, 2015, 210, 712). Several metal complexes, based on phenalenyl ligands, have been reported (Pariyar et al., J. Am. Chem. Soc., 2015, 137, 5955; Raman et al., Nature, 2013, 493, 509 and Mukherjee et al., Chemistry-A European Journal, 2012, 18, 10530-545), but platinum complexes based on phenalenyl ligands are rare (Mochida et al., Eur. J. Inorg. Chem. 2006, 558-565).

The present disclosure aims at overcoming the drawbacks of the prior art and providing for stable, potent and safer phenalenyl-based platinum compounds as anticancer agents.

### SUMMARY

The present invention primarily deals with the synthesis of a variety of phenalenyl-based ligands and their Pt complexes. This molecular design focuses on the substituents that can impart fluorescence to the molecule by intramolecular charge transfer (ICT) mechanism. These complexes have been characterized by standard techniques such as NMR spectroscopy, single crystal X-ray studies and elemental analysis. Lipid functionalization and supramolecular formulation of these compounds have been carried out to make these drugs less toxic and more efficacious. The IC₅₀ values of some of these compounds have been assessed towards a few cancer cell lines.

In one aspect, the disclosure provides a compound of Formula IV:

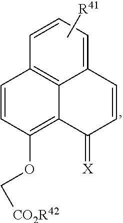

wherein:
X is O or NR⁴³;
R⁴¹ is H, hydroxyl, alkoxy or -linker-lipid or polyethylene glycol;
R⁴² is H, alkyl, cyclyl, heterocyclyl, aryl or heteroaryl; and
R⁴³ is H, alkyl, cyclyl, heterocyclyl, aryl or heteroaryl.

In some embodiments of the various aspects disclosed herein, a compound of Formula IV is of Formula IV′:

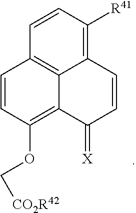

In the various aspects described herein, R⁴¹ can be hydroxyl, alkoxy or -linker-lipid.

In some embodiments, R⁴¹ can be a C₁-C₆alkoxy. In some embodiments, R⁴¹ is methoxy or ethoxy.

In some compounds of Formula IV′, R⁴¹ is hydrogen.

In the various aspects described herein, R⁴² can be hydrogen, methyl, ethyl, propyl, butyl, pentyl or hexanyl.

In some embodiments, R⁴¹ is hydroxyl, alkoxy or -linker-lipid; and R⁴² is C₁-C₆ alkyl.

In another aspect, the disclosure provides a compound of Formula V:

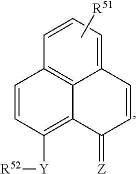

wherein:
Y is O, S or NR⁵³;
Z is O or NR⁵³;
R⁵¹ is H, alkoxy, optionally substituted alkylamino, optionally substituted alkylthio, -linker-carbohydrate, -linker-(anti-cancer agent), or -linker-lipid; or polyethylene glycol
R⁵² is H, alkyl, cyclyl, heterocyclyl, aryl, heteroaryl, optionally substituted PEG, -linker-carbohydrate, -linker-(anti-cancer agent), -linker-NH(CH₂CO₂H)₂, -linker-CO₂H,

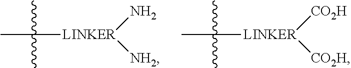

or -linker-lipid; and
each R⁵³ is same or different and selected independently from the group consisting of alkyl, cyclyl, heterocyclyl, aryl, heteroaryl, or -linker-lipid,
provided that at least one of R⁵¹ and R⁵² is -linker-lipid, -linker-(anti-cancer agent) or -linker-carbohydrate, or R⁵² is optionally substituted PEG, -linker-NH(CH₂CO₂H)₂, -linker-CO₂H,

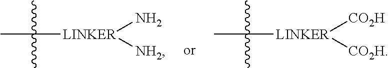

In some embodiments, a compound of Formula V is of Formula V′:

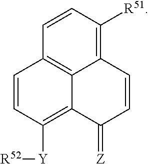

In various compounds of Formula V′, R⁵¹ is hydrogen or -lipid-linker.

In some compounds of Formula V, R⁵¹ is -linker-carbohydrate. In some embodiments, R⁵¹ is optionally substituted alkylamino, optionally substituted alkylthio, -linker-carbohydrate.

In various compounds of Formula V, R⁵² is optionally substituted PEG, -linker-carbohydrate, -linker-(anti-cancer agent), -linker-NH(CH₂CO₂H)₂, -linker-CO₂H,

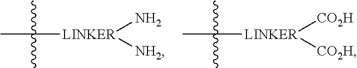

or -linker-lipid.

In some embodiments, a compound of Formula V′ is of Formula V″:

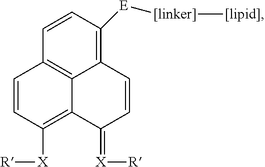

wherein:
E is O, NH or S;
each X is same or different and selected independently from the group consisting of O, N, S, NH and NR
each R′ is same or different and selected independently from the group consisting of H, alkyl, cyclyl, heterocyclyl, aryl and heteroaryl; and
R is alkyl, cyclyl, heterocyclyl, aryl or heteroaryl.

In some embodiments, a compound of Formula V′ is a compound of Formula V″-B:

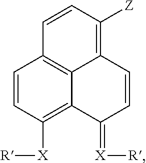

wherein:
Z is -E-linker-carbohydrate or -E-linker-(anti-cancer agent);
E is O, NH or S;
each X is same or different and selected independently from the group consisting of O, N, S, NH and NR
each R′ is same or different and selected independently from the group consisting of H, alkyl, cyclyl, heterocyclyl, aryl and heteroaryl, each of which can be optionally substituted; and
R is alkyl, cyclyl, heterocyclyl, aryl or heteroaryl, each of which can be optionally substituted.

In some other embodiments, a compound of Formula V′ is of Formula V′″:

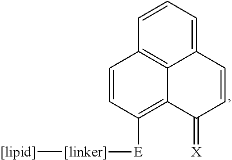

wherein:
E is NH or S;
X is O or NR; and
R is H, alkyl, cyclyl, heterocyclyl, aryl or heteroaryl.

In some embodiments, a compound of Formula V′ is a compound of Formula V′″-B:

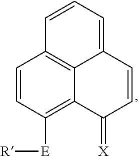

wherein:
E is NH or S;
R′ is optionally substituted PEG, -linker-carbohydrate, -linker-(anti-cancer agent), -linker-NH(CH₂CO₂H)₂, -linker-CO₂H,

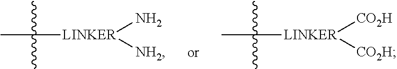

X is O or NR; and
R is H, alkyl, cyclyl, heterocyclyl, aryl or heteroaryl, each of which can be optionally substituted.

In another aspect, the disclosure provide a compound of Formula I″:

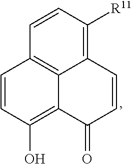

wherein R¹¹ is -linker-carbohydrate or -linker-lipid.

In one aspect, the disclosure provides a compound of Formula II″:

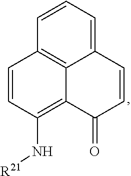

wherein R²¹ is optionally substituted PEG, -linker-carbohydrate, -linker-(anti-cancer agent), -linker-NH(CH₂CO₂H)₂, -linker-CO₂H,

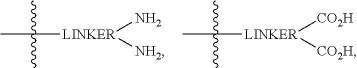

or -linker-lipid.

In another aspect, the disclosure provides a compound of Formula III″:

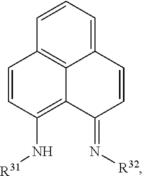

wherein R³¹ and R³² are same or different and selected independently from the group consisting of hydrogen, alkyl, cyclyl, heterocyclyl, aryl, or -linker-lipid, provided that at least one of R³¹ and R³² is a -linker-lipid.

In another aspect, the present disclosure provides a complex comprising: (i) at least one fluorescent molecule; and (ii) a platinum moiety comprising a platinum atom, wherein the platinum atom is conjugated with said at least one fluorescent molecule.

In some embodiments, the fluorescent molecule is a phenalenyl, for example, a phenalenyl substituted with heteroatoms at the 1 and 9 positions. The heteroatoms at the 1 and 9 positions can be same or different and can be selected from the group consisting of O, N and S.

In some embodiments, the fluorescent molecule is a compound of formula I, II, III, IV or V.

In some embodiments, the phenalenyl is conjugated with a lipid. In some embodiments, the phenalenyl is conjugated with a lipid at position 4 or 9 of the phenalenyl.

In some embodiments, the phenalenyl is conjugated with a carbohydrate. In some embodiments, the phenalenyl is conjugated with a carbohydrate at position 4 or 9 of the phenalenyl.

In yet another aspect, the present disclosure provide a compound of Formula XII:

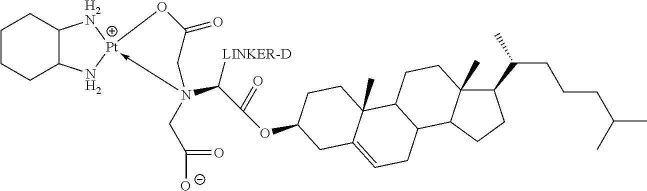

wherein D is a fluorescent molecule.

In some embodiments, the fluorescent molecule is selected from the group consisting of 7-amino-4-methyl coumarin, rhodamine, fluorescein, dansyl, fluorene-1-carboxylic acid, and bimane.

The disclosure also provides particles, such as nanoparticles comprising one or more of the compounds or platinum containing complexes disclosed herein.

The disclosure also provide a method of managing or treating cancer, said method comprising step of administering the platinum containing compounds or the nanoparticles as disclosed to a subject in need thereof.

The compounds and complexes described herein can be used for imaging, such as a tumor in a subject. Thus, the disclosure also provides method for imaging a tumor. Generally, the method comprises a step of administering a compound, platinum containing complex or a nanoparticle disclosed herein to a subject.

### BRIEF DESCRIPTION OF THE DRAWINGS

In order that the invention may be readily understood and put into practical effect, reference will now be made to exemplary embodiments as illustrated with reference to the accompanying figures. The figures together with a detailed description below, are incorporated in and form part of the specification, and serve to further illustrate the embodiments and explain various principles and advantages, in accordance with the present disclosure.

FIG. 1 is a schematic representation of improvements in the current platinum based anticancer drugs.

FIG. 2 is a schematic representation of phenalenyl (PLY) based molecular design for a better anticancer drug according an embodiment of the invention.

FIGS. 3-6 show exemplary platinum compounds of the invention.

FIG. 7 shows single crystal X-ray diffraction structure of compound 2a.

FIGS. 8A-8C show efficacy of Compounds 2a (IO-199_01) and 2b (IO-199_02) versus oxaliplatin in colorectal (HCT-116, FIG. 8A), breast (MDA-MB-213, FIG. 8B) and ovarian (SKOV-3, FIG. 8C) cancer cell lines.

FIG. 9 shows an exemplary compound's (IO-199_16) efficacy in lung cancer (A549) cells.

FIGS. 10A-10C show an exemplary compound's (IO-199_04) efficacy (FIG. 10A), uptake (FIG. 10B) and DNA platinum adduct formation (FIG. 10C) in lung cancer (A549) cells.

FIG. 11 shows an exemplary compound's (IO-200_06) internalization in lung cancer (A549) cells.

FIG. 12 shows cellular internalization of an exemplary compound (IO-199_34) and its ligand (Im-02) in lung cancer (A549) cells.

### DETAILED DESCRIPTION

In one aspect, provided herein are platinum containing compounds. Generally, the platinum compounds comprise a fluorescent molecule conjugated with a platinum atom. The platinum atom can be part of a platinum moiety. For example, the platinum moiety can be a platinum (II) or platinum (IV) compound. In some embodiments, the platinum (II) compound is selected from the group comprising of DACH-platinum, cisplatin, oxaliplatin, carboplatin, paraplatin, sartraplatin, and various combinations thereof. In some embodiments, the platinum containing compound is Pt(II) compound, Pt(IV) compound or halide containing platinum compound.

In some embodiment, the platinum containing compound disclosed herein is of Formula VI:

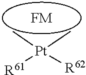

wherein:
FM is fluorescent molecule;
R⁶¹ and R⁶² are same or different and selected independently from halogen, alkyl, amino, alkylamino, dialkylamino, hydroxyl, alkoxy, thiol, thioalkyl, —S(O)(R⁶³)₂, O-acyl, or any combinations thereof, or R⁶¹ and R⁶², together with the Pt atom form an optionally substituted cyclyl or heterocyclyl; and
each R⁶³ is independently a C₁-C₆alkyl.

Without limitations, R⁶¹ and R⁶² can be same or different. In some compounds of Formula VI, R⁶¹ and R⁶² are independently halogen or NH₃. In some embodiments, R⁶¹ and R⁶² are Cl. In some other embodiments, R⁶¹ and R⁶² are NH₃.

In some embodiments, R⁶¹ and R⁶², together with the Pt atom form a cyclyl or heterocyclyl is substituted with or linked to a lipid.

In some embodiments, R⁶³ is selected from the group consisting of methyl, ethyl, propyl, isopropyl, butyl, pentyl and hexanyl.

Without limitations, at least one of R⁶¹ and R⁶² can form a coordination bond with the platinum atom. In some embodiments, the coordination bond is a O->Pt or N->Pt coordination bond.

In some embodiment, the platinum containing compound disclosed herein is of Formula VII:

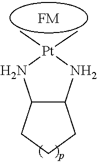

wherein:
p is 0, 1, 2, 3 or 4.

In some compounds of Formula VII, p is 2.

In some embodiments, a compound of Formula VII is of Formula VII′:

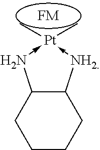

In some embodiments, the platinum containing compound disclosed herein is of Formula VIII:

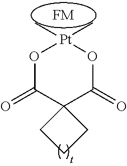

wherein:
FM is a fluorescent molecule; and
t is 0, 1, 2, 3 or 4.

In some embodiments, the platinum containing compound disclosed herein is of Formula IX:

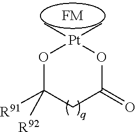

wherein:
FM is a fluorescent molecule;
R⁹¹ and R⁹² are hydrogen or together form a carbonyl (i.e., O═); and
q is 0, 1, 2, 3 or 4.

In some compounds of Formula IX, q is 0 or 1. In some compounds of Formula IX, R⁹¹ and R⁹² are hydrogen and q is 0. In some other compounds of Formula IX, R⁹¹ and R⁹² together form a carbonyl (O═) and q is 0 or 1.

In some embodiments, the platinum containing compound disclosed herein is of Formula X:

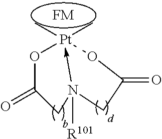

wherein:
FM is a fluorescent molecule;
R¹⁰¹ is H or a -linker-lipid;
b is 1, 2, 3 or 4; and
d is 1, 2, 3, or 4.

In various compounds of Formula X, b and d are same. In other compounds of Formula X, b and d are different. In some embodiments, b and d are 1.

In some compounds of Formula X, R¹⁰¹ is CH₂CH₂OR^{L}, wherein R^{L} is a lipid.

In some embodiments, two fluorescent molecules are linked to the platinum atom. The two fluorescent molecules linked to the platinum atom can be the same or different. In some embodiments, at least one (e.g., one or two) of the fluorescent molecules linked to the platinum atom is a phenalenyl. In some embodiments, at least (e.g., one or two) fluorescent molecule linked to the platinum atom is conjugated with a lipid.

Accordingly, in some embodiments, the platinum containing compound disclosed herein is of Formula XI:

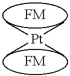

wherein:
each FM is an independently selected fluorescent molecule.

Generally, the fluorescent molecule conjugated with the platinum atom is an optionally substituted phenalenyl moiety. In some embodiments, the phenalenyl moiety is conjugated with a lipid, optionally substituted amino, optionally substituted alkylthio carbohydrate, anti-cancer agent, or optionally substituted PEG.

In some embodiments, the fluorescent molecule in compounds of Formula VI, VII, VIII, IX, X or XI is selected from the group consisting of:
(i) a compound of Formula I:

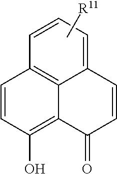

wherein R¹¹ is hydrogen, alkoxy, optionally substituted alkylamino, optionally substituted alkylthio, -linker-(anti-cancer agent), -linker-carbohydrate, or -linker-lipid;
(ii) a compound of Formula II:

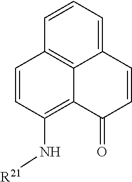

wherein R²¹ is hydrogen, optionally substituted alkyl, cyclyl, heterocyclyl, aryl, heteroaryl, optionally substituted PEG, -linker-carbohydrate, -linker-(anti-cancer agent), -linker-NH(CH₂CO₂H)₂, -linker-CO₂H,

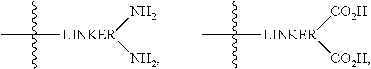

or -linker-lipid;
(iii) a compound of Formula III:

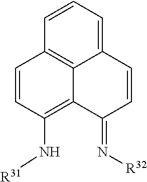

wherein R³¹ and R³² are same or different and selected independently from the group consisting of hydrogen, alkyl, cyclyl, heterocyclyl, aryl, heteroaryl, or -linker-lipid;
(iv) a compound a Formula IV;

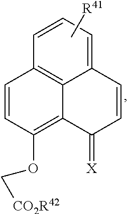

wherein X is O or NR⁴³; R⁴¹ is absent, hydroxyl, alkoxy, -linker-lipid or polyethylene glycol; R⁴² is H, alkyl, cyclyl, heterocyclyl, aryl or heteroaryl; and R⁴³ is H, alkyl, cyclyl, heterocyclyl, aryl or heteroaryl
(v) a compound of Formula V:

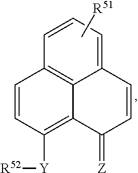

wherein Y is O, S or NR⁵³; Z is O or NR⁵³; R⁵¹ is absent, alkoxy, optionally substituted amino, thiol, optionally substituted alkylthio, -linker-carbohydrate, -linker-(anti-cancer agent), or -linker-lipid; R⁵² is H, alkyl, cyclyl, heterocyclyl, aryl, heteroaryl, optionally substituted PEG, -linker-carbohydrate, -linker-(anti-cancer agent), -linker-NH(CH₂CO₂H)₂, -linker-CO₂H,

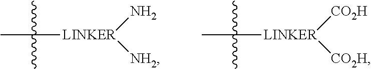

or -linker-lipid; and each R⁵³ is same or different and selected independently from the group consisting of alkyl, cyclyl, heterocyclyl, aryl, heteroaryl, or -linker-lipid, optionally provided that at least one of R⁵¹ and R⁵² is optionally substituted PEG, -linker-carbohydrate, -linker-(anti-cancer agent), -linker-(anti-cancer agent), -linker-NH(CH₂CO₂H)₂,

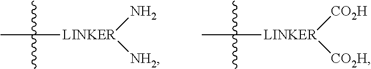

or -linker-lipid; and
(vi) any combinations of (i)-(v).

In some further embodiments, the fluorescent molecule in compounds of Formula VI, VII, VIII, IX, X or XI is selected from the group consisting of:
(i) a compound of Formula I′:

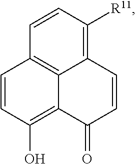

wherein R¹¹ is hydrogen, alkoxy, alkylamino, alkylthio, -linker-(anti-cancer agent), -linker-carbohydrate, or -linker-lipid;
(ii) a compound of Formula II′:

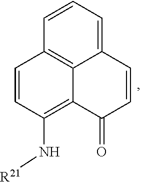

wherein R²¹ is H, optionally substituted alkyl, optionally substituted PEG, -linker-carbohydrate, -linker-(anti-cancer agent), -linker-NH(CH₂CO₂H)₂, -linker-CO₂H,

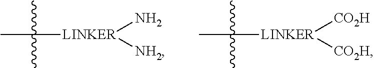

or -linker-lipid;
(iii) a compound of Formula III′:

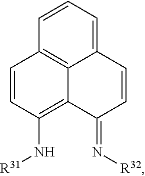

wherein R³¹ and R³² are same or different and independently H, optionally substituted alkyl, or -linker-lipid;
(iv) a compound of Formula IV′:

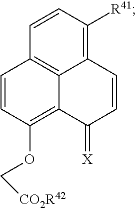

or
(v) a compound of Formula V′:

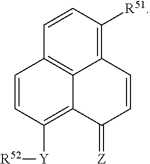

In some embodiments, the fluorescent molecule in compounds of Formula VI, VII, VIII, IX, X or XI is a compound of Formula I″:

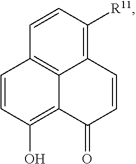

wherein R¹¹ is hydrogen, alkoxy, alkylamino, alkylthio, -linker-carbohydrate, or -linker-lipid.

In some embodiments, the fluorescent molecule in compounds of Formula VI, VII, VIII, IX, X or XI is a compound of Formula II″:

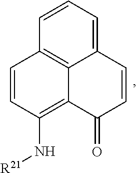

wherein R²¹ is a optionally substituted PEG, -linker-carbohydrate, -linker-(anti-cancer agent), -linker-NH(CH₂CO₂H)₂, -linker-CO₂H,

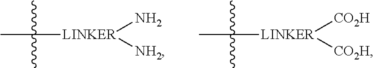

or linker lipid.

In some embodiments, the fluorescent molecule in compounds of Formula VI, VII, VIII, IX, X or XI is a compound of Formula III″:

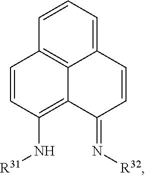

wherein at least one of R³¹ and R³² is a -linker-lipid.

In some further embodiments, the fluorescent molecule in compounds of Formula VI, VII, VIII, IX, X or XI is a compound of Formula V″:

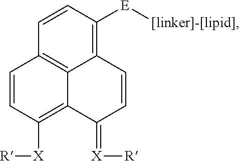

wherein:
E is O, NH or S;
each X is same or different and selected independently from the group consisting of O, N, S, NH and NR
each R′ is same or different and selected independently from the group consisting of H, alkyl, cyclyl, heterocyclyl, aryl and heteroaryl, each of which can be optionally substituted; and
R is alkyl, cyclyl, heterocyclyl, aryl or heteroaryl, each of which can be optionally substituted.

In some embodiments, the fluorescent molecule in compounds of Formula VI, VII, VIII, IX, X or XI is a compound of Formula V″-B:

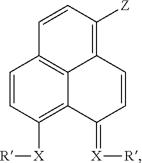

wherein:
Z is alkoxy, alkylamino, alkylthio, -E-linker-(anti-cancer agent) or -E-linker-carbohydrate;
E is O, NH or S;
each X is same or different and selected independently from the group consisting of O, N, S, NH and NR
each R′ is same or different and selected independently from the group consisting of H, alkyl, cyclyl, heterocyclyl, aryl and heteroaryl, each of which can be optionally substituted; and
R is alkyl, cyclyl, heterocyclyl, aryl or heteroaryl, each of which can be optionally substituted.

In some further embodiments, the fluorescent molecule in compounds of Formula VI, VII, VIII, IX, X or XI is a compound of Formula V′″:

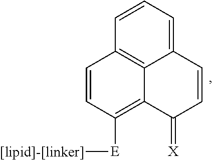

wherein:
E is NH or S;
X is O or NR; and
R is H, alkyl, cyclyl, heterocyclyl, aryl or heteroaryl, each of which can be optionally substituted.

In some further embodiments, the fluorescent molecule in compounds of Formula VI, VII, VIII, IX, X or XI is a compound of Formula V′″-B:

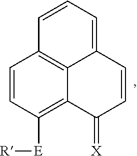

wherein:
E is NH or S;
R′ is optionally substituted PEG, -linker-carbohydrate, -linker-(anti-cancer agent), -linker-NH(CH₂CO₂H)₂, -linker-CO₂H,

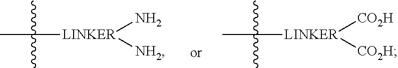

X is O or NR; and
R is H, alkyl, cyclyl, heterocyclyl, aryl or heteroaryl, each of which can be optionally substituted.

In some embodiment, the platinum containing compound disclosed herein is of Formula XIII:

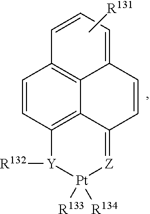

wherein:
Y is O, S or NR¹³⁵;
Z is O or NR¹³⁵;
R¹³¹ is absent, alkoxy, optionally substituted amino, thiol, optionally substituted alkylthio, -linker-(anti-cancer agent), -linker-carbohydrate or -linker-lipid;
R¹³² is H, alkyl, cyclyl, heterocyclyl, aryl, heteroaryl, optionally substituted PEG, -linker-carbohydrate, -linker-(anti-cancer agent), or -linker-lipid;
R¹³³ and R¹³⁴ are same or different and selected independently from halogen, alkyl, amino, alkylamino, dialkylamino, hydroxyl, alkoxy, thiol, thioalkyl, —S(O)(R¹³⁶)₂, O-acyl, or any combinations thereof, or R¹³³ and R¹³⁴, together with the Pt atom form an optionally substituted cyclyl or heterocyclyl;
each R¹³⁵ is same or different and selected independently from the group consisting of alkyl, cyclyl, heterocyclyl, aryl, heteroaryl, or -linker-lipid; and
each R¹³⁶ is independently a C₁-C₆alkyl.

In some embodiments, at least one of R¹³³ and R¹³⁴ forms a coordination bond with the platinum atom. Without limitations, the coordination bond can be a O->Pt or N->Pt coordination bond.

In some embodiments, R¹³³ and R¹³⁴ are independently halogen or —S(O)(R¹³⁶)₂. In some embodiments, R¹³³ and R¹³⁴ are Cl or I. In some embodiments, one of R¹³³ and R¹³⁴ is halogen and the other is —S(O)(CH₃)₂.

In some embodiments, R¹³³ and R¹³⁴, together with the Pt atom, form a cyclyl or heterocyclyl substituted or linked with a lipid. In some embodiments, R¹³³ and R¹³⁴ form the cyclyl

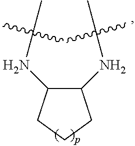

where p is 0, 1, 2, 3 or 4. In some embodiments, p is 1 or 2. In one embodiment, p is 2.

In some embodiments, R¹³³ and R¹³⁴ form the cyclyl

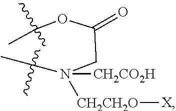

where X is C₁-C₆alkyl or a lipid. In some embodiments, X is methyl or cholesterol.

In some embodiment, the platinum containing compound disclosed herein is of Formula XIV:

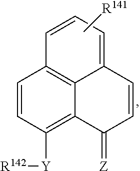

wherein:
Y is O, S or NR¹⁴³;
Z is O or NR¹⁴³;
R¹⁴¹ is absent, alkoxy, optionally substituted amino, thiol, optionally substituted alkylthio, -linker-(anti-cancer agent), -linker-carbohydrate or -linker-lipid;
R¹⁴² is

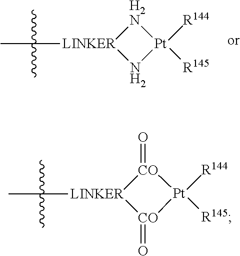

each R¹⁴³ is same or different and selected independently from the group consisting of alkyl, cyclyl, heterocyclyl, aryl, heteroaryl, or -linker-lipid; and
R¹⁴⁴ and R¹⁴⁵ are same or different and selected independently from halogen, alkyl, amino, alkylamino, dialkylamino, hydroxyl, alkoxy, thiol, thioalkyl, O-acyl, or any combinations thereof, or R¹⁴⁴ and R¹⁴⁵, together with the Pt atom form an optionally substituted cyclyl or heterocyclyl.

In some embodiments, R¹⁴⁴ and R¹⁴⁵ are independently halogen. In some embodiments, R¹⁴⁵ and R¹⁴⁵ are Cl.

In some embodiments, R¹⁴⁴ and R¹⁴⁵ form the cyclyl

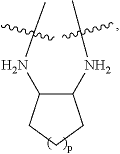

where p is 0, 1, 2, 3 or 4. In some embodiments, p is 1 or 2. In one embodiment, p is 2.

In yet another aspect, the present disclosure provides a compound of Formula XII:

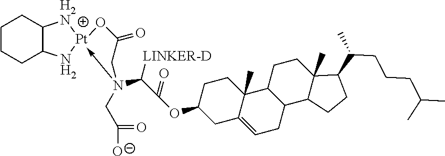

wherein D is an imaging agent.

As used herein, the term “imaging agent” refers to an element or functional group in a molecule that allows for the detection, imaging, and/or monitoring of the presence and/or progression of a condition(s), pathological disorder(s), and/or disease(s). The imaging agent can be an echogenic substance (either liquid or gas), non-metallic isotope, an optical reporter, a boron neutron absorber, a paramagnetic metal ion, a ferromagnetic metal, a gamma-emitting radioisotope, a positron-emitting radioisotope, or an x-ray absorber.

Suitable optical reporters include, but are not limited to, fluorescent reporters and chemiluminescent groups. A wide variety of fluorescent reporter dyes, e.g., fluorophores, are known in the art. Typically, the fluorophore is an aromatic or heteroaromatic compound and can be a pyrene, anthracene, naphthalene, acridine, stilbene, indole, benzindole, oxazole, thiazole, benzothiazole, cyanine, carbocyanine, salicylate, anthranilate, coumarin, fluorescein, rhodamine or other like compound. Suitable fluorescent reporters include xanthene dyes, such as fluorescein or rhodamine dyes. Exemplary fluorophores include, but are not limited to, 1,5 IAEDANS; 1,8-ANS; 4-Methylumbelliferone; 5-carboxy-2,7-dichlorofluorescein; 5-Carboxyfluorescein (5-FAM); 5-Carboxynapthofluorescein (pH 10); 5-Carboxytetramethylrhodamine (5-TAMRA); 5-FAM (5-Carboxyfluorescein); 5-Hydroxy Tryptamine (HAT); 5-ROX (carboxy-X-rhodamine); 5-TAMRA (5-Carboxytetramethylrhodamine); 6-Carboxyrhodamine 6G; 6-CR 6G; 6-JOE; 7-Amino-4-methylcoumarin; 7-Aminoactinomycin D (7-AAD); 7-Hydroxy-4-methylcoumarin; 9-Amino-6-chloro-2-methoxyacridine; ABQ; Acid Fuchsin; ACMA (9-Amino-6-chloro-2-methoxyacridine); Acridine Orange; Acridine Red; Acridine Yellow; Acriflavin; Acriflavin Feulgen SITSA; Aequorin (Photoprotein); Alexa Fluor 350™; Alexa Fluor 430™; Alexa Fluor 488™; Alexa Fluor 532™; Alexa Fluor 546™; Alexa Fluor 568™; Alexa Fluor 594™; Alexa Fluor 633™; Alexa Fluor 647™; Alexa Fluor 660™; Alexa Fluor 680™; Alizarin Complexon; Alizarin Red; Allophycocyanin (APC); AMC, AMCA-S; AMCA (Aminomethylcoumarin); AMCA-X; Aminoactinomycin D; Aminocoumarin; Anilin Blue; Anthrocyl stearate; APC-Cy7; APTS; Astrazon Brilliant Red 4G; Astrazon Orange R; Astrazon Red 6B; Astrazon Yellow 7 GLL; Atabrine; ATTO-TAG™ CBQCA; ATTO-TAG™ FQ; Auramine; Aurophosphine G; Aurophosphine; BAO 9 (Bisaminophenyloxadiazole); BCECF (high pH); BCECF (low pH); Berberine Sulphate; Beta Lactamase; BFP blue shifted GFP (Y66H); BG-647; Bimane; Bisbenzamide; Blancophor FFG; Blancophor SV; BOBO™-1; BOBO™-3; Bodipy 492/515; Bodipy 493/503; Bodipy 500/510; Bodipy 505/515; Bodipy 530/550; Bodipy 542/563; Bodipy 558/568; Bodipy 564/570; Bodipy 576/589; Bodipy 581/591; Bodipy 630/650-X; Bodipy 650/665-X; Bodipy 665/676; Bodipy Fl; Bodipy FL ATP; Bodipy Fl-Ceramide; Bodipy R6G SE; Bodipy TMR; Bodipy TMR-X conjugate; Bodipy TMR-X, SE; Bodipy TR; Bodipy TR ATP; Bodipy TR-X SE; BO-PRO™-1; BO-PRO™-3; Brilliant Sulphoflavin FF; Calcein; Calcein Blue; Calcium Crimson™; Calcium Green; Calcium Green-1 Ca²⁺ Dye; Calcium Green-2 Ca²⁺; Calcium Green-5N Ca²⁺; Calcium Green-C18 Ca²⁺; Calcium Orange; Calcofluor White; Carboxy-X-rhodamine (5-ROX); Cascade Blue™; Cascade Yellow; Catecholamine; CFDA; CFP—Cyan Fluorescent Protein; Chlorophyll; Chromomycin A; Chromomycin A; CMFDA; Coelenterazine; Coelenterazine cp; Coelenterazine f; Coelenterazine fcp; Coelenterazine h; Coelenterazine hcp; Coelenterazine ip; Coelenterazine O; Coumarin Phalloidin; CPM Methylcoumarin; CTC; Cy2™; Cy3.1 8; Cy3.5™; Cy3™; Cy5.1 8; Cy5.5™; Cy5™; Cy7™; Cyan GFP; cyclic AMP Fluorosensor (FiCRhR); d2; Dabcyl; Dansyl; Dansyl Amine; Dansyl Cadaverine; Dansyl Chloride; Dansyl DHPE; Dansyl fluoride; DAPI; Dapoxyl; Dapoxyl 2; Dapoxyl 3; DCFDA; DCFH (Dichlorodihydrofluorescein Diacetate); DDAO; DHR (Dihydorhodamine 123); Di-4-ANEPPS; Di-8-ANEPPS (non-ratio); DiA (4-Di-16-ASP); DIDS; Dihydorhodamine 123 (DHR); DiO (DiOC18(3)); DiR; DiR (DiIC18(7)); Dopamine; DsRed; DTAF; DY-630-NHS; DY-635-NHS; EBFP; ECFP; EGFP; ELF 97; Eosin; Erythrosin; Erythrosin ITC; Ethidium homodimer-1 (EthD-1); Euchrysin; Europium (III) chloride; Europium; EYFP; Fast Blue; FDA; Feulgen (Pararosaniline); FITC; FL-645; Flazo Orange; Fluo-3; Fluo-4; Fluorescein Diacetate; Fluoro-Emerald; Fluoro-Gold (Hydroxystilbamidine); Fluor-Ruby; FluorX; FM 1-43™; FM 4-46; Fura Red™ (high pH); Fura-2, high calcium; Fura-2, low calcium; Genacryl Brilliant Red B; Genacryl Brilliant Yellow 10GF; Genacryl Pink 3G; Genacryl Yellow 5GF; GFP (S65T); GFP red shifted (rsGFP); GFP wild type, non-UV excitation (wtGFP); GFP wild type, UV excitation (wtGFP); GFPuv; Gloxalic Acid; Granular Blue; Haematoporphyrin; Hoechst 33258; Hoechst 33342; Hoechst 34580; HPTS; Hydroxycoumarin; Hydroxystilbamidine (FluoroGold); Hydroxytryptamine; Indodicarbocyanine (DiD); Indotricarbocyanine (DiR); Intrawhite Cf; JC-1; JO-JO-1; JO-PRO-1; LaserPro; Laurodan; LDS 751; Leucophor PAF; Leucophor SF; Leucophor WS; Lissamine Rhodamine; Lissamine Rhodamine B; LOLO-1; LO-PRO-1; Lucifer Yellow; Mag Green; Magdala Red (Phloxin B); Magnesium Green; Magnesium Orange; Malachite Green; Marina Blue; Maxilon Brilliant Flavin 10 GFF; Maxilon Brilliant Flavin 8 GFF; Merocyanin; Methoxycoumarin; Mitotracker Green FM; Mitotracker Orange; Mitotracker Red; Mitramycin; Monobromobimane; Monobromobimane (mBBr-GSH); Monochlorobimane; MPS (Methyl Green Pyronine Stilbene); NBD; NBD Amine; Nile Red; Nitrobenzoxadidole; Noradrenaline; Nuclear Fast Red; Nuclear Yellow; Nylosan Brilliant Iavin E8G; Oregon Green™; Oregon Green 488-X; Oregon Green™ 488; Oregon Green™ 500; Oregon Green™ 514; Pacific Blue; Pararosaniline (Feulgen); PE-Cy5; PE-Cy7; PerCP; PerCP-Cy5.5; PE-TexasRed (Red 613); Phloxin B (Magdala Red); Phorwite AR; Phorwite BKL; Phorwite Rev; Phorwite RPA; Phosphine 3R; PhotoResist; Phycoerythrin B [PE]; Phycoerythrin R [PE]; PKH26; PKH67; PMIA; Pontochrome Blue Black; POPO-1; POPO-3; PO-PRO-1; PO-PRO-3; Primuline; Procion Yellow; Propidium lodid (PI); PyMPO; Pyrene; Pyronine; Pyronine B; Pyrozal Brilliant Flavin 7GF; QSY 7; Quinacrine Mustard; Resorufin; RH 414; Rhod-2; Rhodamine; Rhodamine 110; Rhodamine 123; Rhodamine 5 GLD; Rhodamine 6G; Rhodamine B 540; Rhodamine B 200; Rhodamine B extra; Rhodamine BB; Rhodamine BG; Rhodamine Green; Rhodamine Phallicidine; Rhodamine Phalloidine; Rhodamine Red; Rhodamine WT; Rose Bengal; R-phycoerythrin (PE); red shifted GFP (rsGFP, S65T); S65A; S65C; S65L; S65T; Sapphire GFP; Serotonin; Sevron Brilliant Red 2B; Sevron Brilliant Red 4G; Sevron Brilliant Red B; Sevron Orange; Sevron Yellow L; sgBFP™; sgBFP™ (super glow BFP); sgGFP™; sgGFP™ (super glow GFP); SITS; SITS (Primuline); SITS (Stilbene Isothiosulphonic Acid); SPQ (6-methoxy-N-(3-sulfopropyl)-quinolinium); Stilbene; Sulphorhodamine B can C; Sulphorhodamine G Extra; Tetracycline; Tetramethylrhodamine; Texas Red™; Texas Red-X™ conjugate; Thiadicarbocyanine (DiSC3); Thiazine Red R; Thiazole Orange; Thioflavin 5; Thioflavin S; Thioflavin TCN; Thiolyte; Thiozole Orange; Tinopol CBS (Calcofluor White); TMR; TO-PRO-1; TO-PRO-3; TO-PRO-5; TOTO-1; TOTO-3; TriColor (PE-Cy5); TRITC (TetramethylRodamineIsoThioCyanate); True Blue; TruRed; Ultralite; Uranine B; Uvitex SFC; wt GFP; WW 781; XL665; X-Rhodamine; XRITC; Xylene Orange; Y66F; Y66H; Y66W; Yellow GFP; YFP; YO-PRO-1; YO-PRO-3; YOYO-1; and YOYO-3. Many suitable forms of these fluorescent compounds are available and can be used.

Examples of fluorescent proteins suitable for use as imaging agents include, but are not limited to, green fluorescent protein, red fluorescent protein (e.g., DsRed), yellow fluorescent protein, cyan fluorescent protein, blue fluorescent protein, and variants thereof (see, e.g., U.S. Pat. Nos. 6,403,374, 6,800,733, and 7,157,566). Specific examples of GFP variants include, but are not limited to, enhanced GFP (EGFP), destabilized EGFP, the GFP variants described in Doan et al, Mol. Microbiol, 55:1767-1781 (2005), the GFP variant described in Crameri et al, Nat. Biotechnol., 14:315319 (1996), the cerulean fluorescent proteins described in Rizzo et al, Nat. Biotechnol, 22:445 (2004) and Tsien, Annu. Rev. Biochem., 67:509 (1998), and the yellow fluorescent protein described in Nagal et al, Nat. Biotechnol., 20:87-90 (2002). DsRed variants are described in, e.g., Shaner et al, Nat. Biotechnol., 22:1567-1572 (2004), and include mStrawberry, mCherry, mOrange, mBanana, mHoneydew, and mTangerine. Additional DsRed variants are described in, e.g., Wang et al, Proc. Natl. Acad. Sci. U.S.A., 101:16745-16749 (2004) and include mRaspberry and mPlum. Further examples of DsRed variants include mRFPmars described in Fischer et al, FEBS Lett., 577:227-232 (2004) and mRFPruby described in Fischer et al, FEBS Lett, 580:2495-2502 (2006).

Suitable echogenic gases include, but are not limited to, a sulfur hexafluoride or perfluorocarbon gas, such as perfluoromethane, perfluoroethane, perfluoropropane, perfluorobutane, perfluorocyclobutane, perfluropentane, or perfluorohexane. Suitable non-metallic isotopes include, but are not limited to, ¹¹C, ¹⁴C, ¹³N, ¹⁸F, ¹²³I, ¹²⁴I, ¹²⁵I, and ¹³¹I. Suitable radioisotopes include, but are not limited to, ⁹⁹mTc, ⁹⁵Tc, ¹¹¹In, ⁶²Cu, ⁶⁴Cu, Ga, ⁶⁸Ga, ⁴⁷Sc, ⁶⁴Cu, ⁶⁷Cu, ⁸⁹Sr, ⁸⁶Y, ⁸⁷Y, ⁹⁰Y, ¹⁰⁵Rh, ¹¹¹Ag, ¹¹¹In, ¹¹⁷mSn, ¹⁴⁹Pm, ¹⁵³Sm, ¹⁶⁶Ho, ¹⁷⁷Lu, ¹⁸⁶Re, ¹⁸⁸Re, ²¹¹At, ²¹²Bi, and ¹⁵³Gd. Suitable paramagnetic metal ions include, but are not limited to, Gd(III), Dy(III), Fe(III), and Mn(II). Suitable X-ray absorbers include, but are not limited to, Re, Sm, Ho, Lu, Pm, Y, Bi, Pd, Gd, La, Au, Au, Yb, Dy, Cu, Rh, Ag, and Ir.

In some embodiments, the imaging agent comprises a chelating molecule. Suitable chelating agents include, but are not limited to, 1,4,7,10-tetraazocyclododecane-1,4,7,10-tetraacetic acid (DOTA); dibenzo-DOTA, diethylenetriaminepentaacetic acid (DTPA); 1,4,7,10-tetraazacyclododecane-1,4,7,10-tetrakis(2-propionic acid) (DOTMA); 1,4,8,11-tetrazacyclotetradecane-1,4,8,11-tetraacetic acid (TETA); 1,4,7,-tricarboxymethyl 1,4,7,10 teraazacyclododecane triacetic acid (DO3A); 1,4,7,10-tetraazacyclo-dodecan-1-(2-hydroxypropyl)-4,7,10-triacetic acid (HP-DO3A); ethylenediamine-tetraacetic acid (EDTA); bis-2 (hydroxybenzyl)-ethylene-diaminediacetic acid (HBED); 1,4,7-triazacyclo-nonane N,N′,N″-triacetic acid (NOTA); BAD, EDTA, NTA, HDTA, their phosphonate analogs, and mixtures thereof. In some embodiments, the imaging agent is Alexa Fluor 680™.

In some embodiments, the imaging agent is a fluorescent molecule, i.e., a fluorophore. In some embodiments, the fluorescent molecule is selected from the group consisting of 7-amino-4-methyl coumarin, rhodamine, fluorescein, dansyl, fluorene-1-carboxylic acid, and bimane.

Synthesis of compounds of Formula XII is described in Example 4. Some exemplary compounds of Formula XII are as follows:

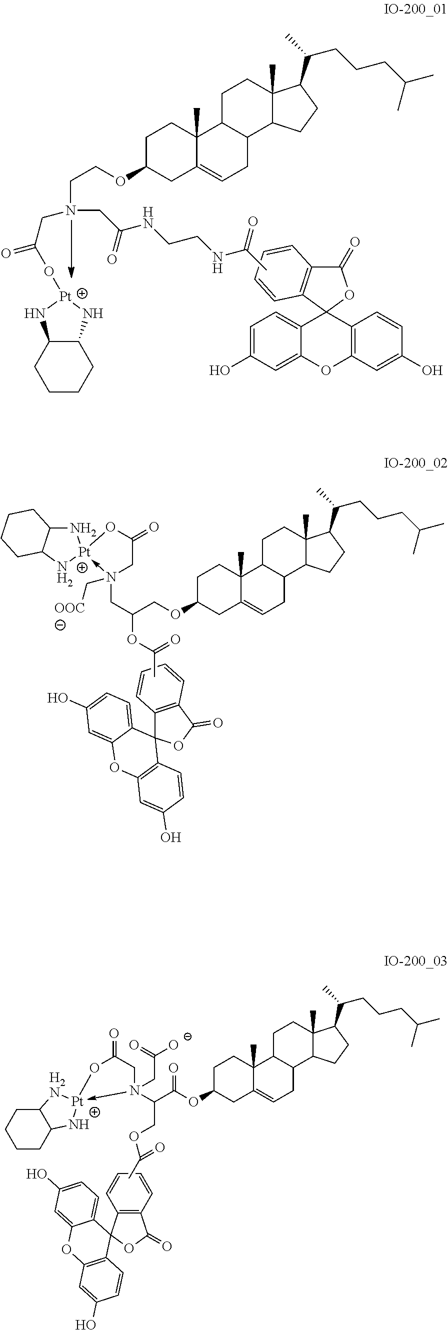

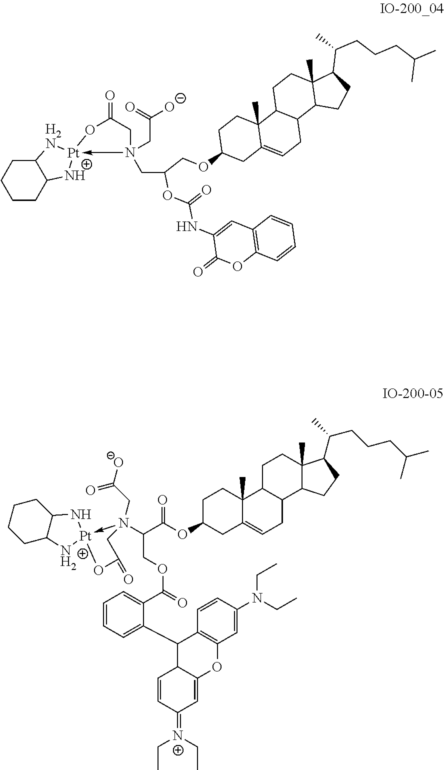

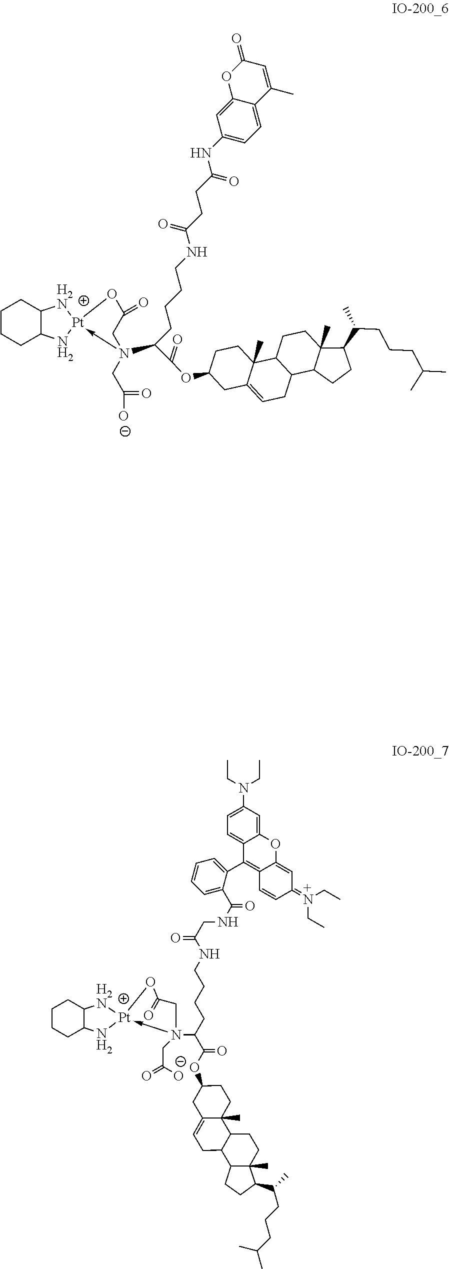

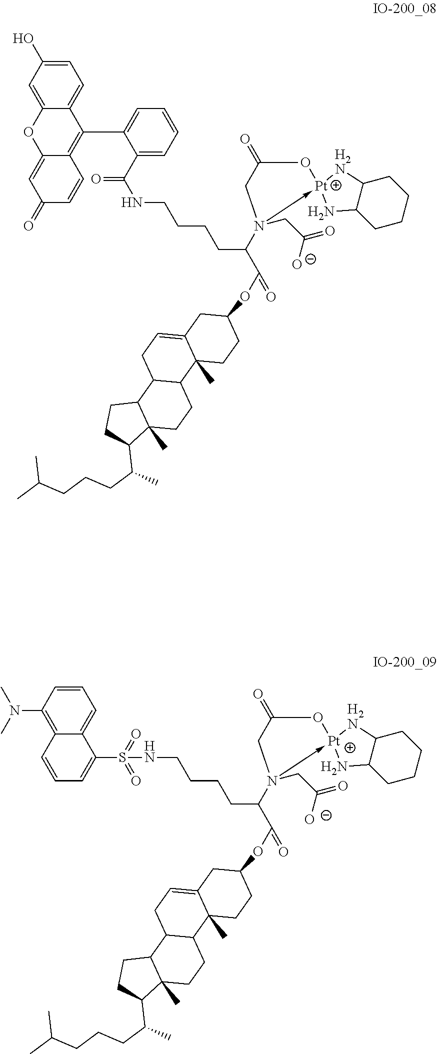

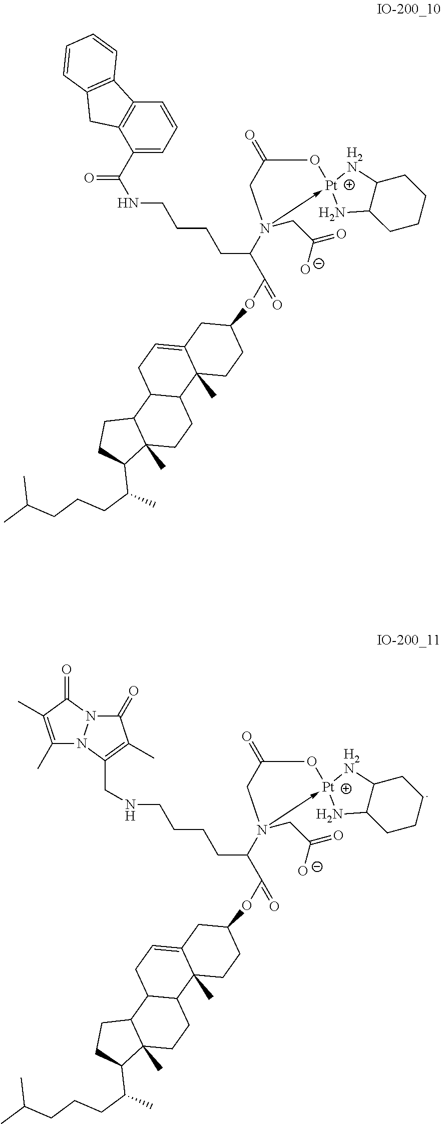

In some embodiments of the various aspects disclosed herein, the platinum atom is linked to the rest of the molecule via at least one coordination bond. In some embodiments, the platinum atom is linked to the rest of the molecule via at least one O→Pt coordination bond. In some embodiments, the platinum atom is linked to the rest of the molecule via at least one N→Pt coordinate or N—Pt coordinate covalent bond. In some embodiments, the platinum atom is linked to the rest of the molecule via one N→Pt coordinate bond and one N—Pt coordinate covalent bond.

In some embodiments, the platinum atom is linked to the rest of the molecule via at least one O—Pt carboxylato covalent bond. In some embodiments, the platinum atom is linked to the rest of the molecule via one O—Pt carboxylato covalent bond and at least one coordination bond. For example, the platinum atom is linked to the rest of the molecule via one O—Pt carboxylato covalent bond and a O→Pt or N→Pt coordination bond.

The coordination bond can be between the platinum atom and the fluorescent molecule or the non-fluorescent part of the compound. Accordingly, in some embodiments, the platinum atom is linked with the fluorescent molecule via at least one O→Pt coordination bond. In some embodiments, the platinum atom is linked with the fluorescent molecule via at least one N→Pt coordination bond. In some embodiments, the platinum atom is linked with the fluorescent molecule via two N→Pt coordination bonds. In some embodiments, the platinum atom is linked with the fluorescent molecule via at least one O—Pt carboxylato covalent bond. In some embodiments, the platinum atom is linked with the fluorescent molecule via one O—Pt carboxylato covalent bond and at least one coordination bond. For example, the platinum atom is linked with the fluorescent molecule via one O—Pt carboxylato covalent bond and a O→Pt or N→Pt coordination bond.

In the various embodiments, a lipid is conjugated with a compound disclosed herein, e.g., a compound of Formula IV or V, or the fluorescent molecule linked with the platinum atom. The term “lipid” is used in the conventional sense and includes compounds of varying chain length, from as short as about 2 carbon atoms to as long as about 28 carbon atoms. Additionally, the compounds may be saturated or unsaturated and in the form of straight- or branched-chains or in the form of unfused or fused ring structures. Exemplary lipids include, but are not limited to, fats, waxes, sterols, steroids, bile acids, fat-soluble vitamins (such as A, D, E, and K), monoglycerides, diglycerides, phospholipids, glycolipids, sulpholipids, aminolipids, chromolipids (lipochromes), glycerophospholipids, sphingolipids, prenollipids, saccharolipids, polyketides, and fatty acids.

Without limitations the lipid can be selected from the group consisting of sterol lipids, fatty acids, fatty alcohols, glycerolipids (e.g., monoglycerides, diglycerides, and triglycerides), phospholipids, glycerophospholipids, sphingolipids, prenol lipids, saccharolipids, polyketides, and any combination thereof. The lipid can be a polyunsaturated fatty acid or alcohol. The term “polyunsaturated fatty acid” or “polyunsaturated fatty alcohol” as used herein means a fatty acid or alcohol with two or more carbon-carbon double bonds in its hydrocarbon chain. The lipid can also be a highly unsaturated fatty acid or alcohol. The term “highly polyunsaturated fatty acid” or “highly polyunsaturated fatty alcohol” as used herein means a fatty acid or alcohol having at least 18 carbon atoms and at least 3 double bonds. The lipid can be an omega-3 fatty acid. The term “omega-3 fatty acid” as used herein means a polyunsaturated fatty acid whose first double bond occurs at the third carbon-carbon bond from the end opposite the acid group.

In some embodiments, the lipid can be selected from the group consisting of 1,3-Propanediol Dicaprylate/Dicaprate; 10-undecenoic acid; 1-dotriacontanol; 1-heptacosanol; 1-nonacosanol; 2-ethyl hexanol; Androstanes; Arachidic acid; Arachidonic acid; arachidyl alcohol; Behenic acid; behenyl alcohol; Capmul MCM C10; Capric acid; capric alcohol; capryl alcohol; Caprylic acid; Caprylic/Capric Acid Ester of Saturated Fatty Alcohol C12-C18; Caprylic/Capric Triglyceride; Caprylic/Capric Triglyceride; Ceramide phosphorylcholine (Sphingomyelin, SPH); Ceramide phosphorylethanolamine (Sphingomyelin, Cer-PE); Ceramide phosphorylglycerol; Ceroplastic acid; Cerotic acid; Cerotic acid; ceryl alcohol; Cetearyl alcohol; Ceteth-10; cetyl alcohol; Cholanes; Cholestanes; cholesterol; cis-11-eicosenoic acid; cis-11-octadecenoic acid; cis-13-docosenoic acid; cluytyl alcohol; coenzyme Q10 (CoQ10); Dihomo-γ-linolenic; Docosahexaenoic acid; egg lecithin; Eicosapentaenoic acid; Eicosenoic acid; Elaidic acid; elaidolinolenyl alcohol; elaidolinoleyl alcohol; elaidyl alcohol; Erucic acid; erucyl alcohol; Estranes; Ethylene glycol distearate (EGDS); Geddic acid; geddyl alcohol; glycerol distearate (type I) EP (Precirol ATO 5); Glycerol Tricaprylate/Caprate; Glycerol Tricaprylate/Caprate (CAPTEX® 355 EP/NF); glyceryl monocaprylate (Capmul MCM C8 EP); Glyceryl Triacetate; Glyceryl Tricaprylate; Glyceryl Tricaprylate/Caprate/Laurate; Glyceryl Tricaprylate/Tricaprate; glyceryl tripalmitate (Tripalmitin); Henatriacontylic acid; Heneicosyl alcohol; Heneicosylic acid; Heptacosylic acid; Heptadecanoic acid; Heptadecyl alcohol; Hexatriacontylic acid; isostearic acid; isostearyl alcohol; Lacceroic acid; Lauric acid; Lauryl alcohol; Lignoceric acid; lignoceryl alcohol; Linoelaidic acid; Linoleic acid; linolenyl alcohol; linoleyl alcohol; Margaric acid; Mead; Melissic acid; melissyl alcohol; Montanic acid; montanyl alcohol; myricyl alcohol; Myristic acid; Myristoleic acid; Myristyl alcohol; neodecanoic acid; neoheptanoic acid; neononanoic acid; Nervonic; Nonacosylic acid; Nonadecyl alcohol; Nonadecylic acid; Nonadecylic acid; Oleic acid; oleyl alcohol; Palmitic acid; Palmitoleic acid; palmitoleyl alcohol; Pelargonic acid; pelargonic alcohol; Pentacosylic acid; Pentadecyl alcohol; Pentadecylic acid; Phosphatidic acid (phosphatidate, PA); Phosphatidylcholine (lecithin, PC); Phosphatidylethanolamine (cephalin, PE); Phosphatidylinositol (PI); Phosphatidylinositol bisphosphate (PIP2); Phosphatidylinositol phosphate (PIP); Phosphatidylinositol triphosphate (PIP3); Phosphatidylserine (PS); polyglyceryl-6-distearate; Pregnanes; Propylene Glycol Dicaprate; Propylene Glycol Dicaprylocaprate; Propylene Glycol Dicaprylocaprate; Psyllic acid; recinoleaic acid; recinoleyl alcohol; Sapienic acid; soy lecithin; Stearic acid; Stearidonic; stearyl alcohol; Tricosylic acid; Tridecyl alcohol; Tridecylic acid; Triolein; Undecyl alcohol; undecylenic acid; Undecylic acid; Vaccenic acid; α-Linolenic acid; γ-Linolenic acid; a fatty acid salt of 10-undecenoic acid, adapalene, arachidic acid, arachidonic acid, behenic acid, butyric acid, capric acid, caprylic acid, cerotic acid, cis-11-eicosenoic acid, cis-11-octadecenoic acid, cis-13-docosenoic acid, docosahexaenoic acid, eicosapentaenoic acid, elaidic acid, erucic acid, heneicosylic acid, heptacosylic acid, heptadecanoic acid, isostearic acid, lauric acid, lignoceric acid, linoelaidic acid, linoleic acid, montanic acid, myristic acid, myristoleic acid, neodecanoic acid, neoheptanoic acid, neononanoic acid, nonadecylic acid, oleic acid, palmitic acid, palmitoleic acid, pelargonic acid, pentacosylic acid, pentadecylic acid, recinoleaic acid (e.g. zinc recinoleate), sapienic acid, stearic acid, tricosylic acid, tridecylic acid, undecylenic acid, undecylic acid, vaccenic acid, valeric acid, α-linolenic acid, γ-linolenic acid; and any combinations thereof.

In some embodiments, the lipid is cholesterol, lumisterol, α-tocopherol or vitamin A.

As used herein, the term “linker” means an organic moiety that connects two parts of a compound. Linkers typically comprise a direct bond or an atom such as oxygen or sulfur, a unit such as NR¹, C(O), C(O)NH, C(O)O, NHC(O)O, OC(O)O, SO, SO₂, SO₂NH or a chain of atoms, such as substituted or unsubstituted alkyl, substituted or unsubstituted alkenyl, substituted or unsubstituted alkynyl, arylalkyl, arylalkenyl, arylalkynyl, heteroarylalkyl, heteroarylalkenyl, heteroarylalkynyl, heterocyclylalkyl, heterocyclylalkenyl, heterocyclylalkynyl, aryl, heteroaryl, heterocyclyl, cycloalkyl, cycloalkenyl, alkylarylalkyl, alkylarylalkenyl, alkylarylalkynyl, alkenylarylalkyl, alkenylarylalkenyl, alkenylarylalkynyl, alkynylarylalkyl, alkynylarylalkenyl, alkynylarylalkynyl, alkylheteroarylalkyl, alkylheteroarylalkenyl, alkylheteroarylalkynyl, alkenylheteroarylalkyl, alkenylheteroarylalkenyl, alkenylheteroarylalkynyl, alkynylheteroarylalkyl, alkynylheteroarylalkenyl, alkynylheteroarylalkynyl, alkylheterocyclylalkyl, alkylheterocyclylalkenyl, alkylhererocyclylalkynyl, alkenylheterocyclylalkyl, alkenylheterocyclylalkenyl, alkenylheterocyclylalkynyl, alkynylheterocyclylalkyl, alkynylheterocyclylalkenyl, alkynylheterocyclylalkynyl, alkylaryl, alkenylaryl, alkynylaryl, alkylheteroaryl, alkenylheteroaryl, alkynylhereroaryl, where one or more methylenes can be interrupted or terminated by O, S, S(O), SO₂, NR¹, C(O), C(O)NH, C(O)O, NHC(O)O, OC(O)O, SO₂NH, cleavable linking group, substituted or unsubstituted aryl, substituted or unsubstituted heteroaryl, substituted or unsubstituted heterocyclic; where R¹ is hydrogen, acyl, aliphatic or substituted aliphatic.

In some embodiments, the linker is a branched linker. The branchpoint of the branched linker may be at least trivalent, but can be a tetravalent, pentavalent or hexavalent atom, or a group presenting such multiple valencies. In some embodiments, the branchpoint is —N, —N(Q)-C, —O—C, —S—C, —SS—C, —C(O)N(Q)-C, —OC(O)N(Q)-C, —N(Q)C(O)—C, or —N(Q)C(O)O—C; wherein Q is independently for each occurrence H or optionally substituted alkyl. In some embodiments, the branchpoint is glycerol or derivative thereof.

A cleavable linking group is one which is sufficiently stable outside the cell, but which upon entry into a target cell is cleaved to release the two parts the linker is holding together. In a preferred embodiment, the cleavable linking group is cleaved at least 10 times or more, preferably at least 100 times faster in the target cell or under a first reference condition (which can, e.g., be selected to mimic or represent intracellular conditions) than in the blood or serum of a subject, or under a second reference condition (which can, e.g., be selected to mimic or represent conditions found in the blood or serum).

Cleavable linking groups are susceptible to cleavage agents, e.g., pH, redox potential or the presence of degradative molecules. Generally, cleavage agents are more prevalent or found at higher levels or activities inside cells than in serum or blood. Examples of such degradative agents include: redox agents which are selected for particular substrates or which have no substrate specificity, including, e.g., oxidative or reductive enzymes or reductive agents such as mercaptans, present in cells, that can degrade a redox cleavable linking group by reduction; esterases; amidases; endosomes or agents that can create an acidic environment, e.g., those that result in a pH of five or lower; enzymes that can hydrolyze or degrade an acid cleavable linking group by acting as a general acid, peptidases (which can be substrate specific) and proteases, and phosphatases.

A linker can include a cleavable linking group that is cleavable by a particular enzyme. The type of cleavable linking group incorporated into a linker can depend on the cell to be targeted. For example, liver targeting ligands can be linked to the cationic lipids through a linker that includes an ester group. Liver cells are rich in esterases, and therefore the linker will be cleaved more efficiently in liver cells than in cell types that are not esterase-rich. Other cell-types rich in esterases include cells of the lung, renal cortex, and testis. Linkers that contain peptide bonds can be used when targeting cell types rich in peptidases, such as liver cells and synoviocytes.

In some embodiments, cleavable linking group is cleaved at least 1.25, 1.5, 1.75, 2, 3, 4, 5, 10, 25, 50, or 100 times faster in the cell (or under in vitro conditions selected to mimic intracellular conditions) as compared to blood or serum (or under in vitro conditions selected to mimic extracellular conditions). In some embodiments, the cleavable linking group is cleaved by less than 90%, 80%, 70%, 60%, 50%, 40%, 30%, 20%, 10%, 5%, or 1% in the blood (or in vitro conditions selected to mimic extracellular conditions) as compared to in the cell (or under in vitro conditions selected to mimic intracellular conditions).

Exemplary cleavable linking groups include, but are not limited to, redox cleavable linking groups (e.g., —S—S— and —C(R)₂—S—S—, wherein R is H or C₁-C₆ alkyl and at least one R is C₁-C₆ alkyl such as CH₃ or CH₂CH₃); phosphate-based cleavable linking groups (e.g., —O—P(O)(OR)—O—, —O—P(S)(OR)—O—, —O—P(S)(SR)—O—, —S—P(O)(OR)—O—, —O—P(O)(OR)—S—, —S—P(O)(OR)—S—, —O—P(S)(ORk)-S—, —S—P(S)(OR)—O—, —O—P(O)(R)—O—, —O—P(S)(R)—O—, —S—P(O)(R)—O—, —S—P(S)(R)—O—, —S—P(O)(R)—S—, —O—P(S)(R)—S—, —O—P(O)(OH)—O—, —O—P(S)(OH)—O—, —O—P(S)(SH)—O—, —S—P(O)(OH)—O—, —O—P(O)(OH)—S—, —S—P(O)(OH)—S—, —O—P(S)(OH)—S—, —S—P(S)(OH)—O—, —O—P(O)(H)—O—, —O—P(S)(H)—O—, —S—P(O)(H)—O—, —S—P(S)(H)—O—, —S—P(O)(H)—S—, and —O—P(S)(H)—S—, wherein R is optionally substituted linear or branched C₁-C₁₀ alkyl); acid cleavable linking groups (e.g., hydrazones, esters, and esters of amino acids, —C═NN— and —OC(O)—); ester-based cleavable linking groups (e.g., —C(O)O—); peptide-based cleavable linking groups, (e.g., linking groups that are cleaved by enzymes such as peptidases and proteases in cells, e.g., —NHCHR^{A}C(O)NHCHR^{B}C(O)—, where R^{A} and R^{B} are the R groups of the two adjacent amino acids). A peptide based cleavable linking group comprises two or more amino acids. In some embodiments, the peptide-based cleavage linkage comprises the amino acid sequence that is the substrate for a peptidase or a protease found in cells.

In some embodiments, an acid cleavable linking group is cleaveable in an acidic environment with a pH of about 6.5 or lower (e.g., about 6.5, 6.0, 5.5, 5.0, or lower), or by agents such as enzymes that can act as a general acid.

Linkers according to the present invention include moieties comprising two or more carbon molecules such as, for example, ethylenediamine, ethyleneglycol, glycine, beta-alanine and polyethylene glycol (PEG) of molecular weight about 44 to about 200 kD. Further, it is to be understood from the present disclosure that the platinum moiety and/or the lipid may be modified to comprise functional groups for linking to the linker molecule.

In some embodiments of the various aspects disclosed herein, the linker is —X—CH₂—X₂—X₁—, wherein X is NH; X₁ is C(O)O, C(O)NH, O(CH₂)—O, NH, or O; X₂ is (CH₂)ₙ or C(O); and n is 0, 1, 2, 3, 4, or 5.

In some other embodiments, the linker is a bond, —(CH₂)ₙ—, —(CH₂)ₙO—, —O(CH₂)ₙO—, —(CH₂)ₙNH—, —O(CH₂)ₙNH—, —NH(CH₂)NH—, —OCH₂(CH₂)ₙC(O)—; —C(O)(CH₂)ₙC(O)—; —(CH₂)ₙNHC(O)O—, —(CH₂)ₙOC(O)NH—, —(CH₂)ₙC(O)NH(CH₂)ₘO—, —(CH₂)ₙO(CH₂)ₘO—, (CH₂)ₙO(O), —(CH₂)ₙNHC(O)(CH₂)ₘO—, —(CH₂)C(O)O—; or —OC(O)(CH₂)C(O)O—; and n and m are independently 0, 1, 2, 3, 4, or 5.

In still some other embodiments, the linker is —X₃—X₄X₅—X₆—, wherein X₃ is CH, CH₂, or O; and X₄, X₅ and X₆ are independently same or different and are —CH₂O- or O.

In yet some other embodiments, the linker is —CH₂O—.

In some embodiments, the linker is selected from the group consisting of a bond, —O—, NHCH₂CH₂NHC(O)—, —NHCH₂CH₂NHC(O)O—, —NHCH₂CH₂—, —NHCH₂CH₂O—, —NHCH₂C(O)—, —NHCH₂C(O)O—, —NHCH₂C(O)OCH₂CH₂CH₂—, —NHCH₂C(O)OCH₂CH₂CH₂O—, —NHCH₂C(O)NH—, —CH₂CH₂—, —CH₂CH₂O—, —CH₂CH₂NHC(O)—, —CH₂CH₂NHC(O)O—, —CH₂CH₂O—, —CH₂C(O)NHCH₂CH₂—, —CH₂C(O)NHCH₂CH₂O—, —CH₂CH₂OCH₂CH₂—, —CH₂CH₂OCH₂CH₂O—, —CH₂C(O)—, —CH₂C(O)O—, —CH₂CH₂CH₂—, —CH₂CH₂CH₂O—, ═CH—CH═CH₂—, ═CH—CH═CHCH₂O—, —CH═CHCH₂—, —CH═CHCH₂O—, —OCH₂CH₂O—, —CH₂—, —CH₂O—, —NHC(O)CH₂—, —NHC(O)CH₂O—, —C(O)CH₂—, —C(O)CH₂O—, —OC(O)CH₂—, —OC(O)CH₂O—, —C(O)CH₂CH₂C(O)NHCH₂CH₂—, —OC(O)CH₂CH₂C(O)NHCH₂CH₂—, —C(O)CH₂CH₂C(O)NHCH₂CH₂O—, —OC(O)CH₂CH₂C(O)NHCH₂CH₂O—, —C(O)CH₂CH₂C(O)NHCH₂CH₂NHC(O)—, —OC(O)CH₂CH₂C(O)NHCH₂CH₂NHC(O)—, —C(O)CH₂CH₂C(O)NHCH₂CH₂NHC(O)O—, —OC(O)CH₂CH₂C(O)NHCH₂CH₂NHC(O)O—, and any combinations thereof.

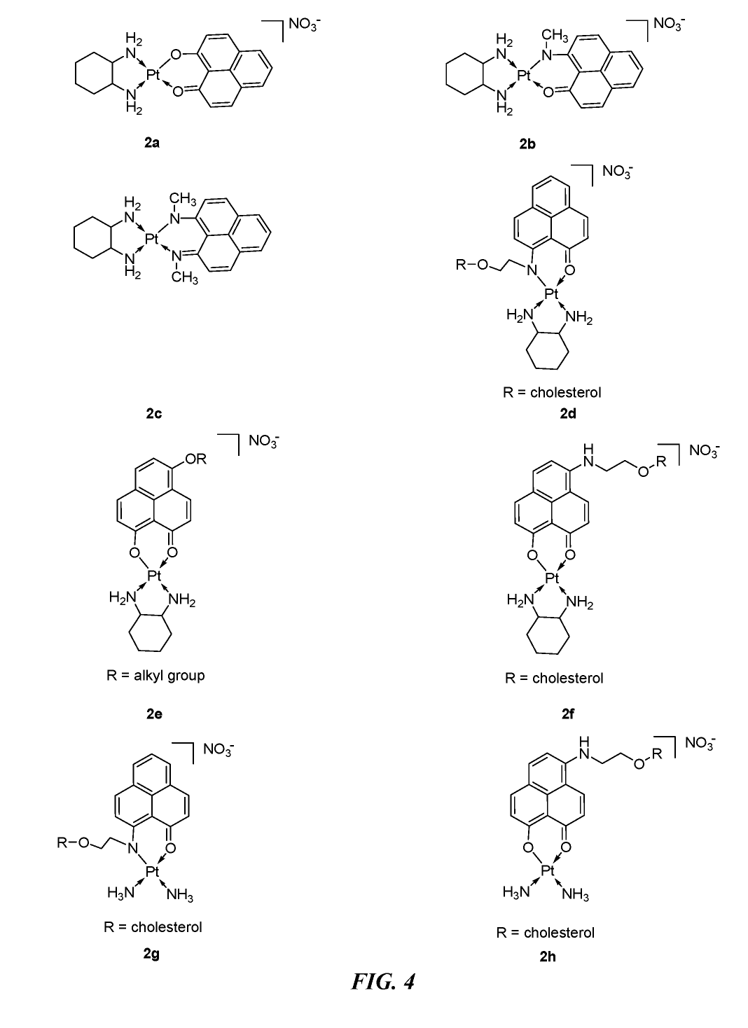

Some exemplary platinum compounds are shown in FIGS. 3-6. Additional exemplary platinum containing compounds include, but are not limited to, the following:

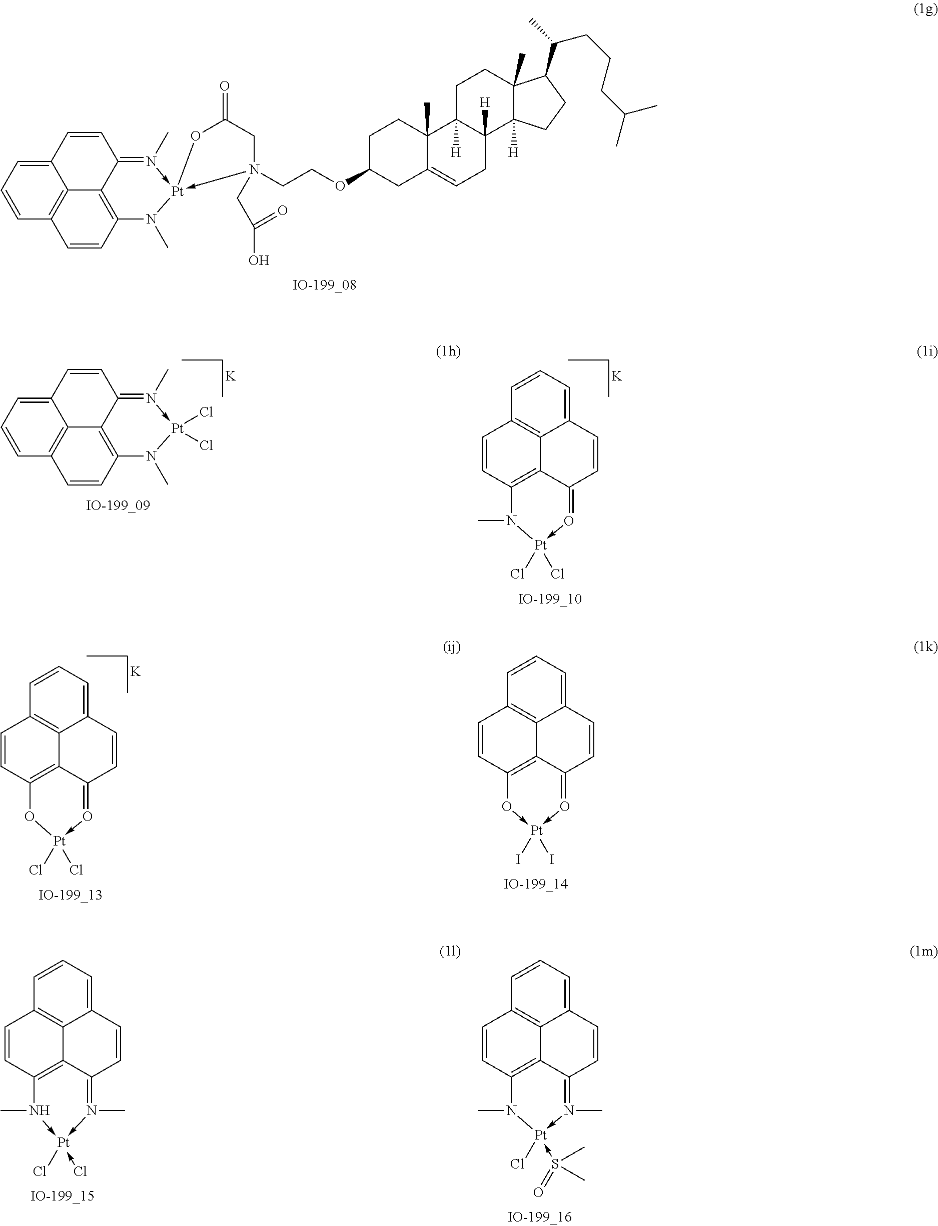

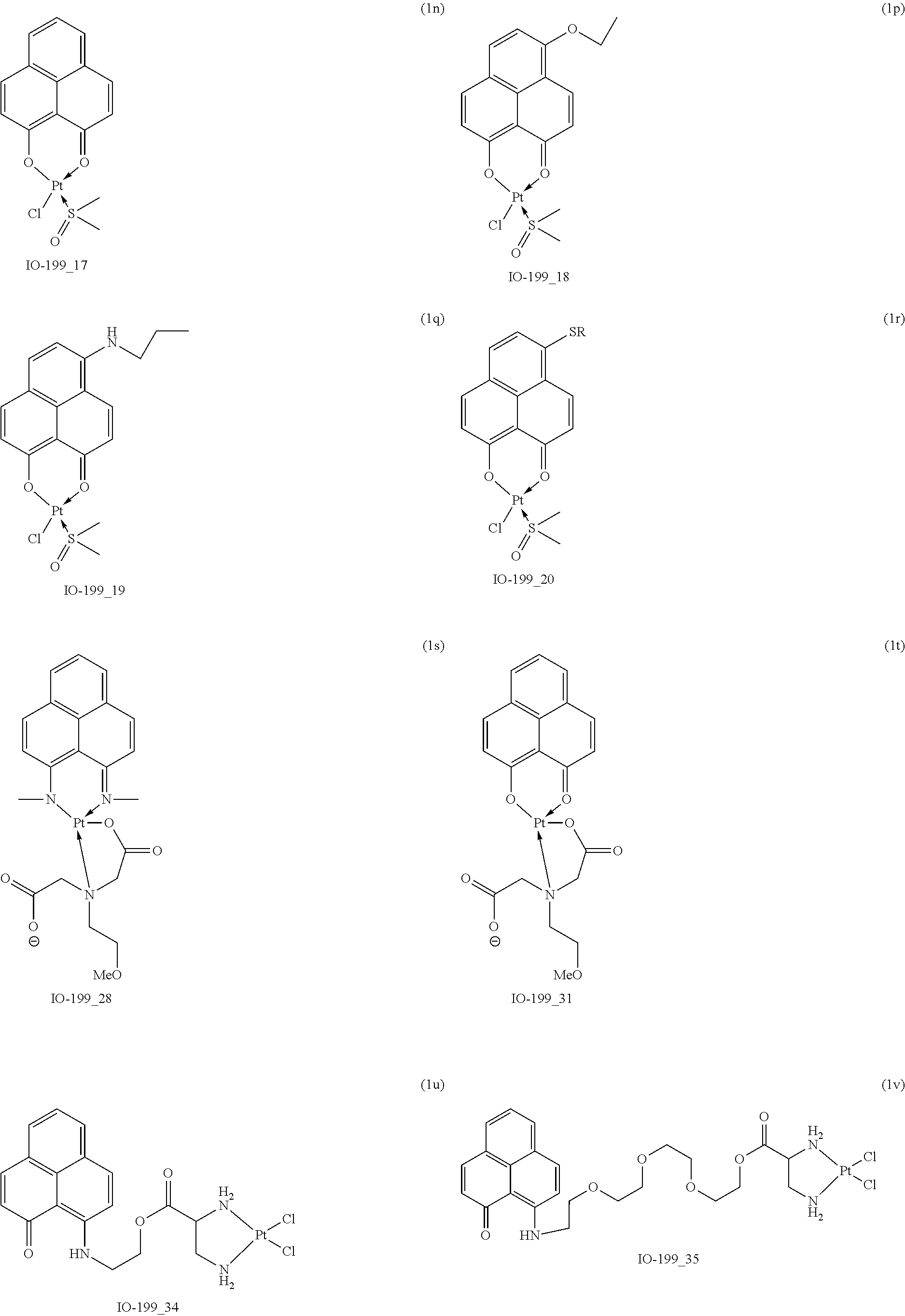

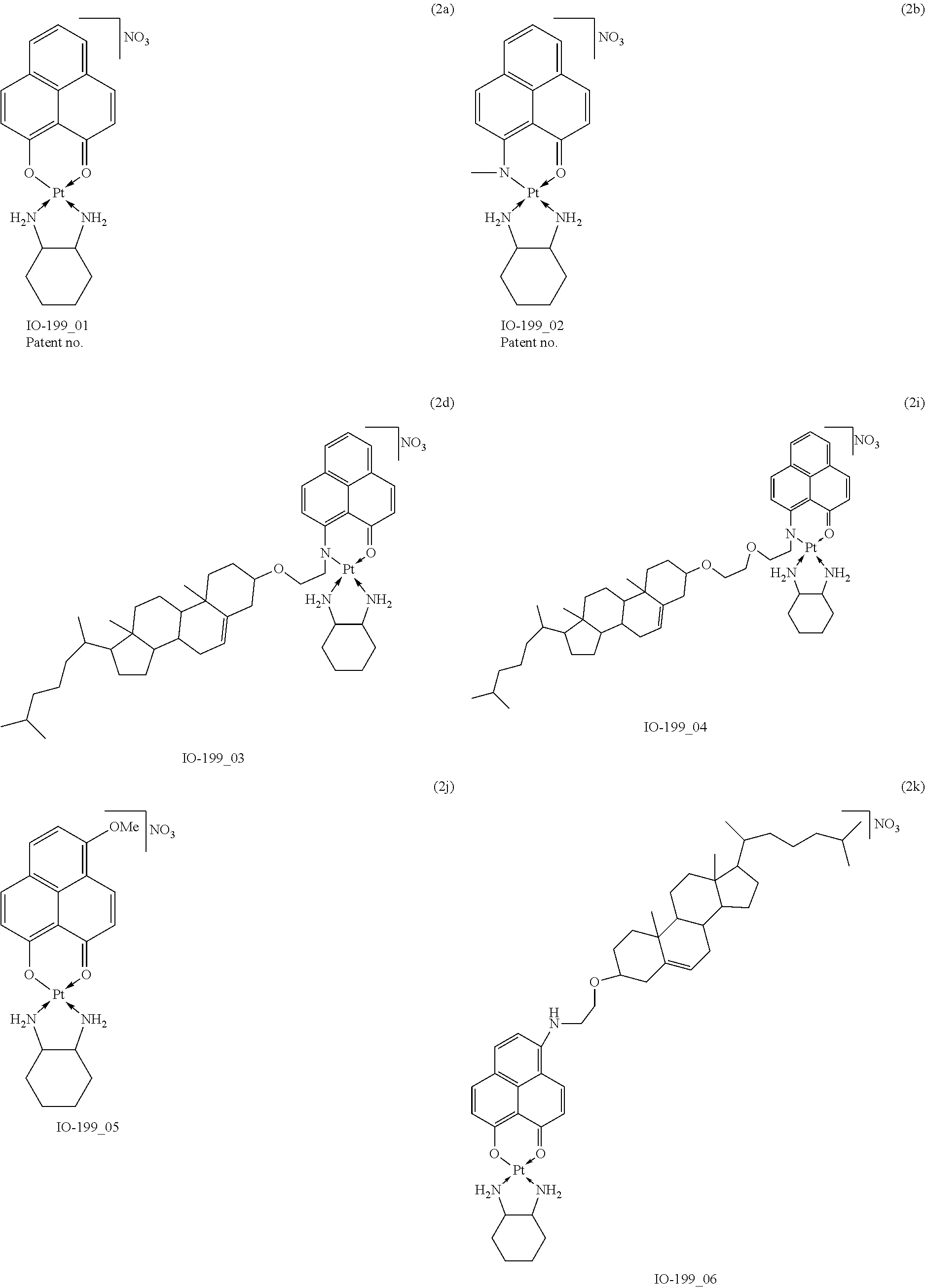

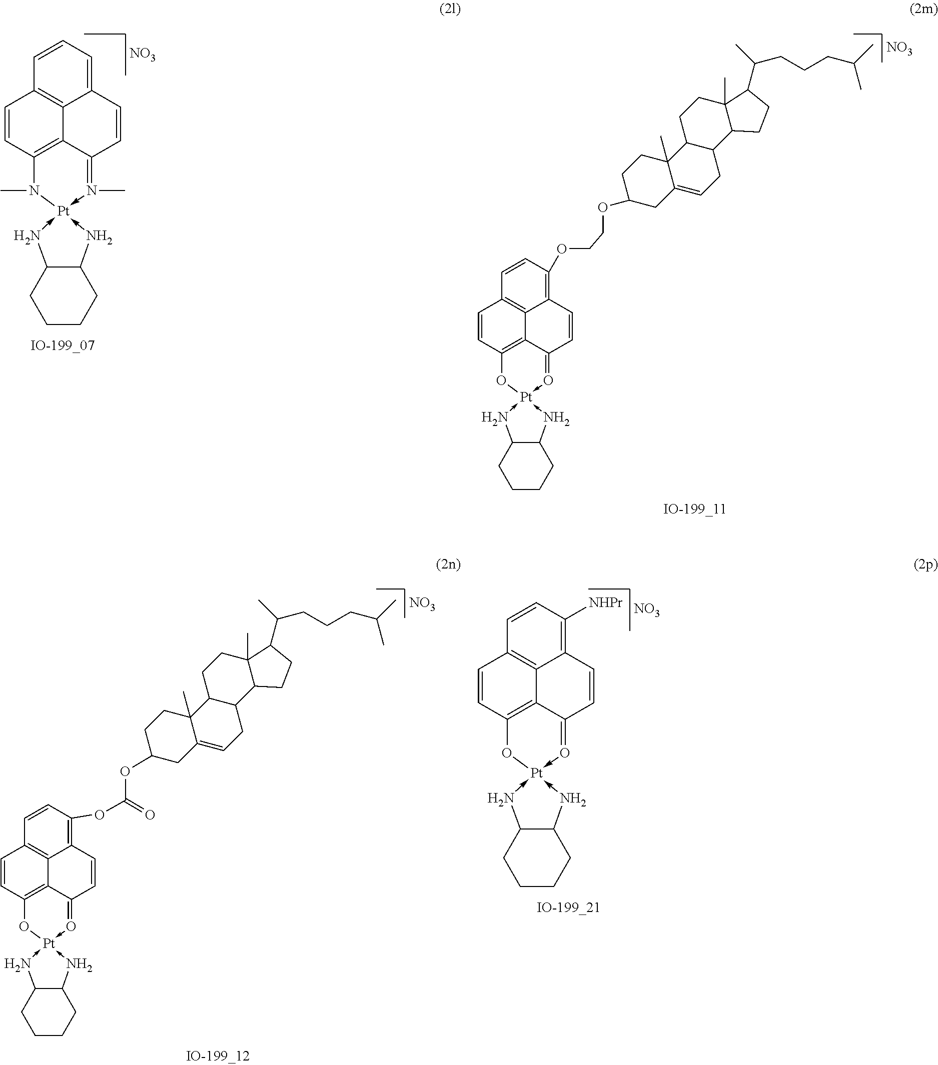

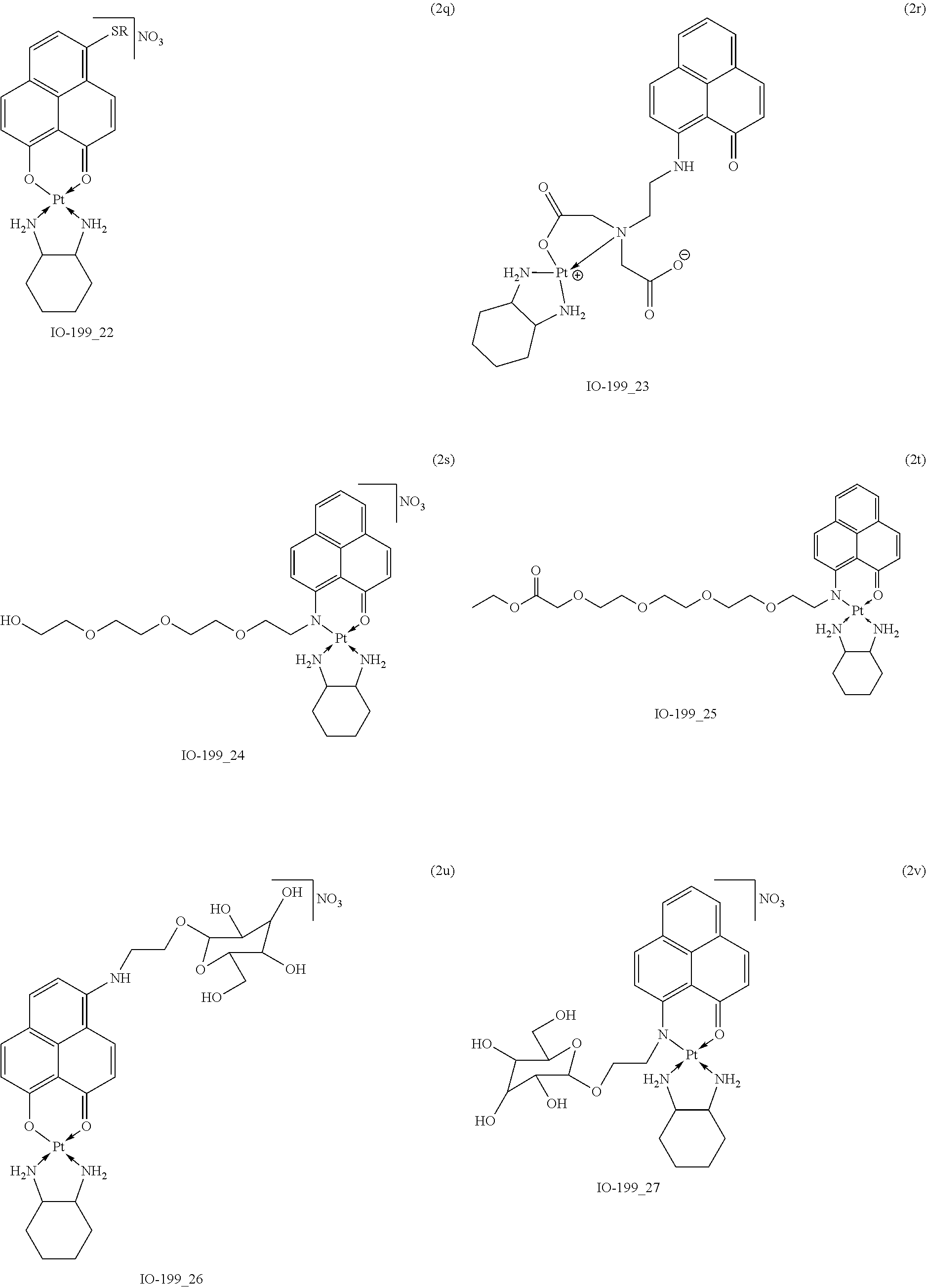

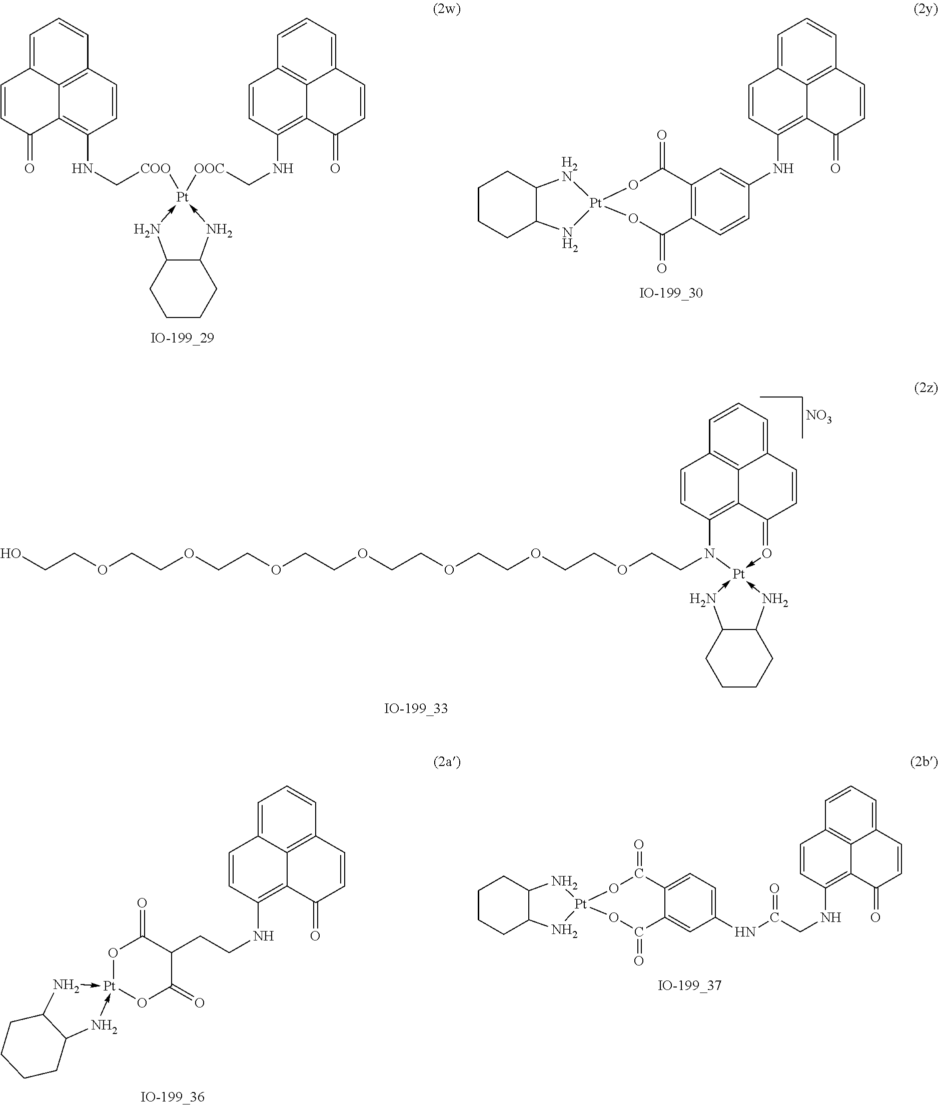

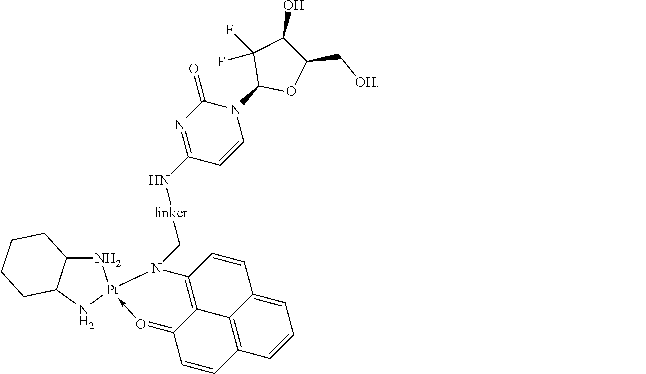

and IO-199_37 to IO-199_40, where R is optionally substituted alkyl.

Some of the compounds above are depicted with a nitrate counter anion. It is to be understood that other counter anions beside nitrate can also be used. Exemplary counter anions include, but are not limited to, nitrate anions, chlorine ions, bromine ions, nitrite anions, phosphate anions, sulfate anions, sulfite anions, acetate anions, and sulfonate anions. In some embodiments, the counter anion, if present, is nitrate or chlorine.

Some of the compounds above are depicted with a potassium counter cation. It is to be understood that other counter cations beside potassium can also be used. Exemplary counter cations include, but are not limited to, alkali metal ions (e.g., sodium, potassium, and lithium), alkaline earth metal ions (e.g., calcium and magnesium), ammonium, alkyl ammonium (e.g., dialkylammonium, trialkylammonium, and tetraalkylammonium wherein alkyl is optionally substituted by hydroxyl, fluoride, or aryl), and five to seven membered heterocyclic groups having a positively charged nitrogen atom (e.g, a pyrrolium ion, pyrazolium ion, pyrrolidinium ion, imidazolium ion, triazolium ion, isoxazolium ion, oxazolium ion, thiazolium ion, isothiazolium ion, oxadiazolium ion, oxatriazolium ion, dioxazolium ion, oxathiazolium ion, pyridinium ion, pyridazinium ion, pyrimidinium ion, pyrazinium ion, piperazinium ion, triazinium ion, oxazinium ion, piperidinium ion, oxathiazinium ion, oxadiazinium ion, and morpholinium ion). In some embodiments, the counter cation, if present, is potassium.

The linker in compounds IO-199_37 to IO-199_40 can be a linker described herein. Without limitations, the linker can be a polyethylene glycol. In some embodiments, the linker is —CH₂CH₂(OCH₂CH₂)ₙOC(O)O—, where n is 0, 1, 2, 3, 4, 5, 6, 7, 8, 9 or 10. In some embodiments, n is 0, 1, 3 or 7.

Synthesis of exemplary compounds disclosed herein is described in the Examples section. One of skill can easily adapt the synthetic methods detailed in the Examples section to prepare the compounds described herein, for example, the compounds of Formula I-V and the platinum containing compounds.

Without wishing to be bound by a theory, the platinum compounds disclosed herein have relatively better efficacy than oxaliplatin in breast and cancer cell lines. In some embodiments, the platinum compounds disclosed herein have about 25%, about 50%, about 75%, about 1-fold, about 5-folds, about 10-folds, about 15-folds, about 20-folds, about 25-folds or higher efficacy in cancer cells relative to cisplatin or oxaliplatin at equivalent dosage.

In some embodiments, the platinum compounds disclosed herein have about 25%, about 50%, about 75%, about 1-fold, about 5-folds, about 10-folds, about 15-folds, about 20-folds, about 25-folds or higher platinum uptake in cancer cells relative to cisplatin or oxaliplatin at equivalent dosage.

In addition, the platinum compounds disclosed herein also have higher accumulation of platinum in tissue, such as, but not limited to a tumor, relative to cisplatin and oxaliplatin when dosed at equivalent amount. For example, the compounds disclosed herein have about 25%, about 50%, about 75%, about 1-fold, about 5-folds, about 10-folds, about 15-folds, about 20-folds, about 25-folds or higher platinum accumulation tissue relative to cisplatin or oxaliplatin when dosed at equivalent amounts.

In an embodiment of the present disclosure, several variants of platinum compounds such as racemates, diastereomers and the likes are also provided.

The disclosure also provides particles comprising one or more of the platinum compounds described herein. Generally, the particle disclosed herein can be of any shape or form, e.g., spherical, rod, elliptical, cylindrical, capsule, or disc; and these particles can be part of a network or an aggregate.

In some embodiments, the particle is a microparticle or a nanoparticle. As used herein, the term “microparticle” refers to a particle having a particle size of about 1 μm to about 1000 μm. As used herein, the term “nanoparticle” refers to particle having a particle size of about 0.1 nm to about 1000 nm. Generally, the particles have any size from nm to millimeters. In some embodiments, the particles can have a size ranging from about 5 nm to about 5000 nm. In some embodiments, the particles have an average diameter of from about 50 nm to about 2500 nm. In some embodiments, the particles have an average diameter of from about 100 nm to about 2000 nm. In some embodiments, the particles have an average diameter of from about 150 nm to about 1700 nm. In some embodiments, the particles have an average diameter of from about 200 nm to about 1500 nm. In some embodiment, the particles have an average diameter of about 260 nm. In one embodiment, the particles have an average diameter of about 30 nm to about 150 nm. In some embodiments, the particles have an average diameter of about 100 nm to about 1000 nm, from about 200 nm to about 800 nm, from about 200 nm to about 700 nm, or from about 300 nm to about 700 nm.

In some embodiments, the particle has an average size of about 50 to about 1000 nm. In a further embodiment, the nanoparticles of the present invention are in the range of about 50 to about 500 nm. In another embodiment, the nanoparticles of the present invention are in the range of about 50 to about 500 nm. In one embodiment, the particle has a size of about 500 nm.

It will be understood by one of ordinary skill in the art that particles usually exhibit a distribution of particle sizes around the indicated “size.” Unless otherwise stated, the term “particle size” as used herein refers to the mode of a size distribution of particles, i.e., the value that occurs most frequently in the size distribution. Methods for measuring the particle size are known to a skilled artisan, e.g., by dynamic light scattering (such as photocorrelation spectroscopy, laser diffraction, low-angle laser light scattering (LALLS), and medium-angle laser light scattering (MALLS)), light obscuration methods (such as Coulter analysis method), or other techniques (such as rheology, and light or electron microscopy).

In some embodiments, the particles can be substantially spherical. What is meant by “substantially spherical” is that the ratio of the lengths of the longest to the shortest perpendicular axes of the particle cross section is less than or equal to about 1.5. Substantially spherical does not require a line of symmetry. Further, the particles can have surface texturing, such as lines or indentations or protuberances that are small in scale when compared to the overall size of the particle and still be substantially spherical. In some embodiments, the ratio of lengths between the longest and shortest axes of the particle is less than or equal to about 1.5, less than or equal to about 1.45, less than or equal to about 1.4, less than or equal to about 1.35, less than or equal to about 1.30, less than or equal to about 1.25, less than or equal to about 1.20, less than or equal to about 1.15 less than or equal to about 1.1. Without wishing to be bound by a theory, surface contact is minimized in particles that are substantially spherical, which minimizes the undesirable agglomeration of the particles upon storage. Many crystals or flakes have flat surfaces that can allow large surface contact areas where agglomeration can occur by ionic or non-ionic interactions. A sphere permits contact over a much smaller area.

In some embodiments, the particles have substantially the same particle size. Particles having a broad size distribution where there are both relatively big and small particles allow for the smaller particles to fill in the gaps between the larger particles, thereby creating new contact surfaces. A broad size distribution can result in larger spheres by creating many contact opportunities for binding agglomeration. The particles described herein are within a narrow size distribution, thereby minimizing opportunities for contact agglomeration. What is meant by a “narrow size distribution” is a particle size distribution that has a ratio of the volume diameter of the 90th percentile of the small spherical particles to the volume diameter of the 10th percentile less than or equal to 5. In some embodiments, the volume diameter of the 90th percentile of the small spherical particles to the volume diameter of the 10th percentile is less than or equal to 4.5, less than or equal to 4, less than or equal to 3.5, less than or equal to 3, less than or equal to 2.5, less than or equal to 2, less than or equal to 1.5, less than or equal to 1.45, less than or equal to 1.40, less than or equal to 1.35, less than or equal to 1.3, less than or equal to 1.25, less than or equal to 1.20, less than or equal to 1.15, or less than or equal to 1.1.

Geometric Standard Deviation (GSD) can also be used to indicate the narrow size distribution. GSD calculations involved determining the effective cutoff diameter (ECD) at the cumulative less than percentages of 15.9% and 84.1%. GSD is equal to the square root of the ratio of the ECD less than 84.17% to ECD less than 15.9%. The GSD has a narrow size distribution when GSD<2.5. In some embodiments, GSD is less than 2, less than 1.75, or less than 1.5. In one embodiment, GSD is less than 1.8.

In addition to the platinum compounds disclosed herein, the particle can comprise co-lipids and/stabilizers. Additional lipids can be included in the particles for a variety of purposes, such as to prevent lipid oxidation, to stabilize the bilayer, to reduce aggregation during formation or to attach ligands onto the particle surface. Any of a number of additional lipids and/or other components can be present, including amphipathic, neutral, cationic, anionic lipids, and programmable fusion lipids. Such lipids and/or components can be used alone or in combination. One or more components of particle can comprise a ligand, e.g., a targeting ligand.

In some embodiments, the particle further comprises of a phospholipid. Without limitations, the phospholipids can be of natural origin, such as egg yolk or soybean phospholipids, or synthetic or semisynthetic origin. The phospholipids can be partially purified or fractionated to comprise pure fractions or mixtures of phosphatidyl cholines, phosphatidyl cholines with defined acyl groups having 6 to 22 carbon atoms, phosphatidyl ethanolamines, phosphatidyl inositols, phosphatidic acids, phosphatidyl serines, sphingomyelin or phosphatidyl glycerols. Suitable phospholipids include, but are not limited to, phosphatidylcholine, phosphatidylglycerol, lecithin, β,γ-dipalmitoyl-α-lecithin, sphingomyelin, phosphatidylserine, phosphatidic acid, N-(2,3-di(9-(Z)-octadecenyloxy))-prop-1-yl-N,N,N-trimethylammonium chloride, phosphatidylethanolamine, lysolecithin, lysophosphatidylethanolamine, phosphatidylinositol, cephalin, cardiolipin, cerebrosides, dicetylphosphate, dioleoylphosphatidylcholine, dipalmitoylphosphatidylcholine, dipalmitoylphosphatidylglycerol, dioleoylphosphatidylglycerol, palmitoyl-oleoyl-phosphatidylcholine, di-stearoyl-phosphatidylcholine, stearoyl-palmitoyl-phosphatidylcholine, di-palmitoyl-phosphatidylethanolamine, di-stearoyl-phosphatidylethanolamine, di-myrstoyl-phosphatidylserine, di-oleyl-phosphatidylcholine, dimyristoyl phosphatidyl choline (DMPC), dioleoylphosphatidylethanolamine (DOPE), palmitoyloleoylphosphatidylcholine (POPC), egg phosphatidylcholine (EPC), distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), phosphatidylethanolamine (POPE), dioleoyl-phosphatidylethanolamine 4-(N-maleimidomethyl)-cyclohexane-1-carboxylate (DOPE-mal), 1-stearoyl-2-oleoyl phosphatidylcholine (SOPC), 1,2-distearoyl-sn-glycem-3-phosphoethanolamine (DSPE), and any combinations thereof. Non-phosphorus containing lipids can also be used. These include, e.g., stearylamine, docecylamine, acetyl palmitate, fatty acid amides, and the like. Other phosphorus-lacking compounds, such as sphingolipids, glycosphingolipid families, diacylglycerols, and β-acyloxyacids, can also be used

In some embodiments, the phospholipid in the particle is selected from the group consisting of 1,2-Didecanoyl-sn-glycero-3-phosphocholine; 1,2-Dierucoyl-sn-glycero-3-phosphate (Sodium Salt); 1,2-Dierucoyl-sn-glycero-3-phosphocholine; 1,2-Dierucoyl-sn-glycero-3-phosphoethanolamine; 1,2-Dierucoyl-sn-glycero-3[Phospho-rac-(1-glycerol) (Sodium Salt); 1,2-Dilinoleoyl-sn-glycero-3-phosphocholine; 1,2-Dilauroyl-sn-glycero-3-phosphate (Sodium Salt); 1,2-Dilauroyl-sn-glycero-3-phosphocholine; 1,2-Dilauroyl-sn-glycero-3-phosphoethanolamine; 1,2-Dilauroyl-sn-glycero-3 [Phospho-rac-(1-glycerol) (Sodium Salt); 1,2-Dilauroyl-sn-glycero-3[Phospho-rac-(1-glycerol) (Ammonium Salt); 1,2-Dilauroyl-sn-glycero-3-phosphoserine (Sodium Salt); 1,2-Dimyristoyl-sn-glycero-3-phosphate (Sodium Salt); 1,2-Dimyristoyl-sn-glycero-3-phosphocholine; 1,2-Dimyristoyl-sn-glycero-3-phosphoethanolamine; 1,2-Dimyristoyl-sn-glycero-3 [Phospho-rac-(1-glycerol) (Sodium Salt); 1,2-Dimyristoyl-sn-glycero-3 [Phospho-rac-(1-glycerol) (Ammonium Salt); 1,2-Dimyristoyl-sn-glycero-3 [Phospho-rac-(1-glycerol) (Sodium/Ammonium Salt); 1,2-Dimyristoyl-sn-glycero-3-phosphoserine (Sodium Salt); 1,2-Dioleoyl-sn-glycero-3-phosphate (Sodium Salt); 1,2-Dioleoyl-sn-glycero-3-phosphocholine; 1,2-Dioleoyl-sn-glycero-3-phosphoethanolamine; 1,2-Dioleoyl-sn-glycero-3 [Phospho-rac-(1-glycerol) (Sodium Salt); 1,2-Dioleoyl-sn-glycero-3-phosphoserine (Sodium Salt); 1,2-Dipalmitoyl-sn-glycero-3-phosphate (Sodium Salt); 1,2-Dipalmitoyl-sn-glycero-3-phosphocholine; 1,2-Dipalmitoyl-sn-glycero-3-phosphoethanolamine; 1,2-Dipalmitoyl-sn-glycero-3 [Phospho-rac-(1-glycerol) (Sodium Salt); 1,2-Dipalmitoyl-sn-glycero-3 [Phospho-rac-(1-glycerol) (Ammonium Salt); 1,2-Dipalmitoyl-sn-glycero-3-phosphoserine (Sodium Salt); 1,2-Distearoyl-sn-glycero-3-phosphate (Sodium Salt); 1,2-Distearoyl-sn-glycero-3-phosphocholine; 1,2-Distearoyl-sn-glycero-3-phosphoethanolamine; 1,2-Distearoyl-sn-glycero-3 [Phospho-rac-(1-glycerol) (Sodium Salt); 1,2-Distearoyl-sn-glycero-3 [Phospho-rac-(1-glycerol) (Ammonium Salt); 1,2-Distearoyl-sn-glycero-3-phosphoserine (Sodium Salt); Egg-PC; Hydrogenated Egg PC; Hydrogenated Soy PC; 1-Myristoyl-sn-glycero-3-phosphocholine; 1-Palmitoyl-sn-glycero-3-phosphocholine; 1-Stearoyl-sn-glycero-3-phosphocholine; 1-Myristoyl-2-palmitoyl-sn-glycero 3-phosphocholine; 1-Myristoyl-2-stearoyl-sn-glycero-3-phosphocholine; 1-Palmitoyl-2-myristoyl-sn-glycero-3-phosphocholine; 1-Palmitoyl-2-oleoyl-sn-glycero-3-phosphocholine; 1-Palmitoyl-2-oleoyl-sn-glycero-3-phosphoethanolamine; 1-Palmitoyl-2-oleoyl-sn-glycero-3 [Phospho-rac-(1-glycerol)](Sodium Salt); 1-Palmitoyl-2-stearoyl-sn-glycero-3-phosphocholine; 1-Stearoyl-2-myristoyl-sn-glycero-3-phosphocholine; 1-Stearoyl-2-oleoyl-sn-glycero-3-phosphocholine; and 1-Stearoyl-2-palmitoyl-sn-glycero-3-phosphocholine. In some embodiments, the phospholipid is SPOC, egg PC, or Hydrogenated Soy PC (HSPC). In one, the phospholipid in the composition is HSPC.

In some embodiments, the particle further comprises a polyethylene glycol (PEG). The PEG can be included in the particle by itself or conjugated with a component present in the particle. For example, the PEG can be conjugated with the platinum based compound or a co-lipid/stabilizer component of the particle. In some embodiments, the PEG is conjugated with a co-lipid component of the particle. Without limitations, the PEG can be conjugated with any co-lipid. For example, the PEG conjugated co-lipid can be selected from the group consisting of PEG conjugated diacylglycerols and dialkylglycerols, PEG-conjugated phosphatidylethanolamine, PEG conjugated to phosphatidic acid, PEG conjugated ceramides (see, U.S. Pat. No. 5,885,613), PEG conjugated dialkylamines, PEG conjugated 1,2-diacyloxypropan-3-amines, and PEG conjugated to 1,2-distearoyl-sn-glycem-3-phosphoethanolamine (DSPE), and any combinations thereof. In some embodiments, the PEG conjugated lipid is 1,2-distearoyl-sn-glycem-3-phosphoethanolamine-N-[amino(polyethylene glycol)-2000] (DSPE-PEG2000).

In some embodiments, the particle further comprises a surfactant. Surfactants find wide application in formulations such as emulsions (including microemulsions) and liposomes. The most common way of classifying and ranking the properties of the many different types of surfactants, both natural and synthetic, is by the use of the hydrophile/lipophile balance (HLB). The nature of the hydrophilic group (also known as the “head”) provides the most useful means for categorizing the different surfactants used in formulations (Rieger, in Pharmaceutical Dosage Forms, Marcel Dekker, Inc., New York, N.Y., 1988, p. 285).

If the surfactant molecule is not ionized, it is classified as a nonionic surfactant. Nonionic surfactants find wide application in pharmaceutical and cosmetic products and are usable over a wide range of pH values. In general, their HLB values range from 2 to about 18 depending on their structure. Nonionic surfactants include nonionic esters such as ethylene glycol esters, propylene glycol esters, glyceryl esters, polyglyceryl esters, sorbitan esters, sucrose esters, and ethoxylated esters. Nonionic alkanolamides and ethers such as fatty alcohol ethoxylates, propoxylated alcohols, and ethoxylated/propoxylated block polymers are also included in this class. The polyoxyethylene surfactants are the most popular members of the nonionic surfactant class.

If the surfactant molecule carries a negative charge when it is dissolved or dispersed in water, the surfactant is classified as anionic. Anionic surfactants include carboxylates such as soaps, acyl lactylates, acyl amides of amino acids, esters of sulfuric acid such as alkyl sulfates and ethoxylated alkyl sulfates, sulfonates such as alkyl benzene sulfonates, acyl isethionates, acyl taurates and sulfosuccinates, and phosphates. The most important members of the anionic surfactant class are the alkyl sulfates and the soaps.

If the surfactant molecule carries a positive charge when it is dissolved or dispersed in water, the surfactant is classified as cationic. Cationic surfactants include quaternary ammonium salts and ethoxylated amines. The quaternary ammonium salts are the most used members of this class.

If the surfactant molecule has the ability to carry either a positive or negative charge, the surfactant is classified as amphoteric. Amphoteric surfactants include acrylic acid derivatives, substituted alkylamides, N-alkylbetaines and phosphatides.

The use of surfactants in drug products, formulations and in emulsions has been reviewed (Rieger, in Pharmaceutical Dosage Forms, Marcel Dekker, Inc., New York, N.Y., 1988, p. 285).

In some embodiments, the particle can further comprise acationic lipid. Exemplary cationic lipids include, but are not limited to, N,N-dioleyl-N,N-dimethylammonium chloride (DODAC), N,N-distearyl-N,N-dimethylammonium bromide (DDAB), N-(1-(2,3-dioleoyloxy)propyl)-N,N,N-trimethylammonium chloride (DOTAP), N-(1-(2,3-dioleyloxy)propyl)-N,N,N-trimethylammonium chloride (DOTMA), N,N-dimethyl-2,3-dioleyloxy)propylamine (DODMA), 1,2-DiLinoleyloxy-N,N-dimethylaminopropane (DLinDMA), 1,2-Dilinolenyloxy-N,N-dimethylaminopropane (DLenDMA), 1,2-Dilinoleylcarbamoyloxy-3-dimethylaminopropane (DLin-C-DAP), 1,2-Dilinoleyoxy-3-(dimethylamino)acetoxypropane (DLin-DAC), 1,2-Dilinoleyoxy-3-morpholinopropane (DLin-MA), 1,2-Dilinoleoyl-3-dimethylaminopropane (DLinDAP), 1,2-Dilinoleylthio-3-dimethylaminopropane (DLin-S-DMA), 1-Linoleoyl-2-linoleyloxy-3-dimethylaminopropane (DLin-2-DMAP), 1,2-Dilinoleyloxy-3-trimethylaminopropane chloride salt (DLin-TMA.C1), 1,2-Dilinoleoyl-3-trimethylaminopropane chloride salt (DLin-TAP.C1), 1,2-Dilinoleyloxy-3-(N-methylpiperazino)propane (DLin-MPZ), or 3-(N,N-Dilinoleylamino)-1,2-propanediol (DLinAP), 3-(N,N-Dioleylamino)-1,2-propanedio (DOAP), 1,2-Dilinoleyloxo-3-(2-N,N-dimethylamino)ethoxypropane (DLin-EG-DMA), 1,2-Dilinolenyloxy-N,N-dimethylaminopropane (DLinDMA), 2,2-Dilinoleyl-4-dimethylaminomethyl-[1,3]-dioxolane (DLin-K-DMA) or analogs thereof, (3aR,5s,6aS)—N,N-dimethyl-2,2-di((9Z,12Z)-octadeca-9,12-dienyl)tetrahydro-3aH-cyclopenta[d][1,3]dioxol-5-amine (ALN100), (6Z,9Z,28Z,31Z)-heptatriaconta-6,9,28,31-tetraen-19-yl 4-(dimethylamino)butanoate (MC3), 1,1′-(2-(4-(2-((2-(bis(2-hydroxydodecyl)amino)ethyl)(2-hydroxydodecyl)amino)ethyl)piperazin-1-yl)ethylazanediyl)didodecan-2-ol (Tech Gi), or a mixture thereof.

In some embodiments, the particle further comprises a non-cationic lipid. The non-cationic lipid can be an anionic lipid or a neutral lipid including, but not limited to, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoylphosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), 16-O-monomethyl PE, 16-O-dimethyl PE, 18-1-trans PE, 1-stearoyl-2-oleoyl-phosphatidyethanolamine (SOPE), cholesterol, or a mixture thereof.

The conjugated lipids that inhibits aggregation of particles can also be included in the particles disclosed herein. Such lipids include, but ar not limited to, a polyethyleneglycol (PEG)-lipid including, without limitation, a PEG-diacylglycerol (DAG), a PEG-dialkyloxypropyl (DAA), a PEG-phospholipid, a PEG-ceramide (Cer), or a mixture thereof. The PEG-DAA conjugate can be, for example, a PEG-dilauryloxypropyl (C₁₂), a PEG-dimyristyloxypropyl (C₁₄), a PEG-dipalmityloxypropyl (C₁₆), or a PEG-distearyloxypropyl (Cis). The conjugated lipid that prevents aggregation of particles can be from 0.01 mol % to about 20 mol % or about 2 mol % of the total lipid present in the particle.

In some embodiments, the particle is in the form of a liposome, vesicle, or emulsion. As used herein, the term “liposome” encompasses any compartment enclosed by a lipid layer. Liposomes can have one or more lipid membranes. Liposomes can be characterized by membrane type and by size. Small unilamellar vesicles (SUVs) have a single membrane and typically range between 0.02 and 0.05 μm in diameter; large unilamellar vesicles (LUVS) are typically larger than 0.05 μm. Oligolamellar large vesicles and multilamellar vesicles have multiple, usually concentric, membrane layers and are typically larger than 0.1 μm. Liposomes with several nonconcentric membranes, i.e., several smaller vesicles contained within a larger vesicle, are termed multivesicular vesicles.

In order to form a liposome the lipid molecules comprise elongated non-polar (hydrophobic) portions and polar (hydrophilic) portions. The hydrophobic and hydrophilic portions of the molecule are preferably positioned at two ends of an elongated molecular structure. When such lipids are dispersed in water they spontaneously form bilayer membranes referred to as lamellae. The lamellae are composed of two mono layer sheets of lipid molecules with their non-polar (hydrophobic) surfaces facing each other and their polar (hydrophilic) surfaces facing the aqueous medium. The membranes formed by the lipids enclose a portion of the aqueous phase in a manner similar to that of a cell membrane enclosing the contents of a cell. Thus, the bilayer of a liposome has similarities to a cell membrane without the protein components present in a cell membrane.

A liposome composition can be prepared by a variety of methods that are known in the art. See e.g., U.S. Pat. No. 4,235,871, No. 4,897,355 and No. 5,171,678; published PCT applications WO 96/14057 and WO 96/37194; Felgner, P. L. et al., Proc. Natl. Acad. Sci., USA (1987) 8:7413-7417, Bangham, et al. M. Mol. Biol. (1965) 23:238, Olson, et al. Biochim. Biophys. Acta (1979) 557:9, Szoka, et al. Proc. Natl. Acad. Sci. (1978) 75: 4194, Mayhew, et al. Biochim. Biophys. Acta (1984) 775:169, Kim, et al. Biochim. Biophys. Acta (1983) 728:339, and Fukunaga, et al. Endocrinol. (1984) 115:757, content of all of which is incorporated herein by reference in its entirety.

The liposomes can be prepared to have substantially homogeneous sizes in a selected size range. One effective sizing method involves extruding an aqueous suspension of the liposomes through a series of polycarbonate membranes having a selected uniform pore size; the pore size of the membrane will correspond roughly with the largest sizes of liposomes produced by extrusion through that membrane. See e.g., U.S. Pat. No. 4,737,323, content of which is incorporated herein by reference in its entirety.

The particles can also be in the form of an emulsion. Emulsions are typically heterogeneous systems of one liquid dispersed in another in the form of droplets (Idson, in Pharmaceutical Dosage Forms, Lieberman, Rieger and Banker (Eds.), 1988, Marcel Dekker, Inc., New York, N.Y., volume 1, p. 199; Rosoff, in Pharmaceutical Dosage Forms, Lieberman, Rieger and Banker (Eds.), 1988, Marcel Dekker, Inc., New York, N.Y., Volume 1, p. 245; Block in Pharmaceutical Dosage Forms, Lieberman, Rieger and Banker (Eds.), 1988, Marcel Dekker, Inc., New York, N.Y., volume 2, p. 335; Higuchi et al., in Remington's Pharmaceutical Sciences, Mack Publishing Co., Easton, Pa., 1985, p. 301). Emulsions are often biphasic systems comprising two immiscible liquid phases intimately mixed and dispersed with each other. In general, emulsions may be of either the water-in-oil (w/o) or the oil-in-water (o/w) variety. When an aqueous phase is finely divided into and dispersed as minute droplets into a bulk oily phase, the resulting composition is called water-in-oil (w/o) emulsion. Alternatively, when an oily phase is finely divided into and dispersed as minute droplets into a bulk aqueous phase, the resulting composition is called an oil-in-water (o/w) emulsion. Emulsions can contain additional components in addition to the dispersed phases, and the conjugate disclosed herein can be present as a solution in either the aqueous phase or the oily phase or itself as a separate phase. Pharmaceutical excipients such as emulsifiers, stabilizers, dyes, and anti-oxidants can also be present in emulsions as needed. Pharmaceutical emulsions can also be multiple emulsions that are comprised of more than two phases such as, for example, in the case of oil-in-water-in-oil (o/w/o) and water-in-oil-in-water (w/o/w) emulsions. Such complex formulations often provide certain advantages that simple binary emulsions do not. Multiple emulsions in which individual oil droplets of an o/w emulsion enclose small water droplets constitute a w/o/w emulsion. Likewise a system of oil droplets enclosed in globules of water stabilized in an oily continuous phase provides an o/w/o emulsion.

Emulsions are characterized by little or no thermodynamic stability. Often, the dispersed or discontinuous phase of the emulsion is well dispersed into the external or continuous phase and maintained in this form through the means of emulsifiers or the viscosity of the formulation. Either of the phases of the emulsion may be a semisolid or a solid, as is the case of emulsion-style ointment bases and creams. Other means of stabilizing emulsions entail the use of emulsifiers that may be incorporated into either phase of the emulsion. Emulsifiers can broadly be classified into four categories: synthetic surfactants, naturally occurring emulsifiers, absorption bases, and finely dispersed solids (Idson, in Pharmaceutical Dosage Forms, Lieberman, Rieger and Banker (Eds.), 1988, Marcel Dekker, Inc., New York, N.Y., volume 1, p. 199).

Synthetic surfactants, also known as surface active agents, have found wide applicability in the formulation of emulsions and have been reviewed in the literature (Rieger, in Pharmaceutical Dosage Forms, Lieberman, Rieger and Banker (Eds.), 1988, Marcel Dekker, Inc., New York, N.Y., volume 1, p. 285; Idson, in Pharmaceutical Dosage Forms, Lieberman, Rieger and Banker (Eds.), Marcel Dekker, Inc., New York, N.Y., 1988, volume 1, p. 199). Surfactants are typically amphiphilic and comprise a hydrophilic and a hydrophobic portion. The ratio of the hydrophilic to the hydrophobic nature of the surfactant has been termed the hydrophile/lipophile balance (HLB) and is a valuable tool in categorizing and selecting surfactants in the preparation of formulations. Surfactants may be classified into different classes based on the nature of the hydrophilic group: nonionic, anionic, cationic and amphoteric (Rieger, in Pharmaceutical Dosage Forms, Lieberman, Rieger and Banker (Eds.), 1988, Marcel Dekker, Inc., New York, N.Y., volume 1, p. 285).

Naturally occurring emulsifiers used in emulsion formulations include lanolin, beeswax, phosphatides, lecithin and acacia. Absorption bases possess hydrophilic properties such that they can soak up water to form w/o emulsions yet retain their semisolid consistencies, such as anhydrous lanolin and hydrophilic petrolatum. Finely divided solids have also been used as good emulsifiers especially in combination with surfactants and in viscous preparations. These include polar inorganic solids, such as heavy metal hydroxides, nonswelling clays such as bentonite, attapulgite, hectorite, kaolin, montmorillonite, colloidal aluminum silicate and colloidal magnesium aluminum silicate, pigments and nonpolar solids such as carbon or glyceryl tristearate.

A large variety of non-emulsifying materials can also be included in emulsion formulations and contribute to the properties of emulsions. These include, but are not limited to, fats, oils, waxes, fatty acids, fatty alcohols, fatty esters, humectants, hydrophilic colloids, preservatives and antioxidants (Block, in Pharmaceutical Dosage Forms, Lieberman, Rieger and Banker (Eds.), 1988, Marcel Dekker, Inc., New York, N.Y., volume 1, p. 335; Idson, in Pharmaceutical Dosage Forms, Lieberman, Rieger and Banker (Eds.), 1988, Marcel Dekker, Inc., New York, N.Y., volume 1, p. 199).

Hydrophilic colloids or hydrocolloids include naturally occurring gums and synthetic polymers such as polysaccharides (for example, acacia, agar, alginic acid, carrageenan, guar gum, karaya gum, and tragacanth), cellulose derivatives (for example, carboxymethylcellulose and carboxypropylcellulose), and synthetic polymers (for example, carbomers, cellulose ethers, and carboxyvinyl polymers). These disperse or swell in water to form colloidal solutions that stabilize emulsions by forming strong interfacial films around the dispersed-phase droplets and by increasing the viscosity of the external phase.

Since emulsions often contain a number of ingredients such as carbohydrates, proteins, sterols and phosphatides that may readily support the growth of microbes, these formulations often incorporate preservatives. Commonly used preservatives included in emulsion formulations include methyl paraben, propyl paraben, quaternary ammonium salts, benzalkonium chloride, esters of p-hydroxybenzoic acid, and boric acid. Antioxidants are also commonly added to emulsion formulations to prevent deterioration of the formulation. Antioxidants used can be free radical scavengers such as tocopherols, alkyl gallates, butylated hydroxyanisole, butylated hydroxytoluene, or reducing agents such as ascorbic acid and sodium metabisulfite, and antioxidant synergists such as citric acid, tartaric acid, and lecithin.

The applications of emulsion formulations via dermatological, oral and parenteral routes and methods for their manufacture have been reviewed in the literature (Idson, in Pharmaceutical Dosage Forms, Lieberman, Rieger and Banker (Eds.), 1988, Marcel Dekker, Inc., New York, N.Y., volume 1, p. 199). Emulsion formulations for oral delivery have been very widely used because of ease of formulation, as well as efficacy from an absorption and bioavailability standpoint (Rosoff, in Pharmaceutical Dosage Forms, Lieberman, Rieger and Banker (Eds.), 1988, Marcel Dekker, Inc., New York, N.Y., volume 1, p. 245; Idson, in Pharmaceutical Dosage Forms, Lieberman, Rieger and Banker (Eds.), 1988, Marcel Dekker, Inc., New York, N.Y., volume 1, p. 199).

Exemplary surfactants for inclusion in the particles disclosed herein include but are not limited to, ionic surfactants, non-ionic surfactants, Brij 96, polyoxyethylene oleyl ethers, polyglycerol fatty acid esters, tetraglycerol monolaurate (ML310), tetraglycerol monooleate (MO310), hexaglycerol monooleate (PO310), hexaglycerol pentaoleate (PO500), decaglycerol monocaprate (MCA750), decaglycerol monooleate (MO750), decaglycerol sequioleate (SO750), decaglycerol decaoleate (DAO750), alone or in combination with cosurfactants. The cosurfactant, usually a short-chain alcohol such as ethanol, 1-propanol, and 1-butanol, serves to increase the interfacial fluidity by penetrating into the surfactant film and consequently creating a disordered film because of the void space generated among surfactant molecules. Microemulsions can, however, be prepared without the use of cosurfactants and alcohol-free self-emulsifying microemulsion systems are known in the art. The aqueous phase can typically be, but is not limited to, water, an aqueous solution of the drug, glycerol, PEG300, PEG400, polyglycerols, propylene glycols, and derivatives of ethylene glycol. The oil phase can include, but is not limited to, materials such as Captex 300, Captex 355, Capmul MCM, fatty acid esters, medium chain (C8-C12) mono, di, and tri-glycerides, polyoxyethylated glyceryl fatty acid esters, fatty alcohols, polyglycolized glycerides, saturated polyglycolized C8-C10 glycerides, vegetable oils and silicone oil.

Microemulsions are particularly of interest from the standpoint of drug solubilization and the enhanced absorption of drugs. Lipid based microemulsions (both o/w and w/o) have been proposed to enhance the oral bioavailability of drugs, including peptides (see e.g., U.S. Pat. Nos. 6,191,105; 7,063,860; 7,070,802; 7,157,099; Constantinides et al., Pharmaceutical Research, 1994, 11, 1385-1390; Ritschel, Meth. Find. Exp. Clin. Pharmacol., 1993, 13, 205). Microemulsions afford advantages of improved drug solubilization, protection of drug from enzymatic hydrolysis, possible enhancement of drug absorption due to surfactant-induced alterations in membrane fluidity and permeability, ease of preparation, ease of oral administration over solid dosage forms, improved clinical potency, and decreased toxicity (see e.g., U.S. Pat. Nos. 6,191,105; 7,063,860; 7,070,802; 7,157,099; Constantinides et al., Pharmaceutical Research, 1994, 11, 1385; Ho et al., J. Pharm. Sci., 1996, 85, 138-143). Often microemulsions can form spontaneously when their components are brought together at ambient temperature. This can be particularly advantageous when formulating thermolabile drugs. Microemulsions have also been effective in the transdermal delivery of active components in both cosmetic and pharmaceutical applications. It is expected that the microemulsion compositions and formulations of the present invention will facilitate the increased systemic absorption of the platinum based compounds from the gastrointestinal tract, as well as improve the local cellular uptake of platinum based compounds disclosed herein.

Without wishing to be bound by a theory, nanoparticles disclosed herein have higher uptake of platinum in cancer cells relative to cisplatin and oxaliplatin. In some embodiments, the nanoparticles disclosed herein have about 25%, about 50%, about 75%, about 1-fold, about 5-folds, about 10-folds, about 15-folds, about 20-folds, about 25-folds or higher platinum uptake in cancer cells relative to cisplatin or oxaliplatin at equivalent dosage.

In addition, the nanoparticles disclosed herein also have higher accumulation of platinum in tissue, such as, but not limited to a tumor, relative to cisplatin and oxaliplatin when dosed at equivalent amount. For example, the nanoparticles disclosed herein have about 25%, about 50%, about 75%, about 1-fold, about 5-folds, about 10-folds, about 15-folds, about 20-folds, about 25-folds or higher platinum accumulation tissue relative to cisplatin or oxaliplatin when dosed at equivalent amounts.

Without wishing to be bound by a theory, the nanoparticle compositions of the present disclosure show significant cancer cell killing efficacy. Exemplary nanoparticles were tested in different cancer cell lines and it was observed that the compounds demonstrated significantly better cell killing efficacy than the control compounds such as conventionally known platinum drugs oxaliplatin, cisplatin, oxaliplatin, carboplatin, paraplatin and sartraplatin.

Accordingly, in another aspect, described herein is a method of treating cancer. Generally, the method comprises administering a therapeutically effective amount of a platinum based compounds disclosed herein to a subject in need thereof.

In yet another aspect, described herein is a method of imaging a cancer or tumor. Generally, the method comprises administering a compound, a platinum based compound or nanoparticle disclosed herein to a subject.

Without limitations, the compounds, complexes and nanoparticles described herein can also be used for in vitro imaging. Thus, in vitro imaging methods using the compounds, complexes and nanoparticles described herein are also encompassed by the present invention.

The phrase “therapeutically-effective amount” as used herein means that amount of a compound, material, or composition comprising a compound of the present invention which is effective for producing some desired therapeutic effect in at least a sub-population of cells in an animal at a reasonable benefit/risk ratio applicable to any medical treatment. Determination of a therapeutically effective amount is well within the capability of those skilled in the art. Generally, a therapeutically effective amount can vary with the subject's history, age, condition, sex, as well as the severity and type of the medical condition in the subject, and administration of other agents alleviate the disease or disorder to be treated.

Usually the amount of active compounds is between 0.1-95% by weight of the preparation, preferably between 0.2-20% by weight in preparations for parenteral use and preferably between 1 and 50% by weight in preparations for oral administration.

Toxicity and therapeutic efficacy can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compositions that exhibit large therapeutic indices are preferred. As used herein, the term ED denotes effective dose and is used in connection with animal models. The term EC denotes effective concentration and is used in connection with in vitro models.

The data obtained from the cell culture assays and animal studies can be used in formulating a range of dosage for use in humans. The dosage of such compounds lies preferably within a range of circulating concentrations that include the ED50 with little or no toxicity. The dosage can vary within this range depending upon the dosage form employed and the route of administration utilized.

The therapeutically effective dose can be estimated initially from cell culture assays. A dose can be formulated in animal models to achieve a circulating plasma concentration range that includes the IC50 (i.e., the concentration of the therapeutic which achieves a half-maximal inhibition of symptoms) as determined in cell culture. Levels in plasma can be measured, for example, by high performance liquid chromatography. The effects of any particular dosage can be monitored by a suitable bioassay.

The dosage can be determined by a physician and adjusted, as necessary, to suit observed effects of the treatment. Generally, the compositions are administered so that the agent is given at a dose from 1 μg/kg to 150 mg/kg, 1 μg/kg to 100 mg/kg, 1 μg/kg to 50 mg/kg, 1 μg/kg to 20 mg/kg, 1 μg/kg to 10 mg/kg, 1 μg/kg to 1 mg/kg, 100 μg/kg to 100 mg/kg, 100 μg/kg to 50 mg/kg, 100 μg/kg to 20 mg/kg, 100 μg/kg to 10 mg/kg, 100 μg/kg to 1 mg/kg, 1 mg/kg to 100 mg/kg, 1 mg/kg to 50 mg/kg, 1 mg/kg to 20 mg/kg, 1 mg/kg to 10 mg/kg, 10 mg/kg to 100 mg/kg, 10 mg/kg to 50 mg/kg, or 10 mg/kg to 20 mg/kg. It is to be understood that ranges given here include all intermediate ranges, for example, the range 1 mg/kg to 10 mg/kg includes 1 mg/kg to 2 mg/kg, 1 mg/kg to 3 mg/kg, 1 mg/kg to 4 mg/kg, 1 mg/kg to 5 mg/kg, 1 mg/kg to 6 mg/kg, 1 mg/kg to 7 mg/kg, 1 mg/kg to 8 mg/kg, 1 mg/kg to 9 mg/kg, 2 mg/kg to 10 mg/kg, 3 mg/kg to 10 mg/kg, 4 mg/kg to 10 mg/kg, 5 mg/kg to 10 mg/kg, 6 mg/kg to 10 mg/kg, 7 mg/kg to 10 mg/kg, 8 mg/kg to 10 mg/kg, 9 mg/kg to 10 mg/kg, and the like. It is to be further understood that the ranges intermediate to the given above are also within the scope of this invention, for example, in the range 1 mg/kg to 10 mg/kg, dose ranges such as 2 mg/kg to 8 mg/kg, 3 mg/kg to 7 mg/kg, 4 mg/kg to 6 mg/kg, and the like.

In some embodiments, the compositions are administered at a dosage so that the agent has an in vivo concentration of less than 500 nM, less than 400 nM, less than 300 nM, less than 250 nM, less than 200 nM, less than 150 nM, less than 100 nM, less than 50 nM, less than 25 nM, less than 20, nM, less than 10 nM, less than 5 nM, less than 1 nM, less than 0.5 nM, less than 0.1 nM, less than 0.05, less than 0.01, nM, less than 0.005 nM, less than 0.001 nM after 15 mins, 30 mins, 1 hr, 1.5 hrs, 2 hrs, 2.5 hrs, 3 hrs, 4 hrs, 5 hrs, 6 hrs, 7 hrs, 8 hrs, 9 hrs, 10 hrs, 11 hrs, 12 hrs or more of time of administration.

With respect to duration and frequency of treatment, it is typical for skilled clinicians to monitor subjects in order to determine when the treatment is providing therapeutic benefit, and to determine whether to increase or decrease dosage, increase or decrease administration frequency, discontinue treatment, resume treatment or make other alteration to treatment regimen. The dosing schedule can vary from once a week to daily depending on a number of clinical factors, such as the subject's sensitivity to the polypeptides. The desired dose can be administered everyday or every third, fourth, fifth, or sixth day. The desired dose can be administered at one time or divided into subdoses, e.g., 2-4 subdoses and administered over a period of time, e.g., at appropriate intervals through the day or other appropriate schedule. Such sub-doses can be administered as unit dosage forms. In some embodiments of the aspects described herein, administration is chronic, e.g., one or more doses daily over a period of weeks or months. Examples of dosing schedules are administration daily, twice daily, three times daily or four or more times daily over a period of 1 week, 2 weeks, 3 weeks, 4 weeks, 1 month, 2 months, 3 months, 4 months, 5 months, or 6 months or more.

In some embodiments, the platinum based compound can be administrated to a subject in combination with a pharmaceutically active agent, e.g., a second therapeutic agent. Exemplary pharmaceutically active compound include, but are not limited to, those found in Harrison's Principles of Internal Medicine, 13^{th} Edition, Eds. T. R. Harrison et al. McGraw-Hill N.Y., NY; Physicians Desk Reference, 50^{th} Edition, 1997, Oradell N.J., Medical Economics Co.; Pharmacological Basis of Therapeutics, 8^{th} Edition, Goodman and Gilman, 1990; and United States Pharmacopeia, The National Formulary, USP XII NF XVII, 1990, the complete contents of all of which are incorporated herein by reference. The platinum based compound and the the second therapeutic agent can be administrated to the subject in the same pharmaceutical composition or in different pharmaceutical compositions (at the same time or at different times).

As used herein, the term “administer” refers to the placement of a composition into a subject by a method or route which results in at least partial localization of the composition at a desired site such that desired effect is produced. A compound or composition described herein can be administered by any appropriate route known in the art including, but not limited to, oral or parenteral routes, including intravenous, intramuscular, subcutaneous, transdermal, airway (aerosol), pulmonary, nasal, rectal, and topical (including buccal and sublingual) administration.

Exemplary modes of administration include, but are not limited to, injection, infusion, instillation, inhalation, or ingestion. “Injection” includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrastemal injection and infusion. In some embodiments, the compositions are administered by intravenous infusion or injection.

As used herein, the term “cancer” refers to an uncontrolled growth of cells that may interfere with the normal functioning of the bodily organs and systems. Cancers that migrate from their original location and seed vital organs can eventually lead to the death of the subject through the functional deterioration of the affected organs. Metastasis is a cancer cell or group of cancer cells, distinct from the primary tumor location resulting from the dissemination of cancer cells from the primary tumor to other parts of the body. At the time of diagnosis of the primary tumor mass, the subject may be monitored for the presence of in transit metastases, e.g., cancer cells in the process of dissemination. As used herein, the term cancer, includes, but is not limited to the following types of cancer, breast cancer, biliary tract cancer, bladder cancer, brain cancer including Glioblastomas and medulloblastomas; cervical cancer; choriocarcinoma; colon cancer; endometrial cancer; esophageal cancer, gastric cancer; hematological neoplasms including acute lymphocytic and myelogenous leukemia; T-cell acute lymphoblastic leukemia/lymphoma; hairy cell leukemia; chronic myelogenous leukemia, multiple myeloma; AIDS-associated leukemias and adult T-cell leukemia lymphoma; intraepithelial neoplasms including Bowen's disease and Paget's disease; liver cancer; lung cancer; lymphomas including Hodgkin's disease and lymphocytic lymphomas; neuroblastomas; oral cancer including squamous cell carcinoma; ovarian cancer including those arising from epithelial cells, stromal cells, germ cells and mesenchymal cells; pancreatic cancer; prostate cancer; rectal cancer; sarcomas including leiomyosarcoma, rhabdomyosarcoma, liposarcoma, fibrosarcoma, and osteosarcoma; skin cancer including melanoma, Merkel cell carcinoma, Kaposi's sarcoma, basal cell carcinoma, and squamous cell cancer; testicular cancer including germinal tumors such as seminoma, non-seminoma (teratomas, choriocarcinomas), stromal tumors, and germ cell tumors; thyroid cancer including thyroid adenocarcinoma and medullar carcinoma; and renal cancer including adenocarcinoma, Wilms tumor. Examples of cancer include but are not limited to, carcinoma, including adenocarcinoma, lymphoma, blastoma, melanoma, sarcoma, and leukemia. More particular examples of such cancers include squamous cell cancer, small-cell lung cancer, non-small cell lung cancer, gastrointestinal cancer, Hodgkin's and non-Hodgkin's lymphoma, pancreatic cancer, Glioblastoma, cervical cancer, ovarian cancer, liver cancer such as hepatic carcinoma and hepatoma, bladder cancer, breast cancer, colon cancer, colorectal cancer, endometrial carcinoma, salivary gland carcinoma, kidney cancer such as renal cell carcinoma and Wilms' tumors, basal cell carcinoma, melanoma, prostate cancer, vulval cancer, thyroid cancer, testicular cancer, esophageal cancer, and various types of head and neck cancer. Other cancers will be known to the artisan.

As used herein, the term “cancer” includes, but is not limited to, solid tumors and blood born tumors. The term cancer refers to disease of skin, tissues, organs, bone, cartilage, blood and vessels. The term “cancer” further encompasses primary and metastatic cancers. Examples of cancers that can be treated with the compounds of the invention include, but are not limited to, carcinoma, including that of the bladder, breast, colon, kidney, lung, ovary, pancreas, stomach, cervix, thyroid, and skin, including squamous cell carcinoma; hematopoietic tumors of lymphoid lineage, including, but not limited to, leukemia, acute lymphocytic leukemia, acute lymphoblastic leukemia, B-cell lymphoma, T-cell lymphoma, Hodgkins lymphoma, non-Hodgkins lymphoma, hairy cell lymphoma, and Burketts lymphoma; hematopoietic tumors of myeloid lineage including, but not limited to, acute and chronic myelogenous leukemias and promyelocytic leukemia; tumors of mesenchymal origin including, but not limited to, fibrosarcoma, rhabdomyosarcoma, and osteosarcoma; other tumors including melanoma, seminoma, tetratocarcinoma, neuroblastoma, and glioma; tumors of the central and peripheral nervous system including, but not limited to, astrocytoma, neuroblastoma, glioma, and schwannomas; and other tumors including, but not limited to, xenoderma, pigmentosum, keratoactanthoma, thyroid follicular cancer, and teratocarcinoma. The methods disclosed herein are useful for treating patients who have been previously treated for cancer, as well as those who have not previously been treated for cancer. Indeed, the methods and compositions of this invention can be used in first-line and second-line cancer treatments.

As used herein, the term “precancerous condition” has its ordinary meaning, i.e., an unregulated growth without metastasis, and includes various forms of hyperplasia and benign hypertrophy. Accordingly, a “precancerous condition” is a disease, syndrome, or finding that, if left untreated, can lead to cancer. It is a generalized state associated with a significantly increased risk of cancer. Premalignant lesion is a morphologically altered tissue in which cancer is more likely to occur than its apparently normal counterpart. Examples of premalignant conditions include, but are not limited to, oral leukoplakia, actinic keratosis (solar keratosis), Barrett's esophagus, atrophic gastritis, benign hyperplasia of the prostate, precancerous polyps of the colon or rectum, gastric epithelial dysplasia, adenomatous dysplasia, hereditary nonpolyposis colon cancer syndrome (HNPCC), Barrett's esophagus, bladder dysplasia, precancerous cervical conditions, and cervical dysplasia.

In some embodiments, the cancer is selected from the group consisting of: breast cancer; ovarian cancer; glioma; gastrointestinal cancer; prostate cancer; carcinoma, lung carcinoma, hepatocellular carcinoma, testicular cancer; cervical cancer; endometrial cancer; bladder cancer; head and neck cancer; lung cancer; gastro-esophageal cancer, and gynecological cancer.

In some embodiments, the methods described herein relate to treating a subject having or diagnosed as having cancer. Subjects having cancer can be identified by a physician using current methods of diagnosing cancer. Symptoms and/or complications of cancer which characterize these conditions and aid in diagnosis are well known in the art and include but are not limited to, growth of a tumor, impaired function of the organ or tissue harboring cancer cells, etc. Tests that may aid in a diagnosis of, e.g. cancer include, but are not limited to, tissue biopsies and histological examination. A family history of cancer, or exposure to risk factors for cancer (e.g. tobacco products, radiation, etc.) can also aid in determining if a subject is likely to have cancer or in making a diagnosis of cancer.

In some embodiments, the method further comprises co-administering one or more additional anti-cancer therapy to the patient. In some embodiments, the additional therapy is selected from the group consisting of surgery, chemotherapy, radiation therapy, thermotherapy, immunotherapy, hormone therapy, laser therapy, anti-angiogenic therapy, and any combinations thereof. In some embodiments, the additional therapy comprises administering an anti-cancer agent to the patient. In some embodiments, the method comprises co-administering the conjugate and an anti-cancer agent or chemotherapeutic agent to the subject.

As used herein, the term “anti-cancer agent” is refers to any compound (including its analogs, derivatives, prodrugs and pharmaceutically salts) or composition, which can be used to treat cancer. Anti-cancer compounds for use in the present invention include, but are not limited to, inhibitors of topoisomerase I and II, alkylating agents, microtubule inhibitors (e.g., taxol), and angiogenesis inhibitors. Exemplary anti-cancer compounds include, but are not limited to, paclitaxel (taxol); docetaxel; germicitibine; Aldesleukin; Alemtuzumab; alitretinoin; allopurinol; altretamine; amifostine; anastrozole; arsenic trioxide; Asparaginase; BCG Live; bexarotene capsules; bexarotene gel; bleomycin; busulfan intravenous; busulfanoral; calusterone; capecitabine; platinate; carmustine; carmustine with Polifeprosan Implant; celecoxib; chlorambucil; cladribine; cyclophosphamide; cytarabine; cytarabine liposomal; dacarbazine; dactinomycin; actinomycin D; Darbepoetin alfa; daunorubicin liposomal; daunorubicin, daunomycin; Denileukin diftitox, dexrazoxane; docetaxel; doxorubicin; doxorubicin liposomal; Dromostanolone propionate; Elliott's B Solution; epirubicin; Epoetin alfa estramustine; etoposide phosphate; etoposide (VP-16); exemestane; Filgrastim; floxuridine (intraarterial); fludarabine; fluorouracil (5-FU); fulvestrant; gemtuzumab ozogamicin; goserelin acetate; hydroxyurea; Ibritumomab Tiuxetan; idarubicin; ifosfamide; imatinib mesylate; Interferon alfa-2a; Interferon alfa-2b; irinotecan; letrozole; leucovorin; levamisole; lomustine (CCNU); mechlorethamine (nitrogenmustard); megestrol acetate; melphalan (L-PAM); mercaptopurine (6-MP); mesna; methotrexate; methoxsalen; mitomycin C; mitotane; mitoxantrone; nandrolone phenpropionate; Nofetumomab; LOddC; Oprelvekin; pamidronate; pegademase; Pegaspargase; Pegfilgrastim; pentostatin; pipobroman; plicamycin; mithramycin; porfimer sodium; procarbazine; quinacrine; Rasburicase; Rituximab; Sargramostim; streptozocin; talbuvidine (LDT); talc; tamoxifen; temozolomide; teniposide (VM-26); testolactone; thioguanine (6-TG); thiotepa; topotecan; toremifene; Tositumomab; Trastuzumab; tretinoin (ATRA); Uracil Mustard; valrubicin; valtorcitabine (monoval LDC); vinblastine; vinorelbine; zoledronate; and any mixtures thereof. In some embodiments, the anti-cancer agent is a paclitaxel-carbohydrate conjugate, e.g., a paclitaxel-glucose conjugate, as described in U.S. Pat. No. 6,218,367, content of which is herein incorporated by reference in its entirety.

In some embodiments, the anti-cancer agent is Gemcitabine.

The methods of the invention are especially useful in combination with anti-cancer treatments that involve administering a second drug that acts in a different phase of the cell cycle.

For administration to a subject, the platinum based compounds and/or particles comprising said platinum based compounds are provided in pharmaceutically acceptable compositions. Accordingly, the disclosure also provides pharmaceutical compositions comprising the platinum based compounds or particles as disclosed herein. These pharmaceutically acceptable compositions comprise a therapeutically effective amount of one or more of the platinum based compounds or particles described herein, formulated together with one or more pharmaceutically acceptable carriers (additives) and/or diluents. The said pharmaceutical compositions of the present invention are specially formulated for administration in solid or liquid form, including those adapted for the following: (1) oral administration, for example, drenches (aqueous or non-aqueous solutions or suspensions), lozenges, dragees, capsules, pills, tablets (e.g., those targeted for buccal, sublingual, and systemic absorption), boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; (8) transmucosally; or (9) nasally. Additionally, the compounds of the present disclosure can be implanted into a patient or injected using a drug delivery system.

As used herein, the term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the term “pharmaceutically-acceptable carrier” means a pharmaceutically-acceptable material, composition or vehicle, such as a liquid or solid filler, diluent, excipient, manufacturing aid (e.g., lubricant, talc magnesium, calcium or zincstearate, or steric acid), or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as com starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (S) excipients, such as cocoa butter and suppository waxes; (9) oils, such as peanut oil, cottonseed oil, safflower oil, sesame oil, olive oil, com oil and soybean oil; (10) glycols, such as propylene glycol; (11) polyols, such as glycerin, sorbitol, mannitol and polyethylene glycol (PEG); (12) esters, such as ethyl oleate and ethyllaurate; (13) agar; (14) buffering agents, such as magnesium hydroxide and aluminum hydroxide; (15) alginic acid; (16) pyrogen-free water; (17) isotonic saline; (IS) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C2-C12 alchols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient”, “carrier”, “pharmaceutically acceptable carrier” or the likes are used interchangeably herein.

In some embodiments, the pharmaceutical composition comprising a platinum based compound can be a parenteral dose form. Since administration of parenteral dosage forms typically bypasses the patient's natural defenses against contaminants, parenteral dosage forms are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, and emulsions. In addition, controlled-release parenteral dosage forms can be prepared for administration of a patient, including, but not limited to, DUROS®-type dosage forms and dose dumping.

Suitable vehicles that can be used to provide parenteral dosage forms of a composition as described herein are well known to those skilled in the art. Examples include, without limitation: sterile water; water for injection USP; saline solution; glucose solution; aqueous vehicles such as but not limited to, sodium chloride injection, Ringer's injection, dextrose Injection, dextrose and sodium chloride injection, and lactated Ringer's injection; water-miscible vehicles such as, but not limited to, ethyl alcohol, polyethylene glycol, and propylene glycol; and non-aqueous vehicles such as, but not limited to, corn oil, cottonseed oil, peanut oil, sesame oil, ethyl oleate, isopropyl myristate, and benzyl benzoate. Compounds that alter or modify the solubility of a pharmaceutically acceptable salt can also be incorporated into the parenteral dosage forms of the disclosure, including conventional and controlled-release parenteral dosage forms.

Pharmaceutical compositions can also be formulated to be suitable for oral administration, for example as discrete dosage forms, such as, but not limited to, tablets (including without limitation scored or coated tablets), pills, caplets, capsules, chewable tablets, powder packets, cachets, troches, wafers, aerosol sprays, or liquids, such as but not limited to, syrups, elixirs, solutions or suspensions in an aqueous liquid, a non-aqueous liquid, an oil-in-water emulsion, or a water-in-oil emulsion. Such compositions contain a predetermined amount of the pharmaceutically acceptable salt of the disclosed compounds, and may be prepared by methods of pharmacy well known to those skilled in the art. See generally, Remington: The Science and Practice of Pharmacy, 21st Ed., Lippincott, Williams, and Wilkins, Philadelphia Pa. (2005).

Conventional dosage forms generally provide rapid or immediate drug release from the formulation. Depending on the pharmacology and pharmacokinetics of the drug, use of conventional dosage forms can lead to wide fluctuations in the concentrations of the drug in a patient's blood and other tissues. These fluctuations can impact a number of parameters, such as dose frequency, onset of action, duration of efficacy, maintenance of therapeutic blood levels, toxicity, side effects, and the like. Advantageously, controlled-release formulations can be used to control a drug's onset of action, duration of action, plasma levels within the therapeutic window, and peak blood levels. In particular, controlled- or extended-release dosage forms or formulations can be used to ensure that the maximum effectiveness of a drug is achieved while minimizing potential adverse effects and safety concerns, which can occur both from under-dosing a drug (i.e., going below the minimum therapeutic levels) as well as exceeding the toxicity level for the drug. In some embodiments, a composition as described herein can be administered in a sustained release formulation.

Controlled-release pharmaceutical products have a common goal of improving drug therapy over that achieved by their non-controlled release counterparts. Ideally, the use of an optimally designed controlled-release preparation in medical treatment is characterized by a minimum of drug substance being employed to cure or control the condition in a minimum amount of time. Advantages of controlled-release formulations include: 1) extended activity of the drug; 2) reduced dosage frequency; 3) increased patient compliance; 4) usage of less total drug; 5) reduction in local or systemic side effects; 6) minimization of drug accumulation; 7) reduction in blood level fluctuations; 8) improvement in efficacy of treatment; 9) reduction of potentiation or loss of drug activity; and 10) improvement in speed of control of diseases or conditions. Kim, Cherng-ju, Controlled Release Dosage Form Design, 2 (Technomic Publishing, Lancaster, Pa.: 2000).

Most controlled-release formulations are designed to initially release an amount of drug (active ingredient) that promptly produces the desired therapeutic effect, and gradually and continually release other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled-release of an active ingredient can be stimulated by various conditions including, but not limited to, pH, ionic strength, osmotic pressure, temperature, enzymes, water, and other physiological conditions or compounds.

A variety of known controlled- or extended-release dosage forms, formulations, and devices can be adapted for use with the salts and compositions of the disclosure. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; 5,733,566; and 6,365,185 B1; each of which is incorporated herein by reference. These dosage forms can be used to provide slow or controlled-release of one or more active ingredients using, for example, hydroxypropylmethyl cellulose, other polymer matrices, gels, permeable membranes, osmotic systems (such as OROS® (Alza Corporation, Mountain View, Calif. USA)), or a combination thereof to provide the desired release profile in varying proportions.

### Some Selected Definitions

For convenience, certain terms employed herein, in the specification, examples and appended claims are collected herein. Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. The definitions are provided to aid in describing particular embodiments, and are not intended to limit the claimed invention, because the scope of the invention is limited only by the claims. Further, unless otherwise required by context, singular terms shall include pluralities and plural terms shall include the singular.

Unless defined otherwise, all technical and scientific terms used herein have the same meaning as those commonly understood to one of ordinary skill in the art to which this invention pertains. Although any known methods, devices, and materials may be used in the practice or testing of the invention, the methods, devices, and materials in this regard are described herein.

As used herein the term “comprising” or “comprises” is used in reference to compositions, methods, and respective component(s) thereof, that are essential to the invention, yet open to the inclusion of unspecified elements, whether essential or not.

The singular terms “a,” “an,” and “the” include plural referents unless context clearly indicates otherwise. Similarly, the word “or” is intended to include “and” unless the context clearly indicates otherwise.

Other than in the operating examples, or where otherwise indicated, all numbers expressing quantities of ingredients or reaction conditions used herein should be understood as modified in all instances by the term “about.” The term “about” when used in connection with percentages may mean±5% of the value being referred to. For example, about 100 means from 95 to 105.

Although methods and materials similar or equivalent to those described herein can be used in the practice or testing of this disclosure, suitable methods and materials are described below. The term “comprises” means “includes.” The abbreviation, “e.g.” is derived from the Latin exempli gratia, and is used herein to indicate a non-limiting example. Thus, the abbreviation “e.g.” is synonymous with the term “for example.”

The terms “decrease”, “reduced”, “reduction”, “decrease” or “inhibit” are all used herein generally to mean a decrease by a statistically significant amount. However, for avoidance of doubt, ““reduced”, “reduction” or “decrease” or “inhibit” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (e.g. absent level as compared to a reference sample), or any decrease between 10-100% as compared to a reference level.

The terms “increased”,“increase” or “enhance” or “activate” are all used herein to generally mean an increase by a statically significant amount; for the avoidance of any doubt, the terms “increased”, “increase” or “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

The term “statistically significant” or “significantly” refers to statistical significance and generally means at least two standard deviation (2SD) away from a reference level. The term refers to statistical evidence that there is a difference. It is defined as the probability of making a decision to reject the null hypothesis when the null hypothesis is actually true.

As used herein, the terms “treat,” “treatment,” “treating,” or “amelioration” refer to therapeutic treatments, wherein the object is to reverse, alleviate, ameliorate, inhibit, slow down or stop the progression or severity of a condition associated with a disease or disorder, e.g. cancer. The term “treating” includes reducing or alleviating at least one adverse effect or symptom of a condition, disease or disorder associated with a cancer. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of, or at least slowing of, progress or worsening of symptoms compared to what would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, remission (whether partial or total), and/or decreased mortality, whether detectable or undetectable. The term “treatment” of a disease also includes providing relief from the symptoms or side-effects of the disease (including palliative treatment).

As used herein, “management” or “managing” refers to preventing a disease or disorder from occurring in a subject, decreasing the risk of death due to a disease or disorder, delaying the onset of a disease or disorder, inhibiting the progression of a disease or disorder, partial or complete cure of a disease or disorder and/or adverse effect attributable to the said disease or disorder, obtaining a desired pharmacologic and/or physiologic effect (the effect may be prophylactic in terms of completely or partially preventing a disorder or disease or condition, or a symptom thereof and/or may be therapeutic in terms of a partial or complete cure for a disease or disorder and/or adverse effect attributable to the disease or disorder), relieving a disease or disorder (i.e. causing regression of the disease or disorder). Further, the present disclosure also envisages treating the said disease by administering the therapeutic composition of the instant disclosure.

The terms “subject” and “individual” are used interchangeably herein, and mean a human or animal. Usually the animal is a vertebrate such as a primate, rodent, domestic animal or game animal. Primates include chimpanzees, cynomolgus monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. Patient or subject includes any subset of the foregoing, e.g., all of the above, but excluding one or more groups or species such as humans, primates or rodents. In certain embodiments, the subject is a mammal, e.g., a primate, e.g., a human. The terms, “patient” and “subject” are used interchangeably herein. The terms, “patient” and “subject” are used interchangeably herein.

Preferably, the subject is a mammal. The mammal can be a human, non-human primate, mouse, rat, dog, cat, horse, or cow, but are not limited to these examples. Mammals other than humans can be advantageously used as subjects that represent animal models of cancer. In addition, the methods described herein can be used to treat domesticated animals and/or pets. A subject can be male or female. A subject can be one who has been previously diagnosed with or identified as suffering from cancer, but need not have already undergone treatment.

As used herein, the term “carbohydrate” refers to a compound which is either a carbohydrate per se made up of one or more monosaccharide units having at least 4, 5 or 6 carbon atoms (which may be linear, branched or cyclic) with an oxygen, nitrogen or sulfur atom bonded to each carbon atom; or a compound having as a part thereof a carbohydrate moiety made up of one or more monosaccharide units. Without limitations, the term “carbohydrate” is intended to include monomeric sugar alcohols, polysaccharides, oligosaccharides and other carbohydrate polymers. The sugar may be optionally substituted. Further, the sugar can have the L- or the D-conformation. Exemplary carbohydrates include, but are not limited to, erythrose, threose, ribose, arabinose, xylose, lyxose, ribulose, xylulose, allose, altrose, glucose, mannose, gulose, idose, galactose, telose, galactosamine, N-acetylgalactose, glucosamine, N-acetylglucosamine, sialic acid, talose, psicose, fructose, sorbose, tagatose, fucose, fuculose, rhamonse, sedoheptulose, octose, sulfoquinovose, nonose (neuraminic acid), sucrose, lactulose, lactose, maltose, trehalose, cellobiose, kojibiose, nigerose, isomaltose, β,β-Trehalose, α,β-Trehalose, sophorose, laminaribiose, gentibiose, turanose, maltulose, palatinose, gentibiulose, mannobiose, melibiose, rutinose, rutinulose, xylobiose, raffinose, melezitose, acarbose, stachyose, and any combinations thereof. In some embodiments, the carbohydrate is pyranose selected from the group consisting of allose, altrose, glucose, mannose, gulose, idose, galactose, and telose.

As used herein, the term “aliphatic” means a moiety characterized by a straight or branched chain arrangement of constituent carbon atoms and can be saturated or partially unsaturated with one or more (e.g., one, two, three, four, five or more) double or triple bonds.

As used herein, the term “alicyclic” means a moiety comprising a nonaromatic ring structure. Alicyclic moieties can be saturated or partially unsaturated with one or more double or triple bonds. Alicyclic moieties can also optionally comprise heteroatoms such as nitrogen, oxygen and sulfur. The nitrogen atoms can be optionally quaternerized or oxidized and the sulfur atoms can be optionally oxidized. Examples of alicyclic moieties include, but are not limited to moieties with C₃-C₈ rings such as cyclopropyl, cyclohexane, cyclopentane, cyclopentene, cyclopentadiene, cyclohexane, cyclohexene, cyclohexadiene, cycloheptane, cycloheptene, cycloheptadiene, cyclooctane, cyclooctene, and cyclooctadiene.

As used herein, the term “alkyl” means a straight or branched, saturated aliphatic radical having a chain of carbon atoms. Cₓ alkyl and Cₓ-C_{y}alkyl are typically used where X and Y indicate the number of carbon atoms in the chain. For example, C₁-C₆alkyl includes alkyls that have a chain of between 1 and 6 carbons (e.g., methyl, ethyl, propyl, isopropyl, butyl, sec-butyl, isobutyl, tert-butyl, pentyl, neopentyl, hexyl, and the like). Alkyl represented along with another radical (e.g., as in arylalkyl) means a straight or branched, saturated alkyl divalent radical having the number of atoms indicated or when no atoms are indicated means a bond, e.g., (C₆-C₁₀)aryl(C₀-C₃)alkyl includes phenyl, benzyl, phenethyl, 1-phenylethyl 3-phenylpropyl, and the like. Backbone of the alkyl can be optionally inserted with one or more heteroatoms, such as N, O, or S.

In preferred embodiments, a straight chain or branched chain alkyl has 30 or fewer carbon atoms in its backbone (e.g., C1-C30 for straight chains, C3-C30 for branched chains), and more preferably 20 or fewer. Likewise, preferred cycloalkyls have from 3-10 carbon atoms in their ring structure, and more preferably have 5, 6 or 7 carbons in the ring structure. The term “alkyl” (or “lower alkyl”) as used throughout the specification, examples, and claims is intended to include both “unsubstituted alkyls” and “substituted alkyls”, the latter of which refers to alkyl moieties having one or more substituents replacing a hydrogen on one or more carbons of the hydrocarbon backbone.

Unless the number of carbons is otherwise specified, “lower alkyl” as used herein means an alkyl group, as defined above, but having from one to ten carbons, more preferably from one to six carbon atoms in its backbone structure. Likewise, “lower alkenyl” and “lower alkynyl” have similar chain lengths. Throughout the application, preferred alkyl groups are lower alkyls. In preferred embodiments, a substituent designated herein as alkyl is a lower alkyl.

Substituents of a substituted alkyl can include halogen, hydroxy, nitro, thiols, amino, azido, imino, amido, phosphoryl (including phosphonate and phosphinate), sulfonyl (including sulfate, sulfonamido, sulfamoyl and sulfonate), and silyl groups, as well as ethers, alkylthios, carbonyls (including ketones, aldehydes, carboxylates, and esters), —CF3, —CN and the like.

As used herein, the term “alkenyl” refers to unsaturated straight-chain, branched-chain or cyclic hydrocarbon radicals having at least one carbon-carbon double bond. Cₓ alkenyl and Cₓ-C_{y}alkenyl are typically used where X and Y indicate the number of carbon atoms in the chain. For example, C₂-C₆alkenyl includes alkenyls that have a chain of between 1 and 6 carbons and at least one double bond, e.g., vinyl, allyl, propenyl, isopropenyl, 1-butenyl, 2-butenyl, 3-butenyl, 2-methylallyl, 1-hexenyl, 2-hexenyl, 3-hexenyl, and the like). Alkenyl represented along with another radical (e.g., as in arylalkenyl) means a straight or branched, alkenyl divalent radical having the number of atoms indicated. Backbone of the alkenyl can be optionally inserted with one or more heteroatoms, such as N, O, or S.

As used herein, the term “alkynyl” refers to unsaturated hydrocarbon radicals having at least one carbon-carbon triple bond. Cₓ alkynyl and Cₓ-C_{y}alkynyl are typically used where X and Y indicate the number of carbon atoms in the chain. For example, C₂-C₆alkynyl includes alkynls that have a chain of between 1 and 6 carbons and at least one triple bond, e.g., ethynyl, 1-propynyl, 2-propynyl, 1-butynyl, isopentynyl, 1,3-hexa-diyn-yl, n-hexynyl, 3-pentynyl, 1-hexen-3-ynyl and the like. Alkynyl represented along with another radical (e.g., as in arylalkynyl) means a straight or branched, alkynyl divalent radical having the number of atoms indicated. Backbone of the alkynyl can be optionally inserted with one or more heteroatoms, such as N, O, or S.

The terms “alkylene,” “alkenylene,” and “alkynylene” refer to divalent alkyl, alkelyne, and alkynylene” radicals. Prefixes Cₓ and Cₓ-C_{y} are typically used where X and Y indicate the number of carbon atoms in the chain. For example, C₁-C₆alkylene includes methylene, (—CH₂—), ethylene (—CH₂CH₂—), trimethylene (—CH₂CH₂CH₂—), tetramethylene (—CH₂CH₂CH₂CH₂—), 2-methyltetramethylene (—CH₂CH(CH₃)CH₂CH₂—), pentamethylene (—CH₂CH₂CH₂CH₂CH₂—) and the like).

As used herein, the term “alkylidene” means a straight or branched unsaturated, aliphatic, divalent radical having a general formula ═CRₐR_{b}. Cₓ alkylidene and Cₓ-C_{y}alkylidene are typically used where X and Y indicate the number of carbon atoms in the chain. For example, C₂-C₆alkylidene includes methylidene (═CH₂), ethylidene (═CHCH₃), isopropylidene (═C(CH₃)₂), propylidene (═CHCH₂CH₃), allylidene (═CH—CH═CH₂), and the like).

The term “heteroalkyl”, as used herein, refers to straight or branched chain, or cyclic carbon-containing radicals, or combinations thereof, containing at least one heteroatom. Suitable heteroatoms include, but are not limited to, O, N, Si, P, Se, B, and S, wherein the phosphorous and sulfur atoms are optionally oxidized, and the nitrogen heteroatom is optionally quaternized. Heteroalkyls can be substituted as defined above for alkyl groups.

As used herein, the term “halogen” or “halo” refers to an atom selected from fluorine, chlorine, bromine and iodine. The term “halogen radioisotope” or “halo isotope” refers to a radionuclide of an atom selected from fluorine, chlorine, bromine and iodine.

A “halogen-substituted moiety” or “halo-substituted moiety”, as an isolated group or part of a larger group, means an aliphatic, alicyclic, or aromatic moiety, as described herein, substituted by one or more “halo” atoms, as such terms are defined in this application. For example, halo-substituted alkyl includes haloalkyl, dihaloalkyl, trihaloalkyl, perhaloalkyl and the like (e.g. halosubstituted (C₁-C₃)alkyl includes chloromethyl, dichloromethyl, difluoromethyl, trifluoromethyl (—CF₃), 2,2,2-trifluoroethyl, perfluoroethyl, 2,2,2-trifluoro-1,1-dichloroethyl, and the like).

The term “aryl” refers to monocyclic, bicyclic, or tricyclic fused aromatic ring system. Cₓ aryl and Cₓ-C_{y}aryl are typically used where X and Y indicate the number of carbon atoms in the ring system. Exemplary aryl groups include, but are not limited to, pyridinyl, pyrimidinyl, furanyl, thienyl, imidazolyl, thiazolyl, pyrazolyl, pyridazinyl, pyrazinyl, triazinyl, tetrazolyl, indolyl, benzyl, phenyl, naphthyl, anthracenyl, azulenyl, fluorenyl, indanyl, indenyl, naphthyl, phenyl, tetrahydronaphthyl, benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH carbazolyl, carbolinyl, chromanyl, chromenyl, cinnolinyl, decahydroquinolinyl, 2H,6H-1,5,2-dithiazinyl, dihydrofuro[2,3 b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxindolyl, pyrimidinyl, phenanthridinyl, phenanthrolinyl, phenazinyl, phenothiazinyl, phenoxathinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl and xanthenyl, and the like. In some embodiments, 1, 2, 3, or 4 hydrogen atoms of each ring can be substituted by a substituent.

The term “heteroaryl” refers to an aromatic 5-8 membered monocyclic, 8-12 membered fused bicyclic, or 11-14 membered fused tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively. Cₓ heteroaryl and Cₓ—C_{y}heteroaryl are typically used where X and Y indicate the number of carbon atoms in the ring system. Heteroaryls include, but are not limited to, those derived from benzo[b]furan, benzo[b]thiophene, benzimidazole, imidazo[4,5-c]pyridine, quinazoline, thieno[2,3-c]pyridine, thieno[3,2-b]pyridine, thieno[2, 3-b]pyridine, indolizine, imidazo[1,2a]pyridine, quinoline, isoquinoline, phthalazine, quinoxaline, naphthyridine, quinolizine, indole, isoindole, indazole, indoline, benzoxazole, benzopyrazole, benzothiazole, imidazo[1,5-a]pyridine, pyrazolo[1,5-a]pyridine, imidazo[1,2-a]pyrimidine, imidazo[1,2-c]pyrimidine, imidazo[1,5-a]pyrimidine, imidazo[1,5-c]pyrimidine, pyrrolo[2,3-b]pyridine, pyrrolo[2,3cjpyridine, pyrrolo[3,2-c]pyridine, pyrrolo[3,2-b]pyridine, pyrrolo[2,3-d]pyrimidine, pyrrolo[3,2-d]pyrimidine, pyrrolo [2,3-b]pyrazine, pyrazolo[1,5-a]pyridine, pyrrolo[1,2-b]pyridazine, pyrrolo[1,2-c]pyrimidine, pyrrolo[1,2-a]pyrimidine, pyrrolo[1,2-a]pyrazine, triazo[1,5-a]pyridine, pteridine, purine, carbazole, acridine, phenazine, phenothiazene, phenoxazine, 1,2-dihydropyrrolo[3,2,1-hi]indole, indolizine, pyrido[1,2-a]indole, 2(1H)-pyridinone, benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH-carbazolyl, carbolinyl, chromanyl, chromenyl, cinnolinyl, decahydroquinolinyl, 2H,6H-1,5,2-dithiazinyl, dihydrofuro[2,3-b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxepanyl, oxetanyl, oxindolyl, pyrimidinyl, phenanthridinyl, phenanthrolinyl, phenazinyl, phenothiazinyl, phenoxathinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydropyranyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl and xanthenyl. Some exemplary heteroaryl groups include, but are not limited to, pyridyl, furyl or furanyl, imidazolyl, benzimidazolyl, pyrimidinyl, thiophenyl or thienyl, pyridazinyl, pyrazinyl, quinolinyl, indolyl, thiazolyl, naphthyridinyl, 2-amino-4-oxo-3,4-dihydropteridin-6-yl, tetrahydroisoquinolinyl, and the like. In some embodiments, 1, 2, 3, or 4 hydrogen atoms of each ring may be substituted by a substituent.

The term “cyclyl” or “cycloalkyl” refers to saturated and partially unsaturated cyclic hydrocarbon groups having 3 to 12 carbons, for example, 3 to 8 carbons, and, for example, 3 to 6 carbons. Cₓcyclyl and Cₓ-C_{y}cylcyl are typically used where X and Y indicate the number of carbon atoms in the ring system. The cycloalkyl group additionally can be optionally substituted, e.g., with 1, 2, 3, or 4 substituents. C₃-C₁₀cyclyl includes cyclopropyl, cyclobutyl, cyclopentyl, cyclohexyl, cyclohexenyl, 2,5-cyclohexadienyl, cycloheptyl, cyclooctyl, bicyclo[2.2.2]octyl, adamantan-1-yl, decahydronaphthyl, oxocyclohexyl, dioxocyclohexyl, thiocyclohexyl, 2-oxobicyclo [2.2.1]hept-1-yl, and the like.

Aryl and heteroaryls can be optionally substituted with one or more substituents at one or more positions, for example, halogen, alkyl, aralkyl, alkenyl, alkynyl, cycloalkyl, hydroxyl, amino, nitro, sulfhydryl, imino, amido, phosphate, phosphonate, phosphinate, carbonyl, carboxyl, silyl, ether, alkylthio, sulfonyl, ketone, aldehyde, ester, a heterocyclyl, an aromatic or heteroaromatic moiety, —CF3, —CN, or the like.

The term “heterocyclyl” refers to a nonaromatic 5-8 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively). Cₓheterocyclyl and Cₓ-C_{y}heterocyclyl are typically used where X and Y indicate the number of carbon atoms in the ring system. In some embodiments, 1, 2 or 3 hydrogen atoms of each ring can be substituted by a substituent. Exemplary heterocyclyl groups include, but are not limited to piperazinyl, pyrrolidinyl, dioxanyl, morpholinyl, tetrahydrofuranyl, piperidyl, 4-morpholyl, 4-piperazinyl, pyrrolidinyl, perhydropyrrolizinyl, 1,4-diazaperhydroepinyl, 1,3-dioxanyl, 1,4-dioxanyland the like.

The terms “bicyclic” and “tricyclic” refers to fused, bridged, or joined by a single bond polycyclic ring assemblies.

The term “cyclylalkylene” means a divalent aryl, heteroaryl, cyclyl, or heterocyclyl.

As used herein, the term “fused ring” refers to a ring that is bonded to another ring to form a compound having a bicyclic structure when the ring atoms that are common to both rings are directly bound to each other. Non-exclusive examples of common fused rings include decalin, naphthalene, anthracene, phenanthrene, indole, furan, benzofuran, quinoline, and the like. Compounds having fused ring systems can be saturated, partially saturated, cyclyl, heterocyclyl, aromatics, heteroaromatics, and the like.

As used herein, the term “carbonyl” means the radical —C(O)—. It is noted that the carbonyl radical can be further substituted with a variety of substituents to form different carbonyl groups including aldehyde (e.g., formyl), acids, acid halides, amides, esters, ketones, and the like. In some embodiments, the carbonyl group is substituted with a heterocyclyl. For example, the carbonyl group can be in the form of an ester or amide when connected to an oxygen or nitrogen atom of heterocyclyl.

The term “carboxy” means the radical —C(O)O—. It is noted that compounds described herein containing carboxy moieties can include protected derivatives thereof, i.e., where the oxygen is substituted with a protecting group. Suitable protecting groups for carboxy moieties include benzyl, tert-butyl, and the like. The term “carboxyl” means —COOH

The term “cyano” means the radical —CN.

The term, “heteroatom” refers to an atom that is not a carbon atom. Particular examples of heteroatoms include, but are not limited to nitrogen, oxygen, sulfur and halogens. A “heteroatom moiety” includes a moiety where the atom by which the moiety is attached is not a carbon. Examples of heteroatom moieties include —N═, —NR^{N}—, —N⁺(O⁻)═, —O—, —S— or —S(O)₂—, —OS(O)₂—, and —SS—, wherein R^{N} is H or a further substituent.

The term “hydroxy” means the radical —OH.

The term “imine derivative” means a derivative comprising the moiety —C(NR)—, wherein R comprises a hydrogen or carbon atom alpha to the nitrogen.

The term “nitro” means the radical —NO₂.

An “oxaaliphatic,” “oxaalicyclic”, or “oxaaromatic” mean an aliphatic, alicyclic, or aromatic, as defined herein, except where one or more oxygen atoms (—O—) are positioned between carbon atoms of the aliphatic, alicyclic, or aromatic respectively.

An “oxoaliphatic,” “oxoalicyclic”, or “oxoaromatic” means an aliphatic, alicyclic, or aromatic, as defined herein, substituted with a carbonyl group. The carbonyl group can be an aldehyde, ketone, ester, amide, acid, or acid halide.

As used herein, the term, “aromatic” means a moiety wherein the constituent atoms make up an unsaturated ring system, all atoms in the ring system are sp² hybridized and the total number of pi electrons is equal to 4n+2. An aromatic ring canbe such that the ring atoms are only carbon atoms (e.g., aryl) or can include carbon and non-carbon atoms (e.g., heteroaryl).

As used herein, the term “substituted” refers to independent replacement of one or more (typically 1, 2, 3, 4, or 5) of the hydrogen atoms on the substituted moiety with substituents independently selected from the group of substituents listed below in the definition for “substituents” or otherwise specified. In general, a non-hydrogen substituent can be any substituent that can be bound to an atom of the given moiety that is specified to be substituted. Examples of substituents include, but are not limited to, acyl, acylamino, acyloxy, aldehyde, alicyclic, aliphatic, alkanesulfonamido, alkanesulfonyl, alkaryl, alkenyl, alkoxy, alkoxycarbonyl, alkyl, alkylamino, alkylcarbanoyl, alkylene, alkylidene, alkylthios, alkynyl, amide, amido, amino, amino, aminoalkyl, aralkyl, aralkylsulfonamido, arenesulfonamido, arenesulfonyl, aromatic, aryl, arylamino, arylcarbanoyl, aryloxy, azido, carbamoyl, carbonyl, carbonyls (including ketones, carboxy, carboxylates, CF₃, cyano (CN), cycloalkyl, cycloalkylene, ester, ether, haloalkyl, halogen, halogen, heteroaryl, heterocyclyl, hydroxy, hydroxy, hydroxyalkyl, imino, iminoketone, ketone, mercapto, nitro, oxaalkyl, oxo, oxoalkyl, phosphoryl (including phosphonate and phosphinate), silyl groups, sulfonamido, sulfonyl (including sulfate, sulfamoyl and sulfonate), thiols, and ureido moieties, each of which may optionally also be substituted or unsubstituted. In some cases, two substituents, together with the carbon(s) to which they are attached to, can form a ring.

The terms “alkoxyl” or “alkoxy” as used herein refers to an alkyl group, as defined above, having an oxygen radical attached thereto. Representative alkoxyl groups include methoxy, ethoxy, propyloxy, tert-butoxy, n-propyloxy, iso-propyloxy, n-butyloxy, iso-butyloxy, and the like. An “ether” is two hydrocarbons covalently linked by an oxygen. Accordingly, the substituent of an alkyl group that renders that alkyl group an ether is or resembles an alkoxyl, such as can be represented by one of —O-alkyl, —O-alkenyl, and —O— alkynyl. Aroxy can be represented by —O-aryl or O-heteroaryl, wherein aryl and heteroaryl are as defined below. The alkoxy and aroxy groups can be substituted as described above for alkyl.

The term “aralkyl”, as used herein, refers to an alkyl group substituted with an aryl group (e.g., an aromatic or heteroaromatic group).

The term “alkylthio” refers to an alkyl group, as defined above, having a sulfur radical attached thereto. In preferred embodiments, the “alkylthio” moiety is represented by one of —S-alkyl, —S-alkenyl, and —S-alkynyl. Representative alkylthio groups include methylthio, ethylthio, and the like. The term “alkylthio” also encompasses cycloalkyl groups, alkene and cycloalkene groups, and alkyne groups. “Arylthio” refers to aryl or heteroaryl groups.

The term “sulfinyl” means the radical —SO—. It is noted that the sulfinyl radical can be further substituted with a variety of substituents to form different sulfinyl groups including sulfinic acids, sulfinamides, sulfinyl esters, sulfoxides, and the like.

The term “sulfonyl” means the radical —SO₂—. It is noted that the sulfonyl radical can be further substituted with a variety of substituents to form different sulfonyl groups including sulfonic acids (—SO₃H), sulfonamides, sulfonate esters, sulfones, and the like.

The term “thiocarbonyl” means the radical —C(S)—. It is noted that the thiocarbonyl radical can be further substituted with a variety of substituents to form different thiocarbonyl groups including thioacids, thioamides, thioesters, thioketones, and the like.

As used herein, the term “amino” means —NH₂. The term “alkylamino” means a nitrogen moiety having at least one straight or branched unsaturated aliphatic, cyclyl, or heterocyclyl radicals attached to the nitrogen. For example, representative amino groups include —NH₂, —NHCH₃, —N(CH₃)₂, —NH(C₁-C₁₀alkyl), —N(C₁-C₁₀alkyl)₂, and the like. The term “alkylamino” includes “alkenylamino,” “alkynylamino,” “cyclylamino,” and “heterocyclylamino.” The term “arylamino” means a nitrogen moiety having at least one aryl radical attached to the nitrogen. For example —NHaryl, and —N(aryl)₂. The term “heteroarylamino” means a nitrogen moiety having at least one heteroaryl radical attached to the nitrogen. For example —NHheteroaryl, and —N(heteroaryl)₂. Optionally, two substituents together with the nitrogen can also form a ring. Unless indicated otherwise, the compounds described herein containing amino moieties can include protected derivatives thereof. Suitable protecting groups for amino moieties include acetyl, tertbutoxycarbonyl, benzyloxycarbonyl, and the like.

The term “aminoalkyl” means an alkyl, alkenyl, and alkynyl as defined above, except where one or more substituted or unsubstituted nitrogen atoms (—N—) are positioned between carbon atoms of the alkyl, alkenyl, or alkynyl. For example, an (C₂-C₆) aminoalkyl refers to a chain comprising between 2 and 6 carbons and one or more nitrogen atoms positioned between the carbon atoms.

The term “alkoxyalkoxy” means —O-(alkyl)-O-(alkyl), such as —OCH₂CH₂OCH₃, and the like.

The term “alkoxycarbonyl” means —C(O)O-(alkyl), such as —C(═O)OCH₃, —C(═O)OCH₂CH₃, and the like.

The term “alkoxyalkyl” means -(alkyl)-O-(alkyl), such as —CH₂OCH₃, —CH₂OCH₂CH₃, and the like.

The term “aryloxy” means —O-(aryl), such as —O-phenyl, —O-pyridinyl, and the like.

The term “arylalkyl” means -(alkyl)-(aryl), such as benzyl (i.e., —CH₂phenyl), —CH₂-pyrindinyl, and the like.

The term “arylalkyloxy” means —O-(alkyl)-(aryl), such as —O-benzyl, —O—CH₂-pyridinyl, and the like.

The term “cycloalkyloxy” means —O-(cycloalkyl), such as —O-cyclohexyl, and the like.

The term “cycloalkylalkyloxy” means —O-(alkyl)-(cycloalkyl, such as —OCH₂cyclohexyl, and the like.

The term “aminoalkoxy” means —O-(alkyl)-NH₂, such as —OCH₂NH₂, —OCH₂CH₂NH₂, and the like.

The term “mono- or di-alkylamino” means —NH(alkyl) or —N(alkyl)(alkyl), respectively, such as —NHCH₃, —N(CH₃)₂, and the like.

The term “mono- or di-alkylaminoalkoxy” means —O-(alkyl)-NH(alkyl) or —O-(alkyl)-N(alkyl)(alkyl), respectively, such as —OCH₂NHCH₃, —OCH₂CH₂N(CH₃)₂, and the like.

The term “arylamino” means —NH(aryl), such as —NH-phenyl, —NH-pyridinyl, and the like.

The term “arylalkylamino” means —NH-(alkyl)-(aryl), such as —NH-benzyl, —NHCH₂-pyridinyl, and the like.

The term “alkylamino” means —NH(alkyl), such as —NHCH₃, —NHCH₂CH₃, and the like.

The term “cycloalkylamino” means —NH-(cycloalkyl), such as —NH-cyclohexyl, and the like.

The term “cycloalkylalkylamino” —NH-(alkyl)-(cycloalkyl), such as —NHCH₂— cyclohexyl, and the like.

It is noted in regard to all of the definitions provided herein that the definitions should be interpreted as being open ended in the sense that further substituents beyond those specified may be included. Hence, a C₁ alkyl indicates that there is one carbon atom but does not indicate what are the substituents on the carbon atom. Hence, a C₁ alkyl comprises methyl (i.e., —CH3) as well as —CRₐR_{b}R_{c} where Rₐ, R_{b}, and R_{c} can each independently be hydrogen or any other substituent where the atom alpha to the carbon is a heteroatom or cyano. Hence, CF₃, CH₂OH and CH₂CN are all C₁ alkyls.

Isomers of the compounds disclosed herein are also provided. “Isomers” mean any compound having identical molecular formulae but differing in the nature or sequence of bonding of their atoms or in the arrangement of their atoms in space. Isomers that differ in the arrangement of their atoms in space are termed “stereoisomers”. Stereoisomers that are not mirror images of one another are termed “diastereomers” and stereoisomers that are nonsuperimposable mirror images are termed “enantiomers” or sometimes “optical isomers”. A carbon atom bonded to four nonidentical substituents is termed a “chiral center”. A compound with one chiral center has two enantiomeric forms of opposite chirality. A mixture of the two enantiomeric forms is termed a “racemic mixture”. A compound that has more than one chiral center has 2ⁿ⁻¹ enantiomeric pairs, where n is the number of chiral centers. Compounds with more than one chiral center may exist as ether an individual diastereomers or as a mixture of diastereomers, termed a “diastereomeric mixture”. When one chiral center is present a stereoisomer may be characterized by the absolute configuration of that chiral center. Absolute configuration refers to the arrangement in space of the substituents attached to the chiral center. Enantiomers are characterized by the absolute configuration of their chiral centers and described by the R- and S-sequencing rules of Cahn, Ingold and Prelog. Conventions for stereochemical nomenclature, methods for the determination of stereochemistry and the separation of stereoisomers are well known in the art (e.g., see “Advanced Organic Chemistry”, 4th edition, March, Jerry, John Wiley & Sons, New York, 1992).

The term “enantiomer” is used to describe one of a pair of molecular isomers which are mirror images of each other and non-superimposable. Other terms used to designate or refer to enantiomers include “stereoisomers” (because of the different arrangement or stereochemistry around the chiral center; although all enantiomers are stereoisomers, not all stereoisomers are enantiomers) or “optical isomers” (because of the optical activity of pure enantiomers, which is the ability of different pure enantiomers to rotate planepolarized light in different directions). Enantiomers generally have identical physical properties, such as melting points and boiling points, and also have identical spectroscopic properties. Enantiomers can differ from each other with respect to their interaction with plane-polarized light and with respect to biological activity.

The designations “R” and “S” are used to denote the absolute configuration of the molecule about its chiral center(s). The designations may appear as a prefix or as a suffix; they may or may not be separated from the isomer by a hyphen; they may or may not be hyphenated; and they may or may not be surrounded by parentheses.

The designations or prefixes “(+)” and “(−)” are employed to designate the sign of rotation of plane-polarized light by the compound, with (−) meaning that the compound is levorotatory (rotates to the left). A compound prefixed with (+) is dextrorotatory (rotates to the right).

The term “racemic mixture,” “racemic compound” or “racemate” refers to a mixture of the two enantiomers of one compound. An ideal racemic mixture is one wherein there is a 50:50 mixture of both enantiomers of a compound such that the optical rotation of the (+) enantiomer cancels out the optical rotation of the (−) enantiomer.

The term “resolving” or “resolution” when used in reference to a racemic mixture refers to the separation of a racemate into its two enantiomorphic forms (i.e., (+) and (−); 65 (R) and (S) forms). The terms can also refer to enantioselective conversion of one isomer of a racemate to a product.

The term “enantiomeric excess” or “ee” refers to a reaction product wherein one enantiomer is produced in excess of the other, and is defined for a mixture of (+)- and (−)-enantiomers, with composition given as the mole or weight or volume fraction F(+) and F(−) (where the sum of F(+) and F(−)=1). The enantiomeric excess is defined as *F(+)-F(−)* and the percent enantiomeric excess by 100×*F(+)-F(−)*. The “purity” of an enantiomer is described by its ee or percent ee value (% ee).

Whether expressed as a “purified enantiomer” or a “pure enantiomer” or a “resolved enantiomer” or “a compound in enantiomeric excess”, the terms are meant to indicate that the amount of one enantiomer exceeds the amount of the other. Thus, when referring to an enantiomer preparation, both (or either) of the percent of the major enantiomer (e.g. by mole or by weight or by volume) and (or) the percent enantiomeric excess of the major enantiomer may be used to determine whether the preparation represents a purified enantiomer preparation.

The term “enantiomeric purity” or “enantiomer purity” of an isomer refers to a qualitative or quantitative measure of the purified enantiomer; typically, the measurement is expressed on the basis of ee or enantiomeric excess.

The terms “substantially purified enantiomer,” “substantially resolved enantiomer” “substantially purified enantiomer preparation” are meant to indicate a preparation (e.g. derived from non-optically active starting material, substrate, or intermediate) wherein one enantiomer has been enriched over the other, and more preferably, wherein the other enantiomer represents less than 20%, more preferably less than 10%, and more preferably less than 5%, and still more preferably, less than 2% of the enantiomer or enantiomer preparation.

The terms “purified enantiomer,” “resolved enantiomer” and “purified enantiomer preparation” are meant to indicate a preparation (e.g. derived from non-optically active starting material, substrates or intermediates) wherein one enantiomer (for example, the R-enantiomer) is enriched over the other, and more preferably, wherein the other enantiomer (for example the S-enantiomer) represents less than 30%, preferably less than 20%, more preferably less than 10% (e.g. in this particular instance, the R-enantiomer is substantially free of the S-enantiomer), and more preferably less than 5% and still more preferably, less than 2% of the preparation. A purified enantiomer may be synthesized substantially free of the other enantiomer, or a purified enantiomer may be synthesized in a stereo-preferred procedure, followed by separation steps, or a purified enantiomer may be derived from a racemic mixture.

The term “enantioselectivity,” also called the enantiomeric ratio indicated by the symbol “E,” refers to the selective capacity of an enzyme to generate from a racemic substrate one enantiomer relative to the other in a product racemic mixture; in other words, it is a measure of the ability of the enzyme to distinguish between enantiomers. A nonselective reaction has an E of 1, while resolutions with E's above 20 are generally considered useful for synthesis or resolution. The enantioselectivity resides in a difference in conversion rates between the enantiomers in question. Reaction products are obtained that are enriched in one of the enantiomers; conversely, remaining substrates are enriched in the other enantiomer. For practical purposes it is generally desirable for one of the enantiomers to be obtained in large excess. This is achieved by terminating the conversion process at a certain degree of conversion.

As used herein, the term “polyethylene glycol” or “PEG” means an ethylene glycol polymer that contains about 2 to about 2000000 linked monomers, typically about 50-1000 linked monomers, usually about 100-300. Polyethylene glycols include ethylene glycol polymer containing various numbers of linked monomers, e.g., PEG20, PEG30, PEG40, PEG60, PEG80, PEG100, PEG115, PEG200, PEG 300, PEG400, PEG500, PEG600, PEG1000, PEG1500, PEG2000, PEG3350, PEG4000, PEG4600, PEG5000, PEG6000, PEG8000, PEG11000, PEG12000, PEG2000000 and any mixtures thereof. In some embodiments, a terminus hydroxyl group of the PEG can be modified to an alkoxy (e.g., methoxy), ester, amide, acetyl and the like. In one embodiment, one terminus hydroxyl of the PEG is modified with a CH₃CH₂OC(O)—CH₂ group. In some embodiments, PEG is diethylene glycol, triethylene glycol, tetraethylenelycol, pentaethylene glycol, hexaehtylene glycol, heptaethylene glycol, octaethylene glycol, nonaethyleneglycol or decaethylene glycol.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

### EXAMPLES

The following examples illustrate some embodiments and aspects of the invention. It will be apparent to those skilled in the relevant art that various modifications, additions, substitutions, and the like can be performed without altering the spirit or scope of the invention, and such modifications and variations are encompassed within the scope of the invention as defined in the claims which follow. The following examples do not in any way limit the invention.

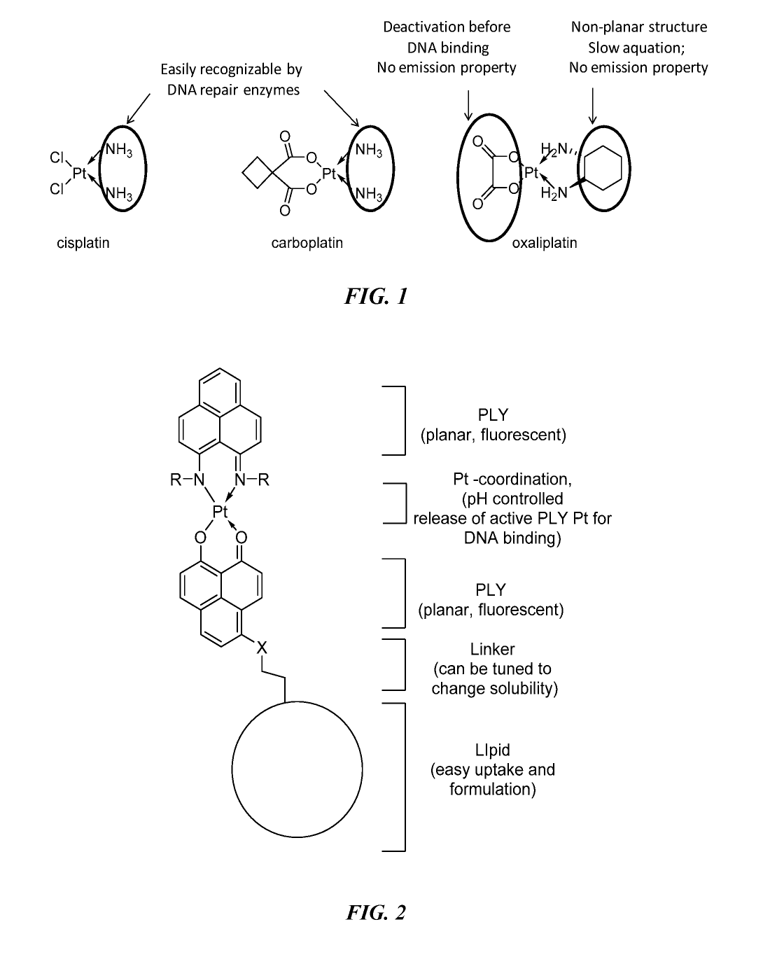

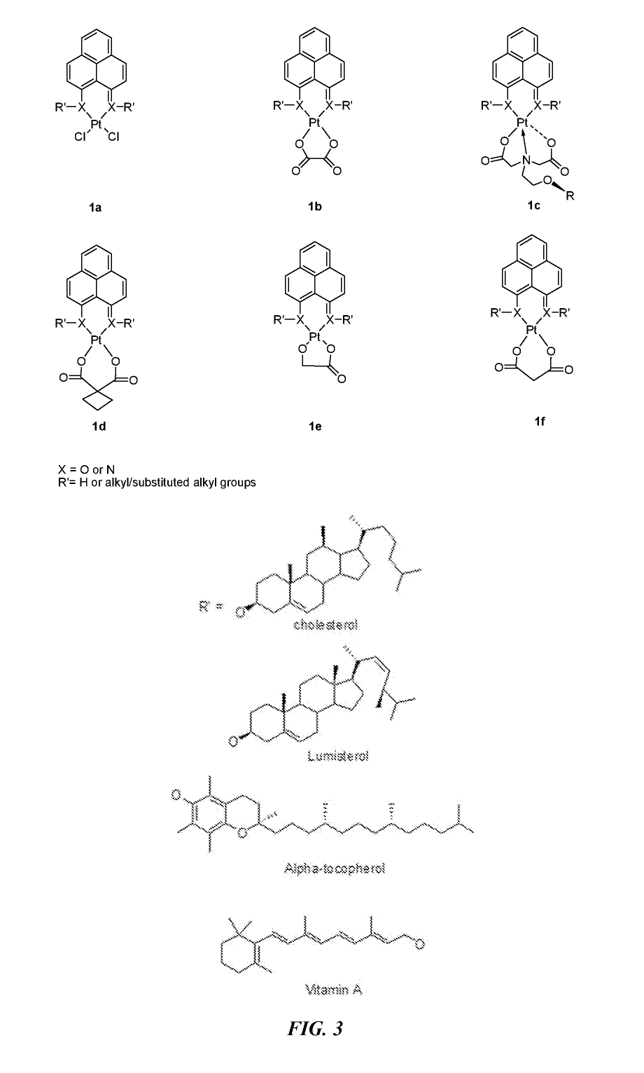

This invention aims at synthesizing phenalenyl based platinum compounds as anticancer agents. The invention primarily deals with the synthesis of a variety of phenalenyl based ligands and their Pt complexes. Main highlight of the molecular design would be to focus on the substituents which can tune fluorescent property to the molecule by intramolecular charge transfer (ICT) mechanism. These complexes have been characterized by standard techniques such as NMR spectroscopy, single crystal X-ray studies and elemental analysis. Excitation and emission wavelengths are determined by UV and fluorescent spectroscopy. Lipid functionalization and supramolecular formulation of these compounds have been carried out to make these drugs less toxic and more efficacious. The IC₅₀ values of some of these compounds have been assessed towards a few cancer cell lines. Detail in vitro and in vivo mechanistic investigation of the drugs will be carried out when its fluorescent property will be probed. FIG. 1 describes the scope of improvements in the current anticancer drugs and FIG. 2 represents the significance of the molecular design of an embodiment of the invention.

### Example 1: Synthesis of Compounds of Formula I, II, III, IV and V

### Synthesis of Compounds of Formula I, II and III (Class I, II and III Ligands)

These compounds are prepared following conventional and reported routes (Scheme 1) (K. D. Franz, J. Org. Chem., Vol. 44, No. 10, 1979; K. D. Franz, R. L. Martin, Tetrahedron 1978, 34, 2147; and Sarkar et al., Chemistry of Materials, 2009, 21, 2226).

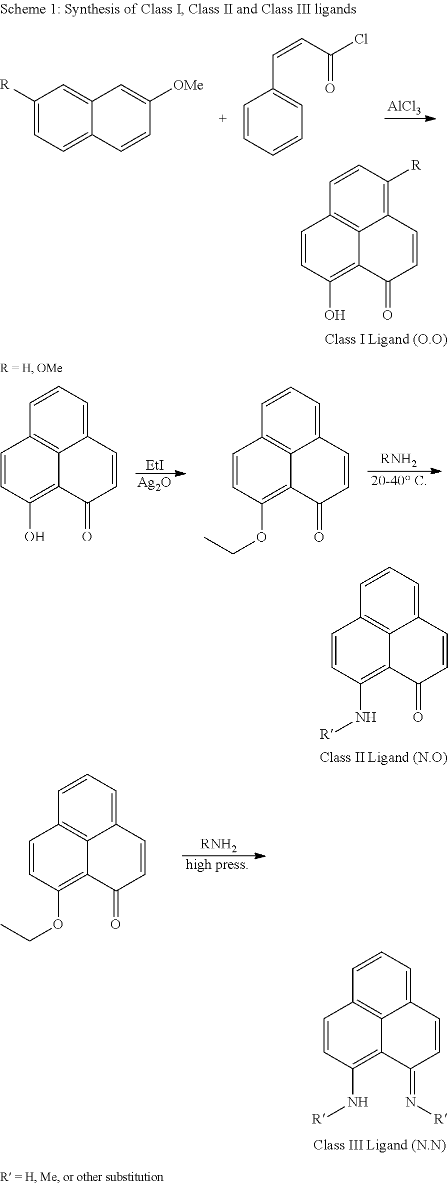

### Synthesis of Compounds of Formula IV (Class IV Ligands)

These compounds are prepared by the synthesis scheme shown in Scheme 2.

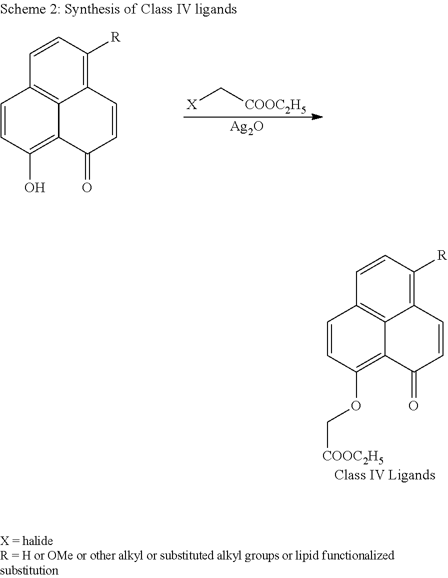

To 2 mmol of bromo ethyl acetate is added dropwise to a mixture of 2 mmol of 9-hydroxyphenalenone (R═H) and silver oxide (3 mmol) in 50 mL ethanol and refluxed for 24 h. The reaction mixture is then concentrated over vacuum to evaporate ethanol. A brown residue obtained which is filtered and passed through a flash silica column to obtain Class IV ligand as a crude product (Scheme 2).

### Synthesis of Compounds of Formula V (Class V Ligands)

These compounds are prepared by the synthesis scheme shown in Scheme 3 (Route 1), Scheme 4 (Route 2) or Scheme 5 (Route 3).

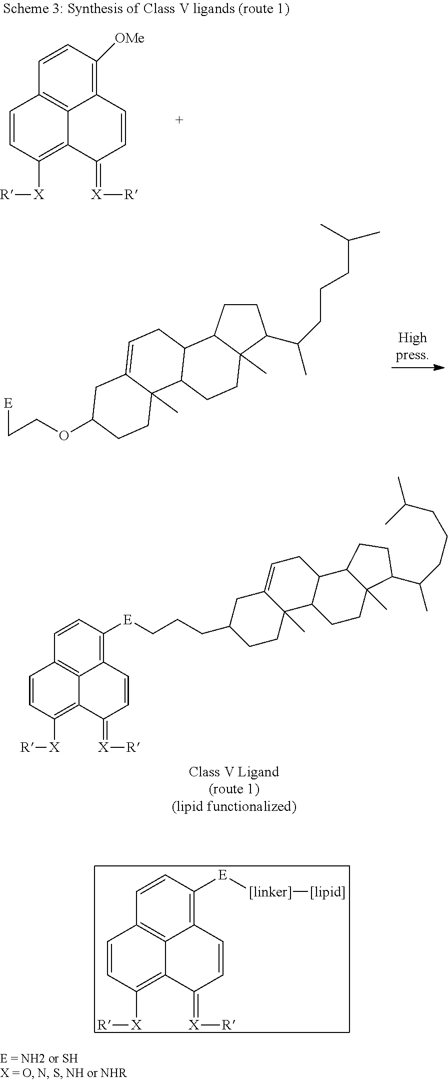

### Procedure (for X=O; E=NH, R′=H)

2 mmol of cholesteryl ethyl amine is mixed with 4 methoxy 9-hydroxyphenalenone (2 mmol) in 10 mL DCM and refluxed for 24 h. The reaction mixture is then concentrated over vacuum to evaporate DCM. A yellow residue obtained which is passed through a flash silica column to obtain Class V ligand as a crude product (Scheme 3).

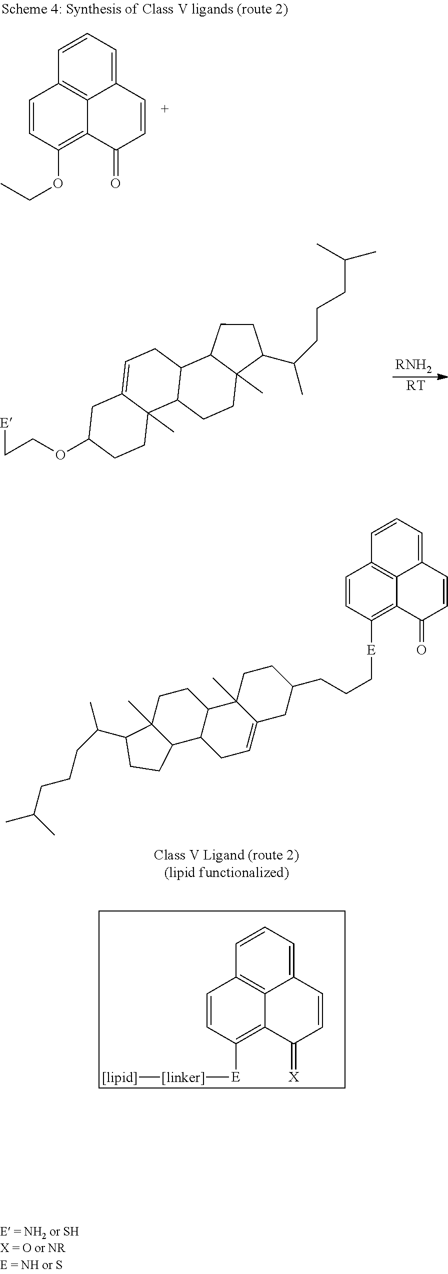

### Procedure (for X=O; E=NH)

2 mmol of cholesteryl ethyl amine is mixed with 9-ethoxy phenalenone (2 mmol) in 10 mL DCM and refluxed for 24 h. The reaction mixture is then concentrated over vacuum to evaporate DCM. A yellow residue obtained which is passed through a flash silica column to obtain Class V ligand as a crude product (Scheme 4).

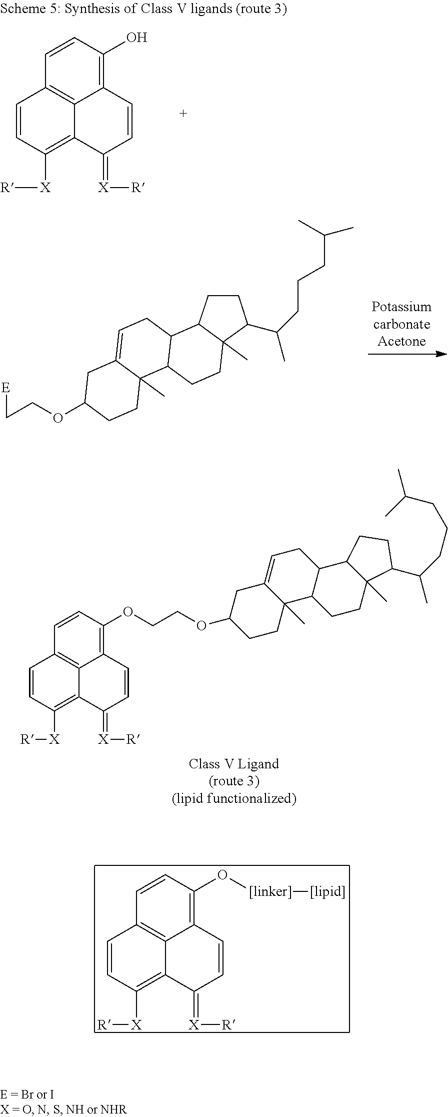

### Procedure (for X=O; E=Br or I)

2 mmol of 4,9-dihydroxy phenalenone is stirred with 2 mmol of potassium carbonate in dmf for 1 h. Cholesteryl ethyl bromide/iodide (2 mmol) is added to the mixture and refluxed for 24 h. The reaction mixture is then concentrated over high vacuum and washed with water. A yellow residue obtained which is passed through a flash silica column to obtain Class V ligand as a crude product (Scheme 5).

### Example 2: Synthesis of Platinum Containing Compounds

### Synthesis of Pt Complexes

Mainly four classes of Pt complexes are being synthesized. In Class 1 complexes, all four classes of ligands are reacted with aquated diamminocyclohexane platinum(II) precursor to yield Pt drugs where active drug is similar to oxaliplatin i.e [(DACH)Pt] whereas the leaving groups are Class I to Class V ligands (e.g., compounds of Formula I, II, III and IV). In Class 2 complexes DACH backbone is replaced by the synthesized class I, II, III phenalenyl ligands (e.g., compounds of Formula I, II and III). In Class 3 complexes, all four coordination sites of the platinum are replaced by Class I to Class V phenalenyl ligands (e.g., compounds of Formula I, II, III and IV). In Class 4 complexes, leaving groups are Class IV ligands (Compounds of Formula IV) which are being linked to either oxaliplatin or cisplatin backbone. Schematic representations of ligands of Classes I-III of platinum complexes are shown in Scheme 1. Conventional synthetic methodologies for the synthesis of platinum drugs can be followed with required modifications as needed.

### Synthesis of Class 1 Complexes

Class 1 complexes are prepared as shown in Scheme 6.

### Synthesis of 1c

Compound 1a (1 mmol) in 5 mL ethanol is treated with 2 mmol of silver nitrate and stirred for 24 h at 20° C. AgCl precipitate is filtered and charged with 1 mmol of the sodium salt of the lipid functionalized diester (in 10 mL water). The reaction mixture is stirred at 20° C. for 24 h. The reaction mixture is then concentrated over vacuum to evaporate ethanol. The brown precipitate is washed with water to obtain crude compound 1c (Scheme 6).

Other compounds of Class 1 have been synthesized from 1a following similar procedure as described for compound 1c.

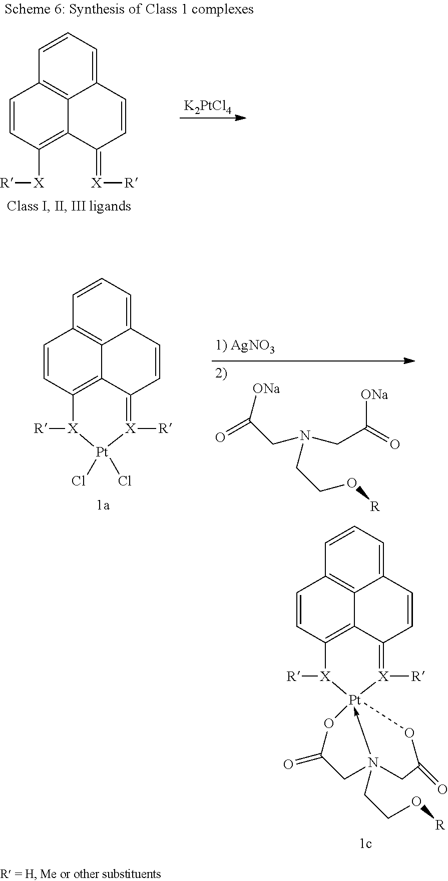

### Synthesis of Class 2 Complexes

Class 2 complexes are prepared as shown in Scheme 7.

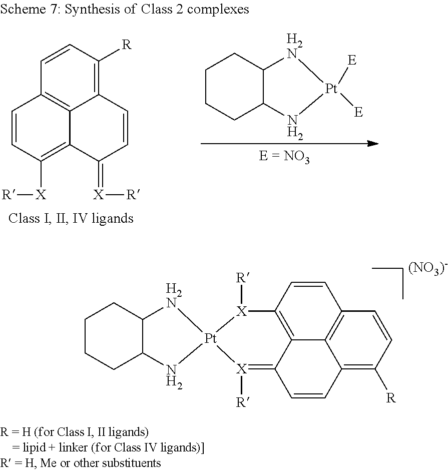

A detailed synthesis of compounds 2a and 2b is disclosed in Example 6.

### Synthesis of Class 3 Complexes

Class 3 complexes are prepared as shown in Scheme 8.

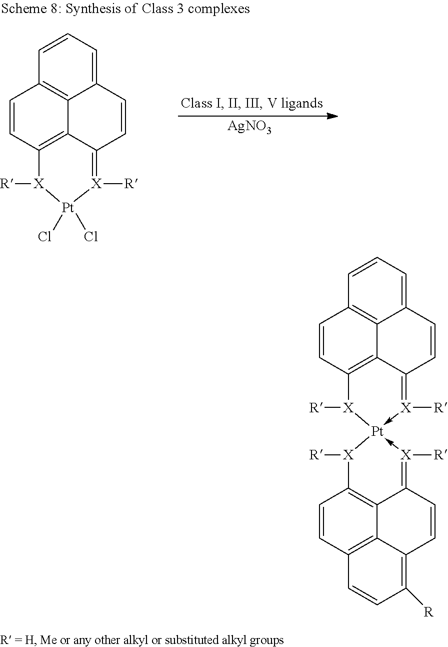

### Synthesis of 3d

Compound 1a (0.5 mmol) in 5 mL ethanol is treated with 1 mmol of silver nitrate and stirred for 24 h at 20° C. AgCl precipitate is filtered and charged with 0.5 mmol of the sodium salt of the lipid functionalized Class V ligand (X=O; E=NH, R′=H, R=cholesterol) prepared from route 1 (in 10 mL water). The reaction mixture is stirred at 20° C. for 24 h. The reaction mixture is then concentrated over vacuum to evaporate ethanol. The brown precipitate is washed with water to obtain crude compound 1c (Scheme 8).

Other compounds of Class 3 have been synthesized from 1a following similar procedure as described for compound 3d.

### Synthesis of Class 4 Complexes

Class 4 complexes are prepared as shown in Scheme 9.

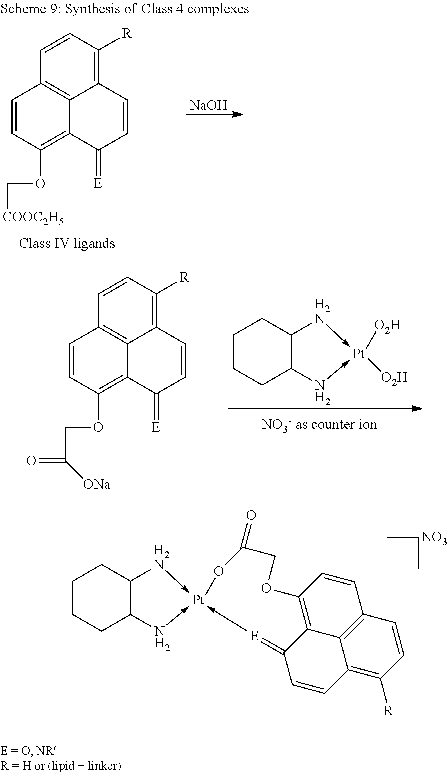

### Synthesis of 4b

Class IV ligand (E=O, R=H) (0.5 mmol) in 5 mL THF/water is treated with 1 mmol of NaOH and stirred for 5 h at 20° C., washed with DCM and the aqueous layer is added dropwise to 0.5 mmol of aquated cyclohexyldiammine platinum(II). The reaction mixture is stirred at 20° C. for 24 h. The brown precipitate is washed with water to obtain crude compound 1c.

Other compounds of Class 4 have been synthesized from the corresponding Class IV ligand following similar procedure as described for compound 4b.

### Example 3: Bioassays

Cell Culture:

Mammalian cells were grown in specific culture media, supplemented with 10% fetal bovine serum (FBS) and antibiotics in a humidified environment containing 5% CO₂ at 37° C.

Cell viability assay: The effects of exemplary molecules on the viability of cancer cells were measured using MTT assay. Cells in 100 μl culture-media were plated in 96-well plates (3000-5000 cells/well) and allowed to adhere overnight under above mentioned cell culture conditions. Fresh media (100 μL) containing different concentrations of compounds were added to cells and incubated for 48 hrs. Following incubation, cell viability was determined using the MTT assay and viability was plotted as dose-response curves using curve fitting.

Cellular Accumulation Assay:

Cellular accumulation of compounds was evaluated in cells by quantifying total platinum concentration in cells. Cells (500,000 cells/well) were seeded in 6-well plate and grown for 24 hours in a humidified chamber. Compounds were added in each well to achieve a final platinum equivalent concentration of 50 μM per well and incubated for 5 additional hours. Cells were harvested by trypsinization, counted and washed once with PBS and pelleted. The pellets were digested with nitric acid (150 μl) at 100° C. for 3 hours in a glass vial. Following digestion, the samples were diluted in 2% HCl, the volume made up to 1 ml for detection of platinum by atomic spectroscopy (AAS). The data was plotted as accumulated platinum normalized to total cell count.

DNA-Platinum Adduct Formation:

DNA-Pt adduct formation was evaluated in cells post treatment with compounds. Cells were seeded in 100 mm cell-culture dish and grown for 24 hours in a humidified chamber. When cells reached a confluency of 60-70%, compounds were added to the culture dish to achieve a final platinum equivalent concentration of 50 μM and incubated further for 24 hours. Cells were harvested by trypsinization and washed once with PBS. Cells were pelleted and DNA isolated using DNAzol® Reagent, according to the manufacture's protocol. The DNA was dissolved in designated solvent and stored overnight at 4° C. The OD (A₂₆₀/A₂₈₀) of dissolved DNA was measured and 30-60 μg of DNA sample was digested with nitric acid and processed for platinum estimation by AAS as described above. The data was plotted as platinum concentration normalized to DNA concentration.

Cellular Uptake of Fluorescent Compounds:

The intracellular localization of molecules was visualized by fluorescence microscopy. Cells were seeded on cover slips to a confluency of 40% and grown for 24 hours in a humidified chamber. Cells were treated with fluorescent compounds and incubated for 2, 5 and 24 hours. Following incubation, cells were washed with PBS, fixed in 4% paraformaldehyde and observed under epifluorescence microscope. Images were recorded in phase contrast and fluorescent channels. Cells treated with vehicle and/or ligand were considered as control and microscopically evaluated.

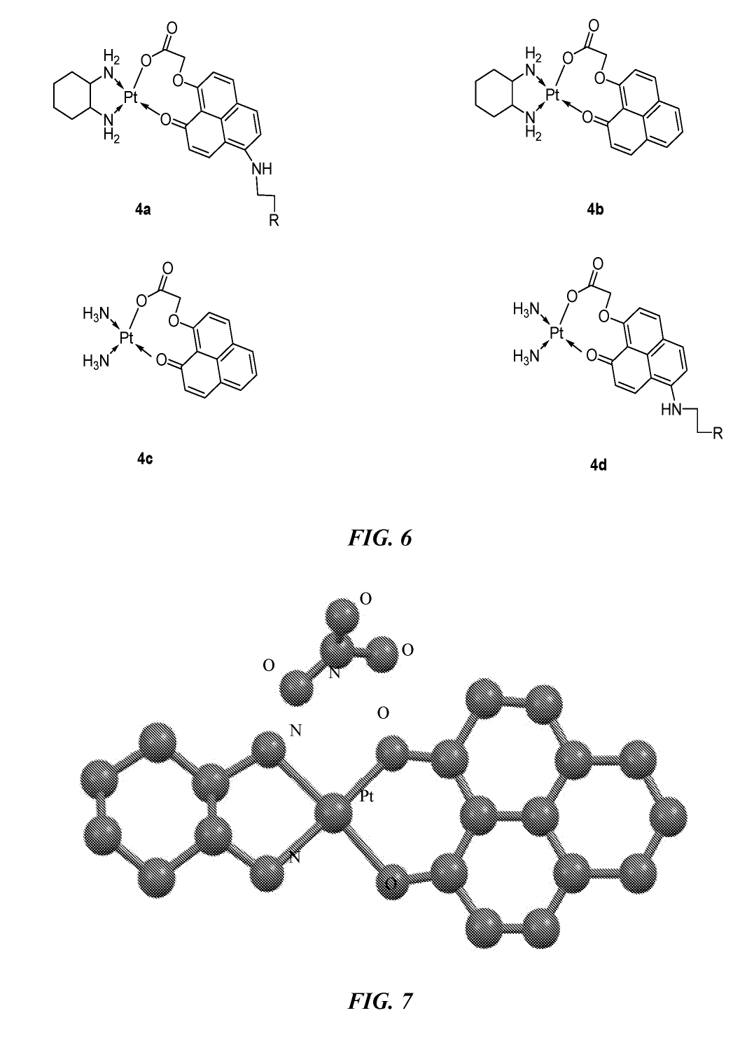

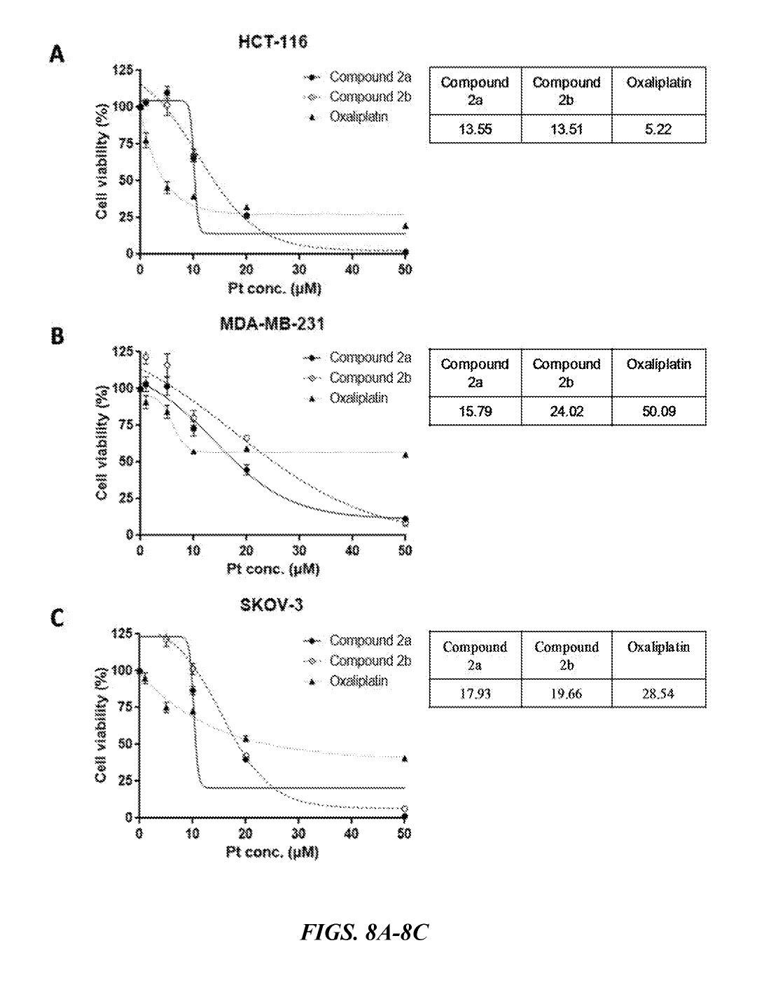

The effects of compounds 2a and 2b were evaluated in vitro in comparison with a standard platinum drug (oxaliplatin) in breast cancer (MDA-MB-231), ovarian cancer (SKOV-3) and colorectal cancer (HCT-116) cell lines. Results are shown in FIGS. 8A-8C. As seen from FIGS. 8A-8C, both compounds showed better efficacy (based on lower IC₅₀ values) than oxaliplatin in breast (FIG. 8B) and ovarian cancer lines (FIG. 8C). The effects of IO-199_16 were evaluated in lung cancer (A549) cell line and results depicted in FIG. 9 demosntrate its better efficacy than oxaliplatin in A549 cells.

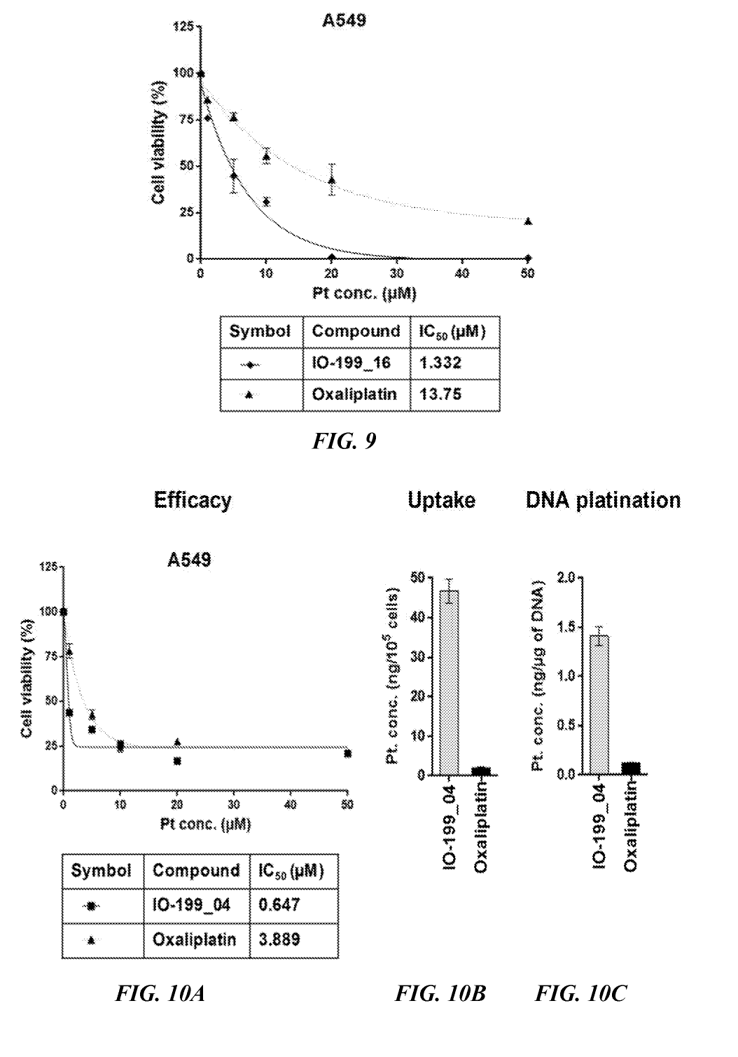

Compound IO-199_04 was formulated and evaluated for its effect, cellular accumulation and DNA-Pt adduct formation in A549 cells. Results demonstrated that IO-199_04 showed 6-fold lower IC₅₀ than oxaliplatin, indicating better efficacy than oxaliplatin in A549 cells (FIG. 10A). IO-199_04 showed a 20-fold higher cellular accumulation (FIG. 10B) and 10-fold higher DNA-Pt adduct formation (FIG. 10C) than oxaliplatin in A549 cells.

### Example 4: Synthesis of Compounds of Formula XII

To understand the mechanism of internalisation of IO-125 into the cell, synthetic schemes (Schemes 10-18) were designed to prepare IO-200 set of molecules (similar to IO-125 with a dye unit attached to it). Different dyes like rhodamine, fluorescein, 7-amino-4-methyl coumarin, dansyl chloride, fluorene-1-carboxylic acid, bromobimane have been used to track the internalisation of the compound IO-125 in to the cells.

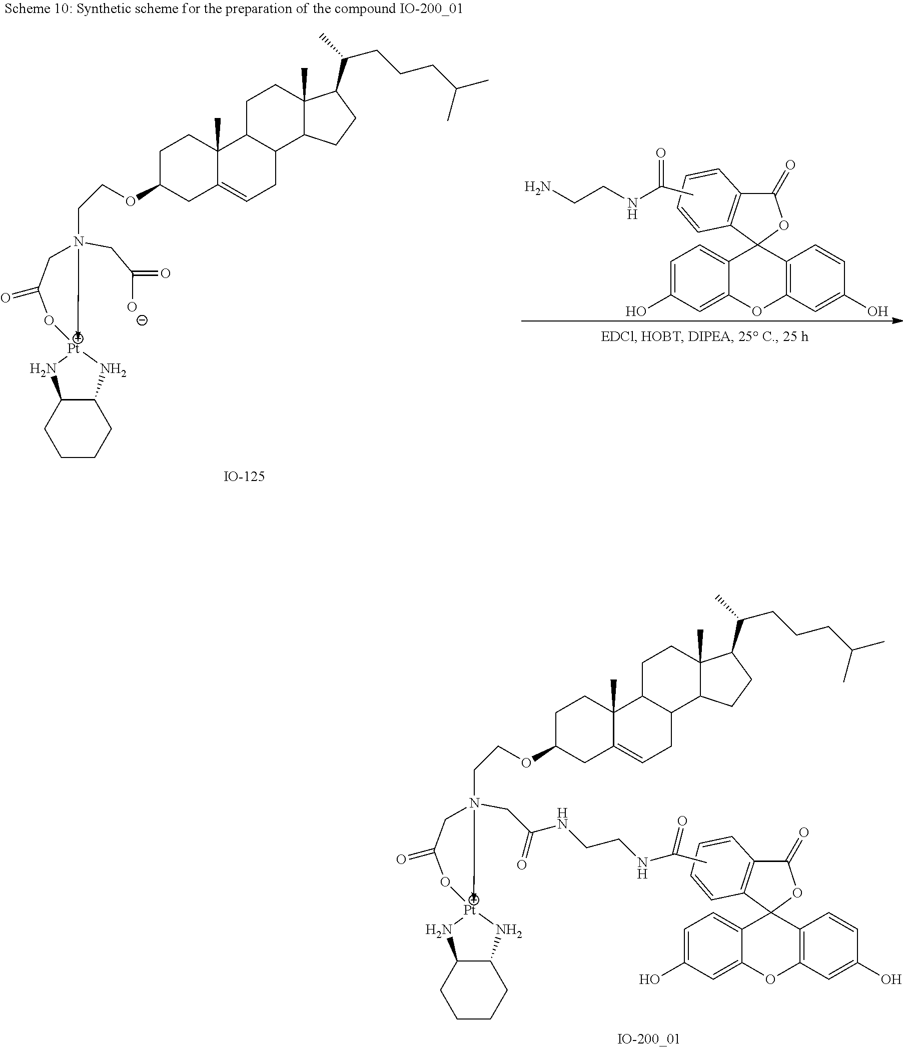

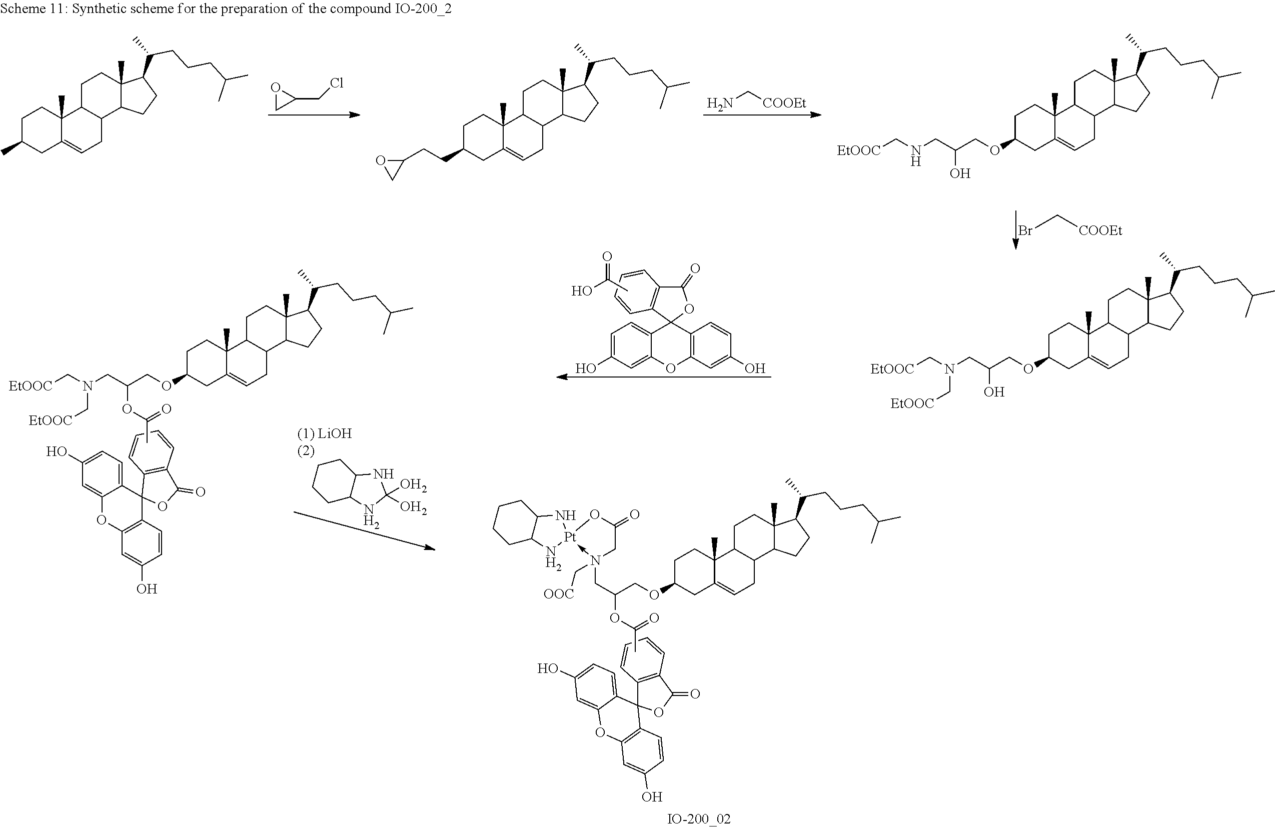

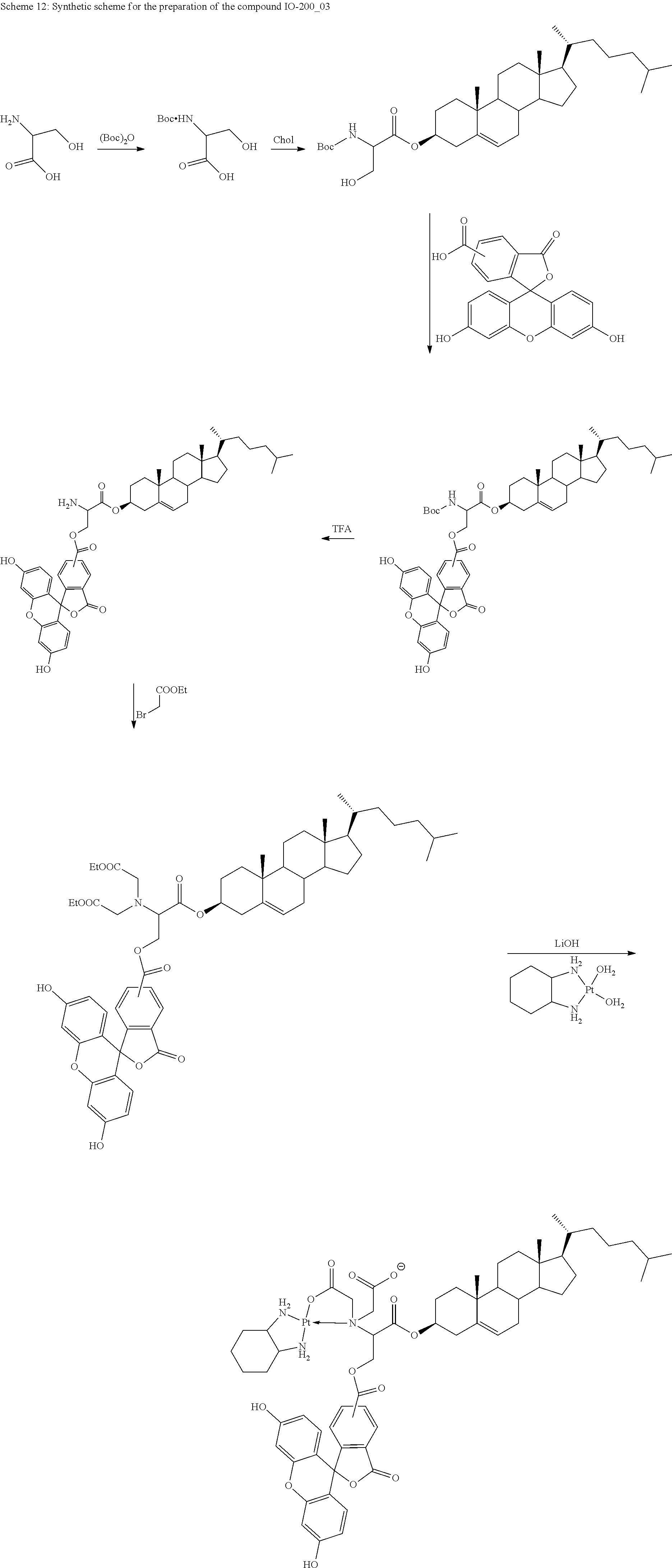

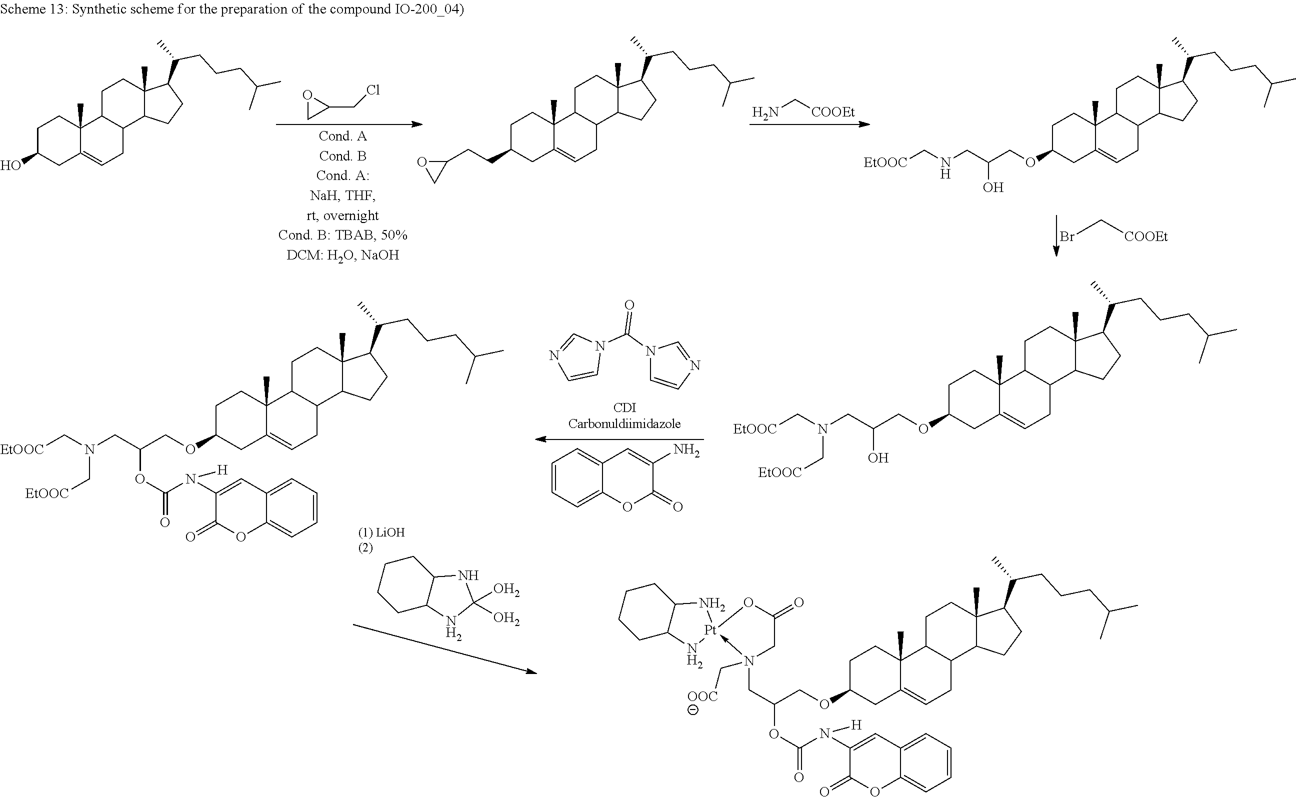

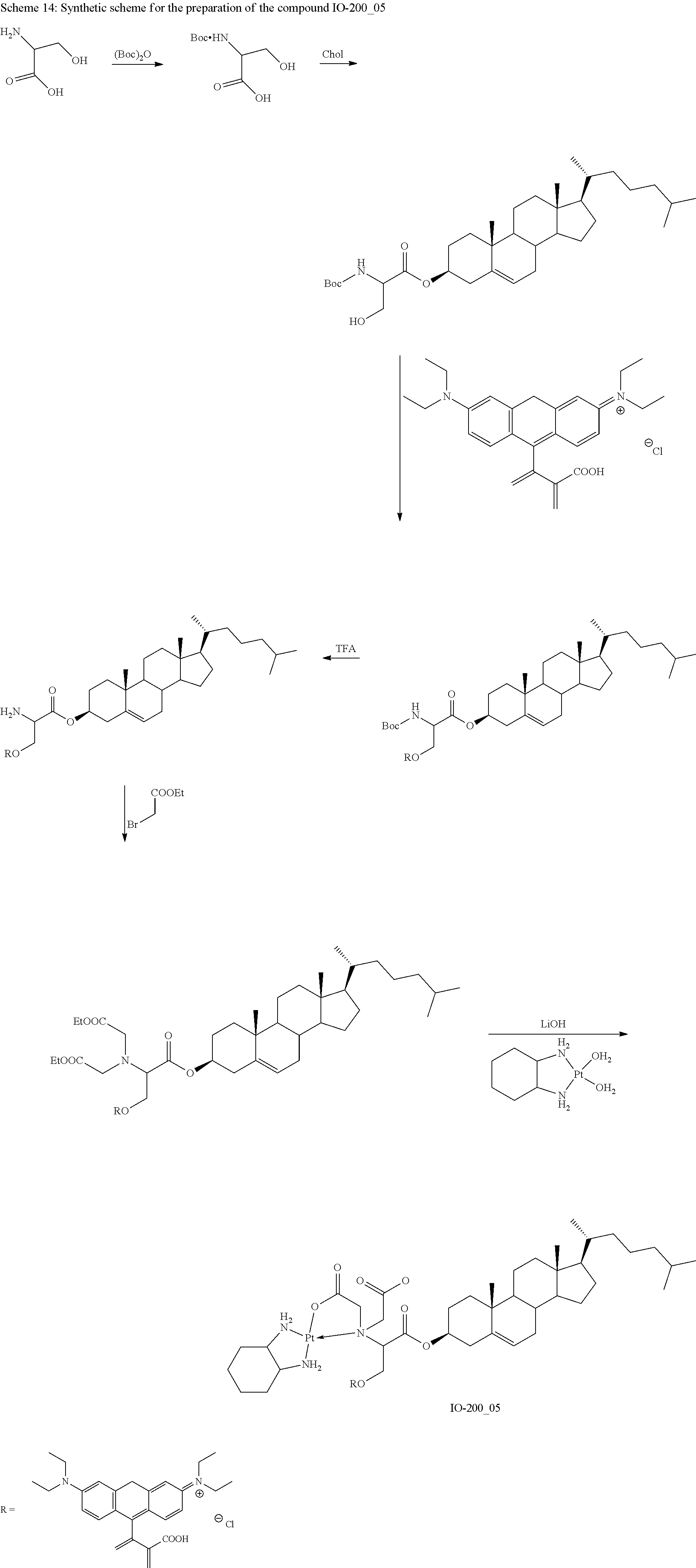

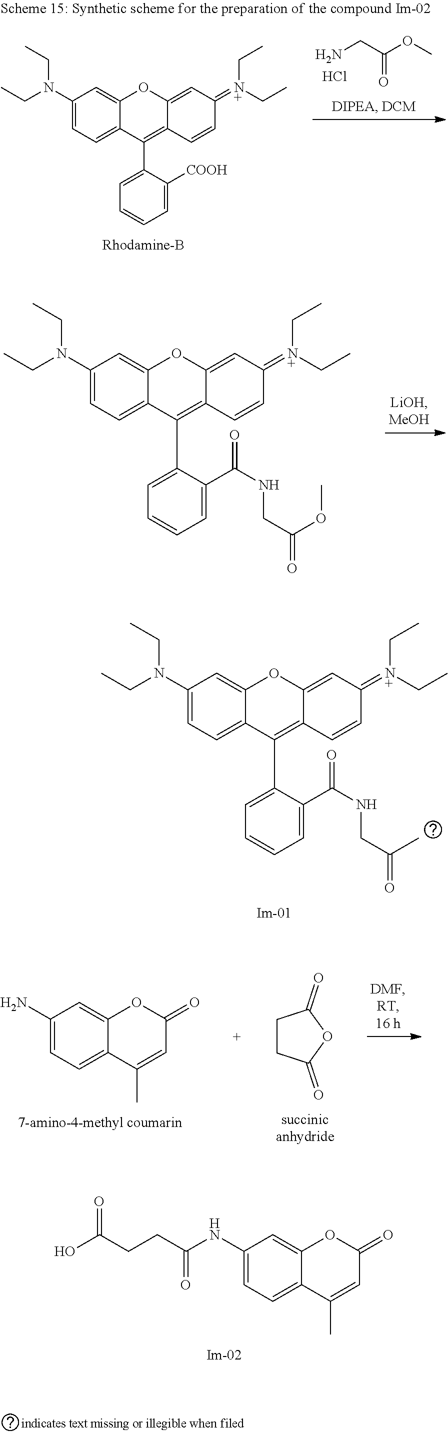

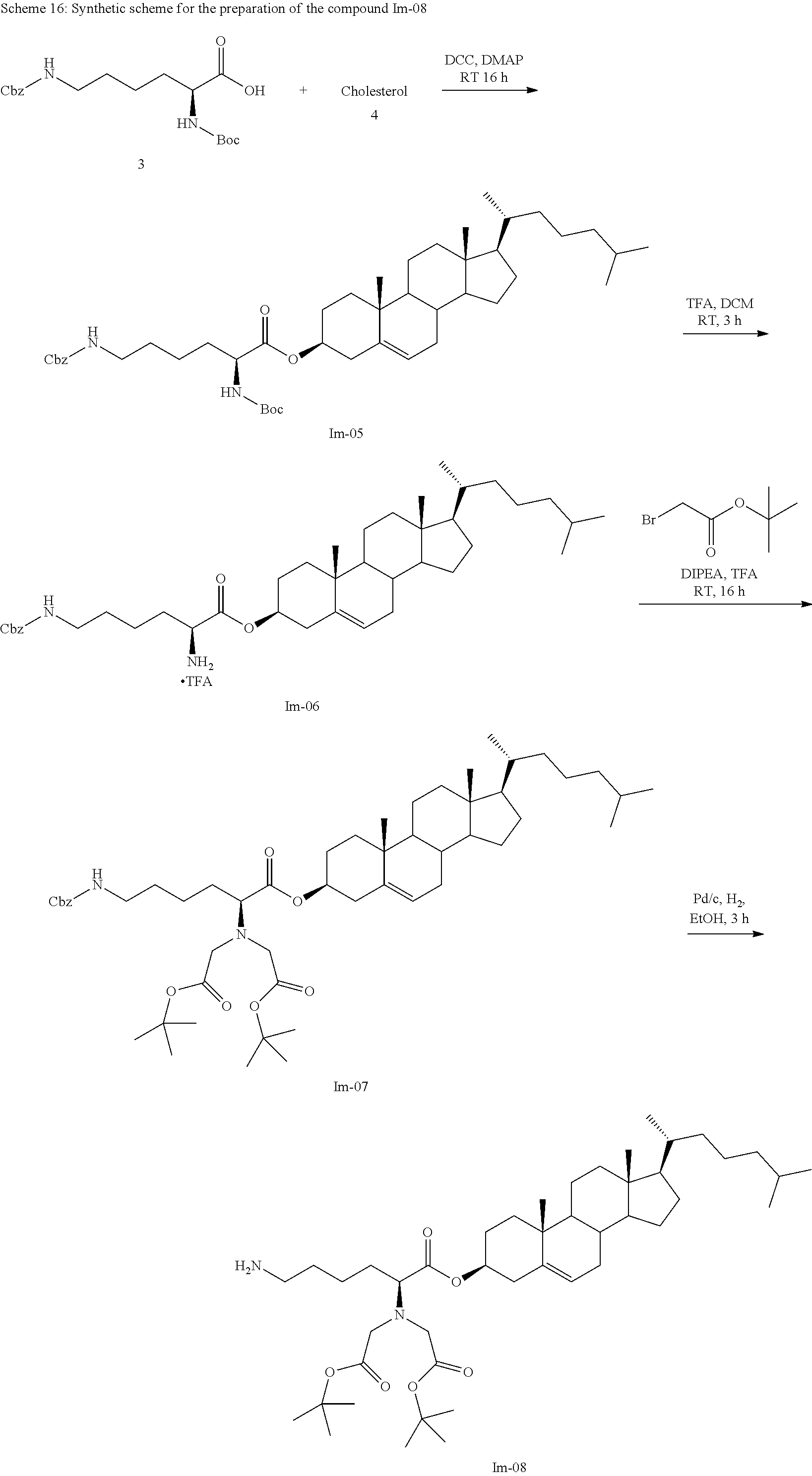

The synthetic methodology for the preparation of the compounds IO-200_06-IO-200_011 was similar.

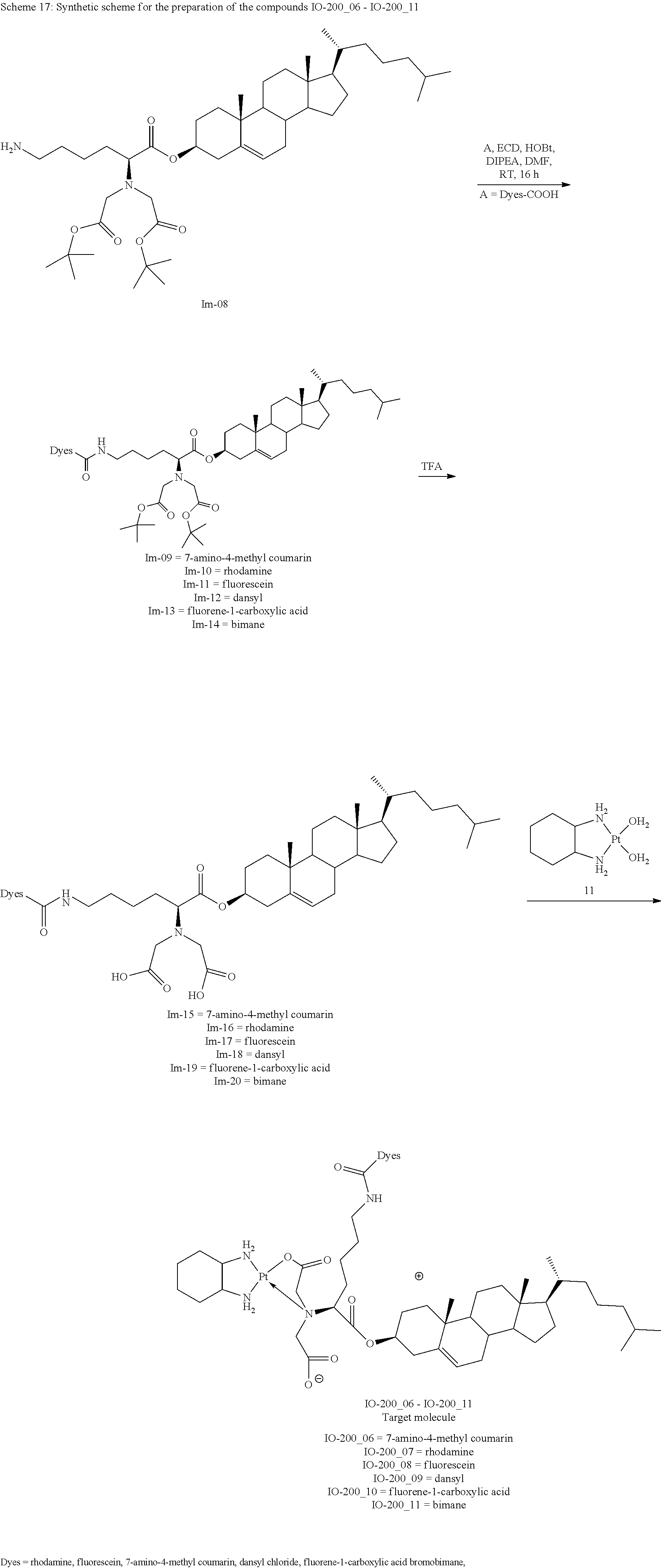

In the first step, commercially available cholesterol was coupled with Boc-Lys(Z)—OH using dicyclohexyl carbodimide (DCC) by stirring overnight at room temperature. The intermediate Im-05, thus obtained was treated with trifluoro acetic acid (TFA) to yield amine salt Im-06. The amine was further dialkylated with tert butyl bromo acetate in presence of diisopropyl ethyl amine (DIPEA) to give compound Im-07. The amine intermediate Im-08 was prepared by the removal of carboxybenzyl (CBz) group using Pd/C (10% over activated charcoal) under hydrogen atmosphere for 3 h. The amino compound was the common intermediate for the preparation of IO-200_06-IO-200_011. The 7-amino-4-methyl coumarin derivative Im-02, was further coupled with the amine derivative Im-08 to yield Im-09. The column purified intermediate Im-09, was treated with 20% TFA in chloroform to give di-acid Im-15 which was further reacted with freshly prepared Pt-DACH complexes at room temperature for 16 h to give compound IO-200_06. The final compound was further solubilised with methanol, filtered and dried to get pure IO-200_06 as a whitish solid. By following the similar way, intermediate Im-08 was treated with rhodamine derivative to yield compound Im-10. The intermediate thus obtained was successively treated with TFA and Pt-DACH complexes to yield Im-16 and IO-200_07 respectively. By following the abovementioned procedure IO-200_08-IO-200_011 are prepared.

### Synthesis of Intermediate Im-05

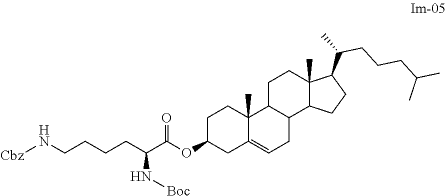 [SMILES: CC(C)CCC[C@@H](C)[C@H]1CCC2C3CC=C4C[C@@H](OC(=O)[C@H](CCCCNC(=O)OCc5ccccc5)NC(=O)OC(C)(C)C)CC[C@]4(C)C3CC[C@@]21C]

To a solution of (S)-6-(benzyloxycarbonylamino)-2-(tert-butoxycarbonylamino) hexanoic acid (1.0 g, 2.62 mmol) in DCM (10 mL) was added DCC (647 mg, 3.14 mmol) followed by N,N-dimethyl amino pyridine (DMAP) (160 mg, 1.31 mmol). The reaction mixture was further stirred at room temperature for 30 min, then cholesterol (1.016 g, 2.62 mmol) was added and stirred at room temperature for 16 h. Reaction was monitored by TLC, water was added and extracted with chloroform. Brine washing was given to the organic layer, dried over anhydrous sodium sulphate and evaporated. Crude compound was purified by column chromatography, compound eluted in 20% ethyl acetate: hexane. Yield: 0.47 g, 24%.

¹H NMR (500 MHz, CDCl₃); δ 7.42-7.36 (m, 5H), 5.42 (m, 1H), 5.18-5.13 (m, 2H), 4.72-4.70 (m, 1H), 4.39-4.36 (m, 1H), 3.15 (m, 2H), 2.38-2.34 (m, 2H), 2.07-1.87 (m, 9H), 1.73-1.51 (m, 9H), 1.48 (m, 11H), 1.42-1.30 (m, 9H), 1.26 (t, J=5.0 Hz, 2H), 1.21-1.10 (m, 6H), 1.07 (s, 3H), 1.05-0.99 (m, 1H), 0.96 (d, J=5.0 Hz, 3H), 0.91 (d, J=6.5, Hz, 3H), 0.90 (d, J=6.5, Hz, 3H). MS (ES-MS) [M+H]⁺ calcd for C₄₆H₇₃N₂O₆ m/z 749.54, found m/z 749.48.

### Synthesis of Intermediate Im-06

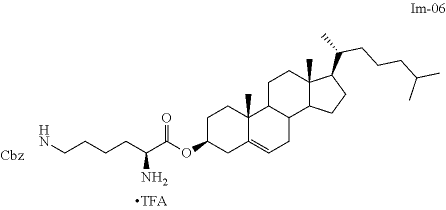

To a solution of compound Im-05 (300 mg, 0.4 mmol) in dry DCM (6 mL) was added trifluoro acetic acid (0.06 mL, 0.8 mmol) at 0° C. and allowed to warm to room temperature for 3 h. Reaction was monitored by TLC. Solvent was evaporated and used as such for next step. Yield: 0.298 g, 100%. MS (ES-MS) [M+H]⁺ calcd for C₄₁H₆₅N₂O₄ m/z 649.49, found m/z 649.47.

### Synthesis of Intermediate Im-7

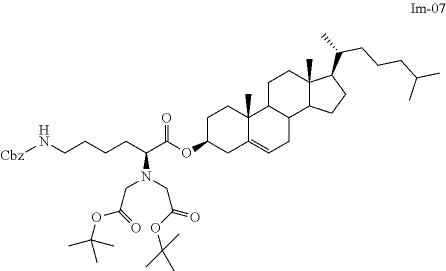 [SMILES: CC(C)CCC[C@@H](C)[C@H]1CCC2C3CC=C4C[C@@H](OC(=O)[C@H](CCCCNC(=O)OCc5ccccc5)N(CC(=O)OC(C)(C)C)CC(=O)OC(C)(C)C)CC[C@]4(C)C3CC[C@@]21C]

To a solution of compound Im-06 (0.29 g, 0.38 mmol) in dry THF (5.8 mL) was added di-isopropyl ethyl amine (0.408 mL, 2.28 mmol) at 0° C. Then tert butyl bromo acetate was added drop wise and stirred at room temperature for 16 h. Reaction was monitored by TLC, water was added and extracted with ethyl acetate, dried and evaporated. Crude compound was purified by column chromatography. Compound Im-07 was eluted in 5-10% ethyl acetate: hexane. Yield:0.29 g, 85%. ¹H NMR (500 MHz, DMSO-d₆) δ 7.72 (d, J=7.5 Hz, 1H), 7.40-7.35 (m, 5H), 5.38 (m, 1H), 5.10-5.02 (m, 2H), 4.51 (m, 1H), 3.98-3.94 (m, 1H), 3.36 (s, 4H), 2.58-2.54 (m, 2H), 2.30-2.21 (m, 2H), 2.01-1.94 (m, 2H), 1.84-1.78 (m, 3H), 1.68-1.51 (m, 8H), 1.43 (m, 19H), 1.39-1.34 (m, 7H), 1.27 (m, 6H), 1.17-1.06 (m, 8H), 1.00 (s, 3H), 0.94-0.93 (m, 3H), 0.89-0.87 (m, 6H). ¹³C NMR (126 MHz, CDCl₃) δ 171.89, 170.70, 155.92, 139.35, 136.39, 128.47, 128.08, 122.88, 80.88, 75.06, 66.83, 56.67, 56.13, 55.92, 54.07, 53.56, 49.99, 42.30, 39.71, 39.50, 37.95, 36.89, 36.55, 36.17, 35.77, 32.47, 31.88, 31.83, 29.67, 28.21, 28.16, 27.99, 27.67, 27.53, 24.26, 23.81, 22.80, 22.70, 22.68, 22.54, 22.28, 21.02, 20.20, 19.29, 18.70, 11.84. MS (ES-MS) [M]⁺ calcd. for C₅₃H₈₄N₂O₈; m/z 876.62, found m/z 877.63.

### Synthesis of Intermediate Im-08

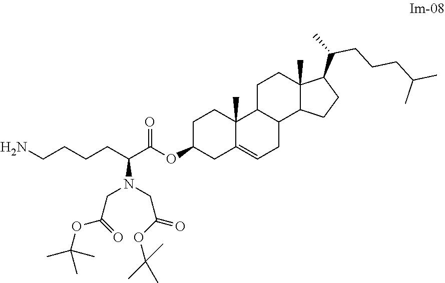 [SMILES: CC(C)CCC[C@@H](C)[C@H]1CCC2C3CC=C4C[C@@H](OC(=O)[C@H](CCCCN)N(CC(=O)OC(C)(C)C)CC(=O)OC(C)(C)C)CC[C@]4(C)C3CC[C@@]21C]

To a solution of compound Im-7 (0.28 g, 0.31 mmol) in ethanol (3 mL) was charged Pd/C (56 mg, 20% w/w) under the nitrogen atmosphere and hydrogenated by bladder pressure for 3 h at room temperature. Reaction was monitored by TLC after the completion the reaction mixture was filtered through celite, and the bed was repeatedly washed with ethanol to get the compound Im-08 as colourless sticky liquid. Yield:0.23 g, 97%. MS (ES-MS) [M+H]⁺ calcd for C₄₅H₇₉N₂O₆ m/z 743.59, found m/z 743.57.

### Synthesis of Intermediate Im-02

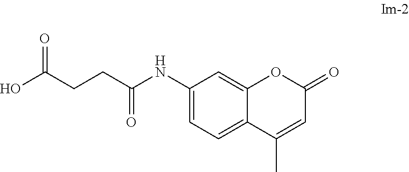 [SMILES: Cc1cc(=O)oc2cc(NC(=O)CCC(=O)O)ccc12]

To a solution of 7-amino-4-methyl-2H-chromen-2-one (0.1 g, 0.57 mmol) and dihydrofuran-2,5-dione (0.057 g, 0.57 mmol) in DMF (0.7 mL) was stirred at room temperature for 16 h. Solvent was evaporated on rota evaporator. The residue was sonicated in ethanol (5 mL) for 1 min. Filter it and dried. Yield:0.13 g, 82.8% (Ref: Synthesis, (6), 932-942; 2008)

### Synthesis of Intermediate Im-09

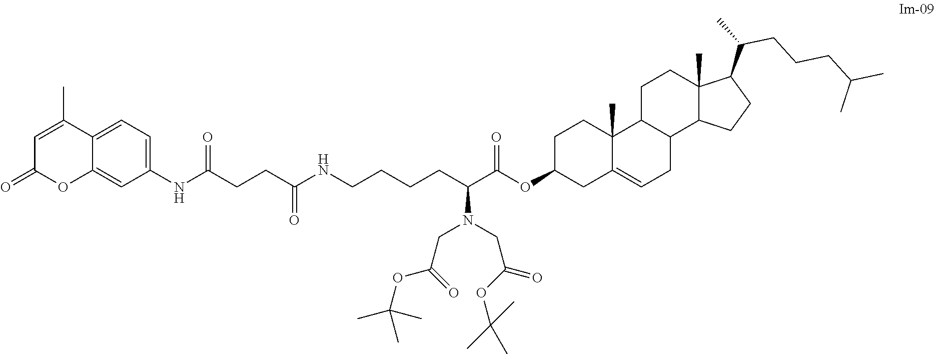 [SMILES: Cc1cc(=O)oc2cc(NC(=O)CCC(=O)NCCCC[C@@H](C(=O)O[C@H]3CC[C@@]4(C)C(=CCC5C4CC[C@@]4(C)C5CC[C@@H]4[C@H](C)CCCC(C)C)C3)N(CC(=O)OC(C)(C)C)CC(=O)OC(C)(C)C)ccc12]

To a solution of compound Im-8 (230 mg, 0.3 mmol), 4-(4-methyl-2-oxo-2H-chromen-7-ylamino)-4-oxobutanoic acid (85 mg, 0.3 mmol) EDC (69 mg, 0.36 mmol) and HOBt (60 mg, 0.45 mmol) was taken in DMF (2.3 mL). Di-isopropyl ethyl amine (0.107 mL, 0.6 mmol) was added and stirred at room temperature for 16 h. Reaction was monitored by TLC, water was added and extracted with ethyl acetate. The organic layer was washed with brine, dried over anhydrous sodium sulfate and evaporated. Crude compound was purified by column chromatography. Compound was eluted in 2% MeOH: CHCl₃. Yield:0.15 g, 48.54%. ¹H NMR (500 MHz, CDCl₃): δ 9.44 (s, 1H); 7.68-7.65 (m, 1H); 7.45-7.35 (m, 2H); 6.79-6.78 (d, 1H); 6.12 (s, 1H); 5.30-5.28 (m, 1H); 4.64-4.56 (m, 1H); 4.45-4.40 (m, 1H); 3.34 (s, 4H); 2.73-2.70 (m, 3H); 2.65-2.59 (m, 2H); 2.36 (s, 3H); 2.32-2.23 (m, 2H); 2.02-1.87 (m, 3H); 1.86-1.74 (m, 6H); 1.71-1.66 (m, 1H); 1.59-1.46 (m, 6H); 1.43 (s, 18H); 1.40-1.37 (m, 3H); 1.34-1.28 (m, 3H); 1.23-1.22 (m, 3H); 1.18-1.02 (m, 8H); 0.97-0.95 (d, 3H); 0.90-0.88 (m, 3H); 0.86-0.82 (m, 6H); 0.65 (s, 3H). ¹³C NMR (101 MHz, CDCl₃) δ 172.87, 171.69, 171.12, 170.99, 161.23, 154.30, 152.30, 141.97, 139.36, 124.97, 122.95, 115.68, 113.17, 107.12, 81.23, 75.22, 56.74, 56.21, 55.97, 53.09, 52.85, 50.06, 42.38, 39.78, 39.59, 38.04, 36.95, 36.62, 36.26, 35.85, 33.18, 31.97, 31.91, 31.28, 31.20, 29.77, 28.25, 28.08, 27.77, 26.81, 24.35, 23.90, 22.89, 22.63, 22.42, 21.08, 19.34, 18.79, 18.59, 11.92. MS (ES-MS) [M+H]⁺ calcd. for C₅₉H₉₀N₃O₁₀ m/z 1000.66, found m/z 1000.63

### Synthesis of Intermediate Im-15

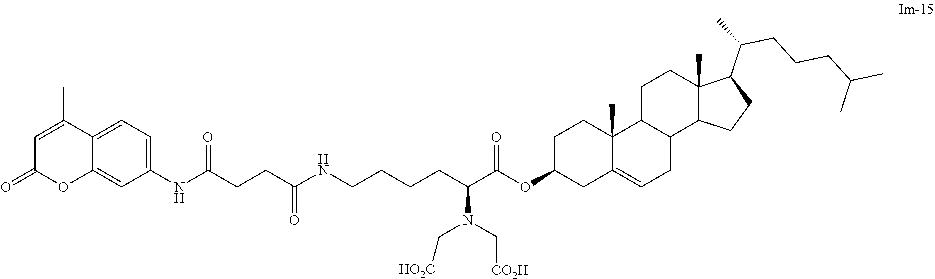

To a solution of compound Im-9 (50 mg, 0.045 mmol) in DCM (5 mL) was added TFA (0.05 mL) drop wise and stirred at room temperature for 16 h. Solvent was evaporated. Diethyl ether was added, solid precipitated out, filter and dried. Yield:44 mg, 100%. MS (ES-MS) [M+H]⁺ calcd. for C₅₁H₇₄N₃O₁₀ m/z 888.54, found m/z 888.54.

### Synthesis of Target Molecule IO-200_06

To a solution of compound Im-15 (44 mg, 0.049 mmol) (pH˜8) was added to a solution of freshly prepared DACH-Pt complex (17 mg, 0.049 mmol) in water and stirred at room temperature for 16 h. A white insoluble precipitate was appeared in the reaction mixture. Then the reaction mixture was filtered and the solid was successively washed with water and dried. Yield: 15 mg, 25.42%. ¹H NMR (500 MHz, DMSO-D₆) δ 7.95-7.89 (m, 1H), 7.76-7.73 (m, 1H), 7.50-7.46 (m, 1H), 6.27 (s, 1H), 5.36-5.18 (m, 1H), 4.58-4.54 (m, 1H), 3.83-3.50 (m, 3H), 2.80-2.71 (m, 4H), 2.50 (s, 3H), 2.36-2.32 (m, 3H), 2.10-2.07 (m, 3H), 1.99-1.80 (m, 8H), 1.63-1.34 (m, 27H), 1.23-0.92 (m, 29H). ¹³C NMR (126 MHz, CDCl₃) δ 182.7, 172.9, 172.7, 172.6, 171.6, 171.5, 171.3, 161.9, 153.6, 153.2, 141.9, 141.8, 138.8, 124.9, 122.5, 115.5, 112.2, 106.5, 75.1, 56.3, 55.7, 49.6, 41.9, 39.3, 39.1, 37.5, 36.5, 36.1, 35.7, 35.4, 31.7, 31.4, 29.2, 27.8, 27.2, 23.8, 23.4, 22.2, 21.9, 20.6, 18.7, 18.1, 17.9, 11.3 ¹⁹⁵Pt NMR (107 MHz, CDCl₃) δ −2302.32, −2332.84. MS (ES-MS) [M]⁺ calcd for C₅₇H₈₆N₅O₁₀Pt; m/z 1195.60, found m/z 1195.6.

### Synthesis of Compound Im-01

The compound Im-01 was synthesized as shown in Scheme 17

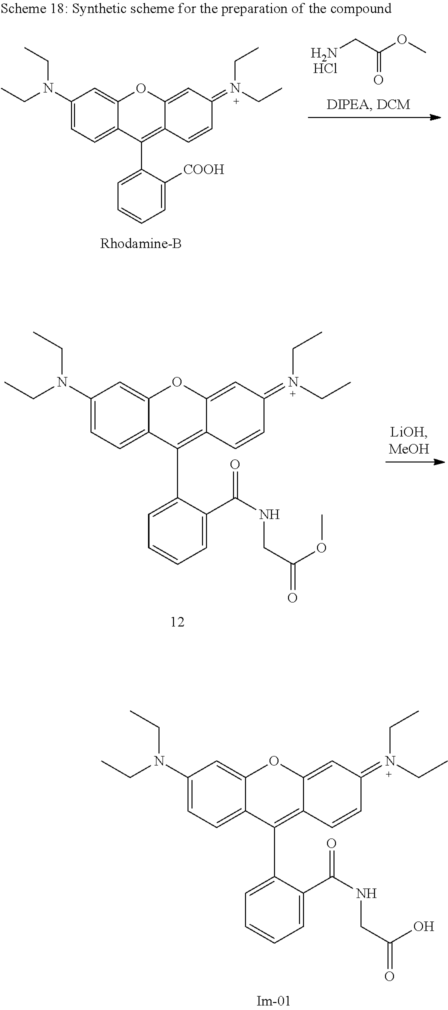

### Synthesis of compound 12

To a solution of Rhodamine B (1.0 g, 2.08 mmol) in DCM (10 mL) was added oxalyl chloride (0.36 mL, 4.17 mmol) followed by 5 drops of DMF under the nitrogen atmosphere. Stirred at room temperature for 16 h. Solvent was evaporated under nitrogen atmosphere and the dissolved residue (in 3 mL DCM) was added to solution of Glycine methyl ester (0.261 g, 2.08 mmol), DIPEA (1.86 mL, 10.4 mmol) in DCM (5 mL) solution drop wise. Stirred at room temperature for 3 h. Then water was added followed by extraction with DCM. The organic was dried over anhydrous sodium sulphate and evaporated. Crude compound was purified by column chromatography in 1% MeOH: CHCl₃ to get 0.57 g, 53.27% of compound 12. ¹H NMR (500 MHz, DMSO-D₆) δ 14.12 (s, 1H), 7.53 (m, 2H), 7.13-7.11 (m, 2H), 6.68-6.18 (m, 5H), 3.51 (s, 2H), 3.47 (s, 3H), 3.44-3.24 (m, 8H), 1.27-1.16 (m, 12H).

### Synthesis of Compound Im-01

To a solution of compound 12 (0.55 g, 1.06 mmol) in mixture of 1:1 MeOH:THF (11 mL) was added a aq. Solution of LiOH (0.089 g, 2.12 mmol) and stirred at room temperature for 16 h. Solvent was evaporated and the residue was taken in water, acidified with dil. HCl, solid get precipitated out. Filter it and dried to get 0.29 g, 54.30% of compound Im-01. ¹H NMR (500 MHz, DMSO-D₆) δ 7.81-7.79 (m, 1H), 7.54-7.50 (m, 2H), 7.03-7.01 (m, 1H), 6.44-6.32 (m, 6H), 3.58 (s, 2H), 3.47 (s, 2H), 3.34-3.28 (q, 8H), 1.11-1.04 (t, 12H). ¹³C NMR (101 MHz, DMSO-D₆) δ 169.34, 167.34, 154.14, 153.26, 148.89, 133.37, 130.31, 129.44, 128.83, 124.16, 122.92, 108.37, 104.83, 97.56, 64.84, 44.16, 41.76, 12.96. MS (ES-MS) [M]⁺ calcd for C₃₀H₃₄N₃O₄ m/z 500.25, found m/z 500.22.

### Synthesis of Compound Im-10

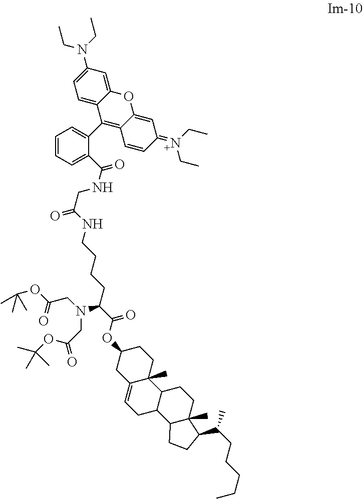 [SMILES: CCCCC[C@@H](C)[C@H]1CCC2C3CC=C4C[C@@H](OC(=O)[C@H](CCCCNC(=O)CNC(=O)c5ccccc5-c5c6ccc(=[N+](CC)CC)cc-6oc6cc(N(CC)CC)ccc56)N(CC(=O)OC(C)(C)C)CC(=O)OC(C)(C)C)CC[C@]4(C)C3CC[C@@]21C]

To a solution of Im-6 (70 mg, 0.14 mmol), EDC (32 mg, 0.169 mmol), HOBt (28 mg, 0.21 mmol) was taken in DMF (2 mL) was stirred at room temperature for 30 min. then compound Im-01 (0.105 g, 0.14 mmol) was added followed by, DIPEA (0.075 mL, 0.42 mmol) and stirred at room temperature for 16 h. Reaction was monitored by TLC, water was added and extracted with ethyl acetate. Organic layer was washed with chilled water and brine, dried over anhydrous sodium sulfate and evaporated. Crude compound was purified by column chromatography. Compound eluted in 2% MeOH/CHCl_{3.} to get 0.1 g, 57.80% of compound Im-10. ¹H NMR (500 MHz, CDCl₃): δ 8.02-8.01 (m, 1H), 7.55 (m, 2H), 7.17-7.15 (m, 2H), 6.70-6.21 (m, 5H), 5.42-5.36 (m, 1H), 4.66-4.60 (m, 1H), 4.33-4.15 (m, 1H), 3.99-3.15 (m, 14H), 2.41-2.13 (m, 3H), 2.12-1.99 (m, 3H), 1.92-1.65 (m, 9H), 1.64-1.53 (m, 6H), 1.50 (s, 18H), 1.43-1.34 (m, 4H), 1.30 (m, 3H), 1.28-1.05 (m, 22H), 1.03 (s, 3H), 0.97-0.94 (d, 3H), 0.92-0.88 (m, 6H), 0.72 (s, 3H). ¹³C NMR (126 MHz, CDCl₃) δ 171.19, 170.60, 168.96, 168.12, 162.55, 153.66, 153.47, 153.42, 148.79, 139.34, 139.27, 132.96, 130.20, 128.66, 128.43, 128.23, 124.05, 123.16, 122.84, 108.27, 97.98, 80.86, 74.94, 65.74, 56.66, 56.11, 55.76, 54.00, 53.47, 52.21, 49.98, 44.63, 44.42, 42.29, 39.70, 39.51, 37.97, 37.90, 36.91, 36.88, 36.54, 36.17, 35.79, 32.63, 31.88, 31.81, 31.44, 29.71, 29.37, 28.24, 28.17, 28.01, 27.66, 27.63, 27.58, 24.28, 23.81, 22.84, 22.75, 22.58, 21.02, 19.33, 18.72, 14.15, 12.60, 11.86. MS (ES-MS) [M]⁺ calcd for C₇₅H₁₁₀N₅O₉ m/z 1224.83, found m/z 1224.84.

### Synthesis of Compound Im-16

To a solution of compound Im-10 (100 mg, 0.081 mmol) in DCM (4 mL), was added TFA (0.5 mL) dropwise and stirred at room temperature for 16 h. Reaction was monitored by TLC, to observe complete conversion. Then the solvent was evaporated under reduced pressure. To the concentrated reaction mixture diethyl ether was added to get clear solution, hexane was added to precipitate out the compound as solid material, then it was decanted and dried to get (88 mg, 98.9%) of compound Im-16 and used as such for next step. MS (ES-MS) [M]⁺ calcd for C₆₇H₉₄N₅O₉ m/z 1112.70, found m/z 1112.72.

### Synthesis of IO-200_07:

To a solution of freshly prepared aquated DACH-Pt complex (24 mg, 0.071 mmol) in water, was added a solution of compound Im-16 (88 mg, 0.071 mmol) in water (not soluble) drop wise, sonicated and stirred the reaction mixture for 16 h. Filter the solid and dried. The solid was taken in methanol, triturated it and filter it. The filtrate was evaporated to get (25 mg, 24.5%) of IO-200_07. ¹H NMR (500 MHz, CDCl₃+1 drop MeOD) δ 8.02-7.98 (m, 1H), 7.71-7.37 (m, 2H), 7.24-7.05 (m, 1H), 7.04-6.63 (m, 1H), 6.61-6.04 (m, 5H), 5.41-5.34 (m, 1H), 4.72-4.53 (m, 1H), 4.51-3.89 (m, 2H), 3.87-3.09 (m, 10H), 3.02-2.38 (m, 2H), 2.37-2.10 (m, 2H), 2.08-1.68 (m, 26H), 1.65-1.42 (m, 10H), 1.41-1.05 (m, 27H), 1.04-0.98 (m, 4H), 0.97-0.92 (m, 3H), 0.91-0.86 (m, 6H), 0.70 (s, 3H). ¹³C NMR (126 MHz, MeOD) δ 185.39, 172.90, 172.27, 170.33, 169.96, 155.03, 154.73, 150.48, 150.38, 140.69, 134.55, 131.60, 129.78, 125.32, 123.95, 123.89, 109.62, 105.66, 105.47, 99.18, 99.07, 76.58, 58.09, 57.56, 52.33, 51.54, 45.41, 43.50, 41.09, 40.69, 39.02, 38.17, 37.75, 37.39, 37.11, 33.36, 33.18, 33.03, 29.34, 29.14, 28.75, 25.54, 25.33, 24.96, 23.24, 22.99, 22.16, 19.88, 19.32, 12.99, 12.39. MS ¹⁹⁵Pt NMR (107 MHz, MeOD) δ −2337.83. (ES-MS) [M]⁺ calcd for C₇₃H₁₀₇N₇O₉Pt m/z 1420.78, found m/z 1420.45.

### Synthesis of Compound Im-11 to Im-14

As mentioned for the previous procedure the common intermediate amine Im-08 is treated with dye derivatives at room temperature for overnight to get the compounds Im-11 to Im-14.

### Synthesis of Compounds Im-17 to Im-20

As mentioned for the previous procedure the common intermediate compounds Im-11 to Im-14 are treated with 25% TFA/CHCl₃ at room temperature for overnight to get the di-acid compounds (Im-17 to Im-20).

### Synthesis of Compounds IO-200_08 to IO-200_11

To a solution of freshly prepared aquated DACH-Pt complex in water, compound Im-17 to Im-20 dissolved in water are added a separately drop wise, sonicated and stirred the reaction mixture for 16 h. Then the solid precipitate is filtered, dissolved with organic solvent, evaporated and dried to get the compound IO-200_08-IO-200_11.

### Example 5: Cell Internalization of Compound IO-200_06

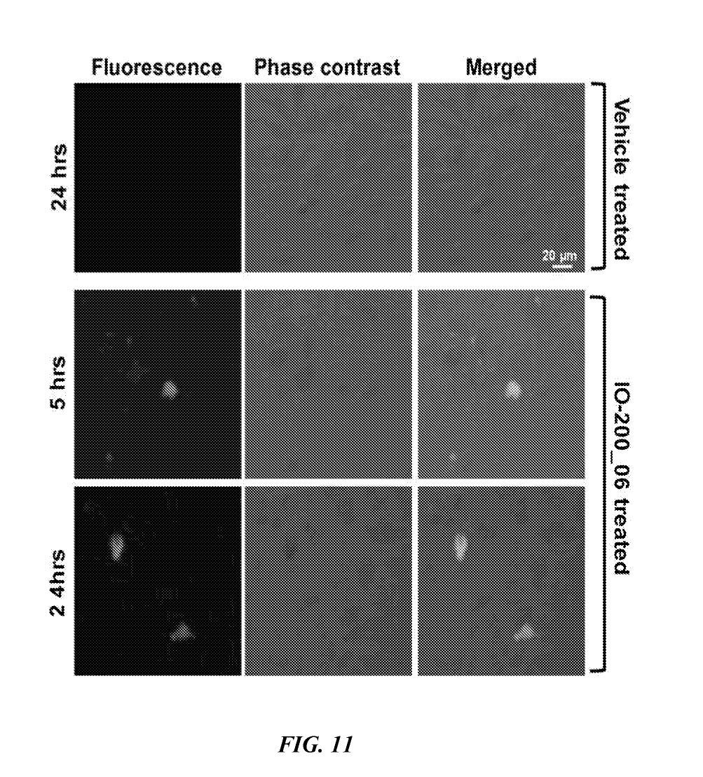

The internalization of IO-200_06 was observed in a lung cancer cell line (A549) using the procedure described in Example 3. Post 5-hours incubation, IO-200_06 could be detected both inside cells and at cell margins, while post 24-hours incubation, it was detected mostly inside cells, while cells treated with vehicle (DMSO) alone did not show any fluorescence (FIG. 11). This shows that after internalization, IO-200_06 retains its fluorescent ability inside cells.

### Example 6: Synthesis and Characterization of Exemplary Compounds

### Synthesis of IO-199_01 (Compound 2a)

Compound IO-199_01 was synthesized following Scheme 19

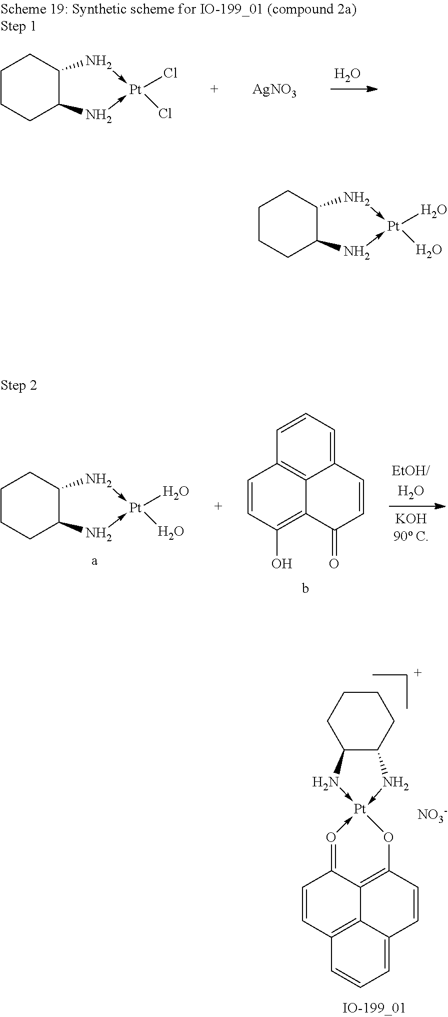

Experimental Procedure (Step 1)

A 250 mL single neck RB flask charged with 1,2-Diaminocyclohexane)platinum(II) chloride (600 mg, 1.5 mmol) was taken in 60 mL water. To the above solution silver nitrate (530 mg, 3.1 mmol) was added. The resulting solution was stirred for 24 hrs. at rt. After completion the reaction mixture was centrifuged and filtered using 0.2 micron filter to remove the precipitate of AgCl. Whole batch of reaction is transferred to next step.

Experimental Procedure (Step 2):

A 100 mL single neck RB flask charged with aquated DACH Platinum a (600 mg, 1.5 mmol) taken in 60 mL water (lyophilized to reduce volume to 5 mL). After this 30 mL EtOH was added. O,O-Phenalenyl b (306 mg, 1.56 mmol) was added along with KOH (86 mg, 1.56 mmol) (solution in EtOH). The resulting solution was stirred for 24 hrs. at 90° C. After completion, reaction mixture was concentrated to remove EtOH and washed with water (30 mL). The precipitate obtained was collected through centrifugation and washed with DCM and centrifuged again to remove the starting material left. The pure compound was obtained by dissolving final precipitate in MeOH. The yield of the compound IO-199_01 was (0.63 g) 80.15%.

### Characterization of IO-199_01 (Compound 2a)

ESIMS m/z=504.14 [M]⁺ for [C19H21N3O5Pt]⁺. 1H NMR (400 MHz, DMSO-d6) δ: 8.43-8.45 (m, 4H), 7.69 (t, J=8 Hz, 1H), 7.23 (d, J=8 Hz, 2H), 6.53 (d, J=4 Hz, 2H), 6.02 (t, J=8 Hz, 2H), 2.35 (m, 2H), 1.95 (d, J=8 Hz, 2H), 1.53 (d, J=4 Hz, 2H), 1.35 (d, J=4 Hz, 2H), 1.07-1.11 (m, 2H) ppm. ¹³C NMR (100 MHz, DMSO-d6) δ: 170.54, 138.96, 132.33, 126.67, 126.26, 125.87, 124.88, 114.42, 62.28, 31.49, 23.97 ppm. ¹⁹⁵Pt NMR (86 MHz, DMSO-d6) δ: −1575 ppm.

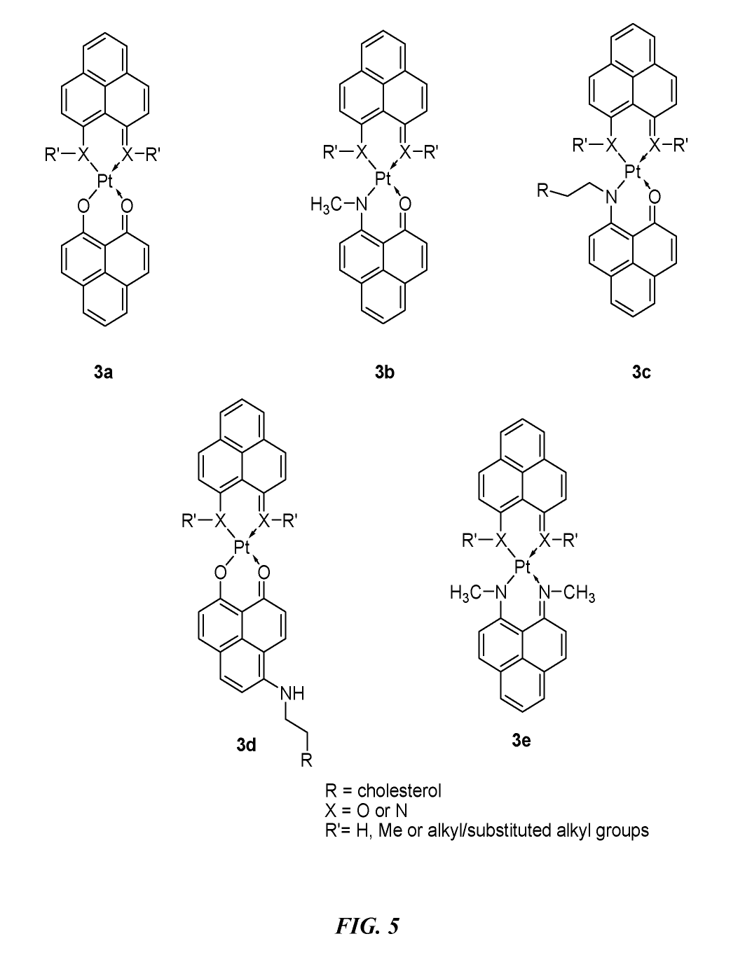

FIG. 7 shows the single crystal X-ray diffraction structure of compound 2a.

### HPLC Analysis of IO-199_01 (Compound 2a)

Equisil-BDS C8 column was used to check the analytical purity of compound 2a with gradient method of 5% MeOH/Water to 100% MeOH with a gradient time of 15 minute. The purity of IO_199-01 was 96.42% (data not shown).

### Synthesis of IO-199_02 (Compound 2b)

The compound IO-199_02 was synthesized following Scheme 20.

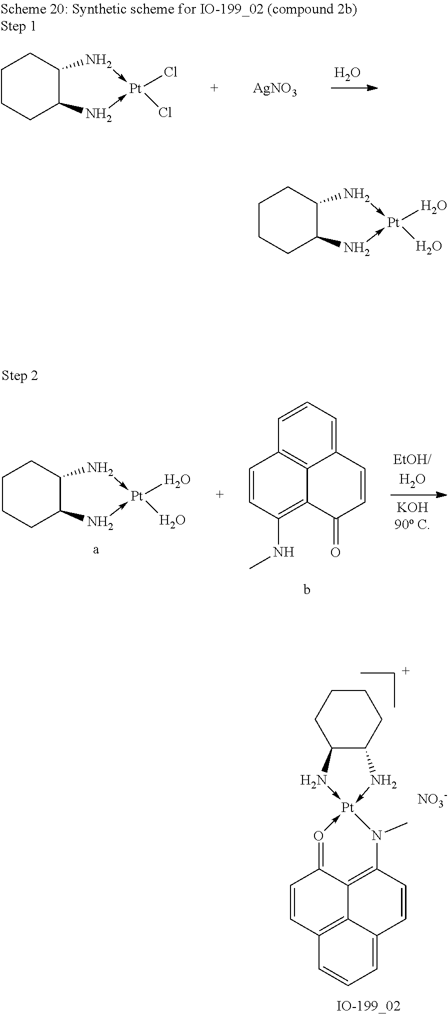

Experimental Procedure (Step 1):

Step 1 was followed as same as that of step 1 of IO-199_01 (compound 2a) above. In this case scale was doubled.

Experimental Procedure (Step 2):

A 250 mL single neck RB flask charged with aquated DACH Platinum a (1.2 g, 3.16 mmol) taken in 30 mL water (lyophilized to reduce 120 ml volume to 30 mL). After this 70 mL EtOH was added. N,O-Phenalenyl b (0.328 g, 1.56 mmol) was added along with KOH (0.86 g, 1.56 mmol) (solution in EtOH). The resulting solution was stirred for 24 hrs. at 90° C. After completion, reaction mixture was concentrated to remove EtOH and washed with water (30 mL). The precipitate obtained was collected through centrifugation and washed with DCM and centrifuged again to remove the starting material left. The pure compound was obtained by dissolving final precipitate in MeOH. The yield of the compound IO-199_02 (compound 2b) was 0.78 g (96%).

### Characterization of IO-199_02 (compound 2b)

ESIMS m/z=518.15 [M+H]⁺ for [C₂₀H₂₅N₃OPt]⁺. ¹H NMR (400 MHz, DMSO-d6) δ: 8.15-8.20 (m, 2H), 8.10 (d, J=8 Hz, 1 H), 8.04 (d, J=8 Hz, 1H), 7.45-7.50 (m, 2H), 7.17 (d, J=4 Hz, 1H), 6.12 (d, J=4 Hz, 1H), 6.02 (d, J=4 Hz, 1H), 5.74 (t, J=8 Hz, 1H), 5.62 (t, J=8 Hz, 1H), 3.75 (s, 3H), 2.37-2.39 (m, 1H), 2.26-2.28 (m, 1H), 1.95-1.97 (m, 2H), 1.54-1.55 (m, 2H), 1.33-1.35 (m, 2H), 1.10-1.11 (m, 2H) ppm. ¹³C NMR (100 MHz, DMSO-d6) δ: 163.54, 151.71, 135.37, 134.04, 130.40, 129.39, 127.39, 126.36, 125.04, 123.43, 119.54, 113.26, 62.51, 60.35, 46.17, 31.92, 31.88, 24.05, 24.03 ppm. ¹⁹⁵Pt NMR (107 MHz, DMSO-d6) δ: −1844.11 ppm.

### HPLC Analysis of IO-199_02 (Compound 2b)

Equisil-BDS C8 column used to check the analytical purity of compound IO-199_02 with a gradient method of 25% MeOH/Water to 100% MeOH and a gradient time of 15 minute. The purity of IO-199_02 was 99.78% (data not shown).

### Synthesis of IO-199_03 (Compound 2d)

The compound IO-199_03 was synthesized following Scheme 21.

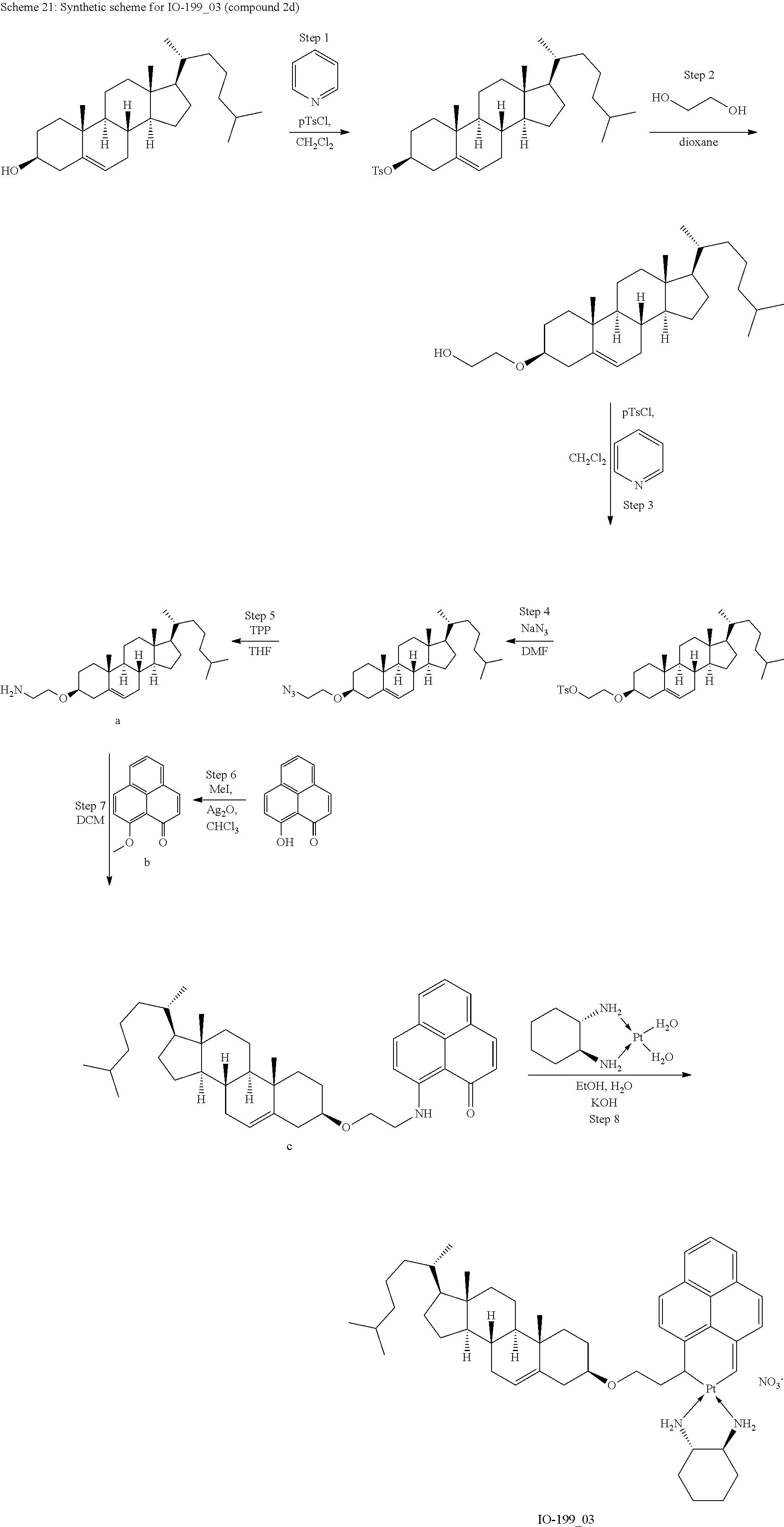

Experimental Procedure (Step 1-5):

Synthesis of compound a was carried out as described in U.S. patent application Ser. No. 14/898,355, titled “LIPID-BASED PLATINUM COMPOUNDS AND NANOPARTICLES,” content of which is incorporated herein by reference in its entirety.

Experimental Procedure (Step 6):

Synthesis of compound b was carried out as described ACS Catalysis, 2014, 4, 4307-4319, content of which is incorporated herein by reference in its entirety.

Experimental Procedure (Step 7):

A 100 mL single neck RB flask charged with cholesterol amine a (1.2 g, 2.8 mmol) taken in 80 mL DCM. To it, DCM solution of O, O-phenalenyl b (600 mg, 2.8 mmol) was added. The resulting solution was refluxed overnight. After completion, reaction mixture was concentrated to remove DCM and compound was purified by column chromatography (Silica 60-120 mesh, Ethyl acetate: Hexane: 10%). The yield of the compound c is 76%.

### Characterization of c

ESIMS m/z=608.42 [M+H]⁺ for [C₄₂H₅₈NO₂]⁺. ¹H NMR (400 MHz, CDCl₃) δ: 12.30 (br s, 1H), 7.99 (d, J=8 Hz, 1H), 7.83-7.89 (m, 3H), 7.41-7.44 (t, J=4 Hz, J=8 Hz, 1H), 7.27 (d, J=8 Hz, 1H), 7.00 (d, J=8 Hz, 1H), 5.35 (s, 1H), 3.80-3.87 (m, 2H), 3.73-3.76 (m, 2H), 3.24-3.30 (m, 1H), 0.83-2.44 (m, 40H), 0.66 (s, 3H) ppm. ¹³C NMR (125 MHz, CDCl₃) δ: 181.81, 157.07, 140.64, 139.32, 138.74, 132.45, 132.04, 127.89, 127.60, 125.33, 124.53, 122.28, 121.81, 114.76, 107.89, 79.94, 66.24, 56.78, 56.18, 50.18, 43.49, 42.33, 39.79, 39.53, 39.00, 37.17, 36.87, 36.21, 35.79, 31.96, 29.71, 28.37, 28.24, 28.02, 24.30, 23.85, 22.82, 22.57, 21.08, 19.41, 18.73, 11.86.

Experimental Procedure (Step 8):

A 250 mL single neck RB flask charged with aquated DACH Platinum d (400 mg, 1.05 mmol) taken in 40 mL water (lyophilized to reduce volume to 10 mL). After this 10 mL EtOH was added. Compound c (319 mg, 0.52 mmol) (dissolved in 62 mL EtOH) was added along with KOH (29.1 mg, 0.52 mmol) (solution in H₂O). The resulting solution was stirred for 24 hrs. at 90° C. After completion, reaction mixture was concentrated to remove EtOH and washed with water (30 mL). The precipitate obtained collected through centrifugation was purified by Column chromatography (Silica 60-120 mesh, MeOH: CHCl₃:3%). The yield of the compound IO-199_03 was 119 mg (23.42%).

### Characterization of IO-199_03 (Compound 2d)

ESIMS m/z=915.43 [M]⁺ for [C₄₈H70N₃O₂Pt]⁺. ¹H NMR (400 MHz, DMSO-d6) δ: 8.20 (t, J=8 Hz, 2H), 8.12 (d, J=8 Hz, 1H), 8.03 (d, J=8 Hz, 1H), 7.48-7.52 (m, 2H), 7.19 (d, J=4 Hz, 1H), 6.17 (s, 1H), 5.82 (s, 1H), 5.73 (t, J=8 Hz, 2H), 5.29 (s, 1H), 4.13-4.40 (m, 2H), 3.77-3.85 (m, 2H), 3.21 (m, 1H), 0.83-2.36 (m, 50H), 0.62 (s, 3H) ppm. ¹³C NMR (126 MHz, DMSO) δ: 163.82, 151.39, 140.01, 135.08, 134.23, 130.40, 129.26, 127.56, 126.51, 126.38, 125.14, 123.58, 121.48, 119.78, 113.24, 78.94, 67.87, 62.54, 60.22, 56.65, 56.12, 55.56, 49.52, 41.83, 38.92, 38.50, 36.48, 36.25, 35.64, 35.17, 31.81, 31.53, 31.37, 31.33, 29.42, 29.04, 27.93, 27.76, 27.38, 24.07, 23.84, 23.18, 22.65, 22.38, 20.57, 19.01, 18.54, 11.65.

### HPLC Analysis of IO-199_03 (Compound 2d)

Equisil-BDS C8 column used to check the analytical purity of compound IO-199_03 with a gradient method of 5% MeOH/Water to 100% MeOH and a gradient time of 15 minute. The purity of IO-199_03 was 93.36% (data not shown).

### Synthesis of IO-199_04 (Compound 2i)

The compound IO-199_04 was synthesized following Scheme 22.

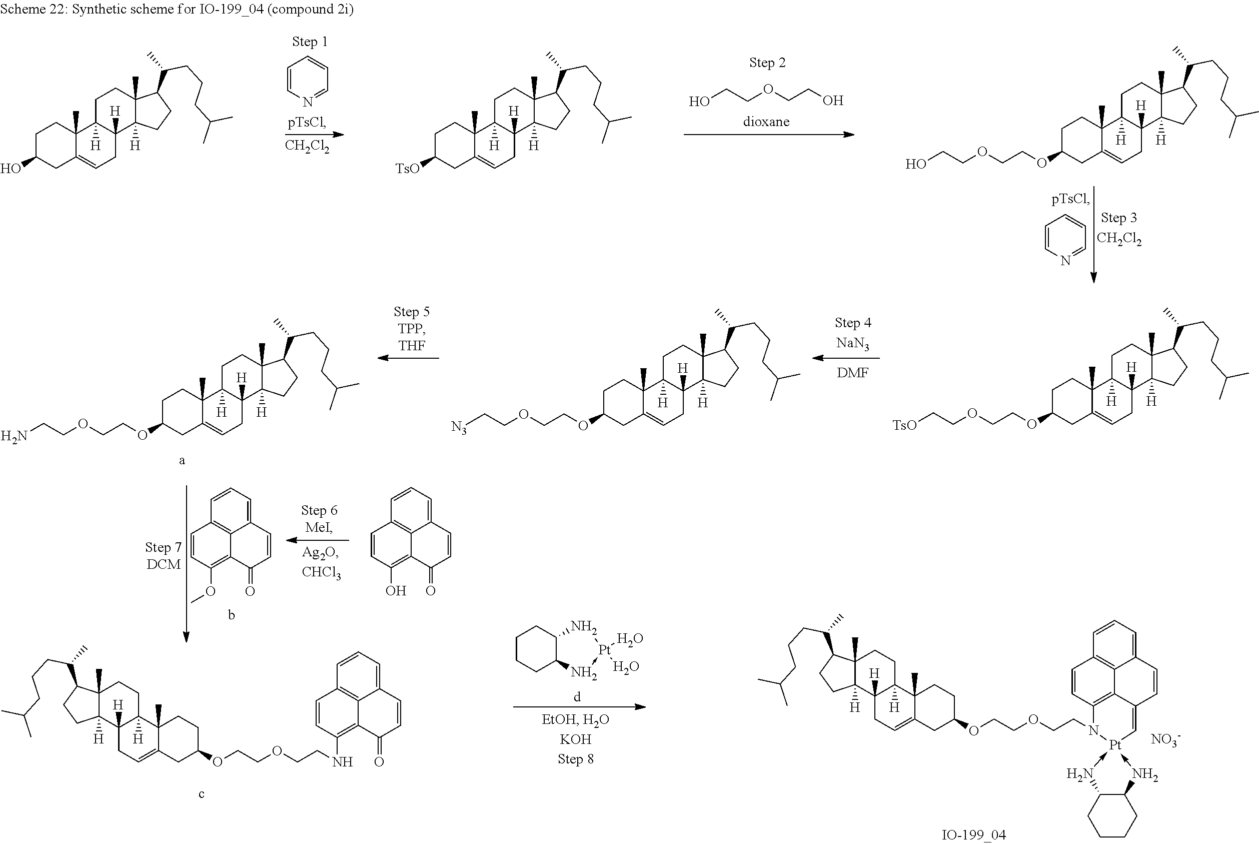

Experimental Procedure (Step 1-5):

Synthesis of compound a was carried out as described in U.S. patent application Ser. No. 14/898,355, titled “LIPID-BASED PLATINUM COMPOUNDS AND NANOPARTICLES.”

Experimental Procedure (Step 6):

Synthesis of compound b was carried out as described ACS Catalysis, 2014, 4, 4307-4319.

Experimental Procedure (Step 7):

A 100 mL single neck RB flask charged with cholesterol digol amine a (648 mg, 1.3 mmol) taken in 50 mL DCM. To it, DCM solution of O, O-phenalenyl b (285 mg, 1.3 mmol) was added. The resulting solution was refluxed overnight. After completion, reaction mixture was concentrated to remove DCM and compound was purified by column chromatography (Silica 60-120 mesh, Ethyl acetate: Hexane: 25%). The yield of the compound c is 66.9%.

### Characterization of c

ESIMS m/z=652.49 [M+H]⁺ for [C₄₄H₆₂NO3]⁺. ¹H NMR (400 MHz, CDCl₃) δ: 12.33 (br s, 1H), 7.99 (d, J=4 Hz, 1H), 7.83-7.89 (m, 3H), 7.43 (t, J=4 Hz, J=8 Hz, 1H), 7.28 (d, J=8 Hz, 1H), 6.99 (d, J=8 Hz, 1H), 5.28 (s, 1H), 3.85-3.87 (m, 2H), 3.76-3.80 (m, 2H), 3.69-3.71 (m, 2H), 3.65-3.67 (m, 2H), 3.17 (m, 1H), 0.85-2.35 (m, 40H), 0.66 (s, 3H) ppm. ¹³C NMR (125 MHz, CDCl₃) δ: 183.02, 155.68, 140.37, 138.37, 138.10, 131.71, 131.44, 128.36, 127.67, 124.39, 123.91, 121.69, 120.88, 115.18, 107.16, 78.34, 70.42, 69.11, 66.62, 56.02, 55.50, 49.40, 42.25, 41.78, 39.06, 38.64, 36.52, 36.12, 35.66, 35.19, 31.26, 31.20, 29.02, 28.08, 27.78, 27.42, 23.82, 23.18, 22.70, 22.43, 20.52, 18.96, 18.57, 11.65.

Experimental Procedure (Step 8):

A 100 mL single neck RB flask charged with aquated DACH Platinum d (387 mg, 1.01 mmol) taken in 40 mL water (lyophilized to reduce volume to 10 mL). After this 20 mL EtOH was added. Ligand c (338 mg, 0.51 mmol) (dissolved in EtOH) was added along with KOH (57.2 mg, 1.02 mmol) (solution in H₂O). The resulting solution was stirred for 24 hrs. at 90° C. After completion, reaction mixture was concentrated to remove EtOH and washed with water (30 mL). The precipitate obtained collected through centrifugation was purified by Column chromatography (Silica 60-120 mesh, MeOH: CHCl₃:3%). The yield of the compound IO-199_04 was 350 mg (44.8%).

### Characterization of IO-199_04 (Compound 2i)

ESIMS m/z=959.62 [M]⁺ for [C₅₀H₇₄N₃O₃Pt]⁺. ¹H NMR (500 MHz, DMSO-d₆) δ 8.19 (t, J=5.0, 10.0 Hz, 2H), 8.11 (d, J=5 Hz, 1H), 8.02 (d, J=10 Hz, 1H), 7.47-7.51 (m, 2H), 7.18 (d, J=10 Hz, 1H), 6.14 (s, 1H), 5.67-5.76 (m, 3H), 5.19 (s, 1H), 4.16-4.45 (m, 2H), 3.79-3.84 (m, 2H), 3.58 (m, 2H), 3.55 (m, 2H), 3.00-3.06 (m, 1H), 0.83-2.40 (m, 50H), 0.62 (s, 3H). ¹³C NMR (125 MHz, DMSO-d6) δ: 163.86, 151.30, 140.26, 135.13, 134.24, 130.42, 129.28, 127.61, 126.54, 126.39, 125.13, 123.56, 121.09, 119.70, 113.26, 78.55, 70.92, 70.70, 66.77, 62.50, 60.30, 56.43, 56.10, 55.55, 49.47, 41.81, 38.93, 38.69, 36.55, 36.16, 35.65, 35.19, 32.11, 31.83, 31.53, 31.32, 31.28, 27.98, 27.78, 27.40, 24.04, 23.85, 23.19, 22.68, 22.40, 20.54, 18.99, 18.56, 13.97, 11.65 ppm. ¹⁹⁵Pt NMR (107 MHz, DMSO-d6) δ: −1824.95 ppm.

### HPLC Analysis of IO-199_04 (Compound 2i)

Equisil-BDS C8 column used to check the analytical purity of compound IO-199_04 with a gradient method of 90% MeOH/Water to 100% MeOH and a gradient time of 15 minute. The purity of IO-199_04 was 96.03% (data not shown).

### Synthesis of IO-199_12 (Compound 2n)

Compound IO-199_12 was synthesized following Scheme 23.

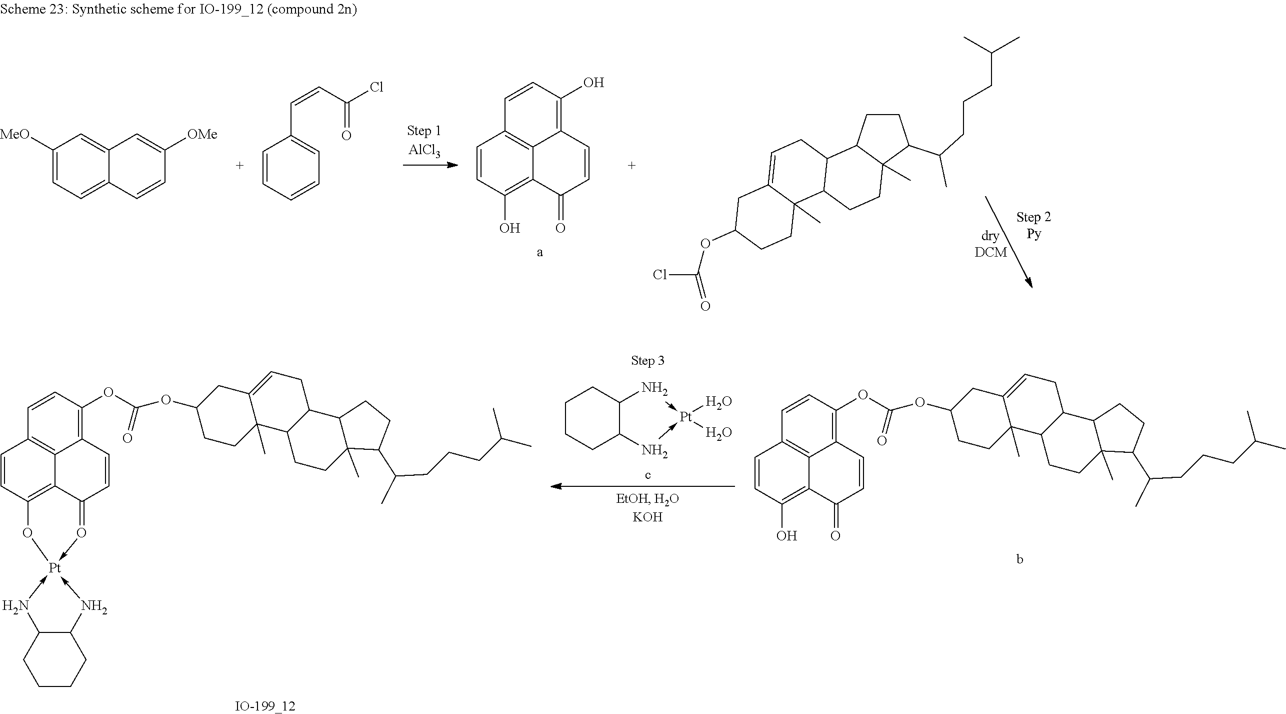

Experimental Procedure (Step 1):

2,7-Dimethoxynapthalene (5 g) and cinnamoyl chloride (4.4 g) were dissolved in dry 1,2-dichlroethane (50 mL) and kept in ice bath. Total amount of AlCl₃ (2.5 eq, 8.7 g) divided in to three portions, add the first portion of AlCl₃ to the reaction mixture and leave it for 1 h string. Then bring it to room temperature and add second portion of AlCl₃ and reflux it for 1 h. After 1 h reflux final portion were added to the reaction mixture and continue the reflux for overnight. At the end of the reaction, a solid started to form that was mechanically broken up and the crude mixture was quenched by ice cold 6N HCl. Total crude compound dissolved in MeOH and precipitate done from methanol and hexane mixture. Dissolve the compound again in acetone, only compound was soluble in acetone and filtered the solution and evaporated the solvent which gave yellow crystalline powder a.

### Characterization of a

¹H NMR (400 MHz, CDCl₃) δ: 16.82 (br s, 1H), 12.00 (br s, 1H), 8.55 (d, J=8 Hz, 1H), 8.24 (d, J=8 Hz, 1H), 8.12 (d, J=8 Hz, 1H), 7.22 (d, J=4 Hz, 1H), 7.07 (d, J=4 Hz, 1H), 7.01 (d, J=8 Hz, 1H).

Experimental Procedure (step 2):

The compound a (48 mg) and Pyridine (25 μL) dissolved in dry DCM and kept under nitrogen atmosphere in 100 ml R.B and stirred it for 10 min. Cholesterol chloroformate (500 mg) dissolved in dry DCM and added to reaction mixture dropwise and continued the reaction for overnight. Compound was purified by Column chromatography (Silica 60-120 mesh, hexane: CHCl₃: 50%).

### Characterization of b

ESIMS m/z=645.30 (M+H)⁺, 663.36 (M+K)⁺. ¹H NMR (400 MHz, CDCl₃) δ: 16.11 (br s, 1H), 8.29 (d, J=8 Hz, 1H), 8.03 (d, J=8 Hz, 1H), 7.99 (d, J=4 Hz, 1H), 7.48 (d, J=4 Hz, 1H), 7.18 (d, J=8 Hz, 1H), 7.12 (d, J=8 Hz, 1H), 5.42 (s, 1H), 4.67-4.61 (m, 1H), 0.83-2.52 (m, 40H), 0.66 (s, 3H) ppm. ¹³C NMR (125 MHz, CDCl₃) δ: 179.52, 179.03, 152.30, 151.09, 140.74, 138.87, 134.09, 133.76, 128.06, 117.60, 117.35, 111.11, 79.82, 56.67, 56.14, 49.98, 42.32, 39.70, 39.53, 37.91, 36.82, 36.56, 36.19, 35.80, 31.91, 31.83, 729.72, 29.39, 28.24, 28.03, 27.65, 24.29, 23.85, 22.85, 22.71, 22.59, 21.07, 19.31, 18.73, 14.15, 11.88.

Experimental Procedure (Step 3):

A 100 mL single neck RB flask charged with aquated DACH Platinum (387 mg, 1.01 mmol) taken in 40 mL water (lyophilized to reduce volume to 10 mL). After this 20 mL EtOH was added. Compound b (318 mg, 0.51 mmol) (dissolved in EtOH) was added along with KOH (57.2 mg, 1.02 mmol) (solution in H₂O). The resulting solution was stirred for 24 hrs. at 90° C. After completion, reaction mixture was concentrated to remove EtOH and washed with water (30 mL).

### Synthesis of IO-199_07 (Compound 2l)

The compound IO-199_07 was synthesized following Scheme 24.

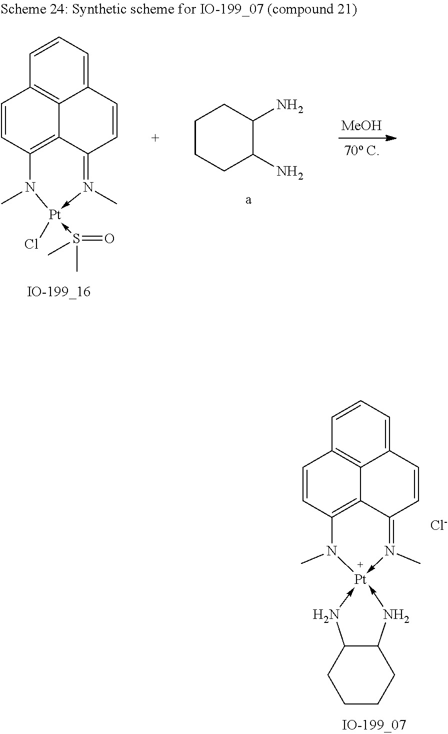

Experimental Procedure:

A 100 mL single neck RB flask charged with PLY-Platinum DMSO complex IO-199_16 (100 mg, 0.188 mmol) taken in 50 mL Methanol. To it, Diamino cyclohexane a (21.46 mg, 0.188 mmol) was added. The resulting solution was stirred for 24 hrs. at 70° C. After completion, reaction mixture was centrifuged to remove precipitate and the Methanol part was concentrated and precipitate obtained was washed with water (20 mL) and collected through centrifugation and lyophilization. The yield of the complex IO-199_07 is 45 mg (45.4%).

### Characterization of IO-199_07 (compound 2l)

ESIMS m/z=530.0936 [M]+ for [C₂₁H₂₇ClN₄Pt]+. ¹H NMR (500 MHz, MeOD) δ: 7.81 (d, J=10 Hz, 2H), 7.73 (d, J=10 Hz, 2H), 7.19-7.25 (m, 3H), 5.44 (bs, 2H), 5.11 (bs, 2H), 3.61 (s, 6H), 2.28 (s, 2H), 2.05 (d, J=10 Hz, 2H), 1.65 (d, J=10 Hz, 2H), 1.27-1.31 (m, 2H), 1.21 (t, J=10 Hz, J=10 Hz 2H). ¹³C NMR (126 MHz, MeOD) δ: 153.20, 134.86, 130.61, 129.81, 127.35, 122.58, 119.03, 62.28, 45.26, 34.09, 25.69.

### HPLC Analysis of IO-199_07 (Compound 2l)

Equisil-BDS C8 column used to check the analytical purity of compound IO-199_07 with a gradient method of 25% MeOH/Water to 100% MeOH and a gradient time of 15 minute. The purity of IO-199_07 was 98.73% (data not shown).

### Synthesis of IO-199_23 (Compound 2r)

The compound IO-199_23 was synthesized following Scheme 25.

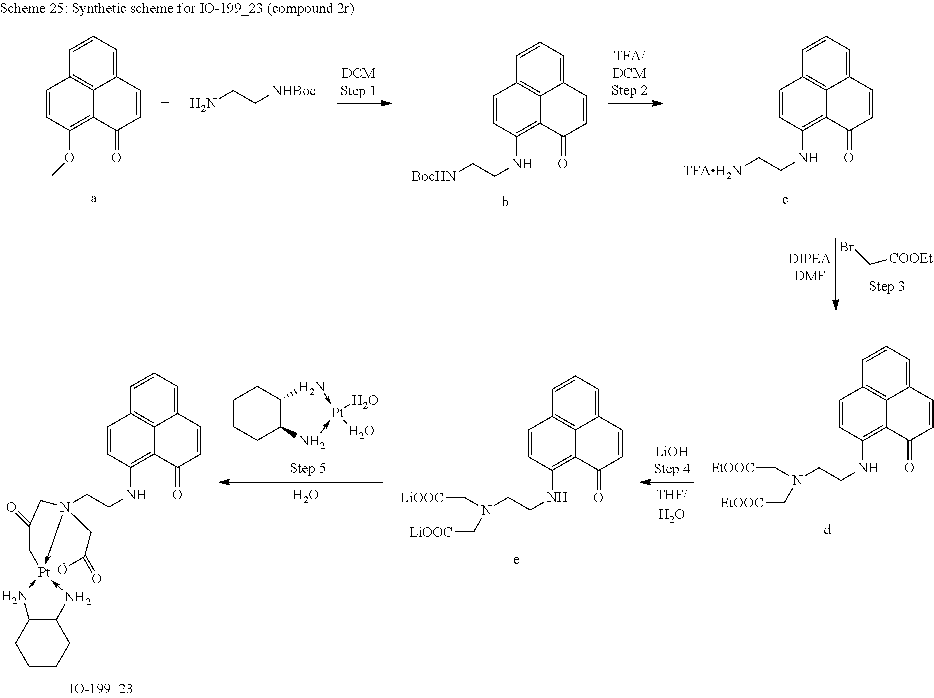

Experimental Procedure (Step 1):

A 100 mL single neck RB flask charged with O-methoxy PLY a (300 mg, 0.056 mmol) with 40 mL DCM. To it, Boc-protected ethylenediamine (228.6 mg, 0.056 mmol) was added. The resulting solution was refluxed for 18 hrs. at 50° C. After completion of reaction, it was washed with water and extracted with CHCl₃. Final product b was purified by column chromatography (Silica 60-120 mesh, Ethyl acetate: Hexane:25%).

### Characterization of b

ESIMS m/z=238.9914 [M-Boc]⁺ for [C₂₀H₂₂N₂O₃]⁺. ¹H NMR (500 MHz, CDCl3) δ: 12.20 (s, 1H), 7.92 (d, J=10 Hz, 1H), 7.84 (d, J=5 Hz, 1H), 7.78 (d, J=10 Hz, 1H), 7.74 (d, J=5 Hz, 1H), 7.39 (t, J=10 Hz, J=10 Hz, 1H), 7.23 (d, J=9.2 Hz, 1H), 6.97 (d, J=10 Hz, 1H), 5.43 (s, 1H), 3.71 (s, 2H), 3.49 (s, 2H), 1.45 (s, 9H).

Experimental Procedure (Step 2):

A 50 mL single neck RB flask charged with Boc-protected amino PLY b (350 mg, 1.032 mmol). To it 20% TFA solution was added dropwise at 0° C. The resulting solution was stirred for 3 hrs. After completion of reaction, DCM was removed. TFA was removed by giving DCM, Diethyl ether and toluene wash. Without further purification, it was used for next reaction.

Experimental Procedure (Step 3):

A 100 mL single neck RB flask charged with Compound c (247 mg, 1.033 mmol) with 20 mL DMF followed by DIEPA (237 μL, 2.066 mmol) and stirred for 20 minutes. To it Ethyl bromo acetate was added dropwise and the resulting solution was stirred for 18 hrs. at rt. After completion of reaction, compound was extracted with Ethyl acetate. Final product was purified by column chromatography (Silica 60-120 mesh, Ethyl acetate: Hexane:25%).

### Characterization of d

ESIMS m/z=410.8907[M]⁺ for [C₂₃H₂₆N₂O₅]⁺. ¹H NMR (500 MHz, CDCl3-d6) δ: 12.12 (s, 1H), 8.00 (d, J=5 Hz, 1H), 7.88-7.90 (m, 3H), 7.45 (t, J=10 Hz, J=5 Hz 1H), 7.29 (d, J=10 Hz, 1H), 7.13 (d, J=10 Hz, 1H), 4.14-4.18 (m, 4H), 3.76 (q, 2H), 3.70 (s, 4H), 3.24 (t, J=10 Hz, J=5 Hz, 2H), 1.25 (d, J=5 Hz, 6H).

Experimental Procedure (Step 4):

A 100 mL single neck RB flask charged with compound d (52.73 mg, 0.128 mmol) with 12 mL THF/H₂O (3:1). The reaction mixture was cooled to 0° C. under ice bath for 10 mins and LiOH (10.74 mg, 0.256) was added. The resulting solution was stirred for 5-6 hrs. at rt. After completion, reaction mixture was concentrated to remove THF and diluted with water (20 mL) and water layer was washed with Ethyl acetate, DCM and hexane successively. The aqueous layer (yellow) was utilized for the next step.

Experimental Procedure (Step 5):

A 100 mL single neck RB flask charged with Aquated DACH platinum f (59.04 mg, 0.144 mmol) in 6 mL water. To the above solution compound e (44.5 mg, 0.144 mmol) in 30 mL water was added. The resulting solution was stirred for 24 hrs at rt. No precipitate was observed even after 24 hrs stirring. Whole of the solution was lyophilized to get the solid.

### Synthesis of IO-199_24 & 33 (Compound 2s and 2z)

The compounds IO-199_24 and IO-199_33 were synthesized as shown in Scheme 26.

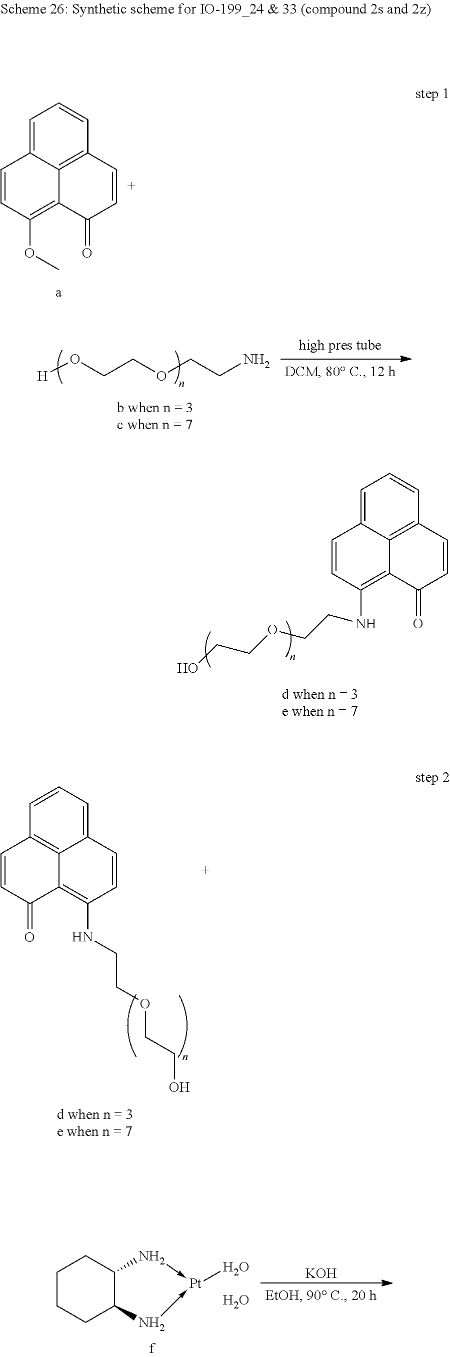

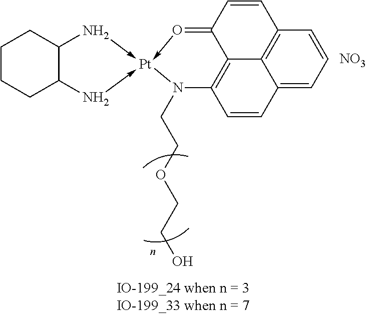

### Synthesis of IO-199_24 (Compound 2s)

Experimental Procedure (Step 1):

A 10 mL Pressure tube was charged with O-Methoxy PLY a (0.459 g, 2.187 mmol) with 5 mL DCM. To it, tetra peg amine alcohol b (0.507 g, 2.625 mmol) was added and heated at 80° C. for 18 hrs. After completion of reaction, compound was washed with water and extracted with CHCl₃. Final product d was purified by column chromatography (Silica 60-120 mesh, Ethyl acetate:Hexane:80:20 and then Methanol: CHCl₃: 10:90). Yield was 63%.

### Characterization of d

ESIMS m/z=372.0175 [M+1]⁺ for [C₂₁H₂₄NO₆]+. ¹H NMR (500 MHz, CDCl3-d6) δ: 12.20 (s, 1H), 8.20 (d, J=10 Hz, 1H), 8.06 (d, J=5 Hz, 1H), 8.01 (t, J=5 Hz, 10 Hz, 2H), 7.49 (t, J=5 Hz, 10 Hz, 2H), 6.85 (d, J=10 Hz, 1H), 4.56 (m, 1H), 3.76 (m, 4H), 3.36-3.62 (m, 11H).

Experimental Procedure (Step 2):

A 250 mL single neck RB flask charged with aquated DACH Platinum f (0.474 g, 1.315 mmol) taken in 40 mL water (lyophilized to reduce volume to 10 mL). After this 10 mL EtOH was added. Tetra peg alcohol PLY Ligand d (0.245 g, 0.65 mmol) (dissolved in 50 mL EtOH) was added along with KOH (29.1 mg, 1.25 mmol) (solution in EtOH). The resulting solution was refluxed for 24 hrs. at 90° C. After completion, reaction mixture was concentrated to remove EtOH and DCM was added to the reaction mixture. It was washed with water (30 mL). The organic layer was concentrated and purified by Column chromatography (Silica 60-120 mesh, MeOH: CHCl₃: 2%-10%). The yield of the compound (IO-199_24) (2s) was 0.446 g (56%).

### Characterization of IO-199_24 (Compound 2s)

ESIMS m/z=679.0167 [M]⁺ for [C₂₇H₃₇N₃O₅Pt]⁺. ¹H NMR (500 MHz, CDCl3-d6) δ: 8.20 (t, J=10 Hz, 10 Hz, 2H), 8.12 (d, J=10 Hz, 1H), 8.03 (d, J=10 Hz, 1H), 7.47-7.52 (m, 2H), 7.18 (d, J=10 Hz, 1H), 6.13 (br s, 1H), 5.80 (br s, 1H), 5.70 (t, 10 Hz, 10 Hz, 2H), 4.61 (s, 1H), 4.46 (s, 1H), 4.17 (s, 1H), 3.75-3.85 (m, 2H), 3.44-3.61 (m, 11H), 2.32-2.39 (m, 2H), 1.94-1.97 (m, 2H), 1.54-1.56 (m, 2H), 1.31-1.38 (m, 2H), 1.08-1.13 (m, 2H) ppm.

### HPLC Analysis of IO-199_24 (Compound 2s)

Equisil-BDS C8 column used to check the analytical purity of compound IO-199_24 with a gradient method of 25% MeOH/Water to 100% MeOH and a gradient time of 15 minute. The purity of IO-199_24 was 91.98% (data not shown).

### Synthesis of IO-199_33 (Compound 2z)

Experimental Procedure (Step 1):

A 10 mL Pressure tube was charged with O-Methoxy PLY a (0.130 g, 0.62 mmol) with 4 mL Methanol. To it, octa peg amine alcohol c (0.260 g, 0.70 mmol) was added and heated at 80° C. for 18 hrs. After completion of reaction, compound was washed with water and extracted with CHCl₃. Final product e was purified by column chromatography (Silica 60-120 mesh, Ethyl acetate: Hexane: 80:20 and then Methanol: CHCl₃: 10:90). Yield was 59%.

### Characterization of e

ESIMS m/z=548.3137 [M+1]⁺ for [C₂₉H₄₁NO₉]⁺. ¹H NMR (500 MHz, CDCl3-d6) δ: 12.18 (s, 1H), 8.20 (d, J=10 Hz, 1H), 8.06 (d, J=10 Hz, 1H), 8.01 (t, J=5 Hz, 10 Hz, 2H), 7.49 (m, 2H), 6.85 (d, J=10 Hz, 1H), 4.60 (m, 2H), 3.76 (m, 4H), 3.60-3.62 (m, 2H), 3.55-3.57 (m, 2H), 3.45-3.50 (m, 22H).

Experimental Procedure (Step 2):

A 250 mL single neck RB flask charged with aquated DACH Platinum f (0.189 g, 0.524 mmol) taken in 20 mL water (lyophilized to reduce volume to 10 mL). After this 10 mL EtOH was added. Octa peg alcohol PLY Ligand e (0.144 g, 0.263 mmol) (dissolved in 30 mL EtOH) was added along with KOH (0.028 g, 5.00 mmol) (solution in EtOH). The resulting solution was refluxed for 24 hrs. at 90° C. After completion, reaction mixture was concentrated to remove EtOH and DCM was added to the reaction mixture. It was washed with water (30 mL). The organic layer was concentrated and purified by Column chromatography (Silica 60-120 mesh, MeOH: CHCl₃: 2%-10%). The yield of the compound was 0.1 g (45%).

### Characterization of IO-199_33 (Compound 2z)

ESIMS m/z=856.4290 [M]⁺ for [C₃₅H₅₄N₃O₉Pt]⁺. ¹H NMR (500 MHz, CDCl3-d6) δ: 8.20 (t, J=10 Hz, 10 Hz, 2H), 8.12 (d, J=5 Hz, 1H), 8.04 (d, J=10 Hz, 1H), 7.47-7.52 (m, 2H), 7.19 (d, J=10 Hz, 1H), 6.13 (m, 1H), 5.80 (m, 1H), 5.70 (t, 10 Hz, 10 Hz, 2H), 4.60 (t, J=5 Hz, 5 Hz, 1H), 3.77-3.85 (m, 1H), 3.56-3.61 (m, 4H), 3.45-3.51 (m, 26H), 2.32-2.39 (m, 2H), 1.95-1.97 (m, 2H), 1.55-1.57 (m, 2H), 1.33-1.35 (m, 2H), 1.08-1.14 (m, 2H) ppm.

### Synthesis of IO-199_29 (Compound 2w)

The compound IO-199_29 was synthesized following Scheme 27.

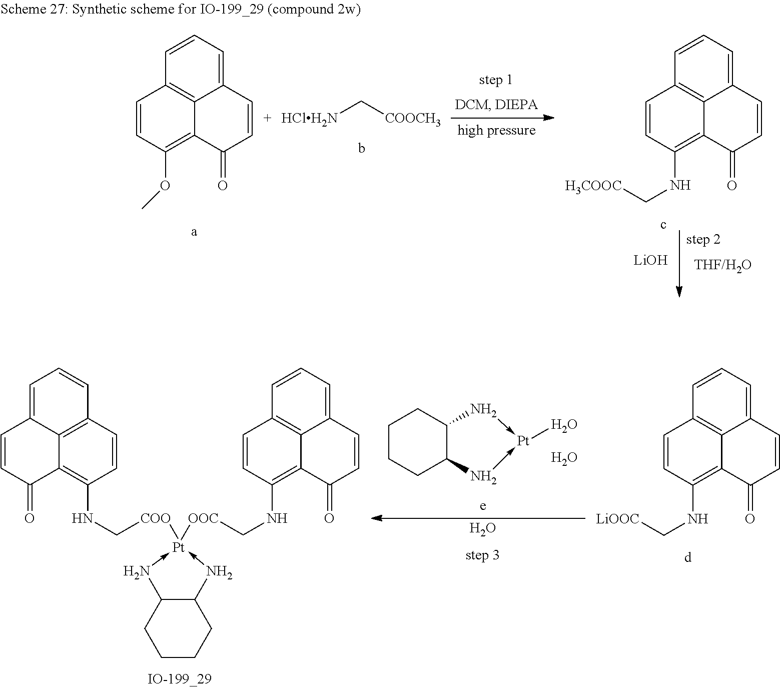

Experimental Procedure (Step 1):

A 10 mL Pressure tube was charged with O-Methoxy PLY a (100 mg, 0.47 mmol) with 2 mL DCM and DIEPA (200 μL, 1.17 mmol). To it Glycine ester b (118 mg, 0.94 mmol) was added was heated at 70° C. for 18 hrs. After completion of reaction, compound was washed with water and extracted with CHCl₃. Final product was purified by column chromatography (Silica 60-120 mesh, Ethyl acetate: Hexane: 20%).

### Characterization of c

ESIMS m/z=267.9583 [M]⁺ for [C₁₆H₁₃NO₃]⁺. ¹H NMR (500 MHz, CDCl3-d6) δ: 12.11 (s, 1H), 8.08 (d, J=10 Hz, 1H), 7.92-7.97 (m, 3H), 7.51 (t, J=10 Hz, J=5 Hz 1H), 7.29 (s, 1H), 7.09 (d, J=5 Hz, 1H), 4.41 (d, J=5 Hz, 2H), 3.85 (s, 3H).

Experimental Procedure (Step 2):

A 100 mL single neck RB flask charged with compound c (46 mg, 0.172 mmol) with 12 mL THF/H₂O (3:1). The reaction mixture was cooled to 0° C. under ice bath for 10 mins and LiOH (8.66 mg, 0.206) was added. The resulting solution was stirred for 5-6 hrs. at rt. After completion, reaction mixture was concentrated to remove THF and diluted with water (20 mL) and water layer was washed with Ethyl acetate, DCM and hexane successively. The aqueous layer was utilized for the next step.

Experimental Procedure (Step 3):

To a 100 mL single neck RBF aquated DACH Pt e (50 mg, 0.13 mmol) in 10 mL water was taken. To the above solution compound d (36.08 mg, 0.13 mmol) in 30 mL water was added. The resulting solution was stirred for 24 hrs at rt. After completion the ppt was collected through centrifugation and given water and ether wash and then dissolved in MeOH. The volume of MeOH was reduced to half and the precipitate and the remaining MeOH layer was separately concentrated.

### Characterization of IO-199_29

ESIMS m/z=814.31 [M+1]⁺ for [C₃₆H₃₄N₄O₆Pt]⁺. ¹H NMR (500 MHz, DMSO-d6) δ: 12.08 (t, 2H, J=5 Hz), 8.15 (d, J=10 Hz, 2H), 7.95-8.02 (m, 6H), 7.45 (t, J=10 Hz, J=5H, 2H), 7.28 (d, J=10 Hz, 2H), 6.83 (d, J=10 Hz, 2H), 6.03 (d, J=10 Hz, 2H), 5.31 (t, J=10 Hz, 2H), 4.23 (d, J=5 Hz, 4H), 2.11-2.18 (m, 2H), 1.92 (d, J=15 Hz, 2H), 1.41-1.46 (m, 2H), 1.25-1.34 (m, 2H), 0.96-1.03 (m, 2H).

### Synthesis of IO-199_36 (Compound 2a′)

The compound IO-199_36 was synthesized following Scheme 28.

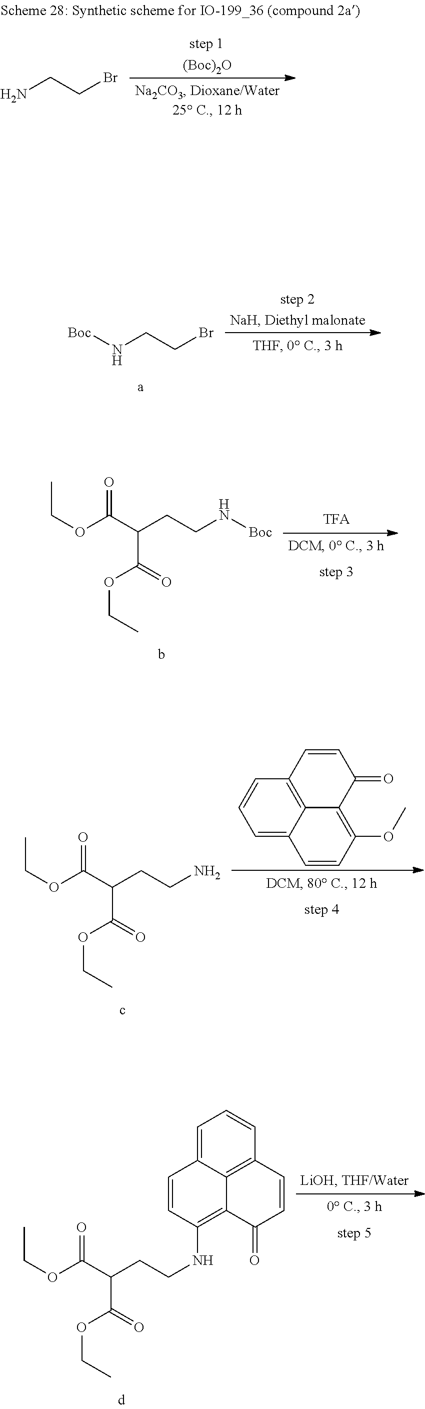

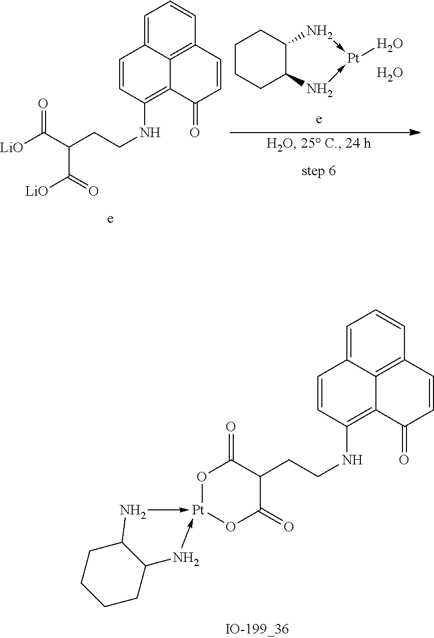

Experimental Procedure (Step 1):

To a 50 mL single neck RBF, 2-bromoethylamine hydrobromide (1 g, 4.88 mmol) was taken in Dioxane (15 mL). To this solution sodium carbonate (0.409 g, 10.22 mmol, in 1 mL water) was added and left for stirring for 15 min. To the above solution Boc anhydride (1.63 g, 7.45 mmol) was added and left for stirring for 2 h. The TLC was checked and after completion the reaction mixture was quenched using water and extracted using ethyl acetate (3×10 mL). The organic layer was dried over sodium sulphate and concentrated under vacuum to get the product a.

Experimental Procedure (Step 2):

A solution of diethyl malonate (0.5 g, 3.12 mmol) in 5 ml THF was added to a suspension of NaH (60 wt % in mineral oil, 0.125 g, 3.12 mmol) in 5 ml THF at 0° C. under N₂ atmosphere. Then a solution of Boc protected 2-bromoethyl amine a (0.538 g, 2.40 mmol) in 1 ml THF was added dropwise at 0° C. After completion, the reaction mixture was quenched using water and extracted using ethyl acetate (3×10 mL). The organic layer was dried over sodium sulphate and concentrated under vacuum. Compound was purified by column chromatography to get the desired product b.

Experimental Procedure (step 3):

A 50 mL single neck RB flask charged with Boc-protected diethyl amino malonate b (0.5 g, 1.648 mmol). To it 20% TFA solution was added dropwise at 0° C. The resulting solution was stirred for 3 hrs. After completion of reaction, DCM was removed. TFA was removed by giving DCM, Diethyl ether and toluene wash. Without further purification, it was used for next reaction.

Experimental Procedure (step 4):

A 10 mL Pressure tube was charged with O-Methoxy PLY (0.2 g, 0.94 mmol) with 2 mL DCM. To it diethyl amino malonate c (0.286 g, 1.41 mmol) was added was heated at 70° C. for 18 hrs. After completion of reaction, compound was washed with water and extracted with CHCl₃. Final product was purified by column chromatography.

Experimental Procedure (Step 5):

A 100 mL single neck RB flask charged with compound d (0.38 g, 1 mmol) with THF/H₂O (3:1). The reaction mixture was cooled to 0° C. under ice bath for 10 mins and LiOH (0.032 g, 2.0 mmol) was added. The resulting solution was stirred for 3 hrs. at rt. After completion, reaction mixture was concentrated to remove THF and diluted with water (20 mL) and water layer was washed with DCM and ethyl acetate successively.

Experimental Procedure (Step 6):

A 100 mL single neck RB flask charged with Aquated DACH platinum f (1 mmol) in mL water. To the above solution compound e (0.337 g, 1 mmol) in mL water was added. The resulting solution was stirred for 24 hrs at rt. After completion the ppt was collected through centrifugation of reaction mixture. The ppts were washed with water (2*20 mL) and then lyophilized.

### Synthesis of IO-199_37 (Compound)

The compound IO-199_37 is synthesized following Scheme 29.

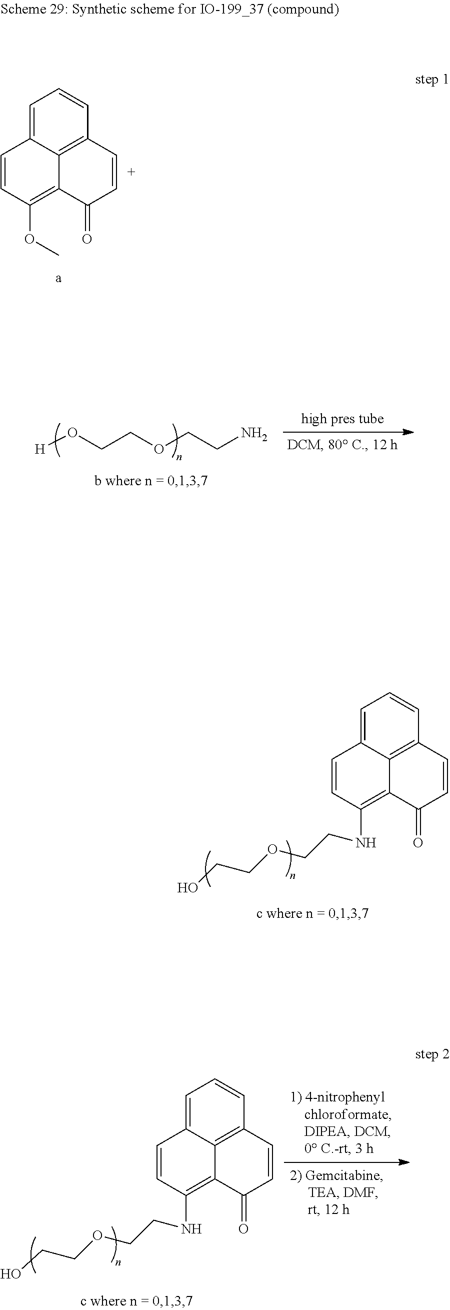

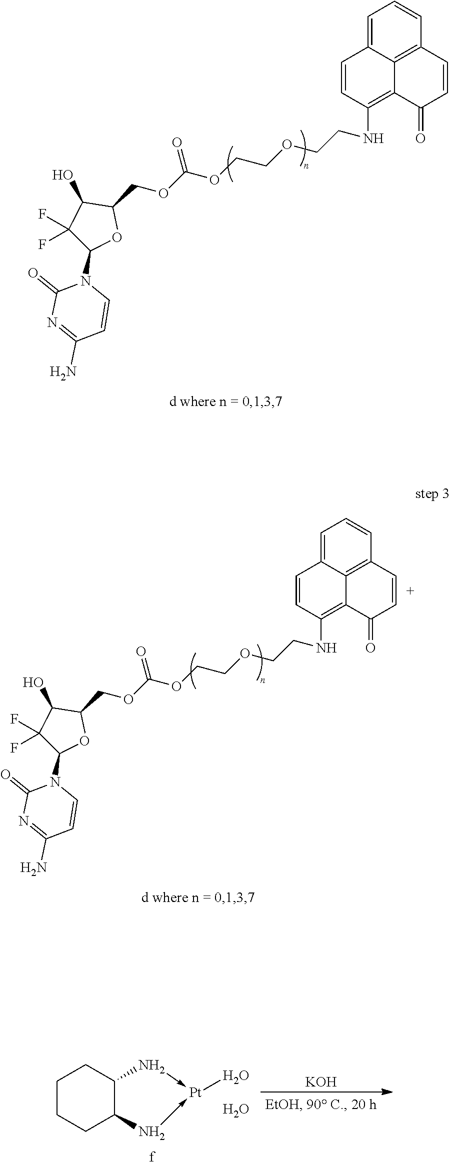

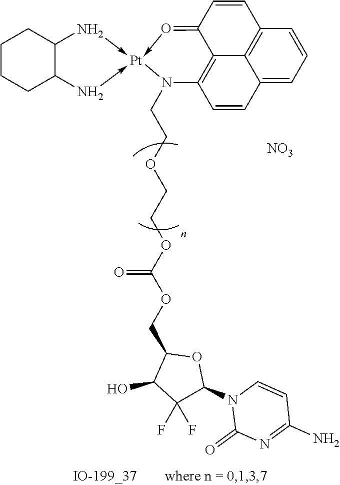

Experimental Procedure (Step 1):

A 10 mL Pressure tube is charged with O-Methoxy PLY a (0.459 g, 2.187 mmol) with 5 mL DCM. To it, tetra peg amine alcohol b (0.507 g, 2.625 mmol) is added and heated at 80° C. for 18 hrs. After completion of reaction, compound is washed with water and extracted with CHCl₃. Final product d is purified by column chromatography (Silica 60-120 mesh, Ethyl acetate: Hexane: 80:20 and then Methanol: CHCl₃: 10:90). Yield is 63%.

### Characterization of c

ESIMS m/z=372.0175 [M+1]⁺ for [C₂₁H₂₄NO₆]⁺. ¹H NMR (500 MHz, CDCl3-d6) δ: 12.20 (s, 1H), 8.20 (d, J=10 Hz, 1H), 8.06 (d, J=5 Hz, 1H), 8.01 (t, J=5 Hz, 10 Hz, 2H), 7.49 (t, J=5 Hz, 10 Hz, 2H), 6.85 (d, J=10 Hz, 1H), 4.56 (m, 1H), 3.76 (m, 4H), 3.36-3.62 (m, 11H).

Experimental Procedure (Step 2):

To a DCM (10.0 mL) solution of c (1.0 mmol) 4-nitrophenyl chloroformate (403.12 mg, 2.0 mmol), DIPEA (516 mg, 4.0 mmol) and catalytic amount of pyridine are added at 0° C. and stirred for 2 h at rt. Then the reaction mixture is concentrated under vacuo. The crude residue is dissolved in 5.0 mL DMF. To this solution gemcitabine (524 mg, 2.0 mmol) in DMF (4.0 mL) and TEA (1.0 mL) are added and continued to stirr for 12 h. After completion of reaction, the reaction mixture is diluted in water. The compound is extracted with EtOAc. The organic layer is dried over anhydrous Na2SO4. The crude compound is passed through silica column chromatography using DCM/MeOH (9:1) as eluent to afford d.

Experimental Procedure (Step 3):

A 250 mL single neck RB flask is charged with aquated DACH Platinum f (0.474 g,1.315 mmol) taken in 40 mL water (lyophilized to reduce volume to 10 mL). After this 10 mL EtOH was added. Tetra peg alcohol PLY Ligand d (0.65 mmol) (dissolved in 50 mL EtOH) is added along with KOH (29.1 mg, 1.25 mmol) (solution in EtOH). The resulting solution is refluxed for 24 hrs. at 90° C. After completion, reaction mixture is concentrated to remove EtOH and DCM is added to the reaction mixture. It was washed with water (30 mL). The organic layer is concentrated and purified by Column chromatography (Silica 60-120 mesh, MeOH: CHCl₃: 2%-10%).

### Synthesis of IO-199_38 (Compound)

The compound IO-199_38 is synthesized following Scheme 31.

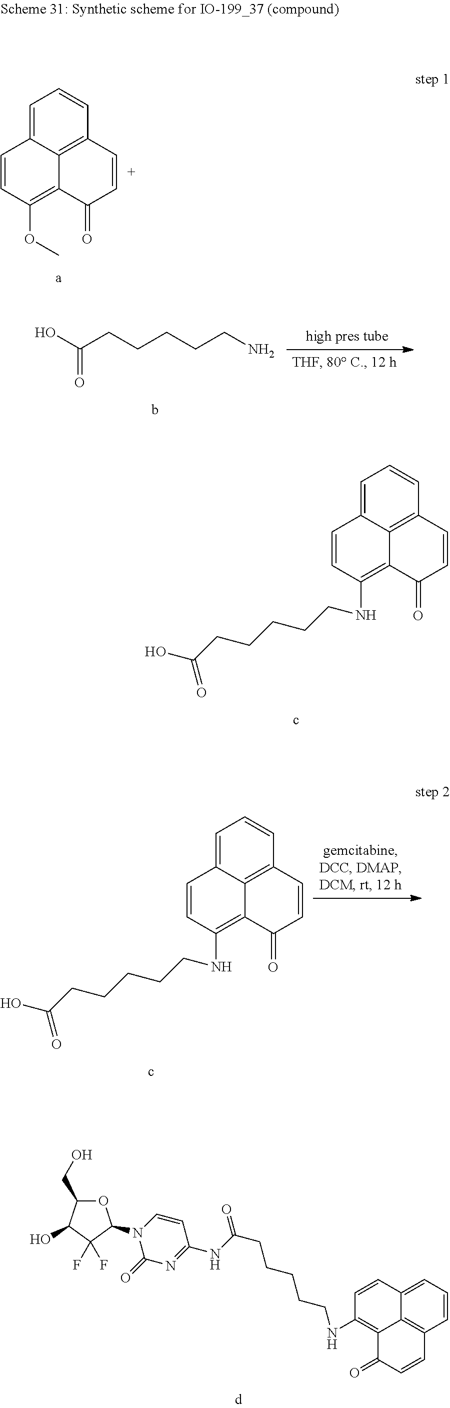 [SMILES: COc1ccc2cccc3c2c1C(=O)C=C3 ; NCCCCCC(=O)O ; O=C(O)CCCCCNc1ccc2cccc3c2c1C(=O)C=C3 ; O=C(O)CCCCCNc1ccc2cccc3c2c1C(=O)C=C3 ; O=C(CCCCCNc1ccc2cccc3c2c1C(=O)C=C3)Nc1ccn([C@@H]2O[C@H](CO)[C@H](O)C2(F)F)c(=O)n1]

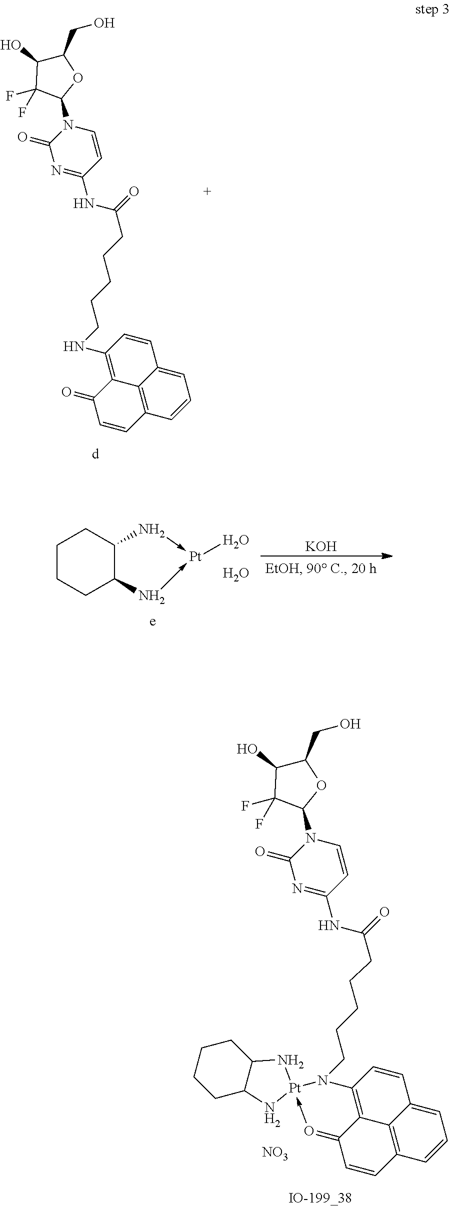

Experimental Procedure (step 1):

A 10 mL Pressure tube is charged with O-Methoxy PLY a (0.2 g, 0.952 mmol) with 5 mL THF and 0.5 ml water. To it, 6-amino caproic acid b (0.25 g, 1.9 mmol) is added and heated at 80° C. for 18 hrs. After completion of reaction, compound is washed with water and extracted with CHCl₃. Final product c is purified by column chromatography.

Experimental Procedure (Step 2):

Compound c (1.0 mmol) is taken in 20 ml anhy. DCM under N₂ atmosphere and to this solution DCC (1.2 mmol) and DMAP (0.05 mmol) is added at 0° C. and stirred for 20 min. To this solution e (1.0 mmol) is added as solid and stirred at r.t. for 12 h and TLC was checked. After completion of the reaction mixture, it mixture is filtered and the filtrate is concentrated and purified by column chromatography.

Experimental Procedure (Step 3):

A 250 mL single neck RB flask is charged with aquated DACH Platinum d (0.474 g,1.315 mmol) taken in 40 mL water (lyophilized to reduce volume to 10 mL). After this 10 mL EtOH was added. PLY Ligand e (0.65 mmol) (dissolved in 50 mL EtOH) is added along with KOH (29.1 mg, 1.25 mmol) (solution in EtOH). The resulting solution is refluxed for 24 hrs. at 90° C. After completion, reaction mixture is concentrated to remove EtOH and DCM is added to the reaction mixture. It is washed with water (30 mL). The organic layer is concentrated and purified by Column chromatography (Silica 60-120 mesh, MeOH: CHCl₃: 2%-10%).

### Synthesis of IO-199_39 (Compound)

The compound IO-199_39 is synthesized following Scheme 32.

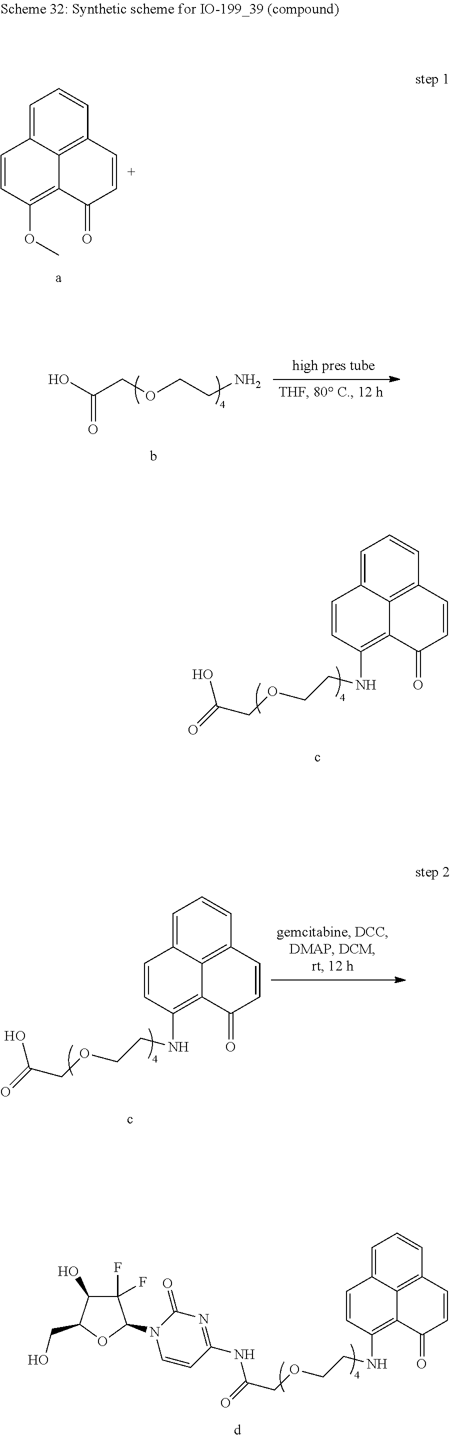

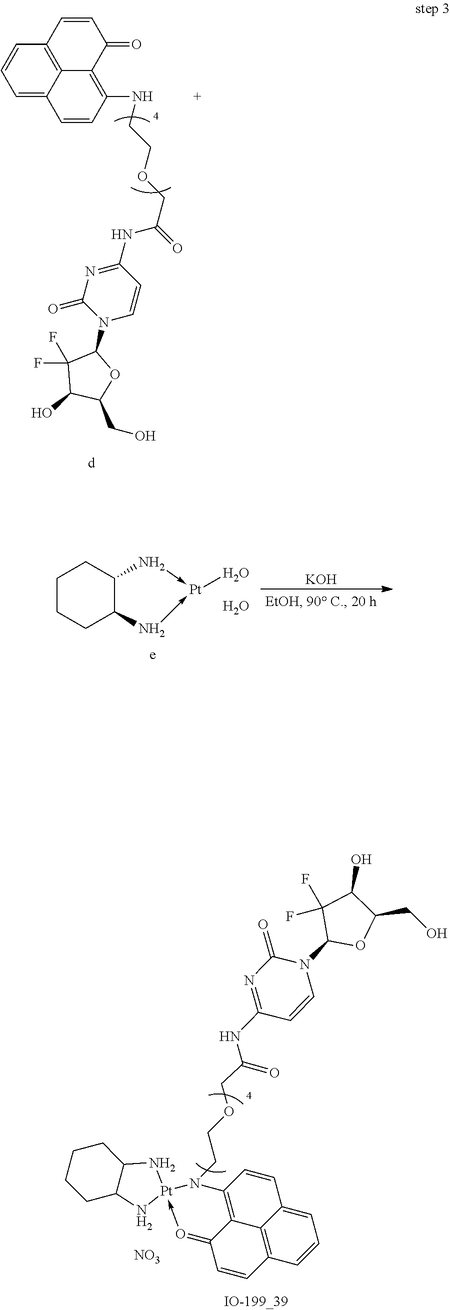

Experimental Procedure (Step 1):

A 10 mL Pressure tube is charged with O-Methoxy PLY a (1.0 mmol) with 5 mL THF. To it, tetra peg amine b (1.2 mmol) is added and heated at 80° C. for 18 hrs. After completion of reaction, compound is washed with water and extracted with CHCl₃. Final product c is purified by column chromatography.

Experimental Procedure (Step 2):

Compound c (1.0 mmol) is taken in 20 ml anhy. DCM under N₂ atmosphere and to this solution DCC (1.2 mmol) and DMAP (0.05 mmol) is added at 0° C. and stirred for 20 min. To this solution e (1.0 mmol) is added as solid and stirred at r.t. for 12 h and TLC is checked. After completion of the reaction mixture, it mixture is filtered and the filtrate is concentrated and purified by column chromatography.

Experimental Procedure (Step 3):

A 250 mL single neck RB flask is charged with aquated DACH Platinum d (0.474 g, 1.315 mmol) taken in 40 mL water (lyophilized to reduce volume to 10 mL). After this 10 mL EtOH is added. PLY Ligand e (0.65 mmol) (dissolved in 50 mL EtOH) is added along with KOH (29.1 mg, 1.25 mmol) (solution in EtOH). The resulting solution is refluxed for 24 hrs. at 90° C. After completion, reaction mixture is concentrated to remove EtOH and DCM is added to the reaction mixture. It is washed with water (30 mL). The organic layer is concentrated and purified by Column chromatography (Silica 60-120 mesh, MeOH: CHCl₃: 2%-10%).

### Synthesis of IO-199_40 (Compound)

The compound IO-199_40 is synthesized following Scheme 33.

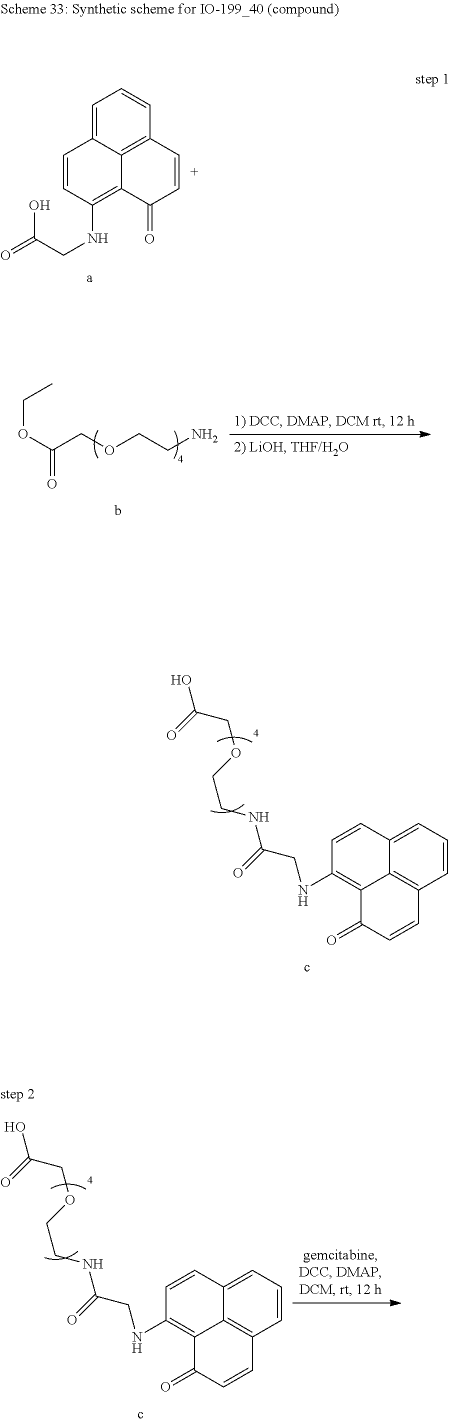

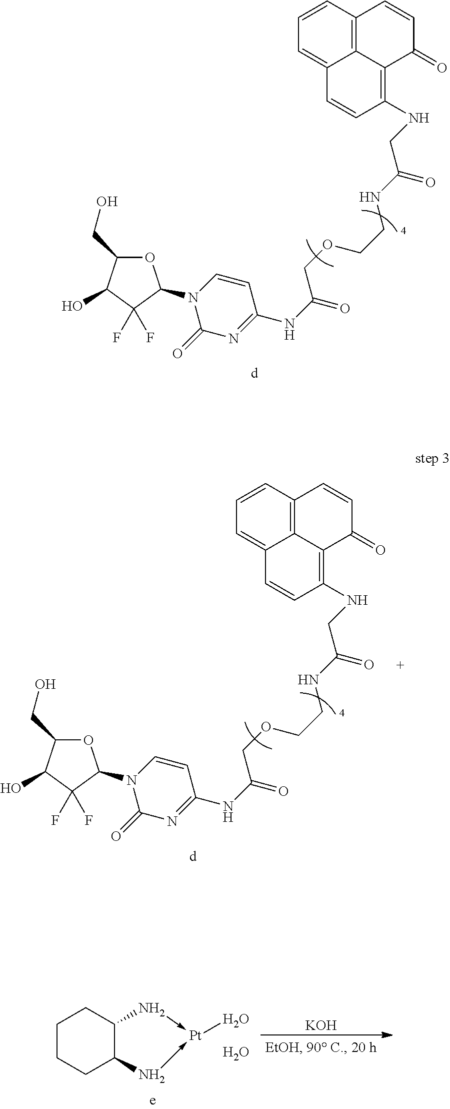

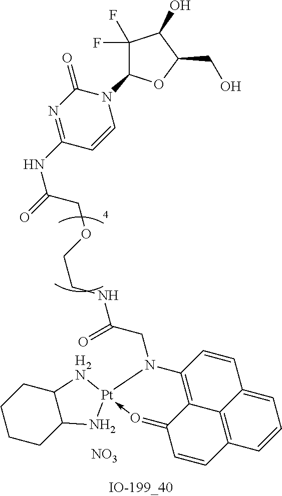

Experimental Procedure (Step 1):

Compound a (1.0 mmol) is taken in 20 ml anhy. DCM under N₂ atmosphere and to this solution DCC (1.2 mmol) and DMAP (0.05 mmol) is added at 0° C. and stirred for 20 min. To this solution b (1.0 mmol) is added as solid and stirred at r.t. for 12 h and TLC is checked. After completion of the reaction mixture, it mixture is filtered and the filtrate is concentrate and purified by column chromatography to afford the ester.

To a 50 mL single neck RBF, ester (1 mmol) is taken in 20 mL of THF/H2O (3:1) and cooled to 0° C. under ice bath. To this ice cooled solution LiOH (1.2 mmol) is added and is stirred at rt for overnight, the TLC is checked. After completion the reaction mixture is extracted with ethyl acetate and washed with sodium dihydrogen sulphate solution (20 mL) and brine (20 mL) successively. The organic layer is dried over anhydrous Na2SO4 and concentrated under vacuum to afford the acid c.

Experimental Procedure (Step 2):

Compound c (1.0 mmol) is taken in 20 ml anhy. DCM under N₂ atmosphere and to this solution DCC (1.2 mmol) and DMAP (0.05 mmol) is added at 0° C. and stirred for 20 min. To this solution e (1.0 mmol) is added as solid and stirred at r.t. for 12 h and TLC is checked. After completion of the reaction mixture, it mixture is filtered and the filtrate is concentrated and purified by column chromatography.

Experimental Procedure (Step 3):

A 250 mL single neck RB flask is charged with aquated DACH Platinum d (0.474 g, 1.315 mmol) taken in 40 mL water (lyophilized to reduce volume to 10 mL). After this 10 mL EtOH is added. PLY Ligand e (0.65 mmol) (dissolved in 50 mL EtOH) is added along with KOH (29.1 mg, 1.25 mmol) (solution in EtOH). The resulting solution is refluxed for 24 hrs. at 90° C. After completion, reaction mixture is concentrated to remove EtOH and DCM is added to the reaction mixture. It is washed with water (30 mL). The organic layer is concentrated and purified by Column chromatography (Silica 60-120 mesh, MeOH: CHCl₃: 2%-10%).

### Example 7: Synthesis and Characterization of Exemplary Compounds

### Synthesis of IO-199_16 (Compound 1m)

The compound IO-199_06 was synthesized following Scheme 34.

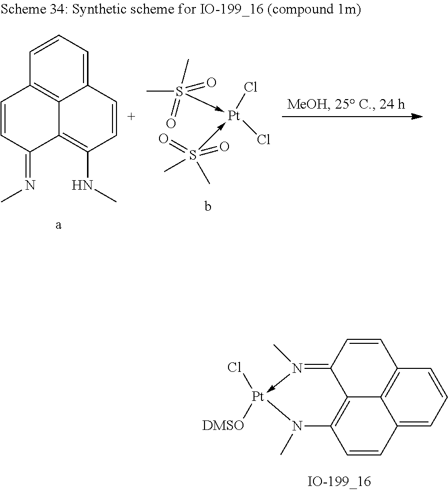

Experimental Procedure:

N,N PLY a (0.105 g, 0.47 mmol) was taken in 100 ml methanol and then Pt(DMSO)₂C1₂ b (0.2 g, 0.47 mmol) was added. The reaction mixture was left for room temperature stirring for 18 hours in nitrogen atmosphere. Some purple ppt was formed. PPT was filtered and dried over vacuum. Yield 0.076 g (30%).

### Characterization of IO-199_16 (Compound 1m)

ESIMS m/z=530.03 [M+H]⁺ for [C₁₇H₂₀N₂OPtS]⁺. ¹H NMR (400 MHz, CDCl3) δ: 7.75 (d, J=8 Hz, 2H), 7.67 (d, J=8 Hz, 1H), 7.60 (d, J=8 Hz, 1H), 7.19-7.27 (m, 3H), 3.79 (s, 3H), 3.55 (s, 3H), 3.35 (s, 6H) ppm. ¹³C NMR (125 MHz, CDCl₃) δ: 153.94, 153.80, 135.16, 134.71, 130.23, 130.14, 128.13, 125.66, 125.46, 121.68, 118.0, 117.81, 114.19, 47.83, 44.93, 43.69 ppm. ¹⁹⁵Pt NMR (107 MHz, DMSO-d6) δ: −2729.08 ppm.

### HPLC Analysis of IO-199_16 (Compound 1m)

Equisil-BDS C8 column used to check the analytical purity of compound IO-199_16 with a gradient method of 5% MeOH/Water to 100% MeOH and a gradient time of 15 minute. The purity of IO-199_16 was 84.68% (data not shown).

### Synthesis of IO-199_17 (Compound 1n)

The compound IO-199_17 was synthesized following Scheme 35.

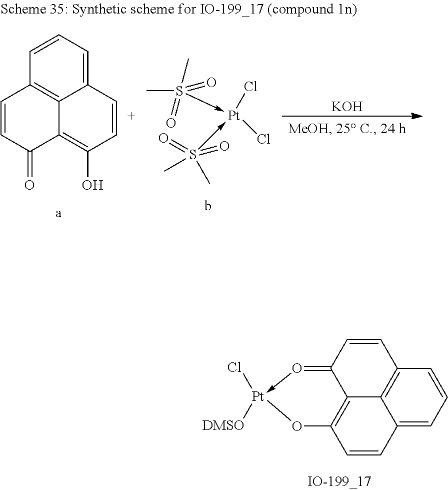

Experimental Procedure:

O,O PLY (compound a, 0.090 g, 0.47 mmol) was taken in 100 ml methanol and then Pt(DMSO)₂Cl₂ (compound b, 0.2 g, 0.47 mmol) was added. Then 26.6 mg KOH in 1 ml MeOH was added. The reaction mixture was left for room temperature stirring for 18 hours in nitrogen atmosphere. Some yellowish ppt was formed. PPT was filtered and dried over vacuum. Yield 0.08 g (35%).

### Characterization of IO-199_17 (compound 1n)

ESIMS m/z=526.9818 [M+Na]⁺ for [C₁₇H₂₀O₃PtS]⁺. ¹H NMR (400 MHz, CDCl3) δ: 8.13-8.17 (t, J=8 Hz, 3H), 8.09 (d, J=8 Hz, 1H), 7.62 (t, J=8 Hz, J=4 Hz, 1H), 7.46 (d, J=8 Hz, 1H), 7.34 (d, J=8 Hz, 1H), 3.59 (s, 6H) ppm. ¹³C NMR (125 MHz, CDCl₃) δ: 172.31, 172.25, 139.85, 139.17, 132.72, 132.62, 126.86, 126.37, 126.22, 125.73, 124.46, 113.88, 44.25 ppm. ¹⁹⁵Pt NMR (107 MHz, DMSO-d6) δ: −2293.25 ppm.

### HPLC Analysis of IO-199_17 (Compound 1n)

Equisil-BDS C8 column was used to check the analytical purity of compound IO-199_17 with a gradient method of 5% MeOH/Water to 100% MeOH and a gradient time of15 minute. The purity of IO-199_17 was 98.67% (data not shown).

### Synthesis of IO-199_18 (Compound 1p)

The compound IO-199_18 was synthesized following Scheme 36.

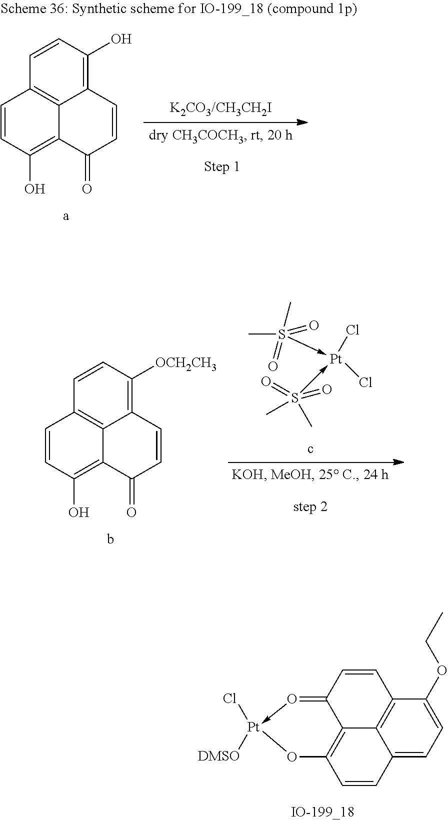

Experimental Procedure (Step 1):

The compound a (0.5 g, 2.36 mmol) and potassium carbonate (0.812 g, 5.88 mmol) were taken in to 100 mL R.B and dissolved in 30 ml dry acetone under nitrogen atmosphere. After 30 minute room temperature stirring, ethyl iodide (2.5 ml, 30.44 mmol) was added dropwise to the reaction mixture and was left it for overnight at room temperature. After completion, reaction mixture was filtered through Whatman filter paper and purified by column chromatography (Silica 60-120 mesh, Ethyl acetate: Hexane: 1:20). The yield of the compound b is 57%.

### Characterization of b

ESIMS m/z=241.07 [M+H]+ for [C₁₅H₁₂O₃]⁺. ¹H NMR (500 MHz, CDCl3) δ: 8.59 (d, J=10 Hz, 1H), 7.96 (m, 2H), 7.16 (d, J=5 Hz, 1H), 7.01 (dd, J=20, 10 Hz, 2H), 4.34 (q, J=7.0 Hz, 2H), 1.59 (t, J=7.5 Hz, 3H) ppm.

Experimental Procedure (Step 2):

Ethoxy PLY (compound b, 0.1 g, 0.42 mmol) was taken in 100 ml methanol and then Pt(DMSO)₂Cl₂ c (0.176 g, 0.42 mmol) was added. Then 23 mg KOH in 0.4 ml MeOH was added. The reaction mixture was left for room temperature stirring for 18 hours in nitrogen atmosphere. Some yellowish brown ppt was formed. PPT was filtered and dried over vacuum. Yield 0.07 g (31%). NMR shows formation of two diastereomer which are non-separable and it is in 3:2 ratio. ESIMS m/z=570.7798 [M+Na]⁺ for [C₁₇H₁₇ClNaO₄PtS]+.

### First Diastereomer

¹H NMR (500 MHz, CDCl₃) δ 8.57 (d, J=10 Hz, 1H), 8.07 (d, J=10 Hz, 1H), 8.01 (d, J=10 Hz, 1H), 7.29 (dd, J=5 Hz, 2H), 7.07 (d, J=10 Hz, 1H), 4.33 (m, 2H), 3.58 (s, 6H), 1.60 (m, 3H).

### Second Diastereomer

¹H NMR (500 MHz, CDCl₃) δ 8.65 (d, J=10 Hz, 0.66H), 8.05 (d, J=10 Hz, 0.65H), 7.95 (d, J=10 Hz, 0.65H), 7.41 (d, J=10 Hz, 0.65H), 7.18 (d, J=10 Hz, 0.65H), 7.07 (d, J=10 Hz, 0.65H), 4.33 (m, 1.3H), 3.57 (s, 4H), 1.60 (m, 2H).

### Synthesis of IO-199_19 (Compound 1q)

The compound IO-199_19 was synthesized following Scheme 37.

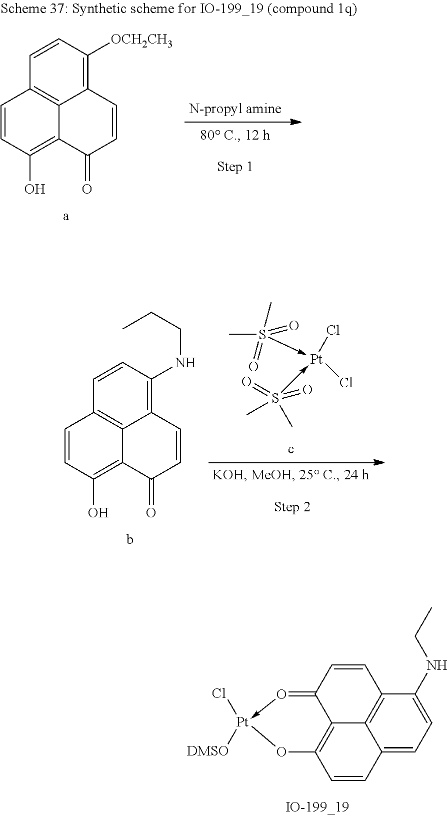

Experimental Procedure (Step 1):

The compound a (0.1 g, 0.042 mmol) and excess N-propyl amine (2 ml, 24.33 mmol) were taken in high pressure tube and refluxed it for 12 h at 80° C. After completion of reaction, it was evaporated and purified by column chromatography (Silica 60-120 mesh, Ethyl acetate: Hexane: 1:10). The yield of the compound b is 60%.

### Characterization of b

ESIMS m/z=254.1087 [M+H]+ for [C₁₆H₁₅NO₂]⁺. ¹H NMR (500 MHz, CDCl3) δ: 8.12 (d, J=10 Hz, 1H), 7.76 (dd, J=35, 10 Hz, 2H), 6.95 (d, J=10 Hz, 1H), 6.84 (d, J=10 Hz, 1H), 6.69 (d, J=10 Hz, 1H), 3.36 (t, J=7.5 Hz, 2H), 1.80 (h, J=8.0 Hz, 2H), 1.07 (t, J=7.5 Hz, 3H) ppm.

Experimental Procedure (Step 2):

Ethoxy PLY (compound b, 0.01 g, 0.039 mmol) was taken in 10 ml methanol and then Pt(DMSO)₂C1₂ (Compound c, 0.018 g, 0.039 mmol) was added. Then 2 mg KOH in 0.4 ml MeOH was added. The reaction mixture was left for room temperature stirring for 18 hours in nitrogen atmosphere. Solvent was evaporated and purified by Preparative Thin Layer Chromatography. 5% Methanol/Chloroform solvent system is used as eluent. The retardation factor (R_{f}) for two diastereomer is 0.2 and 0.4. After purification, two diastereomer were isolated.

### First Diastereomer

ESIMS m/z=584.8109 [M+Na]⁺& 600.7812 [M+K]⁺ for [C₁₈H₂₀ClNO₃PtS]⁺. ¹H NMR (500 MHz, CDCl₃) δ 8.15 (d, J=10 Hz, 1H), 7.88 (d, J=10 Hz, 1H), 7.79 (d, J=10 Hz, 1H), 7.31 (d, J=10 Hz, 1H), 7.02 (d, J=10 Hz, 1H), 6.84 (d, J=10 Hz, 1H), 3.54 (s, 6H), 3.37 (t, J=7.5 Hz, 2H), 1.84 (q, J=7.0 Hz, 2H), 1.10 (t, J=7.5 Hz, 3H).

### Second Diastereomer

¹H NMR (500 MHz, CDCl₃) δ 8.08 (d, J=10 Hz, 1H), 7.82 (d, J=10 Hz, 1H), 7.74 (d, J=10 Hz, 1H), 7.09 (d, J=10 Hz, 1H), 7.01 (d, J=10 Hz, 1H), 6.78 (d, J=10 Hz, 1H), 3.54 (s, 6H), 3.37 (t, J=7.5 Hz, 2H), 1.86 (q, J=7.0 Hz, 2H), 1.10 (t, J=7.5 Hz, 3H).

### Synthesis of IO-199_34 & 35 (Compound 1u and 1v)

The compounds IO-199_34 and IO-199_35 were synthesized following Scheme 38.

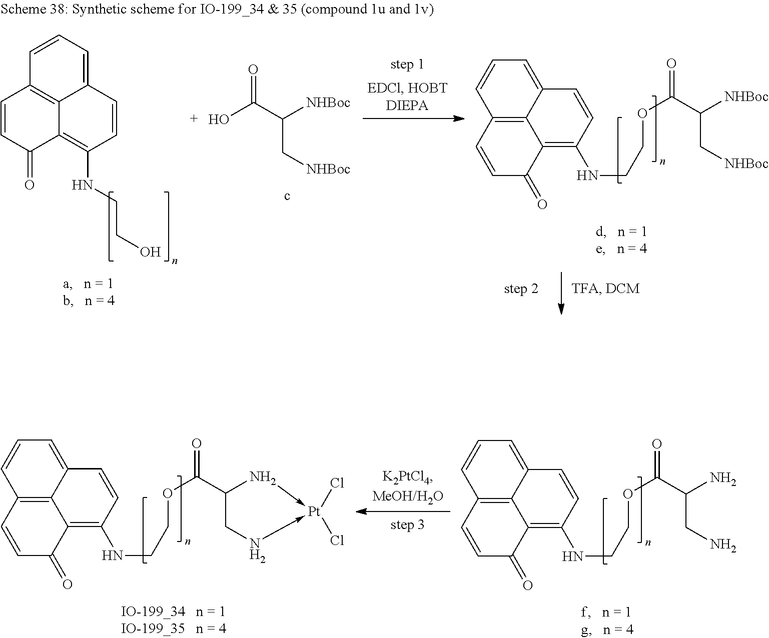

Experimental Procedure for 1u (Step 1):

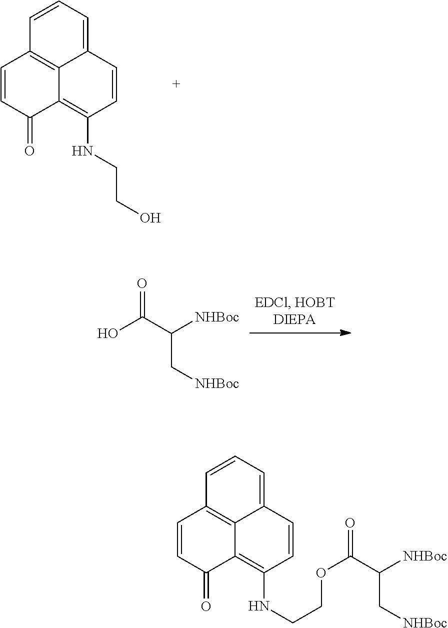 [SMILES: O=C1C=Cc2cccc3ccc(NCCO)c1c23 ; CC(C)(C)OC(=O)NCC(NC(=O)OC(C)(C)C)C(=O)O ; CC(C)(C)OC(=O)NCC(NC(=O)OC(C)(C)C)C(=O)OCCNc1ccc2cccc3c2c1C(=O)C=C3]

1 eq (38 mg) of Boc protected 2,3-diaminopropionate add with 2.5 eq (60 mg) of EDCl and 1.5 eq (17 mg) of HOBt in dry DCM and stirred at 0° C. in ice bath for 1 h under nitrogen atmosphere. To this cooled solution 1 eq (30 mg) of IO-199_32_Im-01 and 3 eq (64 μL) of DIEPA were added and continued the reaction for overnight. After completion of reaction, compound purified by column chromatography (Silica 60-120 mesh, 2% MeOH/DCM).

ESIMS m/z=548.23 [M+Na]⁺ and 326.13 [M-2Boc]. ¹H NMR (400 MHz, DMSO-d6) δ: 12.25 (s, 1H), 8.05 (d, J=8 Hz, 1H), 7.88-7.941 (m, 3H), 7.48 (t, J=8 Hz, 1H), 7.26 (d, J=7 Hz, 1H), 7.11 (d J=4 Hz, 1H), 5.80-5.90 (m, 1H), 5.77 (s, 1H), 4.62 (s, 1H), 4.42 (s, 2H), 3.91-3.88 (m, 2H), 3.71-3.58 (m, 2H), 1.38 (s, 9H), 1.27 (s, 9H) ppm.

Experimental Procedure (step 2):

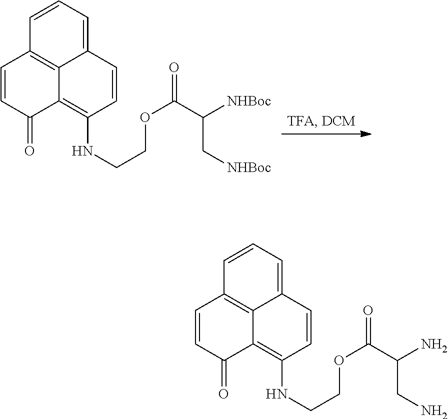 [SMILES: CC(C)(C)OC(=O)NCC(NC(=O)OC(C)(C)C)C(=O)OCCNc1ccc2cccc3c2c1C(=O)C=C3 ; NCC(N)C(=O)OCCNc1ccc2cccc3c2c1C(=O)C=C3]

Deprotection of Boc group by adding 30% TFA in DCM (5 mL) to the IO-199_34_Im-01 give IO-199_34_Im-02. Evaporate the TFA by rotavapor and dissolved it again in DCM and ether 2 times each and evaporate the solvent again. The crude IO-199_34-Im-02 used for the next step. ESIMS m/z=325.36 [M+H]⁺.

Experimental Procedure (Step 3):

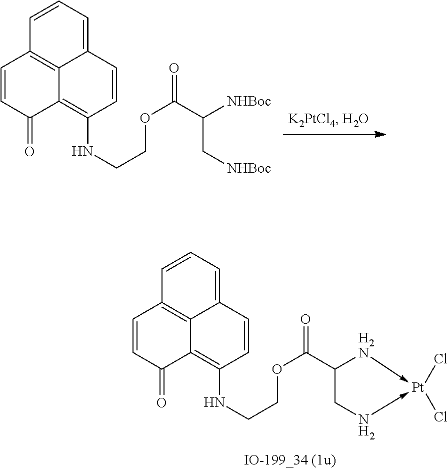

1 eq of (9 mg) K₂PtCl₄ in H2O added dropwise to the IO-199_34-Im-02 in water solution in R.B. Immediate, precipitation occur and this reaction mixture stirred for overnight and the next day lyophilized the reaction mixture to remove water. This crude mixture washed with water, methanol and DCM to get pure product. ESIMS m/z=629.01 [M+DMSO-Cl]⁺. (compound solubilized in DMSO for Mass)

### Example 8: Exemplary Formulations of Compounds IO-199_01 and IO-199_02

### IO-199_01 Formulation Preparation

The IO-199_01 formulation was engineered using thin film hydration method with slight modification (1, 2) (Sengupta et al., Cholesterol-tethered platinum II-based supramolecular nanoparticle increases antitumor efficacy and reduces nephrotoxicity, PNAS (2012) 109(28): 11294-11299 and Szoka & Papahadjopoulos: Comparative properties and methods of Preparation of lipid vesicles (liposomes), Ann. Rev. Biophys. Bioeng. (1980) 9:467-508). Particle size and zeta potential was measured using a Dynamic Light Scattering method using Zetasizer Nano ZS90 (Malvern, UK). Pt equivalent in drug concentrations were quantified using atomic absorption spectrometer (AAS) (PinAAcle 900Z, US).

### Detailed Procedure

Organic solvent was evaporated into a thin and uniform lipid-drug film initially by N₂-flushing and finally under vacuum using a rotary evaporator at 45° C. The dry lipid-drug film was then hydrated with required volume of buffer (or water) to get a phospholipids required concentration of drug and allowed to rotate on rotary evaporator at 65° C. (above the lipid phase transition temperature) for 60 min. Hydrated MLVs (multilamellar vesicles) were sequentially extruded through 400 nm, 200 nm and 100 nm pore size membrane (Whatman® filters) supported by filter support (Avanti No: 230600) for 10 times using 10 ml LIPEX™ extruder (Northern Lipids, Canada) under nitrogen pressure connected to circulating water bath at 65° C. (2-4). Finally, the glass vial containing formulation were cooled at 4° C. for 30 min, then −20° C. for 2 h and at −80° C. for 2 h and finally freeze dried overnight at −80° C.

### Formulation Preparation

Lipid-based IO-199_01 formulation was engineered using thin film hydration method. The compositions of different formulations are shown in Tables 1 and 3. Dynamic light scattering (DLS) data is summarized in Tables 2 and 4.

TABLE 1
IO-199_01 prep-01

| | | Compounds | |
|---|---|---|---|
| Lipids | Mol. wt | used (mg) | mol % |
| L-α-phosphatidylcholine, | 783.7 | 21.94 | 70.00 |
| hydrogenated (soy) HSPC | | | |
| Free-cholesterol | 387 | 2.32 | 15.00 |
| IO-199_01 | 506.48 | 2.03 | 10.00 |
| 1,2-distearoyl-sn-glycero-3- | 2805 | 5.61 | 05.00 |
| phosphoethanolamine-N- | | | |
| [methoxy(polyethylene glycol)- | | | |
| 2000] (ammonium salt) (DSPE- | | | |
| PEG₂₀₀₀) DSPE-PEG₂₀₀₀ | | | |

| | |
|---|---|
| Phos. lipid (mM) = 15.0 | Theoretical |
| | loading |
| | @ 0.39 mg |
| | Pt/ml |

Formulation volume: 2 ml (water)

TABLE 2
DLS data of IO-199_01 prep-01

| Zavg (nm) | PDI | ZP (mV) | Pt (mg/ml) |
|---|---|---|---|
| 131.8 | 0.095 | −22.3 | 0.15 |

TABLE 3
IO-199_02 prep-01

| | | Compounds | |
|---|---|---|---|
| Lipids | Mol. wt | used (mg) | mol % |
| HSPC | 783.7 | 14.07 | 22.00 |
| POPC | 760.0 | 31.01 | 50.01 |
| Chol | 387.0 | 0.94 | 2.99 |
| IO-199_02 | 579.05 | 9.45 | 20.00 |
| DSPE-PEG₂₀₀₀ | 2805.0 | 11.44 | 5.00 |

| | |
|---|---|
| Phos. lipid (mM) = 20.94 | Theoretical |
| | loading |
| | @ @ 0.72 mg |
| | Pt/ml |

Formulation volume: 3.0 ml (5% lactose)

TABLE 4
DLS data of IO-199_02 prep-01
DLS and AAS data

| Zavg (nm) | PDI | ZP (mV) | Pt (mg/ml) | % EE |
|---|---|---|---|---|
| 138.6 | 0.4 | 1.19 | 0.42 | 48.8 |

### Example 9: Exemplary Supramolecular Formulations of Compounds IO-199_03, IO-199_04 and IO-199_07

### Characterization of Supramolecular Formulations

The mean particle size and polydispersity index (PDI) of the formulation was measured by Dynamic Light Scattering method using Zetasizer Nano ZS90 (Malvern, UK). 50 μL of formulation was diluted to 1 ml using DI water and measurement was performed at 90 degree scattering angle at 25° C. to get the average particle size distribution. The zeta potential was estimated on the basis of electrophoretic mobility under an electric field using same instrument a Zetasizer Nano ZS90.

Procedure:

Organic solvent was evaporated into a thin and uniform lipid-drug film initially by N₂-flushing and finally under vacuum using a rotary evaporator at 45° C. The dry lipid-drug film was then hydrated with required volume of buffer to get a phospholipids required concentration of drug and allowed to rotate on rotary evaporator at 65° C. (above the lipid phase transition temperature) for 60 min. Hydrated MLVs (multilamellar vesicles) were sequentially extruded through 400 nm, 200 nm and 100 nm pore size membrane (Whatman® filters) supported by filter support (Avanti No: 230600) for 10 times using 10 ml LIPEX™ extruder (Northern Lipids, Canada) under nitrogen pressure connected to circulating water bath at 65° C. (2-4). Finally, the glass vial containing formulation were cooled at 4° C. for 30 min, then −20° C. for 2 h and at −80° C. for 2 h and finally freeze dried overnight at −80° C.

TABLE 5
IO-199_03 prep-01

| | | Compounds | |
|---|---|---|---|
| Lipids | Mol. wt | used (mg) | mol % |
| L-α-phosphatidylcholine, | 783.7 | 39.97 | 75.00 |
| hydrogenated (soy) HSPC | | | |
| Free-cholesterol | 387 | 1.32 | 05.00 |
| IO-199_03 | 978.2 | 9.34 | 15.00 |
| 1,2-distearoyl-src-glycero-3- | 2805 | 9.54 | 05.00 |
| phosphoethanolamine-N- | | | |
| [methoxy(polyethylene glycol)- | | | |
| 2000] (ammonium salt) (DSPE- | | | |
| PEG₂₀₀₀) DSPE-PEG₂₀₀₀ | | | |

| | |
|---|---|
| Phos. lipid (mM) = 13.6 | Theoretical |
| | loading |
| | @ 0.57 mg |
| | Pt/ml |

Formulation volume: 4. ml (5% lactose as cryo protectant)

TABLE 6
DLS data of IO-199_03 prep-01

| Zavg (nm) | PDI | ZP (mV) | Pt (mg/ml) |
|---|---|---|---|
| 105.9 | 0.060 | −28.0 | 0.34 |

TABLE 7
IO-199_04 prep-02

| | | Compounds | |
|---|---|---|---|
| Lipids | Mol. wt | used (mg) | mol % |
| HSPC | 783.7 | 79.94 | 75.00 |
| Chol | 387 | 2.63 | 5.00 |
| IO-199_04 (S04/F01) | 1022.2 | 20.85 | 15.00 |
| PEG-DSPE₂₀₀₀ | 2805 | 19.07 | 5.00 |
| | Phos. lipid (mM) | 27.2 | @ 1.19 mg |
| | | | Pt/ml |

Formulation volume: 4. ml (5% lactose as cryo protectant)

TABLE 8
DLS data of IO-199_04 prep-02

| Zavg (nm) | PDI | ZP (mV) | Pt (mg/ml) |
|---|---|---|---|
| 134.0 | 0.116 | 50.0 | 0.55 |

TABLE 9
IO-199_07 prep-01

| | | Compounds | |
|---|---|---|---|
| Lipids | Mol. wt | used (mg) | mol % |
| HSPC | 783.7 | 43.71 | 72.00 |
| Chol | 387 | 0.90 | 3.00 |
| IO-199_07 (S06/F01) | 566.0 | 8.77 | 20.00 |
| PEG-DSPE₂₀₀₀ | 2805 | 10.86 | 5.00 |
| | Phos. lipid (mM) | 23.8 | @ 0.80 mg |
| | | | Pt/ml |

Formulation volume: 2.5 ml (5% lactose as cryo protectant)

TABLE 10
DLS data IO-199_07 prep-01

| Zavg (nm) | PDI | ZP (mV) | Pt (mg/ml) |
|---|---|---|---|
| 149.2 | 0.125 | −13.6 | 0.30 |

### Example 10: Exemplary Formulations of Compounds IO-199_24 and IO-199_33

### Formulation Preparation

The IO-199_24 and IO-199_33 formulations were engineered using thin film hydration method with slight modification (Sengupta et al., Cholesterol-tethered platinum II-based supramolecular nanoparticle increases antitumor efficacy and reduces nephrotoxicity, PNAS (2012) 109(28): 11294-11299). Particle size and zeta potential was measured using a Dynamic Light Scattering method using Zetasizer Nano ZS90 (Malvern, UK). Pt equivalent in drug concentrations were quantified using atomic absorption spectrometer (AAS) (PinAAcle 900Z, US).

### Detailed Procedure

Organic solvent ((1:1) MeOH:DCM) was evaporated into a thin and uniform lipid-drug film initially by N₂-flushing and finally under vacuum using a rotary evaporator at 45° C. The dry lipid-drug film was then hydrated with required volume of buffer (or water) to get a phospholipids required concentration of drug and allowed to rotate on rotary evaporator at 65° C. (above the lipid phase transition temperature) for 60 min. Hydrated MLVs (multilamellar vesicles) were sequentially extruded through 400 nm, 200 nm and 100 nm pore size membrane (Whatman® filters) supported by filter support (Avanti No: 230600) for 10 times using 10 ml LIPEX™ extruder (Northern Lipids, Canada) under nitrogen pressure connected to circulating water bath at 65° C. (2-4). Finally, the glass vial containing formulation were cooled at 4° C. for 30 min, then −20° C. for 2 h and at −80° C. for 2 h and finally freeze dried overnight at −80° C.

### Supramolecular Formulation Preparation

The supramolecular formulations were engineered using thin film hydration method (Sengupta et al., Cholesterol-tethered platinum II-based supramolecular nanoparticle increases antitumor efficacy and reduces nephrotoxicity, PNAS (2012) 109(28): 11294-11299). The compositions of different formulations are shown in Tables 11 and 13. DLS data is summarized in Table 11.

### Characterization of Supramolecular Formulation

The mean particle size and polydispersity index (PDI) of the formulations were measured by Dynamic Light Scattering method using Zetasizer Nano ZS90 (Malvern, UK). 50 μL of formulation was diluted to 1 ml using DI water and measurement was performed at 90 degree scattering angle at 25° C. to get the average particle size distribution. The zeta potential was estimated on the basis of electrophoretic mobility under an electric field using same instrument a Zetasizer Nano ZS90.

TABLE 11
IO-199_24 prep-02

| | | Compounds | |
|---|---|---|---|
| Lipids | Mol. wt | used (mg) | mol % |
| HSPC | 783.7 | 13.95 | 20.00 |
| POPC | 760.07 | 33.82 | 50.00 |
| Chol | 387 | 1.03 | 3.00 |
| IO-199_24 (S02/F02) | 678 | 13.28 | 22.00 |
| PEG-DSPE₂₀₀₀ | 2805 | 12.48 | 5.00 |

| | |
|---|---|
| Phos. lipid (mM) = 22.25 | Th. Loading |
| | @ 1.27 mg |
| | Pt/ml |

Formulation volume: 3.0 ml (5% sucrose)

TABLE 12
DLS data

| Zavg (nm) | PDI | ZP (mV) | Pt (mg/ml) |
|---|---|---|---|
| 162.9 | 0.174 | 3.45 | 0.92 |

TABLE 13
IO-199_33 prep-01

| | | Compounds | |
|---|---|---|---|
| Lipids | Mol. wt | used (mg) | mol % |
| L-α-phosphatidylcholine, | 783.7 | 27.08 | 70.00 |
| hydrogenated (soy) HSPC | | | |
| Free-cholesterol | 387 | 1.91 | 10.00 |
| IO-199_33 | 917.91 | 9.06 | 20.00 |

| | |
|---|---|
| Phos. lipid (mM) = 17.28 | Theoretical |
| | loading |
| | @ 1.0 mg |
| | Pt/ml |

Formulation volume: 2 ml (5% Sucrose)

### Example 11: Cell Internalization of Compound IO-199_34

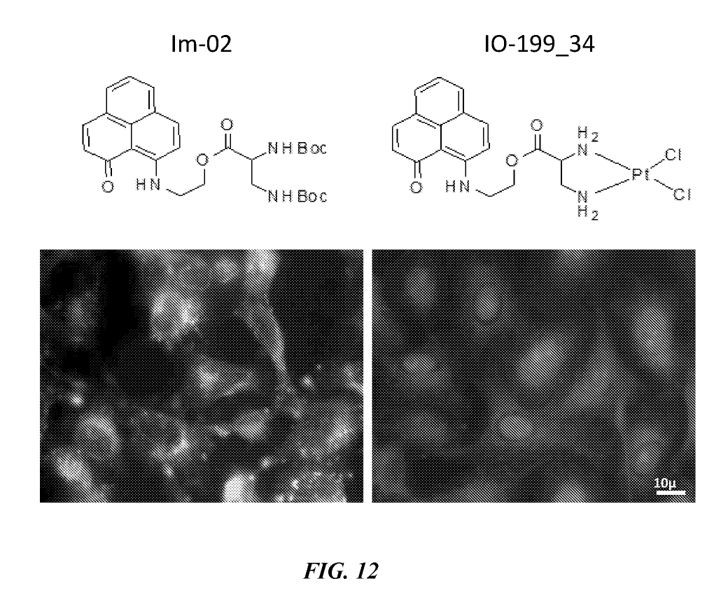

The internalization of IO-199_34 and its ligand (Im-02) was observed in a lung cancer cell line (A549) using the procedure described in Example 3. Post 5-hours incubation, IO-199_34 was detected as uniform staining in the cytoplasm and nucleus of cells, while the intermediate Im-02 could be detected as punctate dots in the cytoplasm or along cell margins (FIG. 12). This shows that post cellular internalization, IO-199_34 retains its fluorescence and stains nucleus, unlike its intermediate, which does not localize to nucleus.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow. Further, to the extent not already indicated, it will be understood by those of ordinary skill in the art that any one of the various embodiments herein described and illustrated can be further modified to incorporate features shown in any of the other embodiments disclosed herein.

All patents and other publications identified in the specification and examples are expressly incorporated herein by reference for all purposes. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

1-53. (canceled)

54. A platinum containing complex comprising:
(a) a fluorescent molecule; and
(b) platinum atom conjugated with the fluorescent molecule.

55. The platinum containing complex of claim 54 which is:
(i) a complex of Formula VI: 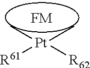
wherein:
FM is fluorescent molecule optionally conjugated with a -linker-lipid;
R⁶¹ and R⁶² are same or different and selected independently from halogen, alkyl, amino, alkylamino, dialkylamino, hydroxyl, alkoxy, thiol, thioalkyl, —S(O)(R⁶³)₂, O-acyl, or any combinations thereof, or R⁶¹ and R⁶², together with the Pt atom form an optionally substituted cyclyl or heterocyclyl; and
each R⁶³ is independently a C₁-C₆alkyl;
(ii) a complex of Formula VII: 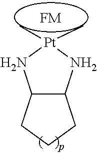
wherein:
FM is fluorescent molecule optionally conjugated with a -linker-lipid; and
p is 0, 1,2, 3 or 4;
(iii) a complex of Formula VIII: 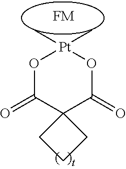
wherein:
FM is fluorescent molecule optionally conjugated with a -linker-lipid; and
t is 0, 1, 2, 3 or 4;
(iv) a complex of Formula IX: 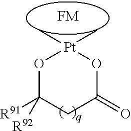
wherein:
FM is fluorescent molecule optionally conjugated with a -linker-lipid;
R⁹¹ and R⁹² are hydrogen or together form a carbonyl; and
q is 0, 1, 2, 3 or 4;
(v) a complex of Formula X: 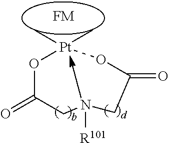
wherein:
FM is fluorescent molecule optionally conjugated with a -linker-lipid;
R¹⁰¹ is H or a -linker-lipid;
b is 1, 2, 3 or 4; and
d is 1, 2, 3, or 4; or
(vi) a complex of Formula XI: 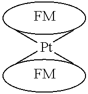
wherein:
each FM is an independently selected fluorescent molecule optionally conjugated with a -linker-lipid.

56. The complex of claim 54, wherein the fluorescent molecule is selected from the group consisting of:
(i) a compound of Formula V: 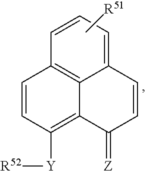
wherein Y is O, S or NR⁵³; Z is O or NR⁵³; R⁵¹ is absent, alkoxy, optionally substituted amino, thiol, optionally substituted alkylthio, -linker-(anti-cancer agent), -linker-carbohydrate or -linker-lipid; R⁵² is H, alkyl, cyclyl, heterocyclyl, aryl, heteroaryl, or -linker-lipid; and each R⁵³ is same or different and selected independently from the group consisting of alkyl, cyclyl, heterocyclyl, aryl, heteroaryl, optionally substituted PEG, -linker-carbohydrate, -linker-(anti-cancer agent), -linker-NH(CH₂CO₂H)₂, -linker-CO₂H, 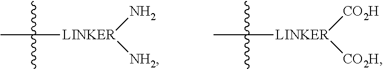
or -linker-lipid, each of which can be optionally substituted, optionally provided that at least one of R⁵¹ and R⁵² is optionally substituted PEG, -linker-carbohydrate, -linker-(anti-cancer agent), -linker-NH(CH₂CO₂H)₂, -linker-CO₂H, 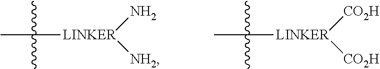
or -linker-lipid;
(ii) a compound of Formula I: 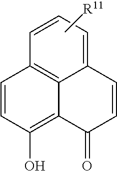
wherein R¹¹ is hydrogen, alkoxy, optionally substituted alkylamino, optionally substituted alkylthio, -linker-(anti-cancer agent), -linker-carbohydrate, or -linker -lipid;
(iii) a compound of Formula II: 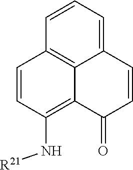
wherein R²¹ is Hydrogen, alkyl, cyclyl, heterocyclyl, aryl, heteroaryl, optionally substituted PEG, -linker-carbohydrate, -linker-(anti-cancer agent), -linker-NH(CH₂CO₂)₂, -linker-CO₂, 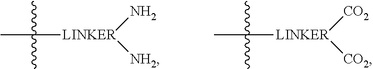
or -linker-lipid, each of which can be optionally substituted;
(iv) a compound of Formula III: 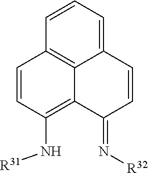
wherein R³¹ and R³² are same or different and selected independently from the group consisting of hydrogen, alkyl, cyclyl, heterocyclyl, aryl, heteroaryl, or -linker-lipid, each of which can be optionally substituted;
(v) a compound a Formula IV: 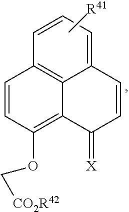
wherein X is O or NR⁴³; R⁴¹ is absent, hydroxyl, alkoxy, -linker-lipid or polyethylene glycol; R⁴² is H, alkyl, cyclyl, heterocyclyl, aryl or heteroaryl; and R⁴³ is H, alkyl, cyclyl, heterocyclyl, aryl or heteroaryl, each of which can be optionally substituted;
(i) a compound of Formula V′: 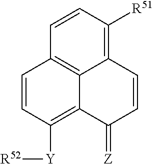
wherein Y, Z, R⁵¹ and R⁵² are as defined in compound V above;
(ii) a compound of Formula I′: 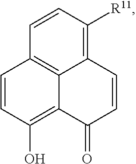
wherein R¹¹ is hydrogen, alkoxy, alkylamino, alkylthio, -linker-carbohydrate, or -linker-lipid;
(iii) a compound of Formula II′: 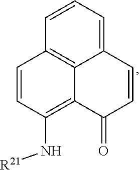
wherein R²¹ is H, optionally substituted alkyl, optionally substituted PEG, -linker-carbohydrate, -linker-(anti-cancer agent), -linker-NH(CH₂CO₂H)₂, -linker-CO₂H, 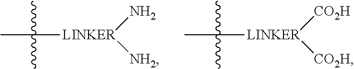
or -linker-lipid;
(iv) a compound of Formula III′: 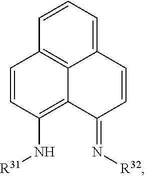
wherein R³¹ and R³² are same or different and independently H, optionally substituted alkyl, or -linker-lipid;
(v) a compound of Formula IV′: 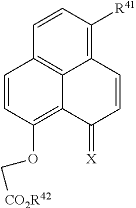
wherein X, R⁴¹ and R⁴² are as defined in compound of formula IV above;
(vi) a compound of Formula I″: 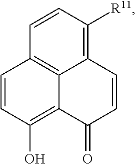
wherein R¹¹ is hydrogen, alkoxy, alkylamino, alkylthio, -linker-(anti-cancer agent), -linker-carbohydrate, or -linker-lipid;
(vii) a compound of Formula II″: 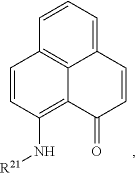
wherein R²¹ is a optionally substituted PEG, -linker-carbohydrate, -linker-(anti-cancer agent), -linker-NH(CH₂CO₂)₂, -linker-CO₂, 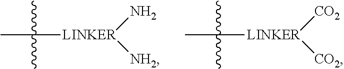
or linker-lipid;
(viii) a compound of Formula III″: 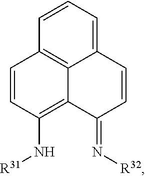
wherein at least one of R³¹ and R³² is a -linker-lipid;
(ix) a compound of Formula V″: 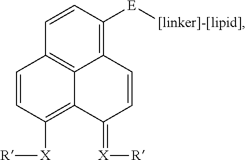
wherein:
each X is same or different and selected independently from the group consisting of O, N, S, NH and NR
each R′ is same or different and selected independently from the group consisting of H, alkyl, cyclyl, heterocyclyl, aryl and heteroaryl, each of which can be optionally substituted; and
R is alkyl, cyclyl, heterocyclyl, aryl or heteroaryl, each of which can be optionally substituted;
(x) a compound of Formula V″-B: 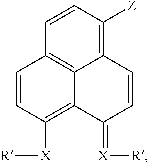
wherein:
Z is alkoxy, alkylamino, alkylthio or -E-linker-carbohydrate;
E is O, NH or S;
each X is same or different and selected independently from the group consisting of O, N, S, NH and NR
each R′ is same or different and selected independently from the group consisting of H, alkyl, cyclyl, heterocyclyl, aryl and heteroaryl, each of which can be optionally substituted; and
R is alkyl, cyclyl, heterocyclyl, aryl or heteroaryl, each of which can be optionally substituted.
(xi) a compound of Formula V′″: 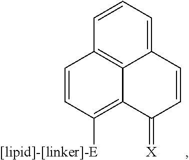
wherein:
E is NH or S;
X is O or NR; and
R is H, alkyl, cyclyl, heterocyclyl, aryl or heteroaryl, each of which can be optionally substituted; or
(xii) a compound Formula V′″-B: 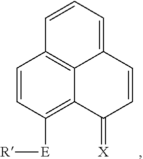
wherein:
E is NH or S;
R′ is optionally substituted PEG, -linker-carbohydrate, -linker-(anti-cancer agent), -linker-NH(CH₂CO₂)₂, -linker-CO₂, 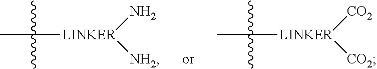
X is O or NR;
R is H, alkyl, cyclyl, heterocyclyl, aryl or heteroaryl, each of which can be optionally substituted; and
wherein the fluorescent molecule is conjugated with a lipid.

57. The complex of claim 54, wherein the platinum atom is conjugated to the fluorescent molecule via a covalent bond, coordinate bond or a combination thereof.

58. The complex of claim 54, wherein the complex is selected from the group consisting of: 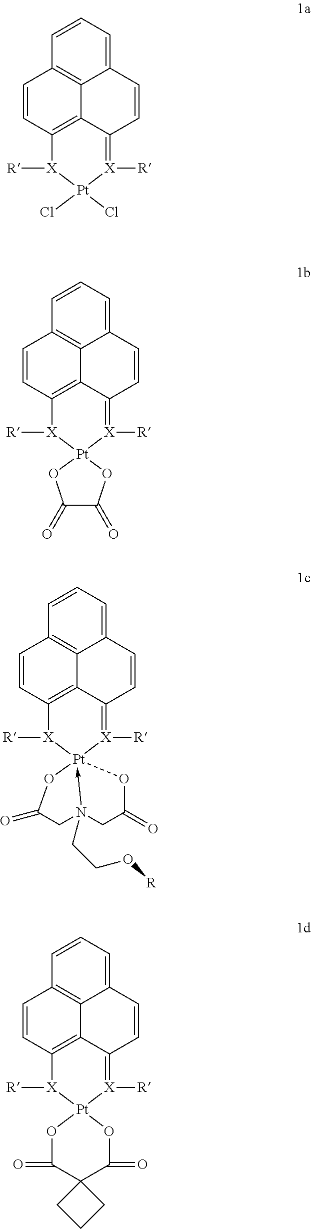 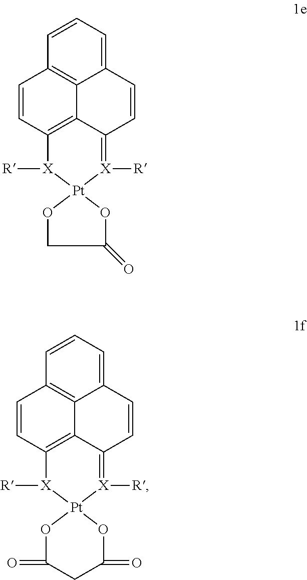
where X is O or N; R′ is H or optionally substituted alkyl; and R is cholesterol, lumisterol, alpha-tocopherol or vitamin A; 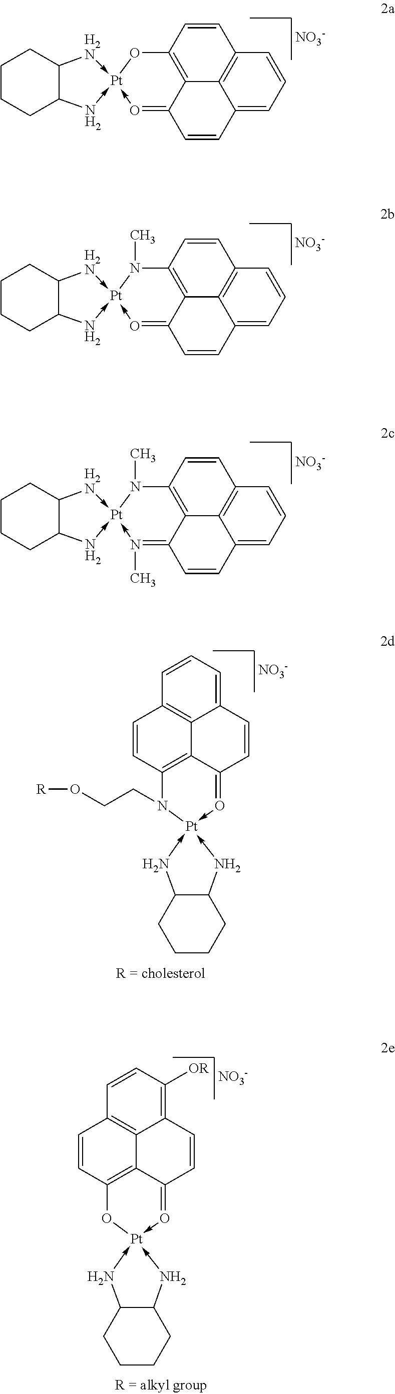 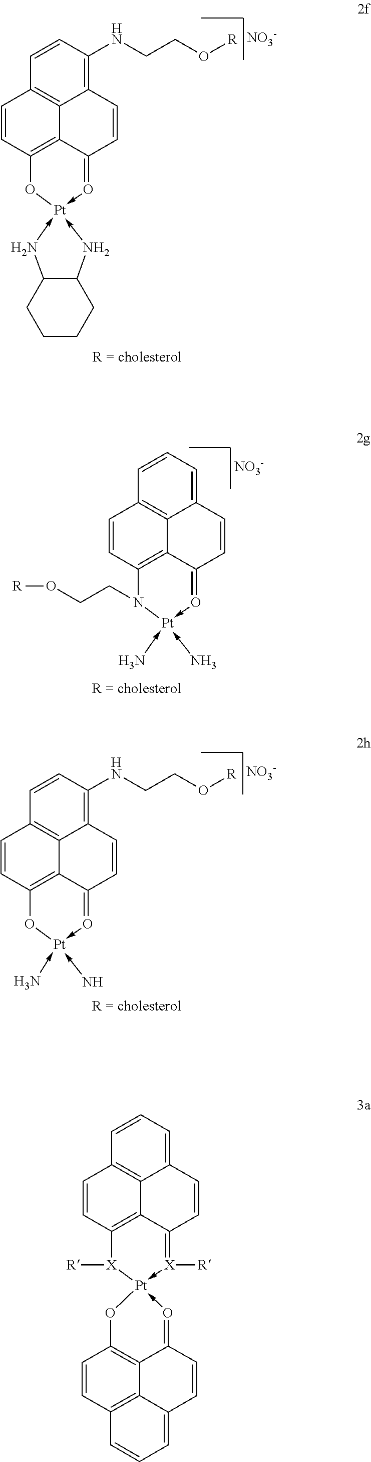 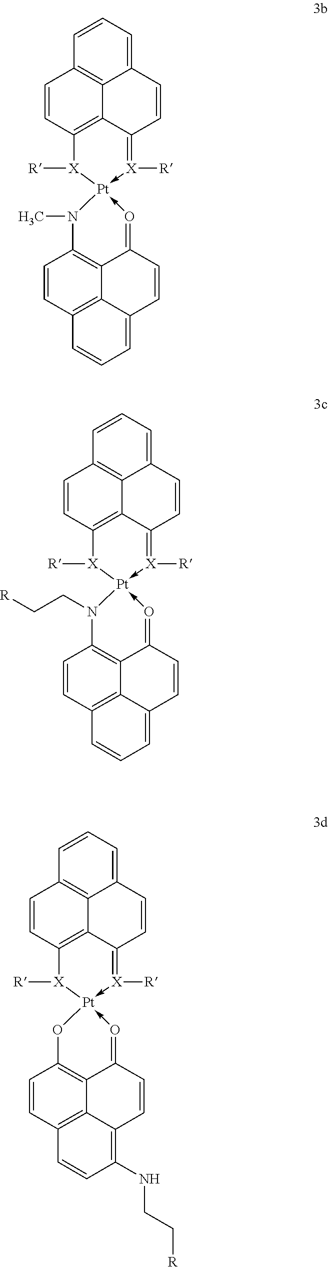 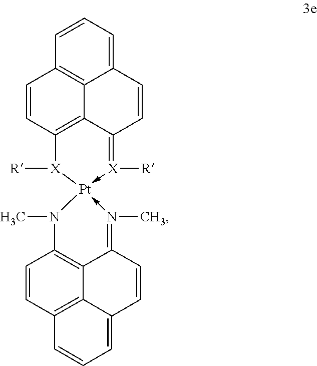
wherein R is cholesterol, X is O or N, and R′ is H, methyl or optionally substituted alkyl; 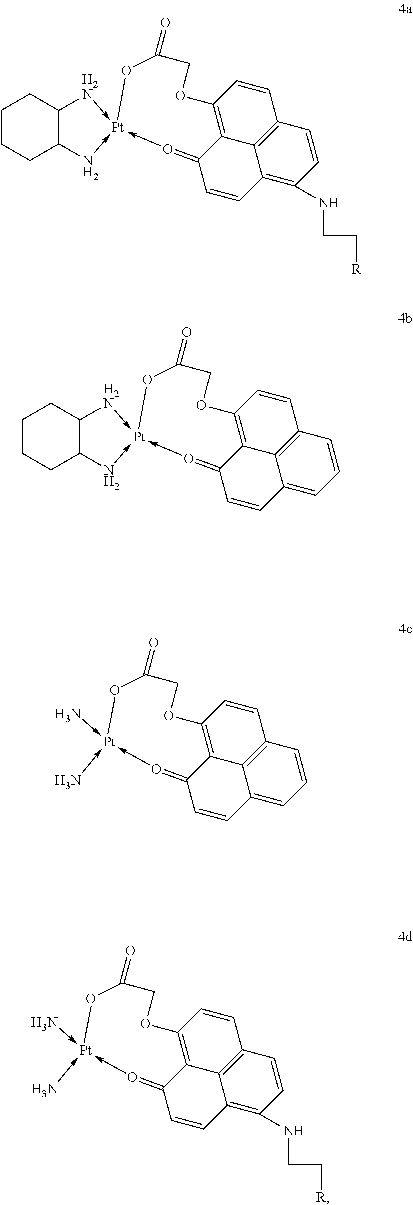
wherein R is a lipid.

59. A platinum-containing complex selected from the group consisting of: 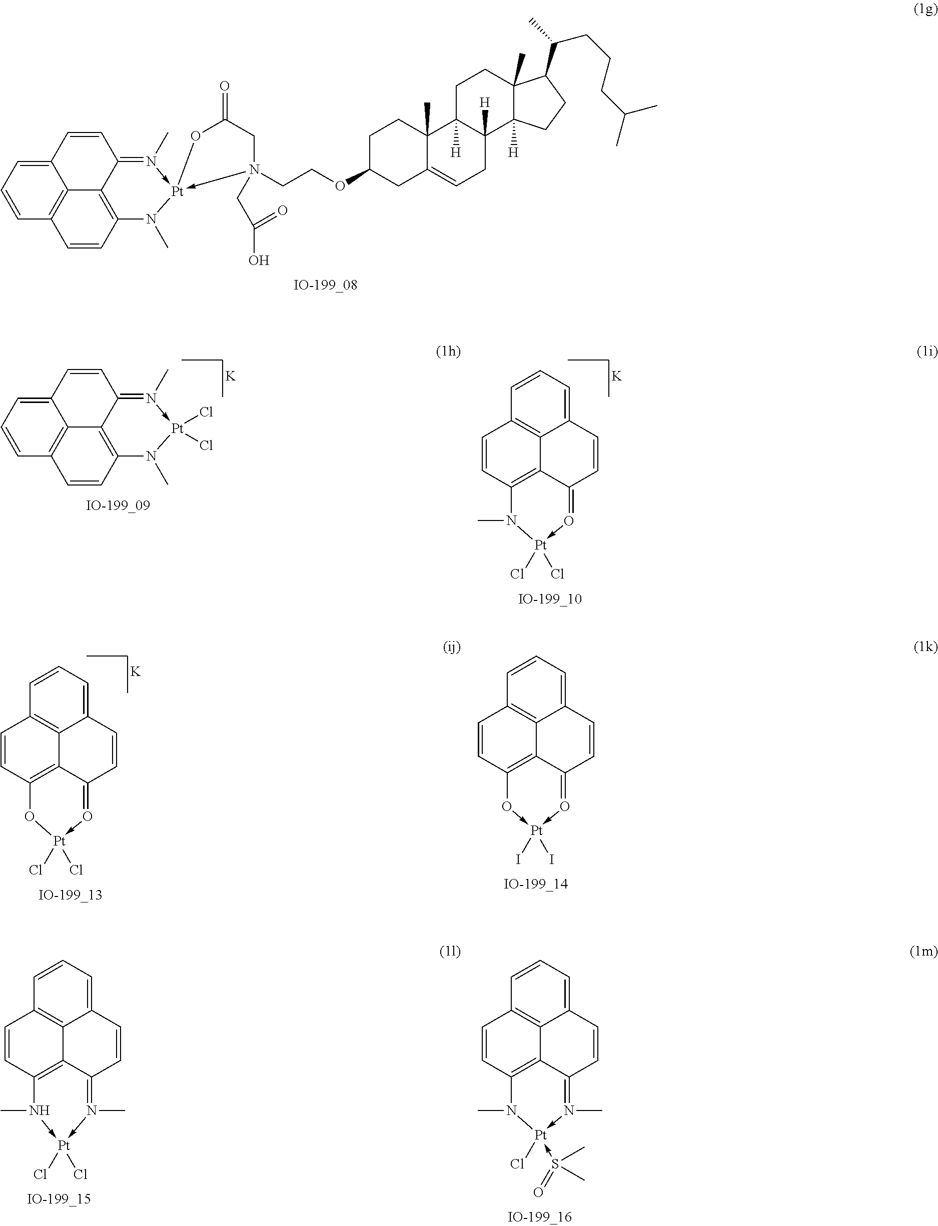 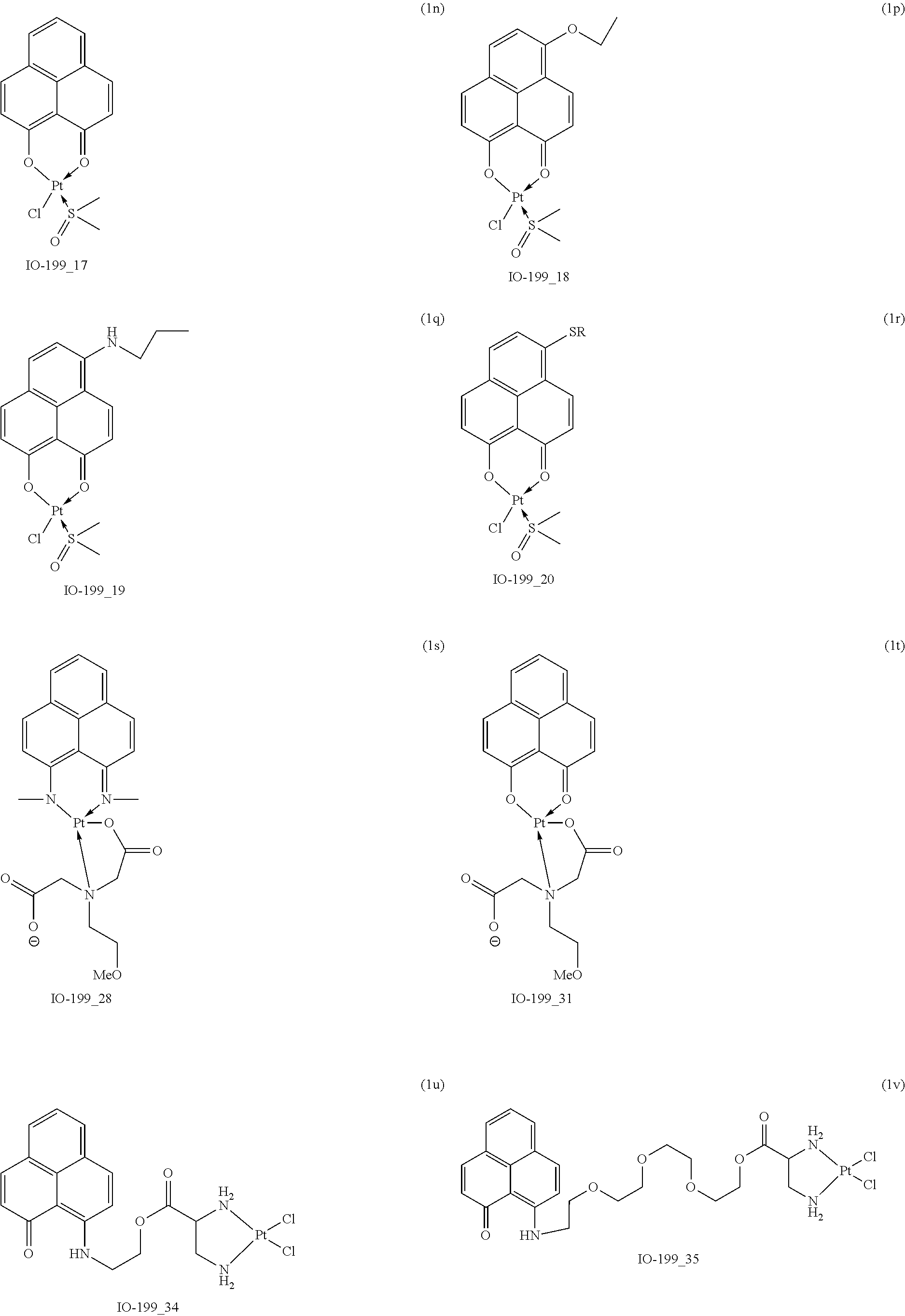 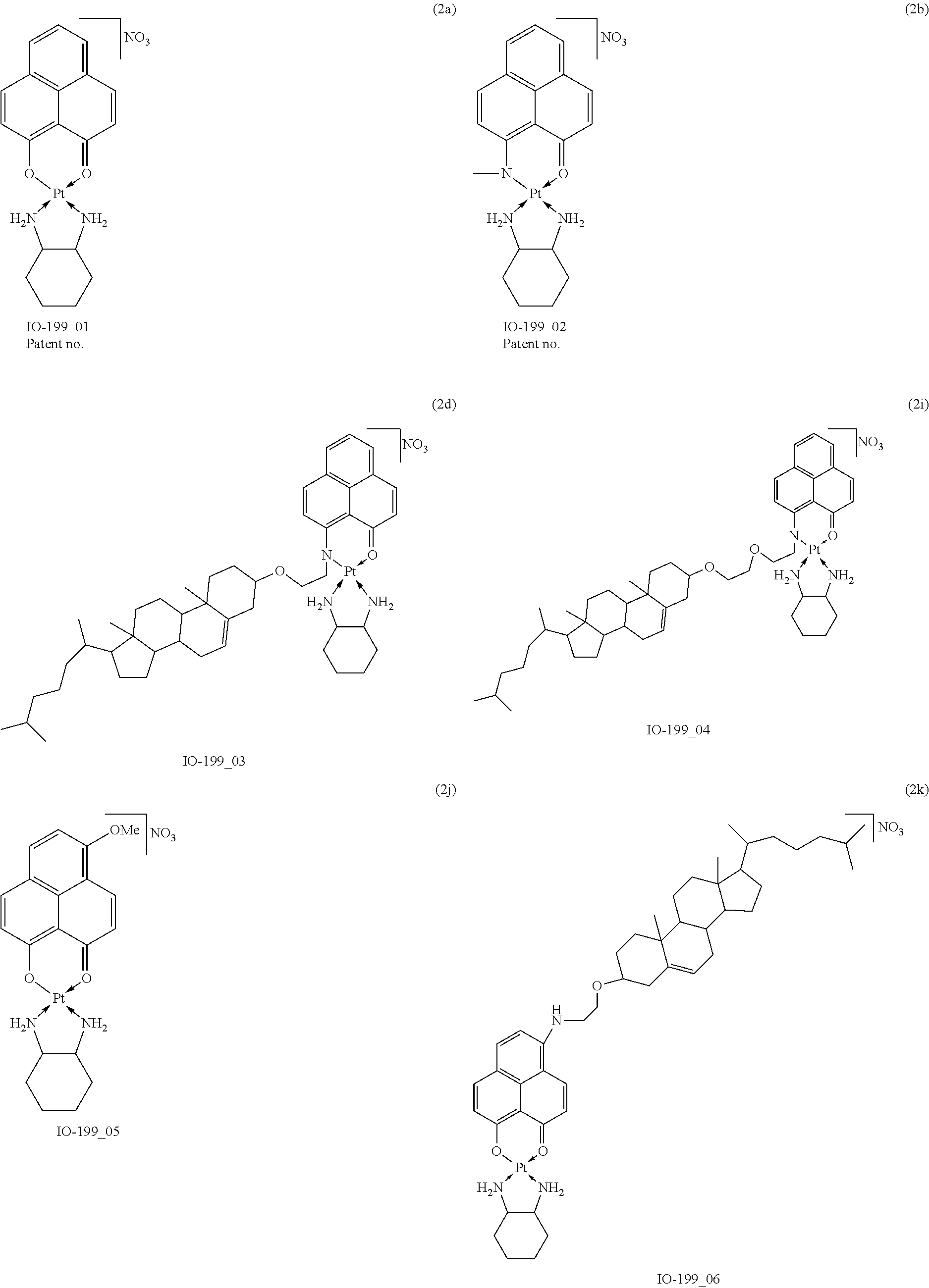 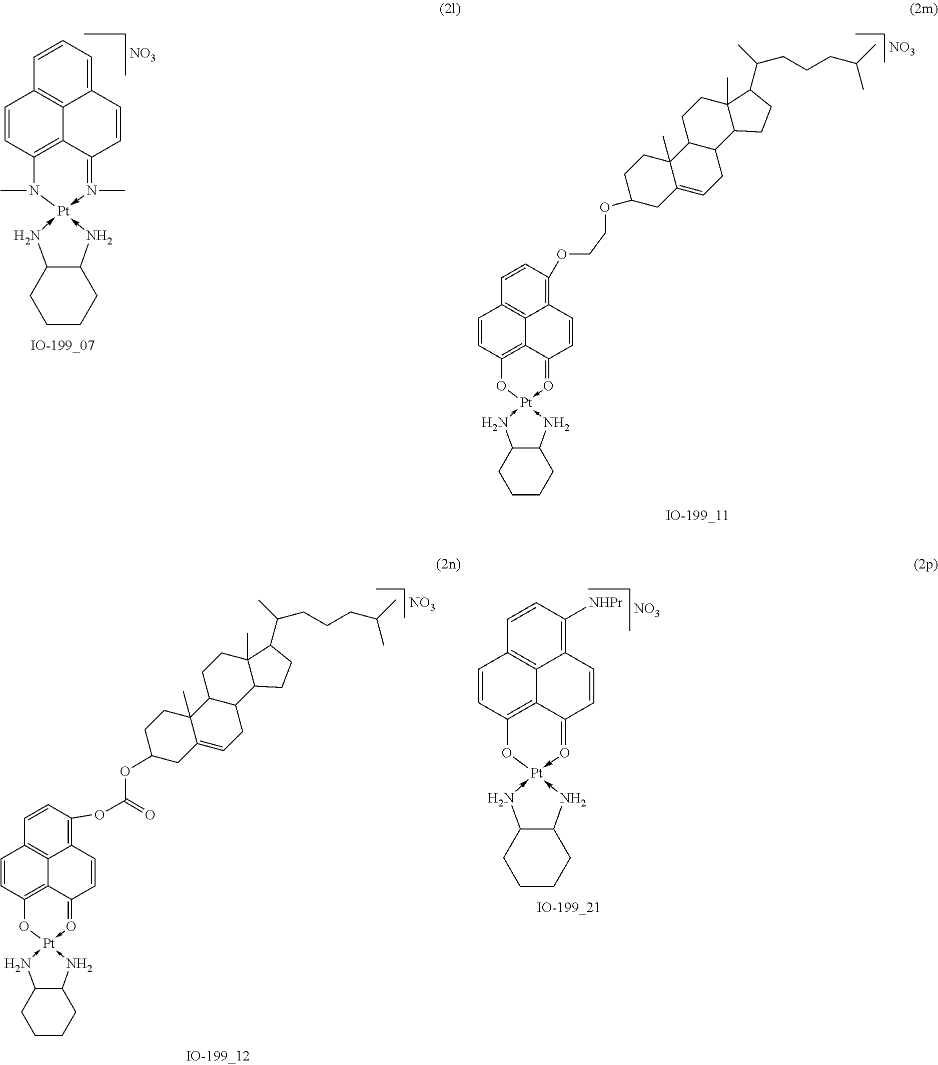 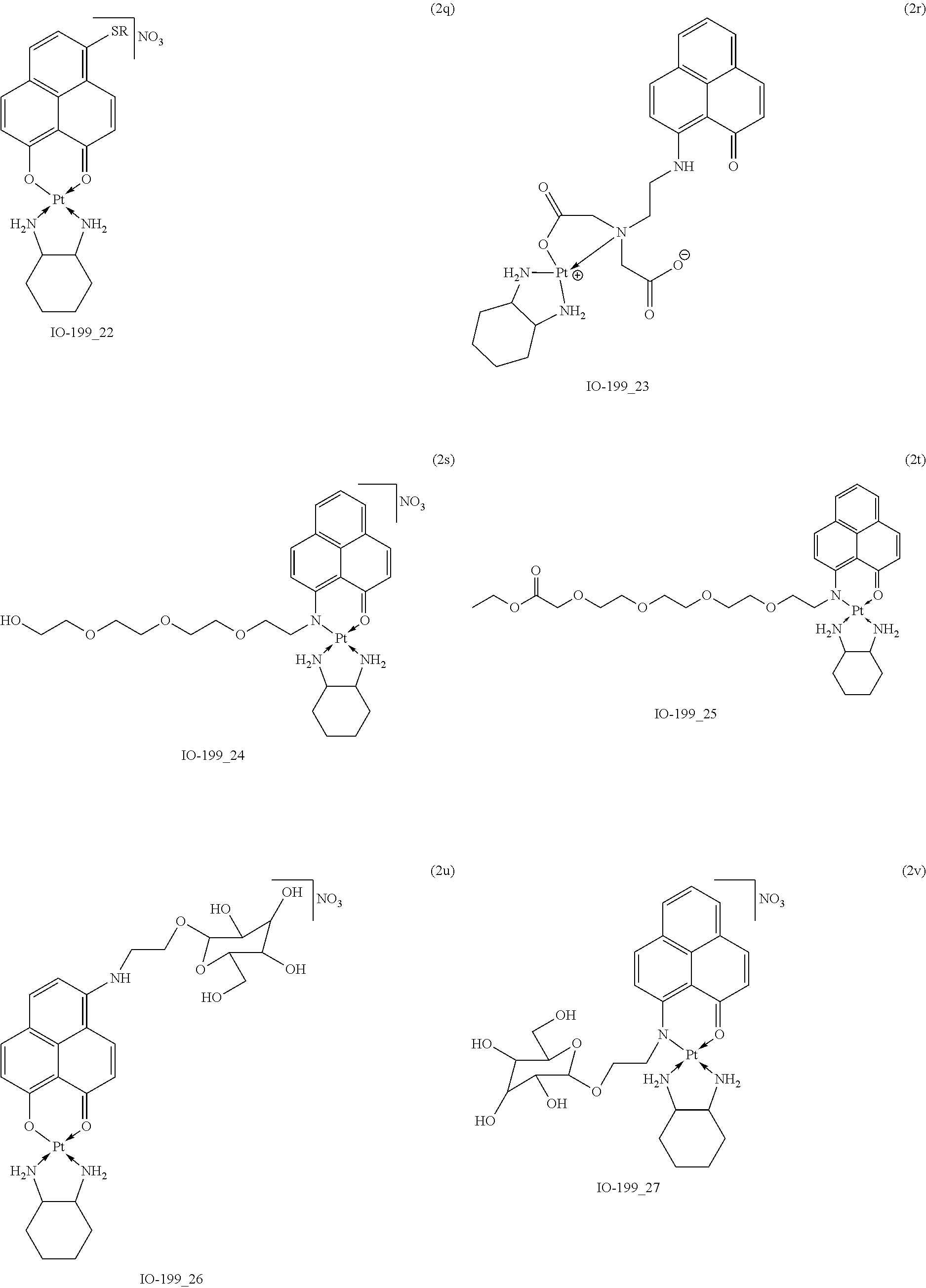 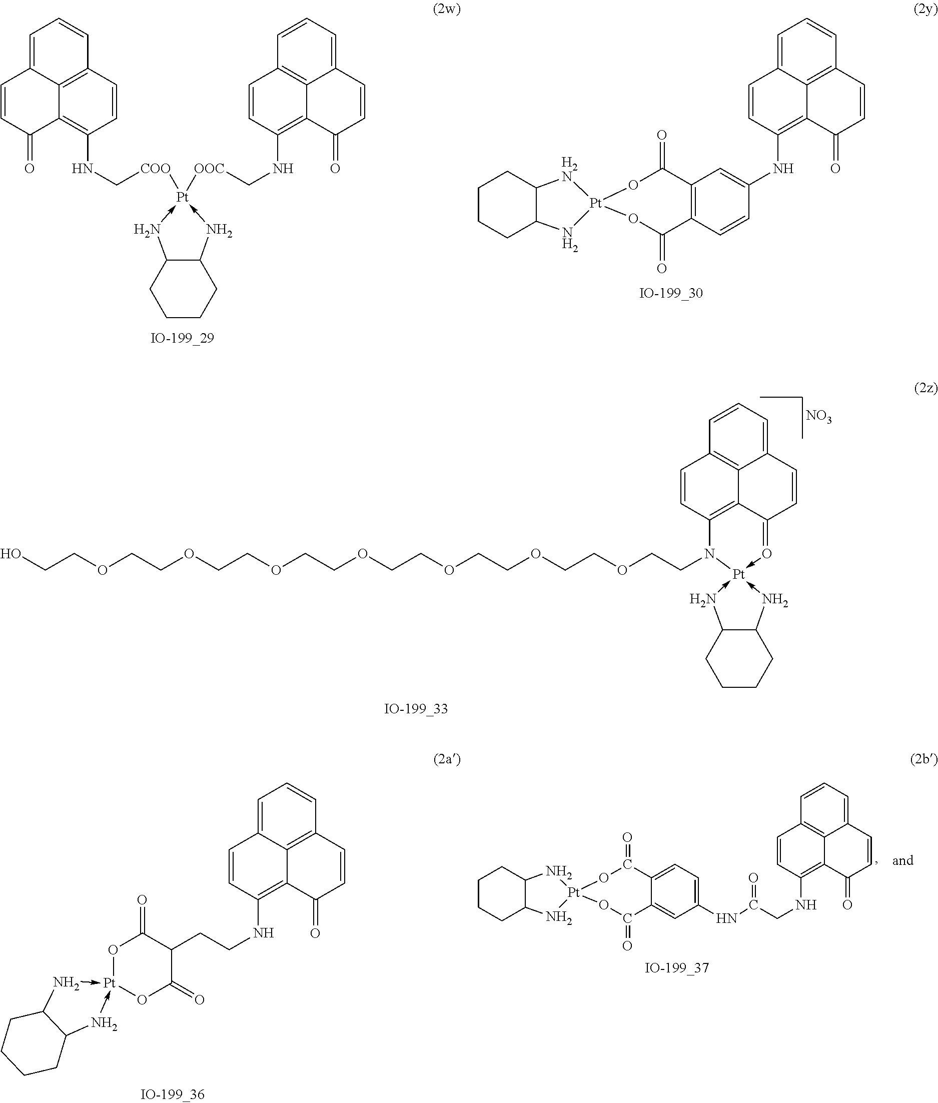 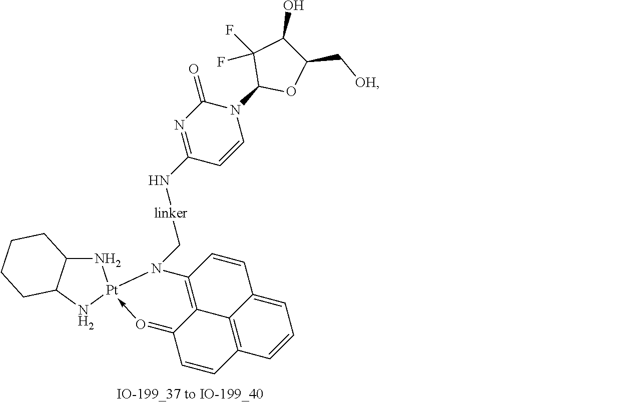
wherein R is optionally substituted alkyl, and counter anion if present can be nitrate or chloride; the linker is —CH₂CH₂(OCH₂CH₂)ₙOC(O)O—; and n is 0 or an integer from 1 to 10.

60. A platinum-containing complex of Formula XII: 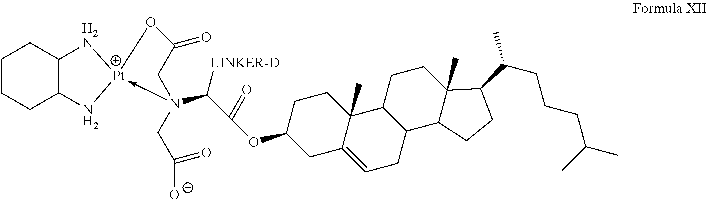
wherein D is a fluorescent molecule.

61. The complex of claim 60, wherein the complex comprises one or more of:

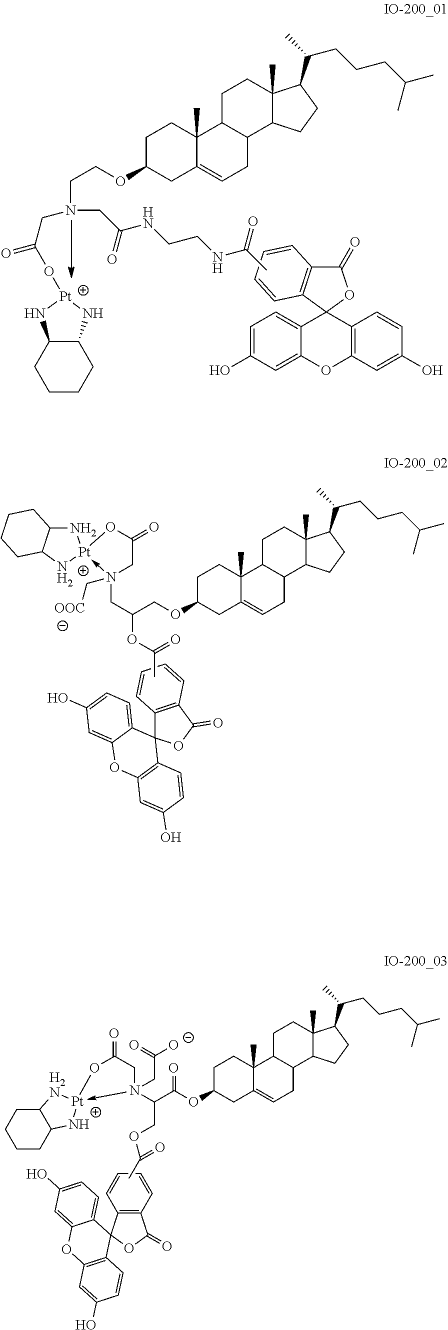

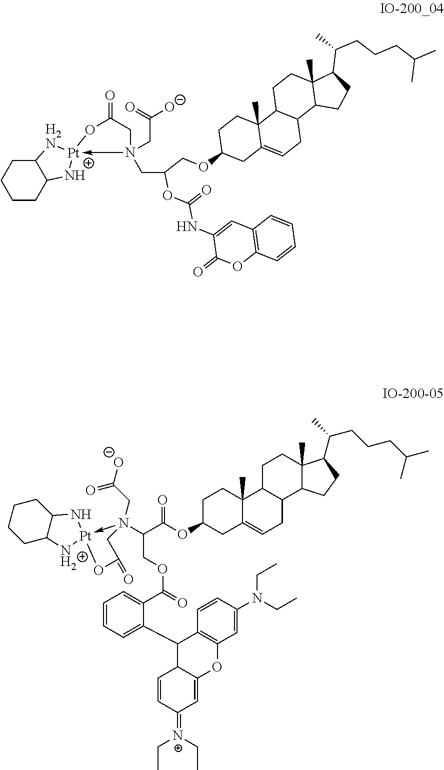

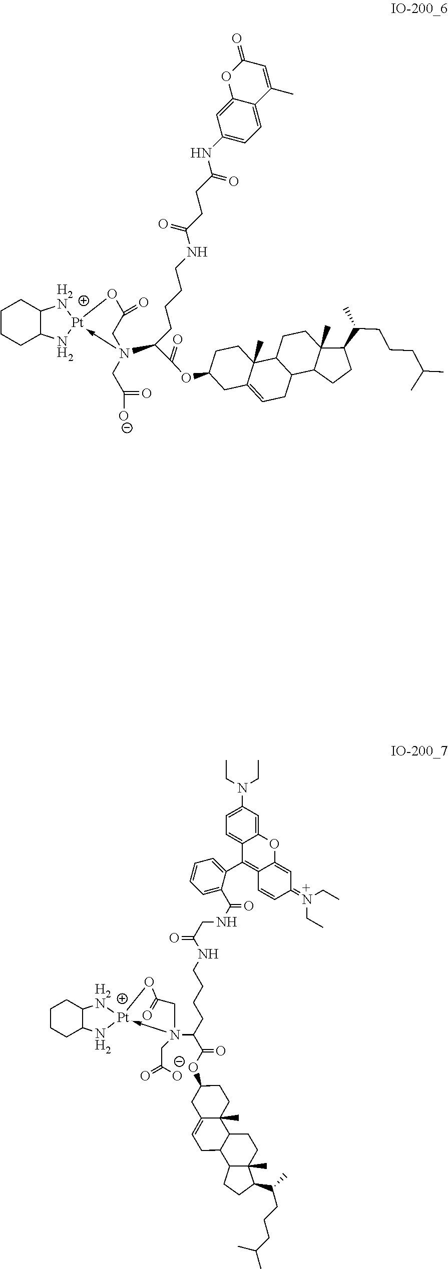

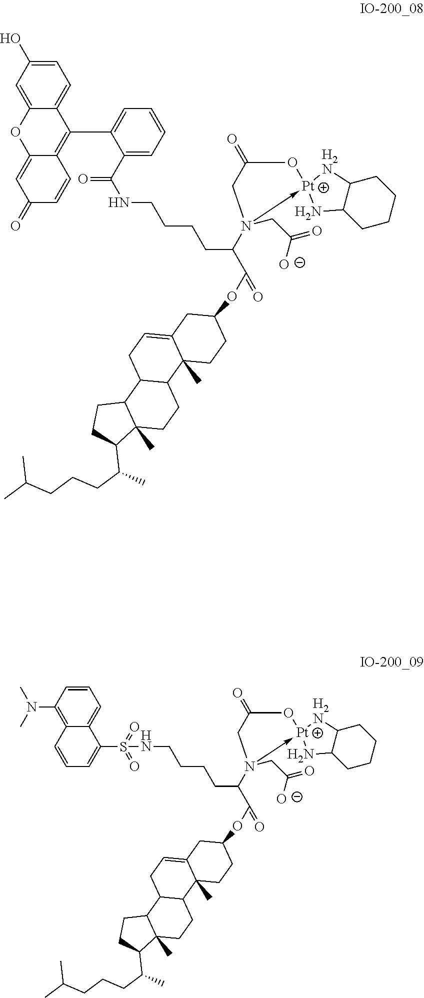

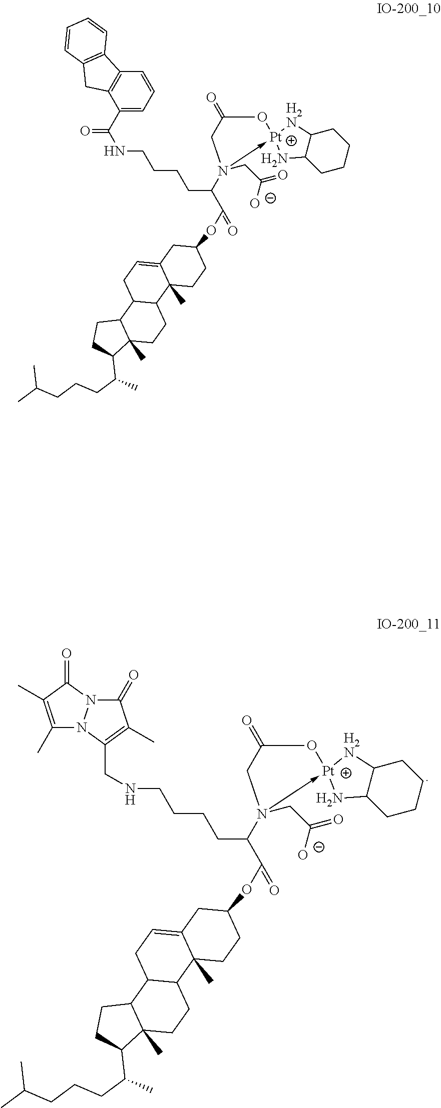

62. The complex of claim 54, wherein the compound exhibits increased cellular uptake of platinum relative to cisplatin or oxaliplatin in cancer cells; or the complex exhibits a higher accumulation of platinum in a tumor relative to cisplatin or oxaliplatin at an equivalent dosage amount of amount of cisplatin or oxaliplatin.

63. (i) A compound of Formula IV: 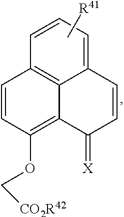
wherein:
X is O or NR⁴³;
R⁴¹ is H, hydroxyl, alkoxy, -linker-lipid or polyethylene glycol;
R⁴² is H, alkyl, cyclyl, heterocyclyl, aryl or heteroaryl;
R⁴³ is H, alkyl, cyclyl, heterocyclyl, aryl or heteroaryl, and
wherein in R⁴¹, R⁴² and R⁴³ can be optionally substituted;
(ii) a compound of Formula IV′: 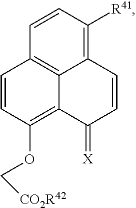
(iii) a compound of Formula V: 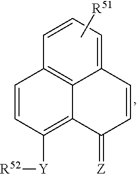
wherein:
Y is O, S or NR⁵³;
Z is O or NR⁵³;
R⁵¹ is H, alkoxy, optionally substituted alkylamino, optionally substituted alkylthio, -linker-carbohydrate, -linker-(anti-cancer agent) or -linker-lipid;
R⁵² is H, alkyl, cyclyl, heterocyclyl, aryl, heteroaryl, optionally substituted PEG, -linker-carbohydrate, -linker-(anti-cancer agent), -linker-NH(CH₂CO₂H)₂, -linker-CO₂H, 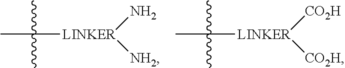
or -linker-lipid; and
each R⁵³ is same or different and selected independently from the group consisting of alkyl, cyclyl, heterocyclyl, aryl, heteroaryl, or -linker-lipid,
provided that at least one of R⁵¹ and R⁵² is -linker-lipid, -linker-(anti-cancer agent), or -linker-carbohydrate, or R⁵² is optionally substituted PEG, -linker-NH(CH₂CO₂H)₂, -linker-CO₂H, 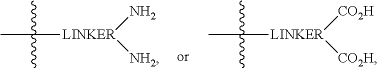
and
wherein in R⁵¹, R⁵² and R⁵³ can be optionally substituted;
(iv) a compound of Formula V′: 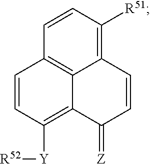
(v) a compound of Formula V″: 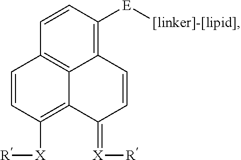
wherein:
each X is same or different and selected independently from the group consisting of O, N, S, NH and NR;
each R′ is same or different and selected independently from the group consisting of H, alkyl, cyclyl, heterocyclyl, aryl and heteroaryl;
R is alkyl, cyclyl, heterocyclyl, aryl or heteroaryl, and
wherein in R and R can be optionally substituted;
(vi) a compound of Formula V″-B: 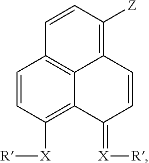
wherein:
Z is -E-linker-(anti-cancer agent) or -E-linker-carbohydrate;
E is O, NH or S;
each X is same or different and selected independently from the group consisting of O, N, S, NH and NR;
each R′ is same or different and selected independently from the group consisting of H, alkyl, cyclyl, heterocyclyl, aryl and heteroaryl, each of which can be optionally substituted; and
R is alkyl, cyclyl, heterocyclyl, aryl or heteroaryl, each of which can be optionally substituted;
(vii) a compound is of Formula V′″: 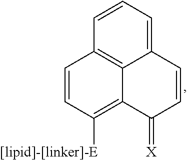
wherein:
E is NH or S;
X is O or NR; and
R is H, alkyl, cyclyl, heterocyclyl, aryl or heteroaryl, each of which can be optionally substituted; or
(viii) a compound of Formula V′″-B: 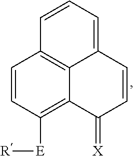
wherein:
E is NH or S;
R′ is optionally substituted PEG, -linker-carbohydrate, -linker-(anti-cancer agent), linker-NH(CH₂CO₂H)₂, -linker-CO₂H, 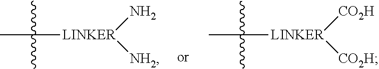
X is O or NR; and
R is H, alkyl, cyclyl, heterocyclyl, aryl or heteroaryl, each of which can be optionally substituted.

64. (i) A compound of Formula I″: 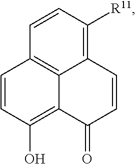
wherein R¹¹ is a -linker-carbohydrate or -linker-lipid;
(ii) a compound of Formula II″: 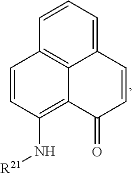
wherein R²¹ is a optionally substituted PEG, -linker-carbohydrate, -linker-(anti-cancer agent), -linker-NH(CH₂CO₂H)₂, -linker-CO₂H 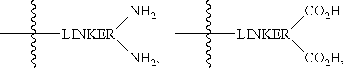
or linker-lipid; or
(iii) a compound of Formula III″: 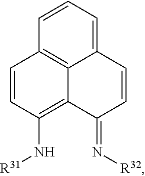
wherein R³¹ and R³² are same or different and selected independently from the group consisting of hydrogen, alkyl, cyclyl, heterocyclyl, aryl, heteroaryl, or -linker-lipid, each of which can be optionally substituted, provided that at least one of R³¹ and R³² is a -linker-lipid.

65. The compound of claim 63, wherein the wherein the lipid is selected from fats, waxes, sterols, steroids, bile acids, fat-soluble vitamins, monoglycerides, diglycerides, phospholipids, glycolipids, sulpholipids, aminolipids, chromolipids, glycerophospholipids, sphingolipids, prenol lipids, saccharolipids, polyketides and fatty acids or any combination thereof, preferably the lipid is cholesterol; lumisterol, alpha-tocopherol or vitamin A; wherein the sterol is selected from cholesterol, cholesterol chloroformate or derivatives thereof, and any combination thereof; wherein the linker is selected from the group consisting of a bond, —CH₂CH₂—, —(CH₂)ₙ—, —(CH₂)ₙO—, —O(CH₂)ₙO—, —(CH₂)ₙNH—, —O(CH₂)ₙNH—, —NH(CH₂)ₙNH—, —OCH₂(CH₂)ₙC(O)—; —C(O)(CH₂)ₙC(O)—; —(CH₂)ₙNHC(O)O—, —(CH₂)ₙOC(O)NH—, —(CH₂)ₙC(O)NH(CH₂)ₘO—, (CH₂)ₙO(CH₂)ₘO—, —(CH₂)ₙO(O)—, —(CH₂)ₙNHC(O)(CH₂)ₘO—, —(CH₂)ₙC(O)O—; or —OC(O)(CH₂)ₙC(O)O—; wherein n and m are independently 0, 1, 2, 3, 4, or 5.

66. A nanoparticle comprising the complex claim 54.

67. The nanoparticle of claim 66, wherein the nanoparticle further comprises a co-lipid or a stabilizer or combination thereof; wherein the co-lipid is Soy-phosphatidyl choline and 1,2-Distearoyl-sn-Glycero-3-Phosphoethalonamine-N-[Methoxy(Polyethylene glycol)-2000].

68. The nanoparticle of claim 66, wherein the nanoparticle exhibits increased cellular uptake of platinum relative to cisplatin or oxaliplatin in cancer cells, or wherein the nanoparticle exhibits a higher accumulation of platinum in a tumor relative to cisplatin or oxaliplatin at an equivalent dosage amount of amount of cisplatin or oxaliplatin.

69. A pharmaceutical composition comprising the nanoparticle of claim 66 and a pharmaceutically acceptable excipient or carrier.

70. The pharmaceutical composition of claim 69, wherein the excipient is selected from the group consisting of granulating agents, binding agents, lubricating agents, disintegrating agents, sweetening agents, glidants, anti-adherents, anti-static agents, surfactants, anti-oxidants, gums, coating agents, coloring agents, flavouring agents, coating agents, plasticizers, preservatives, suspending agents, emulsifying agents, plant cellulosic material, spheronization agents, and any combination thereof; wherein the composition is formulated into a dosage form selected from the group consisting of injectable, tablet, lyophilized powder, liposomal suspension, and any combinations thereof.

71. A method for treating or managing cancer in a subject, the method comprising administering a therapeutically effective amount of the complex of claim 54 to a subject in need thereof.

72. The method of claim 71, wherein the cancer is selected from the group consisting of breast, head and neck, ovarian, testicular, pancreatic, oral-esophageal, gastrointestinal, liver, gall bladder, lung, melanoma, skin, sarcoma, blood, brain, glioblastoma, tumor of neuroectodermal origin and any combinations thereof, wherein said administration is via intravenous administration, intra articular administration, pancreatic duodenal artery administration, intraperitoneal administration, hepatoportal administration, intramuscular administration, or a combination of any two or more thereof.

73. A method for imaging a tumor, the method comprising administering the complex of claim 54 to subject in need thereof.
